(19) United States
(12) Patent Application Publication
(10) Pub. No.: US 2021/0336151 A1
(43) Pub. Date: Oct. 28, 2021

(54) Light-Emitting Device Material, Electron-Transport Layer Material, Organic Compound, Light-Emitting Device, Light-Emitting Apparatus, Electronic Device, and Lighting Device

(71) Applicant: Semiconductor Energy Laboratory Co., Ltd., Kanagawa-ken (JP)

(72) Inventors: Keito Tosu, lsehara (JP); Yuta Kawano, Yokohama (JP); Hiroshi Kadoma, Sagamihara (JP); Harue Osaka, Atsugi (JP); Ryo Narukawa, Hadano (JP); Airi Ueda, Sagamihara (JP); Takeyoshi Watabe, Atsugi (JP); Nobuharu Ohsawa, Zama (JP); Satoshi Seo, Sagamihara (JP)

(73) Assignee: Semiconductor Energy Laboratory Co., Ltd., Kanagawa-ken (JP)

(21) Appl. No.: 17/239,916

(22) Filed: Apr. 26, 2021

(30) Foreign Application Priority Data
Apr. 28, 2020 (JP) 2020-078909

(51) Int. Cl. H01L 51/00; C09K 11/06; C07D 251/24; C07D 239/26; C07D 401/10; C07D 401/14

(52) U.S. Cl. CPC H01L 51/0067; C09K 11/06; C07D 251/24; C07D 239/26; H01L 51/5016; C07D 401/14; C09K 2211/1018; C09K 2211/1007; C07D 401/10

(57) ABSTRACT

A light-emitting device material with a low refractive index is provided. The light-emitting device material contains an organic compound which has a pyridine skeleton, a diazine skeleton, or a triazine skeleton and in which the proportion of carbon atoms forming a bond by sp³ hybrid orbitals is within a certain range. The light-emitting device material contains an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. The ordinary refractive index of a layer containing the organic compound with respect to light with a wavelength in the range from 455 nm to 465 nm is 1.5 to 1.75.

### BACKGROUND OF THE INVENTION

### 1. Field of the Invention

One embodiment of the present invention relates to an organic compound, a light-emitting device, a display module, a lighting module, a display device, a light-emitting apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting apparatus, a lighting device, a power storage device, a memory device, an imaging device, a driving method thereof, and a manufacturing method thereof.

### 2. Description of the Related Art

Light-emitting devices (organic EL devices) including organic compounds and utilizing electroluminescence (EL) have been put to more practical use. In the basic structure of such light-emitting devices, an organic compound layer containing a light-emitting material (an EL layer) is interposed between a pair of electrodes. Carriers are injected by application of voltage to the device, and recombination energy of the carriers is used, whereby light emission can be obtained from the light-emitting material.

Such light-emitting devices are of self-luminous type and thus have advantages over liquid crystal displays, such as high visibility and no need for backlight when used as pixels of a display, and are suitable as flat panel display devices. Displays including such light-emitting devices are also highly advantageous in that they can be thin and lightweight. Moreover, such light-emitting devices also have a feature that response speed is extremely fast.

Since light-emitting layers of such light-emitting devices can be successively formed two-dimensionally, planar light emission can be achieved. This feature is difficult to realize with point light sources typified by incandescent lamps and LEDs or linear light sources typified by fluorescent lamps; thus, the light-emitting devices also have great potential as planar light sources, which can be used for lighting devices and the like.

Displays or lighting devices including light-emitting devices are suitably used for a variety of electronic devices as described above, and research and development of light-emitting devices have progressed for more favorable characteristics.

Low outcoupling efficiency is often a problem in an organic EL device. In particular, the attenuation due to reflection which is caused by a difference in refractive index between adjacent layers is a main cause of a reduction in device efficiency. In order to reduce this effect, a structure including a layer formed using a low refractive index material in an EL layer (see Non-Patent Document 1, for example) has been proposed.

A light-emitting device having this structure can have higher outcoupling efficiency and higher external quantum efficiency than a light-emitting device having a conventional structure; however, it is not easy to form such a layer with a low refractive index in an EL layer without adversely affecting other critical characteristics of the light-emitting device. This is because a low refractive index is in a trade-off relationship with a high carrier-transport property or high reliability of a light-emitting device including a layer with a low refractive index. This problem is caused because the carrier-transport property and reliability of an organic compound largely depend on an unsaturated bond, and an organic compound having many unsaturated bonds tends to have a high refractive index.

### REFERENCE

### Non-Patent Document

[Non-Patent Document 1] Jaeho Lee et al., “Synergetic electrode architecture for efficient graphene-based flexible organic light-emitting diodes”, Nature COMMUNICATIONS, Jun. 2, 2016, DOI: 10.1038/ncomms 11791.

### SUMMARY OF THE INVENTION

An object of one embodiment of the present invention is to provide a novel light-emitting device material or a novel electron-transport layer material. Another object of one embodiment of the present invention is to provide a novel light-emitting device material or an electron-transport layer material with a low refractive index. Another object of one embodiment of the present invention is to provide a novel light-emitting device material or an electron-transport layer material with a low refractive index and a carrier-transport property. Another object of one embodiment of the present invention is to provide a novel light-emitting device material or an electron-transport layer material with a low refractive index and an electron-transport property.

An object of one embodiment of the present invention is to provide a novel organic compound. Another object of one embodiment of the present invention is to provide a novel organic compound having a carrier-transport property. Another object of one embodiment of the present invention is to provide a novel organic compound having an electron-transport property. Another object of one embodiment of the present invention is to provide an organic compound with a low refractive index. Another object of one embodiment of the present invention is to provide an organic compound with a low refractive index and a carrier-transport property. Another object of one embodiment of the present invention is to provide an organic compound with a low refractive index and an electron-transport property.

Another object of one embodiment of the present invention is to provide a light-emitting device having high emission efficiency. Another object of one embodiment of the present invention is to provide a light-emitting device, a light-emitting apparatus, an electronic appliance, a display device, and an electronic device each having low power consumption.

Note that the description of these objects does not preclude the existence of other objects. One embodiment of the present invention does not necessarily achieve all the objects listed above. Other objects will be apparent from and can be derived from the description of the specification, the drawings, the claims, and the like.

It is only necessary that at least one of the above-described objects be achieved in the present invention.

One embodiment of the present invention is a light-emitting device material containing an organic compound which has a pyridine skeleton, a diazine skeleton, or a triazine skeleton and in which the proportion of carbon atoms forming a bond by the sp³ hybrid orbitals, which form a saturated hydrocarbon group, is within a certain range. Since the light-emitting device material has both the electron-transport property and the optical characteristics such as a low refractive index, the light-emitting device material is suitable for an electron-transport layer of a photoelectronics device such as a light-emitting device or a photoelectric conversion device, and can also be used as an electron-transport layer material.

One embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. The refractive index of a layer containing the organic compound is higher than or equal to 1.5 and lower than or equal to 1.75. Another embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. The proportion of carbon atoms forming a bond by the sp³ hybrid orbitals in the total number of carbon atoms in a molecule of the organic compound is higher than or equal to 10% and lower than or equal to 60%. Another embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. In the results of ¹H-NMR measurement conducted on the organic compound, the integral value of signals at lower than 4 ppm is equal to or more than half the integral value of signals at 4 ppm or higher.

Note that the heteroaromatic ring in the organic compound is preferably a triazine ring or a diazine ring, further preferably a triazine ring or a pyrimidine ring. The glass transition temperature is preferably higher than or equal to 100° C., further preferably higher than or equal to 110° C., still further preferably higher than or equal to 120° C.

One embodiment of the present invention is a light-emitting device material or an electron-transport layer material that contains an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. The ordinary refractive index of a layer containing the organic compound with respect to light with a wavelength in the range from 455 nm to 465 nm is higher than or equal to 1.5 and lower than or equal to 1.75. The benzene rings are each preferably a monocyclic benzene ring, i.e., a benzene ring to which no aromatic hydrocarbon ring is bonded.

Another embodiment of the present invention is a light-emitting device material or an electron-transport layer material that contains an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. The proportion of carbon atoms forming a bond by the sp³ hybrid orbitals in the total number of carbon atoms in a molecule of the organic compound is higher than or equal to 10% and lower than or equal to 60%.

In the above structure, the proportion of carbon atoms forming a bond by the sp³ hybrid orbitals in the total number of carbon atoms in a molecule of the organic compound is higher than or equal to 10% and lower than or equal to 50%.

Another embodiment of the present invention is a light-emitting device material or an electron-transport layer material that contains an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. In the results of ¹H-NMR measurement conducted on the organic compound, the integral value of signals at lower than 4 ppm is equal to or more than half the integral value of signals at 4 ppm or higher.

In any of the above structures, the molecular weight of the organic compound contained in the light-emitting device material or the electron-transport layer material is preferably greater than or equal to 500 and less than or equal to 2000.

In any of the above structures, it is preferable that the hydrocarbon groups in the molecule of the organic compound contained in the light-emitting device material or the electron-transport layer material be each bonded to the aromatic hydrocarbon rings in which the LUMO (Lowest Unoccupied Molecular Orbital) is not distributed, i.e., the LUMO be distributed in a ring other than the aromatic hydrocarbon rings to which the hydrocarbon groups are bonded in the molecule of the organic compound. Note that in this specification, the expression “the LUMO is not distributed in the aromatic hydrocarbon rings to which the hydrocarbon groups are bonded” means that the isovalue of the LUMO distribution density in the aromatic hydrocarbon rings to which the hydrocarbon groups are bonded is less than 0.06 [electrons/au³], preferably less than 0.02 [electrons/au³].

In any of the above structures, it is preferable that at least one of the aromatic hydrocarbon rings to which the hydrocarbon groups are bonded in the molecule of the organic compound contained in the light-emitting device material or the electron-transport layer material be a benzene ring.

In any of the above structures, it is preferable that the organic compound contained in the light-emitting device material or the electron-transport layer material include at least three benzene rings and the three benzene rings be each bonded to the six-membered heteroaromatic ring.

In any of the above structures, it is preferable that the organic compound contained in the light-emitting device material or the electron-transport layer material include at least three benzene rings, the three benzene rings be each bonded to the six-membered heteroaromatic ring, and two of the three benzene rings be each a substituted or unsubstituted phenyl group and include no hydrocarbon group.

In any of the above structures, the organic compound contained in the light-emitting device material or the electron-transport layer material includes a substituted or unsubstituted pyridyl group.

In the above structure, the six-membered heteroaromatic ring is preferably a triazine ring.

Alternatively, in the above structure, the six-membered heteroaromatic ring is preferably a pyrimidine ring.

In any of the above structures, the hydrocarbon groups forming a bond by the sp³ hybrid orbitals are each preferably an alkyl group or a cycloalkyl group.

In the above structure, the alkyl group is preferably a branched alkyl group having 3 to 5 carbon atoms.

Note that in the light-emitting device material or the electron-transport layer material, the glass transition temperature of the organic compound is preferably higher than or equal to 90° C. The glass transition temperature is further preferably higher than or equal to 100° C., still further preferably higher than or equal to 110° C., particularly preferably higher than or equal to 120° C.

Another embodiment of the present invention is an organic compound represented by General Formula (G1).

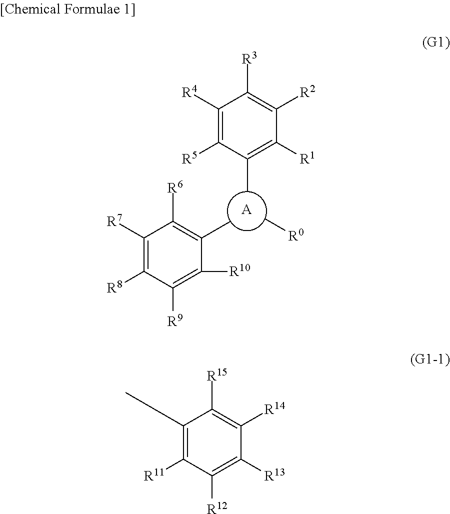

In General Formula (G1), A represents a six-membered heteroaromatic ring having 1 to 3 nitrogen atoms. R⁰ represents any of hydrogen, an alkyl group having 1 to 6 carbon atoms, an alicyclic group having 3 to 10 carbon atoms, and a substituent represented by Formula (G1-1). At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G1) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Another embodiment of the present invention is an organic compound represented by General Formula (G2).

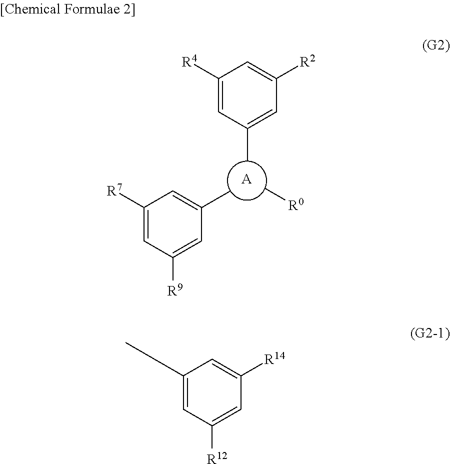

In General Formula (G2), A represents a six-membered heteroaromatic ring having 1 to 3 nitrogen atoms. R⁰ represents any of hydrogen, an alkyl group having 1 to 6 carbon atoms, an alicyclic group having 3 to 10 carbon atoms, and a substituent represented by Formula (G2-1). At least one of R², R⁴, R⁷, R⁹, R¹², and R¹⁴ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G2) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

In any of the above structures, Ain General Formula (G2) is preferably any one of a pyridine ring, a pyrimidine ring, a pyrazine ring, a pyridazine ring, and a triazine ring.

Another embodiment of the present invention is an organic compound represented by General Formula (G3).

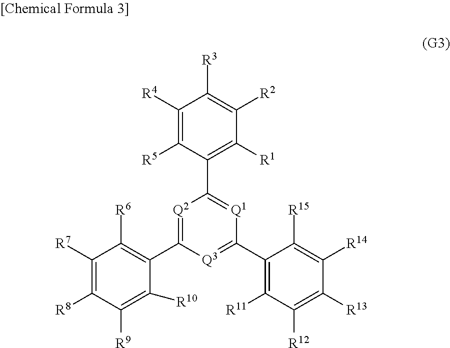

Two or three of Q¹ to Q³ represent N in General Formula (G3). When two of Q¹ to Q³ represent N, the remaining one of Q¹ to Q³ represents CH. At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G3) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Another embodiment of the present invention is an organic compound represented by General Formula (G4).

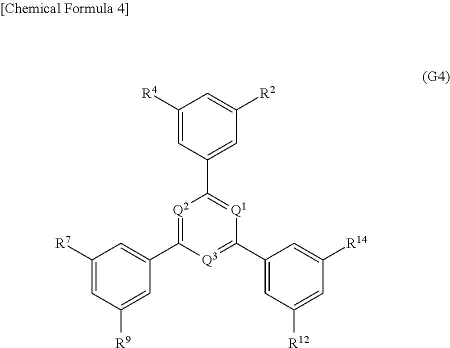

Two or three of Q¹ to Q³ represent N in General Formula (G4). When two of Q¹ to Q³ represent N, the remaining one of Q¹ to Q³ represents CH. At least one of R², R⁴, R⁷, R⁹, R¹², and R¹⁴ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G4) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

In any of the above structures, the phenyl group having a substituent in each of the organic compounds represented by General Formula (G1), General Formula (G2), General Formula (G3), and General Formula (G4) is preferably represented by Formula (G1-2) below.

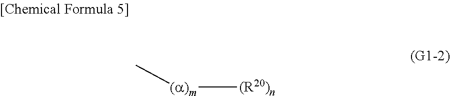

In General Formula (G1-2), a represents a substituted or unsubstituted phenylene group. R²⁰ represents an alkyl group having 1 to 6 carbon atoms, an alicyclic group having 3 to 10 carbon atoms, or a substituted or unsubstituted aromatic hydrocarbon group having 6 to 14 carbon atoms in a ring. In addition, m and n each represent 1 or 2. In the case where m is 2, a plurality of a may be the same or different from each other. In the case where n is 2, a plurality of R²⁰ may be the same or different from each other.

In the above structure, the group represented by General Formula (G1-2) is preferably used as one or both of R² and R⁴. Note that in the case where the group represented by General Formula (G1-2) is used as both of R² and R⁴, the two groups represented by General Formula (G1-2) may be the same or different from each other.

In any of the above structures, the phenyl group having a substituent in each of the organic compounds represented by General Formula (G1), General Formula (G2), General Formula (G3), and General Formula (G4) is preferably represented by General Formula (G1-3) below.

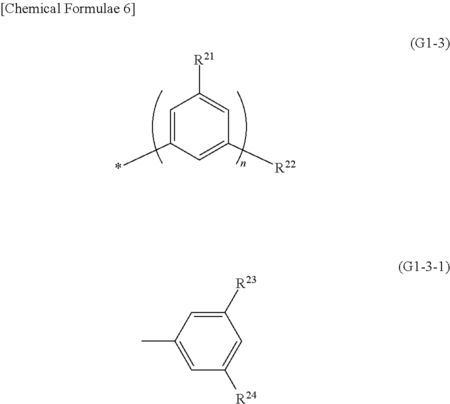

In General Formula (G1-3), R²¹ represents any one of hydrogen, an alkyl group having 1 to 6 carbon atoms, an alicyclic group having 3 to 10 carbon atoms, and a substituent represented by General Formula (G1-3-1). In addition, R²² represents the substituent represented by General Formula (G1-3-1). In General Formula (G1-3-1), R²³ and R²⁴ each represent any one of hydrogen, an alkyl group having 1 to 6 carbon atoms, and an alicyclic group having 3 to 10 carbon atoms, and at least one of R²³ and R²⁴ represents an alkyl group having 1 to 6 carbon atoms or an alicyclic group having 3 to 10 carbon atoms. Note that both of R²³ and R²⁴ preferably represent an alkyl group having 1 to 6 carbon atoms or an alicyclic group having 3 to 10 carbon atoms. Furthermore, n represents 0 to 2. In the case where n is 2, a plurality of R²¹ may be the same or different from each other. In the case where n is 0, at least one of R²³ and R²⁴ represents an alkyl group having 1 to 6 carbon atoms or an alicyclic group having 3 to 10 carbon atoms.

In the above structure, the group represented by General Formula (G1-3) is preferably used as one or both of R² and R⁴ in each of the organic compounds represented by General Formula (G1), General Formula (G2), General Formula (G3), and General Formula (G4). Note that in the case where the group represented by General Formula (G1-3) is used as both of R² and R⁴, the two groups represented by General Formula (G1-3) may be the same or different from each other.

In any of the above structures, when the aromatic hydrocarbon group having 6 to 14 carbon atoms in a ring in each of the organic compounds represented by General Formula (G1), General Formula (G2), General Formula (G3), and General Formula (G4) has a substituent, the substituent is preferably any one of an alkyl group having 1 to 6 carbon atoms, an alicyclic group having 3 to 10 carbon atoms, an unsubstituted aromatic hydrocarbon group having 6 to 14 carbon atoms, and an aromatic hydrocarbon group having 6 to 14 carbon atoms in a ring, to which an alkyl group having 1 to 6 carbon atoms or an alicyclic group having 3 to 10 carbon atoms is bonded.

In any of the above structures, the aromatic hydrocarbon group having 6 to 14 carbon atoms in a ring in the organic compound represented by any of General Formula (G1), General Formula (G2), General Formula (G3), and General Formula (G4) and in the phenyl group having a substituent that is represented by either of General Formula (G1-2) and General Formula (G1-3) is preferably any one of a phenyl group, a naphthyl group, a phenanthrenyl group, and a fluorenyl group.

In any of the above structures, the aromatic hydrocarbon group having 6 to 14 carbon atoms in a ring in the organic compound represented by any of General Formula (G1), General Formula (G2), General Formula (G3), and General Formula (G4) and in the phenyl group having a substituent that is represented by either of General Formula (G1-2) and General Formula (G1-3) is preferably represented by any one of Formulae (ra-1) to (ra-15) below.

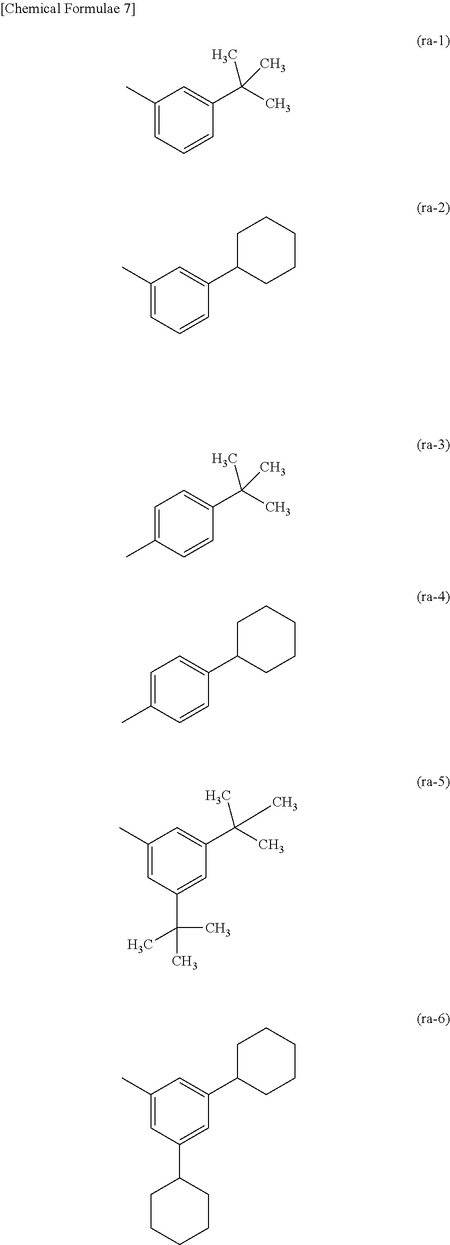

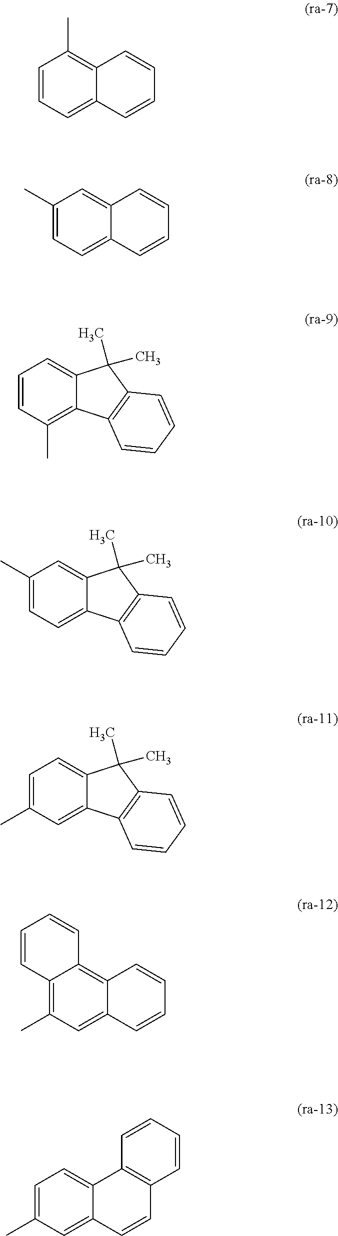

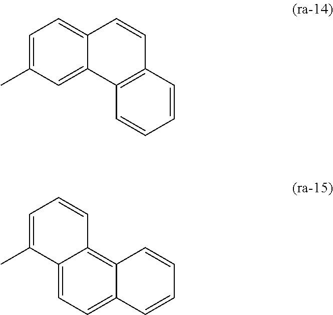

In any of the above structures, the substituted or unsubstituted pyridyl group in each of the organic compounds represented by General Formula (G1), General Formula (G2), General Formula (G3), and General Formula (G4) is preferably an unsubstituted pyridyl group or a pyridyl group to which one or more methyl groups are bonded.

In any of the above structures, the alicyclic group in each of the organic compounds represented by General Formula (G1), General Formula (G2), General Formula (G3), and General Formula (G4) is preferably a cycloalkyl group having 3 to 6 carbon atoms.

In any of the above structures, the alkyl group having 1 to 6 carbon atoms in each of the organic compounds represented by General Formula (G1), General Formula (G2), General Formula (G3), and General Formula (G4) is preferably a branched alkyl group having 3 to 5 carbon atoms.

Another embodiment of the present invention is an organic compound represented by General Formula (G4′).

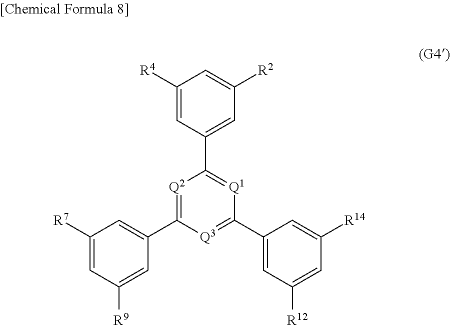

Two or three of Q¹ to Q³ represent N in General Formula (G4′). When two of Q¹ to Q³ represent N, the remaining one of Q¹ to Q³ represents CH. Furthermore, R² is represented by Formula (R²-1) below, and R⁴, R⁷, R⁹, R¹² and R¹⁴ each independently represent any one of Formulae (r-1) to (r-20) below. Note that in Formula (R²-1), represents a substituted or unsubstituted phenylene group or a substituted or unsubstituted biphenyldiyl group, R²⁵ represents any one of Formulae (r-1) to (r-17), and n represents 1 or 2. Note that the organic compound represented by General Formula (G4′) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

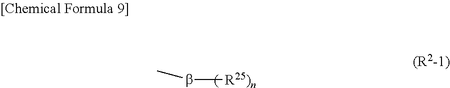

Formula (R²-1) that represents R² in the organic compound represented by General Formula (G4′) is represented by any one of Formulae (r-1) to (r-20).

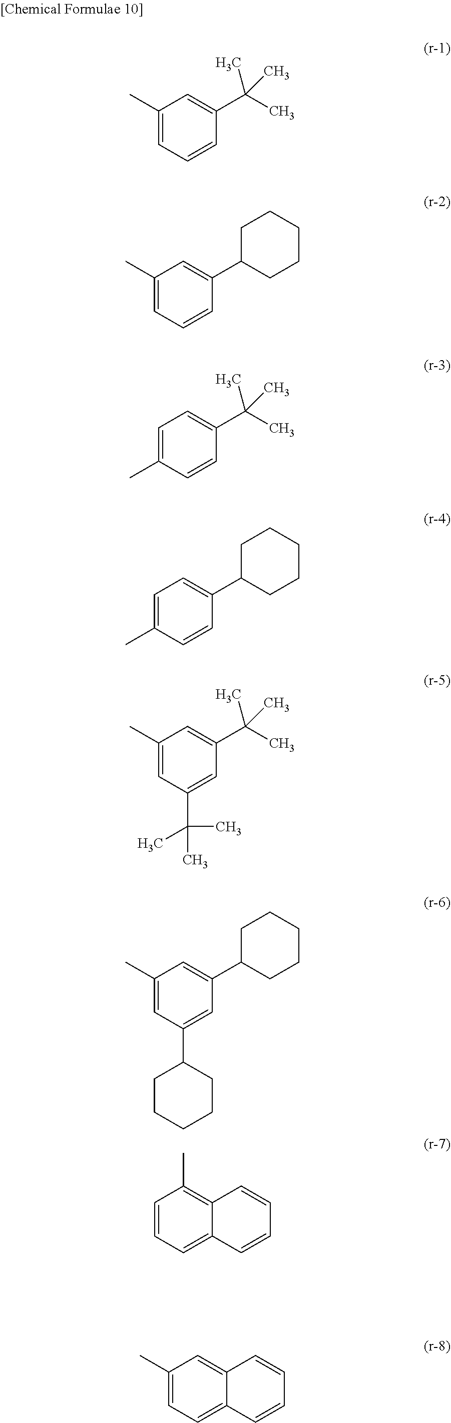

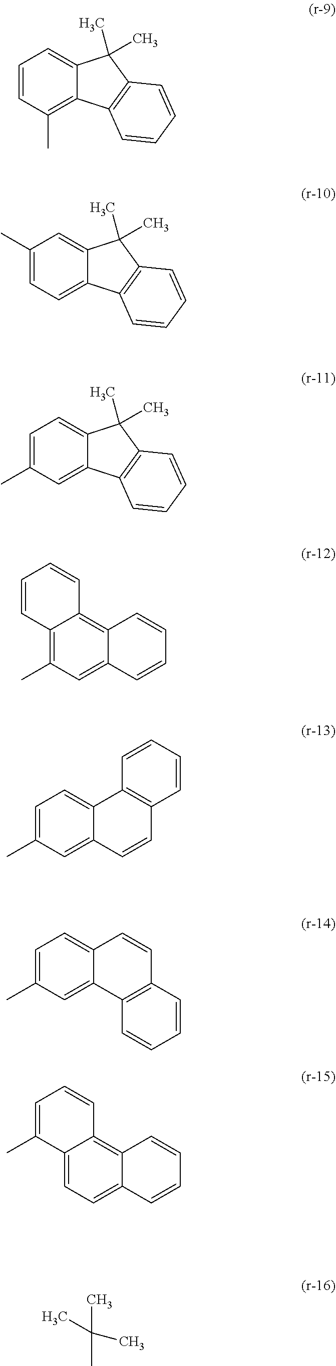

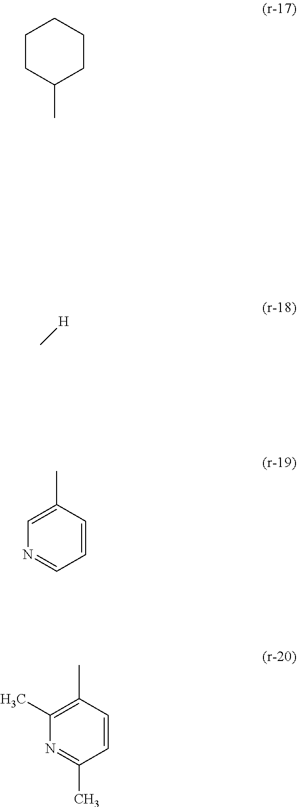

An alkyl group or a cycloalkyl group is preferably substituted at the meta-position of the phenyl group as shown in Formulae (r-1), (r-2), (r-5), and (r-6), in which case the film density is reduced and the refractive index can be lowered. It is further preferable that two alkyl groups or two cycloalkyl groups be bonded to the phenyl group as shown in Formulae (r-5) and (r-6) because the total number of carbon atoms forming a bond by the sp³ hybrid orbitals can be easily increased, which can reduce the synthesis cost. An alkyl group or a cycloalkyl group is preferably substituted at the para-position of the phenyl group as shown in Formulae (r-3) and (r-4), in which case carrier mobility should be high. A pyridyl group is preferably contained as shown in Formulae (r-19) and (r-20) because injection of electrons from a cathode, an electron-injection layer, or the like is facilitated, which can decrease the driving voltage.

In the above structure, β in Formula (R²-1) is preferably represented by any one of Formulae (β-1) to (β-14) below.

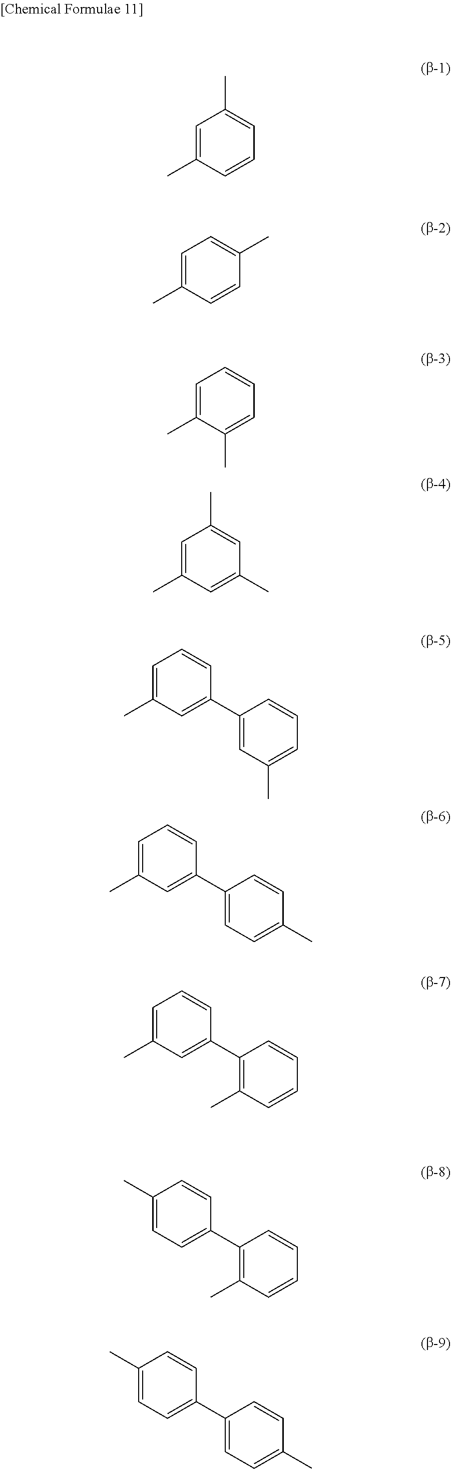

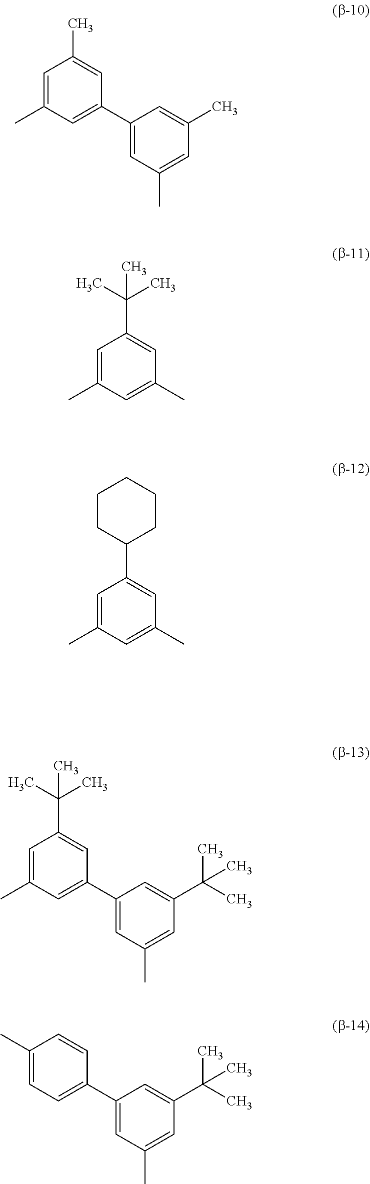

Another embodiment of the present invention is an organic compound represented by any one of Structural Formulae (100), (120), (121), (123), (200), and (412).

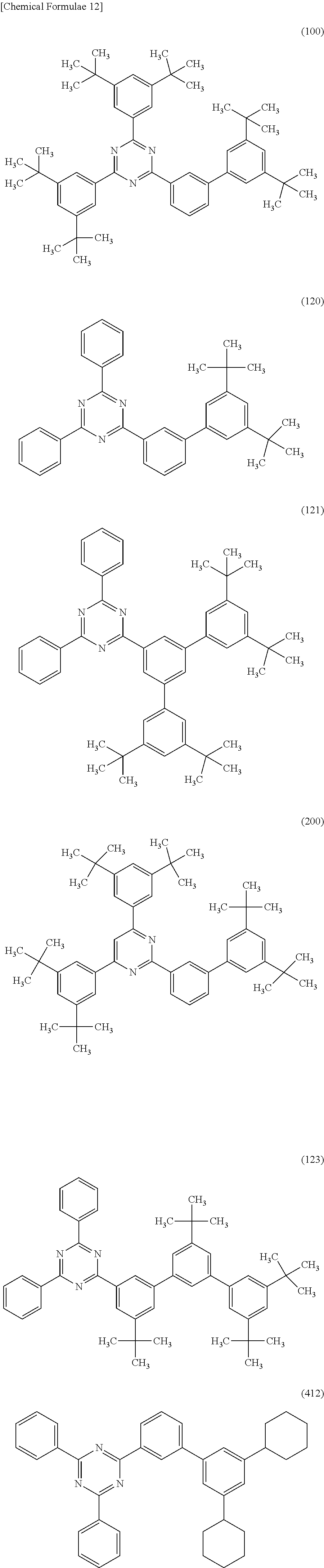

Another embodiment of the present invention is a light-emitting device using the above-described organic compound of one embodiment of the present invention. The present invention also includes a light-emitting device containing a guest material as well as the above-described organic compound.

Note that the present invention also includes a light-emitting device in which an EL layer provided between a pair of electrodes or a light-emitting layer included in the EL layer contains the organic compound of one embodiment of the present invention. In addition to the aforementioned light-emitting device, the present invention includes a light-emitting device including a layer (e.g., a cap layer) that is in contact with an electrode and contains an organic compound. In addition to the light-emitting devices, a light-emitting apparatus including a transistor, a substrate, and the like is also included in the scope of the invention. Furthermore, an electronic device or a lighting device including any of these light-emitting devices and any of a sensor, an operation button, a speaker, a microphone, and the like is also included in the scope of the invention.

In addition, the scope of one embodiment of the present invention includes a light-emitting apparatus including a light-emitting device, and a lighting device including the light-emitting apparatus. Accordingly, the light-emitting apparatus in this specification refers to an image display device and a light source (including a lighting device). In addition, the light-emitting apparatus includes the following in its category: a module in which a connector such as a flexible printed circuit (FPC) or a tape carrier package (TCP) is attached to a light-emitting apparatus; a module in which a printed wiring board is provided at the end of a TCP; and a module in which an integrated circuit (IC) is directly mounted on a light-emitting device by a chip on glass (COG) method.

According to one embodiment of the present invention, a novel light-emitting device material or a novel electron-transport layer material can be provided. According to another embodiment of the present invention, a novel light-emitting device material or an electron-transport layer material with a low refractive index can be provided. According to another embodiment of the present invention, a novel light-emitting device material or an electron-transport layer material with a low refractive index and a carrier-transport property can be provided. According to another embodiment of the present invention, a novel light-emitting device material or an electron-transport layer material with a low refractive index and an electron-transport property can be provided.

According to one embodiment of the present invention, a novel organic compound can be provided. According to another embodiment of the present invention, a novel organic compound having a carrier-transport property can be provided. According to another embodiment of the present invention, a novel organic compound having an electron-transport property can be provided. According to another embodiment of the present invention, an organic compound with a low refractive index can be provided. According to another embodiment of the present invention, an organic compound with a low refractive index and a carrier-transport property can be provided. According to another embodiment of the present invention, an organic compound with a low refractive index and an electron-transport property can be provided.

According to another embodiment of the present invention, a light-emitting device having high emission efficiency can be provided. According to another embodiment of the present invention, a light-emitting device, a light-emitting apparatus, an electronic appliance, a display device, and an electronic device each having low power consumption can be provided.

Note that the description of these effects does not preclude the existence of other effects. One embodiment of the present invention does not necessarily have all the effects listed above. Other effects will be apparent from and can be derived from the description of the specification, the drawings, the claims, and the like.

### BRIEF DESCRIPTION OF THE DRAWINGS

In the accompanying drawings:

FIGS. 1A to 1C are schematic views of light-emitting devices;

FIGS. 2A and 2B are conceptual views of an active matrix light-emitting apparatus;

FIGS. 3A and 3B are conceptual views of active matrix light-emitting apparatuses;

FIG. 4 is a conceptual view of an active matrix light-emitting apparatus;

FIGS. 5A and 5B are conceptual views of a passive matrix light-emitting apparatus;

FIGS. 6A and 6B illustrate a lighting device;

FIGS. 7A, 7B1, 7B2, and 7C illustrate electronic devices;

FIGS. 8A to 8C illustrate electronic devices;

FIG. 9 illustrates a lighting device;

FIG. 10 illustrates a lighting device;

FIG. 11 illustrates in-vehicle display devices and lighting devices;

FIGS. 12A and 12B illustrate an electronic device;

FIGS. 13A to 13C illustrate an electronic device;

FIG. 14 shows an MS spectrum of mmtBumBP-dmmtBuPTzn;

FIG. 15 shows measurement data of refractive indices of mmtBumBP-dmmtBuPTzn;

FIG. 16 shows an MS spectrum of mmtBumBPTzn;

FIG. 17 shows measurement data of refractive indices of mmtBumPTzn;

FIG. 18 shows an MS spectrum of mmtBumTPTzn;

FIG. 19 shows measurement data of refractive indices of mmtBumTPTzn;

FIG. 20 shows an MS spectrum of mmtBumBP-dmmtBuPPm;

FIG. 21 shows measurement data of refractive indices of mmtBumBP-dmmtBuPPm;

FIG. 22 shows measurement data of refractive indices of mmtBumBP-dmmtBuPTzn, mPn-mDMePyPTzn, Li-6mq, and Liq;

FIG. 23 shows luminance-current density characteristics of a light-emitting device 1 and a comparative light-emitting device 1;

FIG. 24 shows current efficiency-luminance characteristics of the light-emitting device 1 and the comparative light-emitting device 1;

FIG. 25 shows luminance-voltage characteristics of the light-emitting device 1 and the comparative light-emitting device 1;

FIG. 26 shows current-voltage characteristics of the light-emitting device 1 and the comparative light-emitting device 1;

FIG. 27 shows blue index-luminance characteristics of the light-emitting device 1 and the comparative light-emitting device 1;

FIG. 28 shows emission spectra of the light-emitting device 1 and the comparative light-emitting device 1;

FIG. 29 shows measurement data of refractive indices of mmtBumBPTzn and mPn-mDMePyPTzn;

FIG. 30 shows luminance-current density characteristics of a light-emitting device 2 and a comparative light-emitting device 2;

FIG. 31 shows current efficiency-luminance characteristics of the light-emitting device 2 and the comparative light-emitting device 2;

FIG. 32 shows luminance-voltage characteristics of the light-emitting device 2 and the comparative light-emitting device 2;

FIG. 33 shows current-voltage characteristics of the light-emitting device 2 and the comparative light-emitting device 2;

FIG. 34 shows blue index-luminance characteristics of the light-emitting device 2 and the comparative light-emitting device 2;

FIG. 35 shows emission spectra of the light-emitting device 2 and the comparative light-emitting device 2;

FIG. 36 shows luminance-current density characteristics of a light-emitting device 3, a light-emitting device 4, and a light-emitting device 5;

FIG. 37 shows current efficiency-luminance characteristics of the light-emitting device 3, the light-emitting device 4, and the light-emitting device 5;

FIG. 38 shows luminance-voltage characteristics of the light-emitting device 3, the light-emitting device 4, and the light-emitting device 5;

FIG. 39 shows current density-voltage characteristics of the light-emitting device 3, the light-emitting device 4, and the light-emitting device 5;

FIG. 40 shows power efficiency-luminance characteristics of the light-emitting device 3, the light-emitting device 4, and the light-emitting device 5;

FIG. 41 shows emission spectra of the light-emitting device 3, the light-emitting device 4, and the light-emitting device 5;

FIG. 42 shows external quantum efficiency-luminance characteristics of the light-emitting device 3, the light-emitting device 4, and the light-emitting device 5;

FIG. 43 shows reliability of the light-emitting device 3, the light-emitting device 4, and the light-emitting device 5;

FIG. 44 shows an absorption spectrum and an emission spectrum of Li-6mq in a dehydrated acetone solution;

FIG. 45 shows luminance-current density characteristics of a light-emitting device 6, a light-emitting device 7, and a comparative light-emitting device 3;

FIG. 46 shows current efficiency-luminance characteristics of the light-emitting device 6, the light-emitting device 7, and the comparative light-emitting device 3;

FIG. 47 shows luminance-voltage characteristics of the light-emitting device 6, the light-emitting device 7, and the comparative light-emitting device 3;

FIG. 48 shows current density-voltage characteristics of the light-emitting device 6, the light-emitting device 7, and the comparative light-emitting device 3;

FIG. 49 shows external quantum efficiency-luminance characteristics of the light-emitting device 6, the light-emitting device 7, and the comparative light-emitting device 3;

FIG. 50 shows emission spectra of the light-emitting device 6, the light-emitting device 7, and the comparative light-emitting device 3;

FIG. 51 shows time dependence of normalized luminance of the light-emitting device 6, the light-emitting device 7, and the comparative light-emitting device 3;

FIG. 52 shows an MS spectrum of mmtBumTPTzn-02;

FIG. 53 shows measurement data of refractive indices of mmtBumTPTzn-02;

FIG. 54 shows an MS spectrum of mmtBumTPTzn-04;

FIG. 55 shows measurement data of refractive indices of mmtBumTPTzn-04;

FIG. 56 shows an MS spectrum of mmtBuPh-mDMePyPTzn;

FIG. 57 shows measurement data of refractive indices of mmtBuPh-mDMePyPTzn;

FIG. 58 shows an MS spectrum of mmchmBPTzn;

FIG. 59 shows measurement data of refractive indices of mmchmBPTzn;

FIG. 60 shows an MS spectrum of mmtBuTPTzn-03;

FIG. 61 shows measurement data of refractive indices of mmtBuTPTzn-03;

FIG. 62 shows an MS spectrum of mmtBumBP2Tzn;

FIG. 63 shows measurement data of refractive indices of mmtBumBP2Tzn;

FIG. 64 shows an MS spectrum of oBP-mmtBumBPTzn;

FIG. 65 shows measurement data of refractive indices of oBP-mmtBumBPTzn;

FIG. 66 shows an MS spectrum of oBP-mmtBuBPTzn;

FIG. 67 shows measurement data of refractive indices of oBP-mmtBuBPTzn;

FIG. 68 shows an MS spectrum of mmtBuPh-mPyPTzn;

FIG. 69 shows measurement data of refractive indices of mmtBuPh-mPyPTzn;

FIG. 70 shows an MS spectrum of mmtBuBP-mDMePyPTzn;

FIG. 71 shows measurement data of refractive indices of mmtBuBP-mDMePyPTzn;

FIG. 72 shows luminance-current density characteristics of a light-emitting device 8, a light-emitting device 9, and a comparative light-emitting device 4;

FIG. 73 shows luminance-voltage characteristics of the light-emitting device 8, the light-emitting device 9, and the comparative light-emitting device 4;

FIG. 74 shows current efficiency-luminance characteristics of the light-emitting device 8, the light-emitting device 9, and the comparative light-emitting device 4;

FIG. 75 shows current density-voltage characteristics of the light-emitting device 8, the light-emitting device 9, and the comparative light-emitting device 4;

FIG. 76 shows external quantum efficiency-luminance characteristics of the light-emitting device 8, the light-emitting device 9, and the comparative light-emitting device 4;

FIG. 77 shows emission spectra of the light-emitting device 8, the light-emitting device 9, and the comparative light-emitting device 4;

FIG. 78 shows luminance-current density characteristics of a light-emitting device 10 and a comparative light-emitting device 5;

FIG. 79 shows luminance-voltage characteristics of the light-emitting device 10 and the comparative light-emitting device 5;

FIG. 80 shows current efficiency-luminance characteristics of the light-emitting device 10 and the comparative light-emitting device 5;

FIG. 81 shows current density-voltage characteristics of the light-emitting device 10 and the comparative light-emitting device 5;

FIG. 82 shows external quantum efficiency-luminance characteristics of the light-emitting device 10 and the comparative light-emitting device 5;

FIG. 83 shows emission spectra of the light-emitting device 10 and the comparative light-emitting device 5;

FIG. 84 shows an MS spectrum of mmtBuBP-mDMePyPTzn-02;

FIG. 85 shows measurement data of refractive indices of mmtBuBP-mDMePyPTzn-02;

FIG. 86 shows an MS spectrum of oBP2-mmtBuPh-mDMePyPTzn;

FIG. 87 shows measurement data of refractive indices of oBP2-mmtBuPh-mDMePyPTzn;

FIG. 88 shows an MS spectrum of oBP-mmtBuPh-mDMePyPTzn;

FIG. 89 shows measurement data of refractive indices of oBP-mmtBuPh-mDMePyPTzn;

FIG. 90 shows an MS spectrum of tBu-TmPPPyTz; and

FIG. 91 shows an MS spectrum of tBu-TmPPPyTz-02.

### DETAILED DESCRIPTION OF THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

Among organic compounds having a carrier-transport property that can be used for an organic EL device, 1,1-bis-(4-bis(4-methyl-phenyl)-amino-phenyl)-cyclohexane (abbreviation: TAPC) is one of materials with a low refractive index. The use of a material with a low refractive index for an EL layer can increase the external quantum efficiency of a light-emitting device; thus, with TAPC, a light-emitting device with high external quantum efficiency should be obtained. However, TAPC has a heat resistance problem because of its low glass transition temperature. In addition, TAPC can transport holes but cannot transport electrons substantially.

In order to obtain a material with a low refractive index, an atom with low atomic refraction or a substituent with low molecular refraction is preferably introduced into the molecule. Examples of the substituent with low molecular refraction include a saturated hydrocarbon group and a cyclic saturated hydrocarbon group.

In general, a carrier-transport property and a refractive index have a trade-off relationship; an increase in a carrier-transport property causes an increase in a refractive index. This is because the carrier-transport property of an organic compound largely depends on an unsaturated bond, and an organic compound having many unsaturated bonds tends to have a high refractive index.

It has been originally known that an electron-transport organic compound is difficult to have sufficient characteristics, such as mobility and stability, required for an organic EL device as compared with a hole-transport organic compound because the required LUMO level is low. Thus, introduction of a saturated hydrocarbon group, which adversely affects those characteristics, has been considered undesirable.

Contrary to the commonly accepted theory, the present inventors have developed, as a compound having both a carrier-transport property and a low refractive index, a light-emitting device material containing an organic compound which has a pyridine skeleton, a diazine skeleton, or a triazine skeleton and in which the proportion of carbon atoms forming a bond by the sp³ hybrid orbitals, which form a saturated hydrocarbon group, is within a certain range. Since the light-emitting device material has both the electron-transport property and the optical characteristics such as a low refractive index, the light-emitting device material is suitable for an electron-transport layer of a photoelectronics device such as a light-emitting device or a photoelectric conversion device, and can also be used as an electron-transport layer material. The organic compound contained in the light-emitting device material and the electron-transport layer material can achieve a low refractive index while maintaining a high electron-transport property when the number of substituents containing carbon atoms forming a bond by the sp³ hybrid orbitals or the site of substitution in the organic compound is adjusted. When the proportion of carbon atoms forming a bond by the sp³ hybrid orbitals in the organic compound is within a certain range, a light-emitting device material and an electron-transport layer material each having not only a low refractive index and a high electron-transport property but also heat resistance such as a high glass transition temperature can be obtained.

The use of the light-emitting device material of one embodiment of the present invention for an EL layer of a light-emitting device can increase the outcoupling efficiency of the EL layer because of the low refractive index, which can improve the emission efficiency of the light-emitting device.

The electron-transport layer material of one embodiment of the present invention has a high electron-transport property and thus is suitable for an electron-transport layer of an EL layer in a light-emitting device. In addition, the electron-transport layer material can increase the outcoupling efficiency of the EL layer because of the low refractive index, which can improve the emission efficiency of the light-emitting device. Moreover, the electron-transport layer material of one embodiment of the present invention has a high electron-transport property and a property of transmitting light (in particular, visible light) and thus is suitable for an electron-transport layer of a photoelectric conversion device.

That is, one embodiment of the present invention is a light-emitting device material that can be used for an EL layer in a light-emitting device, or an electron-transport layer material that can be used for an electron-transport layer of an EL layer in a light-emitting device or an electron-transport layer in a photoelectric conversion device. The light-emitting device material or the electron-transport layer material contains an organic compound including at least one six-membered heteroaromatic ring having 1 to 3 nitrogen atoms. The glass transition temperature of the organic compound is higher than or equal to 90° C. The refractive index of a layer containing the organic compound is higher than or equal to 1.5 and lower than or equal to 1.75. Another embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. The proportion of carbon atoms forming a bond by the sp³ hybrid orbitals in the total number of carbon atoms in a molecule of the organic compound is higher than or equal to 10% and lower than or equal to 60%. Another embodiment of the present invention is a light-emitting device material or an electron-transport layer material that contains an organic compound including at least one six-membered heteroaromatic ring having 1 to 3 nitrogen atoms. The glass transition temperature of the organic compound is higher than or equal to 90° C. In the results of ¹H-NMR measurement conducted on the organic compound, the integral value of signals at lower than 4 ppm is equal to or more than half the integral value of signals at 4 ppm or higher.

Note that the heteroaromatic ring in the organic compound is preferably a triazine ring or a diazine ring, further preferably a triazine ring or a pyrimidine ring. The glass transition temperature is preferably higher than or equal to 100° C., further preferably higher than or equal to 110° C., still further preferably higher than or equal to 120° C.

In the case where the material has anisotropy, the refractive index with respect to an ordinary ray might differ from that with respect to an extraordinary ray. When a thin film to be measured is in such a state, anisotropy analysis can be performed to separately calculate the ordinary refractive index and the extraordinary refractive index. In this specification, when the measured material has both the ordinary refractive index and the extraordinary refractive index, the ordinary refractive index is used as an indicator.

Another embodiment of the present invention is a light-emitting device material that can be used for an EL layer in a light-emitting device, or an electron-transport layer material that can be used for an electron-transport layer of an EL layer in a light-emitting device or an electron-transport layer in a photoelectric conversion device. The light-emitting device material or the electron-transport layer material contains an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. The ordinary refractive index of a layer containing the organic compound with respect to light with a wavelength in the range from 455 nm to 465 nm is higher than or equal to 1.5 and lower than or equal to 1.75. The benzene rings are each preferably a monocyclic benzene ring, i.e., a benzene ring to which no aromatic ring is bonded.

Note that the proportion of carbon atoms forming a bond by the sp³ hybrid orbitals in the above structure affects the refractive index of the organic compound. That is, an increase in the number of carbon atoms forming a bond by the sp³ hybrid orbitals lowers the refractive index; thus, the outcoupling efficiency of a light-emitting device containing the above-described material can be improved.

Another embodiment of the present invention is a light-emitting device material that can be used for an EL layer in a light-emitting device, or an electron-transport layer material that can be used for an electron-transport layer of an EL layer in a light-emitting device or an electron-transport layer in a photoelectric conversion device. The materials each contain an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. The proportion of carbon atoms forming a bond by the sp³ hybrid orbitals in the total number of carbon atoms in a molecule of the organic compound is preferably higher than or equal to 10% and lower than or equal to 60%.

Note that the proportion of carbon atoms forming a bond by the sp³ hybrid orbitals in the above structure affects the refractive index of the organic compound. That is, an increase in the number of carbon atoms forming a bond by the sp³ hybrid orbitals lowers the refractive index; thus, the outcoupling efficiency of a light-emitting device containing the above-described material can be improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, the proportion of carbon atoms forming a bond by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming a bond by the sp³ hybrid orbitals in the total number of carbon atoms in a molecule of the organic compound is preferably higher than or equal to 20% and lower than or equal to 40%.

Another embodiment of the present invention is a light-emitting device material that can be used for an EL layer in a light-emitting device, or an electron-transport layer material that can be used for an electron-transport layer of an EL layer in a light-emitting device or an electron-transport layer in a photoelectric conversion device. The materials each contain an organic compound including at least one six-membered heteroaromatic ring having 1 to 3 nitrogen atoms and a plurality of aromatic hydrocarbon rings each having 6 to 14 carbon atoms in a ring. At least two of the plurality of aromatic hydrocarbon rings are benzene rings. The organic compound has a plurality of hydrocarbon groups forming a bond by the sp³ hybrid orbitals. In the results of ¹H-NMR measurement conducted on the organic compound, the integral value of signals at lower than 4 ppm is preferably equal to or more than half the integral value of signals at 4 ppm or higher.

Note that the proportion of carbon atoms forming a bond by the sp³ hybrid orbitals in the above structure affects the refractive index of the organic compound. That is, an increase in the number of carbon atoms forming a bond by the sp³ hybrid orbitals lowers the refractive index; thus, the outcoupling efficiency of a light-emitting device containing the above-described material can be improved. Furthermore, the number of carbon atoms forming a bond by the sp³ hybrid orbitals is preferably large, in which case heat resistance such as a glass transition temperature is improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, in the results of ¹H-NMR measurement conducted on the organic compound, the integral value of signals at lower than 4 ppm derived from protons of an alkyl group and an alicyclic group is preferably more than or equal to half and less than or equal to twice, further preferably more than or equal to one times and less than or equal to one and a half times the integral value of signals at 4 ppm or higher derived from an aryl group or a heteroaromatic group.

The molecular weight of the organic compound contained in the light-emitting device material or the electron-transport layer material is preferably greater than or equal to 500 and less than or equal to 2000. The molecular weight is further preferably greater than or equal to 700 and less than or equal to 1500, in which case the thermophysical property (glass transition temperature) is high and decomposition is less likely to occur at the time of sublimation (vapor deposition).

In the molecule of the organic compound contained in the light-emitting device material or the electron-transport layer material, it is preferable that the hydrocarbon groups forming a bond by the sp³ hybrid orbitals be each bonded to the aromatic hydrocarbon rings in which the LUMO is not distributed, i.e., the LUMO be distributed in a ring other than the aromatic hydrocarbon rings to which the hydrocarbon groups are bonded in the molecule of the organic compound. Note that in this specification, the expression “the LUMO is not distributed in the aromatic hydrocarbon rings to which the hydrocarbon groups are bonded” means that the isovalue of the LUMO distribution density in the aromatic hydrocarbon rings to which the hydrocarbon groups are bonded is less than 0.06 [electrons/au³], preferably less than 0.02 [electrons/au³].

It is further preferable that the LUMO be mainly distributed in the heteroaromatic ring and a substituent directly bonded to the heteroaromatic ring. With such a molecule, an overlap of the LUMO between adjacent organic compound molecules in the solid (film) state is likely to occur and thus transportation of electrons is facilitated, which can decrease the driving voltage.

Note that the isovalue of the LUMO can be obtained through molecular orbital calculation with Gaussian, for example.

In the molecule of the organic compound contained in the light-emitting device material or the electron-transport layer material, it is preferable that at least one of the aromatic hydrocarbon rings to which the hydrocarbon groups forming a bond by the sp³ hybrid orbitals are bonded be a benzene ring.

It is preferable that the organic compound contained in the light-emitting device material or the electron-transport layer material include at least three benzene rings, the three benzene rings be each bonded to the six-membered heteroaromatic ring, and two of the three benzene rings be each a substituted or unsubstituted phenyl group and include no hydrocarbon group. The six-membered heteroaromatic ring is preferably a triazine ring or a pyrimidine ring.

The organic compound contained in the light-emitting device material or the electron-transport layer material preferably includes a substituted or unsubstituted pyridyl group, in which case the property of electron injection from a cathode or an electron-injection layer can be increased.

It is preferable that the hydrocarbon groups forming a bond by the sp³ hybrid orbitals in the organic compound contained in the light-emitting device material or the electron-transport layer material be each an alkyl group or a cycloalkyl group, and the alkyl group be a branched alkyl group having 3 to 5 carbon atoms.

Note that in the light-emitting device material or the electron-transport layer material, the glass transition temperature of the organic compound is preferably higher than or equal to 90° C. The glass transition temperature is further preferably higher than or equal to 100° C., still further preferably higher than or equal to 110° C., particularly preferably higher than or equal to 120° C.

Next, organic compounds of one embodiment of the present invention, each of which can be used as one mode of the organic compound contained in the light-emitting device material or the electron-transport layer material, will be described below.

That is, one embodiment of the present invention is an organic compound represented by General Formula (G1).

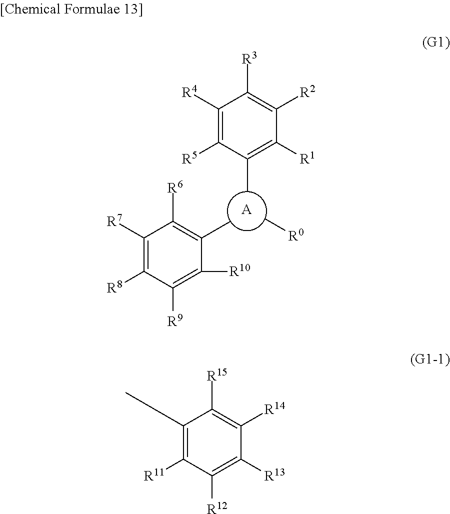

In General Formula (G1), A represents a six-membered heteroaromatic ring having 1 to 3 nitrogen atoms. R⁰ represents any of hydrogen, an alkyl group having 1 to 6 carbon atoms, an alicyclic group having 3 to 10 carbon atoms, and a substituent represented by Formula (G1-1). At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G1) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Note that in the structure of the organic compound represented by General Formula (G1), the proportion of carbon atoms forming a bond by the sp³ hybrid orbitals affects the refractive index of the organic compound. That is, an increase in the number of carbon atoms forming a bond by the sp³ hybrid orbitals lowers the refractive index; thus, the outcoupling efficiency of a light-emitting device containing the organic compound can be improved. Furthermore, the number of carbon atoms forming a bond by the sp³ hybrid orbitals is preferably large, in which case heat resistance such as a glass transition temperature is improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, the proportion of carbon atoms forming a bond by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming a bond by the sp³ hybrid orbitals in the total number of carbon atoms in a molecule of the organic compound is preferably higher than or equal to 20% and lower than or equal to 40%.

The organic compound represented by General Formula (G1) is preferably formed of only a six-membered heteroaromatic ring having 1 to 3 nitrogen atoms, a six-membered aromatic ring (i.e., a substituted or unsubstituted phenyl group), and a hydrocarbon group/hydrocarbon groups forming a bond/bonds by the sp³ hybrid orbitals (e.g., an alkyl group or an alicyclic group), that is, the organic compound preferably includes no condensed ring, in which case the refractive index is lowered and the transport property of carriers (electrons) is increased.

A pyridine ring, a pyrimidine ring, a pyrazine ring, a pyridazine ring, or a triazine ring can be used as A in the organic compound represented by General Formula (G1). In the case where the organic compound represented by General Formula (G1) is used for a light-emitting layer or a layer in contact with an active layer, a triazine ring, a pyrazine ring, or a pyrimidine ring, which easily injects electrons into these layers and has a high electron-transport property, is preferably used, and a triazine ring is particularly preferable.

The total number of substituents (an alkyl group and an alicyclic group) per molecule in the organic compound represented by General Formula (G1) is preferably greater than or equal to 4 and less than or equal to 10 in consideration of the synthesis cost, further preferably greater than or equal to 6 in order to lower the refractive index. Similarly, using as large substituents (an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, naphthyl group, phenanthryl group, or fluorenyl group can be used as the aromatic hydrocarbon group having 6 to 14 carbon atoms in a ring in the organic compound represented by General Formula (G1). The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the organic compound represented by General Formula (G1), a pyridyl group, a pyrimidinyl group, a pyrazinyl group, a triazinyl group, a quinolyl group, a quinazolinyl group, a quinoxalinyl group, or the like can be used as the heteroaromatic ring group having 3 to 9 carbon atoms in a ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G1). As the alicyclic group having 3 to 10 carbon atoms, a cyclopropyl group, a cyclohexyl group, a cyclodecanyl group, a bicyclooctyl group, an adamantyl group, or the like can be used.

Another embodiment of the present invention is an organic compound represented by General Formula (G2).

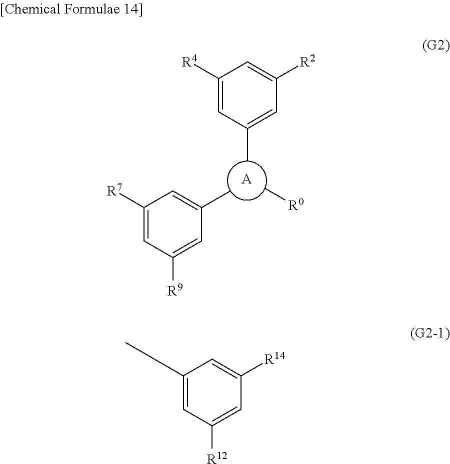

In General Formula (G2), A represents a six-membered heteroaromatic ring having 1 to 3 nitrogen atoms. R⁰ represents any of hydrogen, an alkyl group having 1 to 6 carbon atoms, an alicyclic group having 3 to 10 carbon atoms, and a substituent represented by Formula (G2-1). At least one of R², R⁴, R⁷, R⁹, R¹² and R¹⁴ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G2) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Note that in the structure of the organic compound represented by General Formula (G2), the proportion of carbon atoms forming a bond by the sp³ hybrid orbitals affects the refractive index of the organic compound. That is, an increase in the number of carbon atoms forming a bond by the sp³ hybrid orbitals lowers the refractive index; thus, the outcoupling efficiency of a light-emitting device containing the organic compound can be improved. Furthermore, the number of carbon atoms forming a bond by the sp³ hybrid orbitals is preferably large, in which case heat resistance such as a glass transition temperature is improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, the proportion of carbon atoms forming a bond by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming a bond by the sp³ hybrid orbitals in the total number of carbon atoms in a molecule of the organic compound is preferably higher than or equal to 20% and lower than or equal to 40%.

The organic compound represented by General Formula (G2) is preferably formed of only a six-membered heteroaromatic ring having 1 to 3 nitrogen atoms, a six-membered aromatic ring (i.e., a substituted or unsubstituted phenyl group), and a hydrocarbon group/hydrocarbon groups forming a bond/bonds by the sp³ hybrid orbitals (e.g., an alkyl group or an alicyclic group), that is, the organic compound preferably includes no condensed ring, in which case the refractive index is lowered and the transport property of carriers (electrons) is increased.

A pyridine ring, a pyrimidine ring, a pyrazine ring, a pyridazine ring, or a triazine ring can be used as A in the organic compound represented by General Formula (G2). In the case where the organic compound represented by General Formula (G2) is used for a light-emitting layer or a layer in contact with an active layer, a triazine ring, a pyrazine ring, or a pyrimidine ring, which easily injects electrons into these layers and has a high electron-transport property, is preferably used, and a triazine ring is particularly preferable.

The total number of substituents (an alkyl group and an alicyclic group) per molecule in the organic compound represented by General Formula (G2) is preferably greater than or equal to 4 and less than or equal to 10 in consideration of the synthesis cost, further preferably greater than or equal to 6 in order to lower the refractive index. Similarly, using as large substituents (an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, naphthyl group, phenanthryl group, or fluorenyl group can be used as the aromatic hydrocarbon group having 6 to 14 carbon atoms in a ring in the organic compound represented by General Formula (G2). The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the organic compound represented by General Formula (G2), a pyridyl group, a pyrimidinyl group, a pyrazinyl group, a triazinyl group, a quinolyl group, a quinazolinyl group, a quinoxalinyl group, or the like can be used as the heteroaromatic ring group having 3 to 9 carbon atoms in a ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G2). As the alicyclic group having 3 to 10 carbon atoms, a cyclopropyl group, a cyclohexyl group, a cyclodecanyl group, a bicyclooctyl group, an adamantyl group, or the like can be used.

The substituents are preferably bonded at the meta-positions of the phenyl groups like R², R⁴, R⁷, R⁹, R¹², and R¹⁴, in which case the film density is decreased and the refractive index can be lowered.

At least two of R², R⁴, R⁷, R⁹, R¹², and R¹⁴ are preferably hydrogen because the steric hindrance around A can be reduced, i.e., the steric hindrance between A in which the LUMO is mainly distributed and the substituents of A can be reduced, in which case the electron-transport property can be high and the driving voltage can be decreased.

Another embodiment of the present invention is an organic compound represented by General Formula (G3).

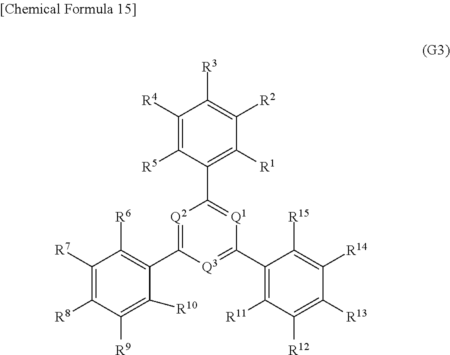

Two or three of Q¹ to Q³ represent N in General Formula (G3). When two of Q¹ to Q³ represent N, the remaining one of Q¹ to Q³ represents CH. At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G3) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Note that in the structure of the organic compound represented by General Formula (G3), the proportion of carbon atoms forming a bond by the sp³ hybrid orbitals affects the refractive index of the organic compound. That is, an increase in the number of carbon atoms forming a bond by the sp³ hybrid orbitals lowers the refractive index; thus, the outcoupling efficiency of a light-emitting device containing the organic compound can be improved. Furthermore, the number of carbon atoms forming a bond by the sp³ hybrid orbitals is preferably large, in which case heat resistance such as a glass transition temperature is improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, the proportion of carbon atoms forming a bond by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming a bond by the sp³ hybrid orbitals in the total number of carbon atoms in a molecule of the organic compound is preferably higher than or equal to 20% and lower than or equal to 40%.

The organic compound represented by General Formula (G3) is preferably formed of only a six-membered heteroaromatic ring including Q¹ to Q³, a six-membered aromatic ring (i.e., a substituted or unsubstituted phenyl group), and a hydrocarbon group/hydrocarbon groups forming a bond/bonds by the sp³ hybrid orbitals (e.g., an alkyl group or an alicyclic group), that is, the organic compound preferably includes no condensed ring, in which case the refractive index is lowered and the transport property of carriers (electrons) is increased.

A pyridine ring or a triazine ring can be used as the six-membered ring including Q¹ to Q³ in the organic compound represented by General Formula (G3). In the case where the organic compound represented by General Formula (G3) is used for a light-emitting layer or a layer in contact with an active layer, a triazine ring, a pyrazine ring, or a pyrimidine ring, which easily injects electrons into these layers and has a high electron-transport property, is preferably used, and a triazine ring is particularly preferable.

The total number of substituents (an alkyl group and an alicyclic group) per molecule in the organic compound represented by General Formula (G3) is preferably greater than or equal to 4 and less than or equal to 10 in consideration of the synthesis cost, further preferably greater than or equal to 6 in order to lower the refractive index. Similarly, using as large substituents (an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, naphthyl group, phenanthryl group, or fluorenyl group can be used as the aromatic hydrocarbon group having 6 to 14 carbon atoms in a ring in the organic compound represented by General Formula (G3). The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the organic compound represented by General Formula (G3), a pyridyl group, a pyrimidinyl group, a pyrazinyl group, a triazinyl group, a quinolyl group, a quinazolinyl group, a quinoxalinyl group, or the like can be used as the heteroaromatic ring group having 3 to 9 carbon atoms in a ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G3). As the alicyclic group having 3 to 10 carbon atoms, a cyclopropyl group, a cyclohexyl group, a cyclodecanyl group, a bicyclooctyl group, an adamantyl group, or the like can be used.

Another embodiment of the present invention is an organic compound represented by General Formula (G4).

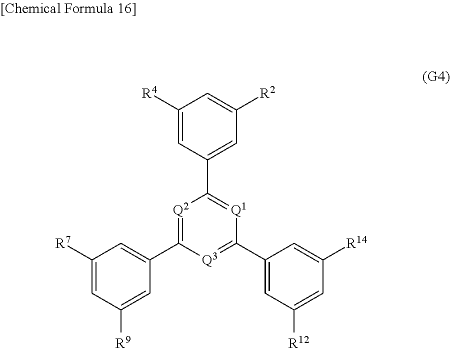

Two or three of Q¹ to Q³ represent N in General Formula (G4). When two of Q¹ to Q³ represent N, the remaining one of Q¹ to Q³ represents CH. At least one of R², R⁴, R⁷, R⁹, R¹², and R¹⁴ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G4) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Note that in the structure of the organic compound represented by General Formula (G4), the proportion of carbon atoms forming a bond by the sp³ hybrid orbitals affects the refractive index of the organic compound. That is, an increase in the number of carbon atoms forming a bond by the sp³ hybrid orbitals lowers the refractive index; thus, the outcoupling efficiency of a light-emitting device containing the organic compound can be improved. Furthermore, the number of carbon atoms forming a bond by the sp³ hybrid orbitals is preferably large, in which case heat resistance such as a glass transition temperature is improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, the proportion of carbon atoms forming a bond by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming a bond by the sp³ hybrid orbitals in the total number of carbon atoms in a molecule of the organic compound is preferably higher than or equal to 20% and lower than or equal to 40%.

The organic compound represented by General Formula (G4) is preferably formed of only a six-membered heteroaromatic ring including Q¹ to Q³, a six-membered aromatic ring (i.e., a substituted or unsubstituted phenyl group), and a hydrocarbon group/hydrocarbon groups forming a bond/bonds by the sp³ hybrid orbitals (e.g., an alkyl group or an alicyclic group), that is, the organic compound preferably includes no condensed ring, in which case the refractive index is lowered and the transport property of carriers (electrons) is increased.

A pyridine ring or a triazine ring can be used as the six-membered ring including Q¹ to Q³ in the organic compound represented by General Formula (G4). In the case where the organic compound represented by General Formula (G4) is used for a light-emitting layer or a layer in contact with an active layer, a triazine ring, a pyrazine ring, or a pyrimidine ring, which easily injects electrons into these layers and has a high electron-transport property, is preferably used, and a triazine ring is particularly preferable.

The total number of substituents (an alkyl group and an alicyclic group) per molecule in the organic compound represented by General Formula (G4) is preferably greater than or equal to 4 and less than or equal to 10 in consideration of the synthesis cost, further preferably greater than or equal to 6 in order to lower the refractive index. Similarly, using as large substituents (an alkyl group and an alicyclic group) as possible effectively lowers the refractive index even when the number of substituents is small, and the number of carbon atoms in the alkyl group is preferably greater than or equal to 4 in consideration of the synthesis cost. The number of carbon atoms in the alicyclic group is preferably greater than or equal to 6.

A substituted or unsubstituted phenyl group, naphthyl group, phenanthryl group, or fluorenyl group can be used as the aromatic hydrocarbon group having 6 to 14 carbon atoms in a ring in the organic compound represented by General Formula (G4). The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the organic compound represented by General Formula (G4), a pyridyl group, a pyrimidinyl group, a pyrazinyl group, a triazinyl group, a quinolyl group, a quinazolinyl group, a quinoxalinyl group, or the like can be used as the heteroaromatic ring group having 3 to 9 carbon atoms in a ring.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the organic compound represented by General Formula (G4). As the alicyclic group having 3 to 10 carbon atoms, a cyclopropyl group, a cyclohexyl group, a cyclodecanyl group, a bicyclooctyl group, an adamantyl group, or the like can be used.

In the above structures, the phenyl group having a substituent in each of the organic compounds represented by General Formula (G1), General Formula (G2), General Formula (G3), and General Formula (G4) is preferably represented by General Formula (G1-2).

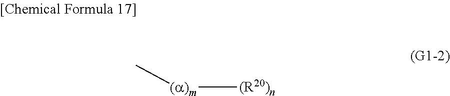

In General Formula (G1-2), a represents a substituted or unsubstituted phenylene group. R²⁰ represents an alkyl group having 1 to 6 carbon atoms, an alicyclic group having 3 to 10 carbon atoms, a substituted or unsubstituted aromatic hydrocarbon group having 6 to 14 carbon atoms in a ring, or a substituted or unsubstituted heteroaromatic ring group having 3 to 9 carbon atoms in a ring. In addition, m and n each represent 1 or 2. In the case where m is 2, a plurality of a may be the same or different from each other. In the case where n is 2, a plurality of R²⁰ may be the same or different from each other.

In the above structure, the phenyl group having a substituent that is represented by General Formula (G1-2) is preferably used as one or both of R² and R⁴ in each of the organic compounds represented by General Formula (G1), General Formula (G2), General Formula (G3), and General Formula (G4). Note that in the case where the group represented by General Formula (G1-2) is used as both of R² and R⁴, the two groups represented by General Formula (G1-2) may be the same or different from each other.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the phenyl group having a substituent that is represented by General Formula (G1-2). As the alicyclic group having 3 to 10 carbon atoms, a cyclopropyl group, a cyclohexyl group, a cyclodecanyl group, a bicyclooctyl group, an adamantyl group, or the like can be used.

A substituted or unsubstituted phenyl group, naphthyl group, phenanthryl group, or fluorenyl group can be used as the aromatic hydrocarbon group having 6 to 14 carbon atoms in a ring in the phenyl group having a substituent that is represented by General Formula (G1-2). The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the above structures, the phenyl group having a substituent in each of the organic compounds represented by General Formula (G1), General Formula (G2), General Formula (G3), and General Formula (G4) is preferably represented by General Formula (G1-3).

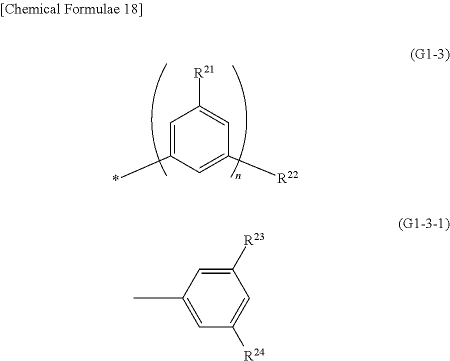

In General Formula (G1-3), R²¹ represents any one of hydrogen, an alkyl group having 1 to 6 carbon atoms, an alicyclic group having 3 to 10 carbon atoms, and a substituent represented by General Formula (G1-3-1). In addition, R²² represents the substituent represented by General Formula (G1-3-1). In General Formula (G1-3-1), R²³ and R²⁴ each represent any one of hydrogen, an alkyl group having 1 to 6 carbon atoms, and an alicyclic group having 3 to 10 carbon atoms, and at least one of R²³ and R²⁴ represents an alkyl group having 1 to 6 carbon atoms or an alicyclic group having 3 to 10 carbon atoms. Note that both of R²³ and R²⁴ preferably represent an alkyl group having 1 to 6 carbon atoms or an alicyclic group having 3 to 10 carbon atoms. Furthermore, n represents 0 to 2. In the case where n is 2, a plurality of R²¹ may be the same or different from each other. In the case where n is 0, at least one of R²³ and R²⁴ represents an alkyl group having 1 to 6 carbon atoms or an alicyclic group having 3 to 10 carbon atoms.

The phenyl group having a substituent that is represented by General Formula (G1-3) is preferably used as one or both of R² and R⁴ in each of General Formulae (G1) to (G4). Note that in the case where the group represented by General Formula (G1-3) is used as both of R² and R⁴, the two groups represented by General Formula (G1-3) may be the same or different from each other.

A methyl group, an ethyl group, a propyl group, an isopropyl group, a butyl group, an isobutyl group, a tert-butyl group, a pentyl group, a hexyl group, or the like can be used as the alkyl group having 1 to 6 carbon atoms in the phenyl group having a substituent that is represented by General Formula (G1-3). As the alicyclic group having 3 to 10 carbon atoms, a cyclopropyl group, a cyclohexyl group, a cyclodecanyl group, a bicyclooctyl group, an adamantyl group, or the like can be used.

A substituted or unsubstituted phenyl group, naphthyl group, phenanthryl group, or fluorenyl group can be used as the aromatic hydrocarbon group having 6 to 14 carbon atoms in a ring in the phenyl group having a substituent that is represented by General Formula (G1-3). The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the above structures, when the aromatic hydrocarbon group having 6 to 14 carbon atoms in a ring or the heteroaromatic ring group having 3 to 9 carbon atoms in a ring in each of the organic compounds represented by General Formula (G1), General Formula (G2), General Formula (G3), and General Formula (G4) has a substituent, the substituent is preferably any one of an alkyl group having 1 to 6 carbon atoms, an alicyclic group having 3 to 10 carbon atoms, an unsubstituted aromatic hydrocarbon group having 6 to 14 carbon atoms, and an aromatic hydrocarbon group having 6 to 14 carbon atoms in a ring, to which an alkyl group having 1 to 6 carbon atoms or an alicyclic group having 3 to 10 carbon atoms is bonded. As the alkyl group having 1 to 6 carbon atoms, a methyl group, an ethyl group, a propyl group, an isopropyl group, a butyl group, an isobutyl group, a tert-butyl group, a pentyl group, a hexyl group, or the like can be used. As the alicyclic group having 3 to 10 carbon atoms, a cyclopropyl group, a cyclohexyl group, a cyclodecanyl group, a bicyclooctyl group, an adamantyl group, or the like can be used. As the aromatic hydrocarbon group having 6 to 14 carbon atoms, a phenyl group, a naphthyl group, a phenanthryl group, or a fluorenyl group can be used. The phenyl group is particularly preferable because the refractive index can be made low. In addition, the naphthyl group, the phenanthryl group, and the fluorenyl group are preferable because a glass transition temperature can be increased. A branched alkyl group or a branched cycloalkyl group that has 3 to 5 carbon atoms is preferably bonded to the aromatic hydrocarbon group having 6 to 14 carbon atoms in a ring, in which case an effect of suppressing an increase in the refractive index caused by an increase in the glass transition temperature, i.e., an effect of maintaining a low refractive index, can be produced. In order to lower the refractive index, any of an alkyl group having 1 to 6 carbon atoms, an alicyclic group having 3 to 10 carbon atoms, and an aromatic hydrocarbon group having 6 to 14 carbon atoms in a ring, to which an alkyl group having 1 to 6 carbon atoms or an alicyclic group having 3 to 10 carbon atoms is bonded, is preferably bonded to the aromatic hydrocarbon group having 6 to 14 carbon atoms in a ring. For example, a phenyl group to which an alkyl group having 1 to 6 carbon atoms or an alicyclic group having 3 to 10 carbon atoms is bonded, such as a 1,3-di(t-butyl)phenyl group or a 1,3-dicyclohexylphenyl group, is preferable. In addition, a phenyl group bonded with a phenyl group to which an alkyl group having 1 to 6 carbon atoms or an alicyclic group having 3 to 10 carbon atoms is bonded, such as a 3-t-butyl-5-[1,3-di(t-butyl)phenyl]phenyl group or a 3-cyclohexyl-5-[1,3-dicyclohexylphenyl]phenyl group, is preferable. In the case where a condensed ring is used and the number of condensed rings is three or more, one six-membered ring is preferably bonded with the other six-membered rings only at the a-position, c-position, and e-position, in which case the refractive index can be lowered as compared with the case of polyacene. For example, the refractive index of the case of using a phenanthrene ring can be lower than that of the case of using an anthracene ring.

In the above structures, the aromatic hydrocarbon group having 6 to 14 carbon atoms in a ring in the organic compound represented by any of General Formula (G1), General Formula (G2), General Formula (G3), and General Formula (G4) and in the phenyl group having a substituent that is represented by either of General Formula (G1-2) and General Formula (G1-3) is preferably represented by any one of Formulae (ra-1) to (ra-15).

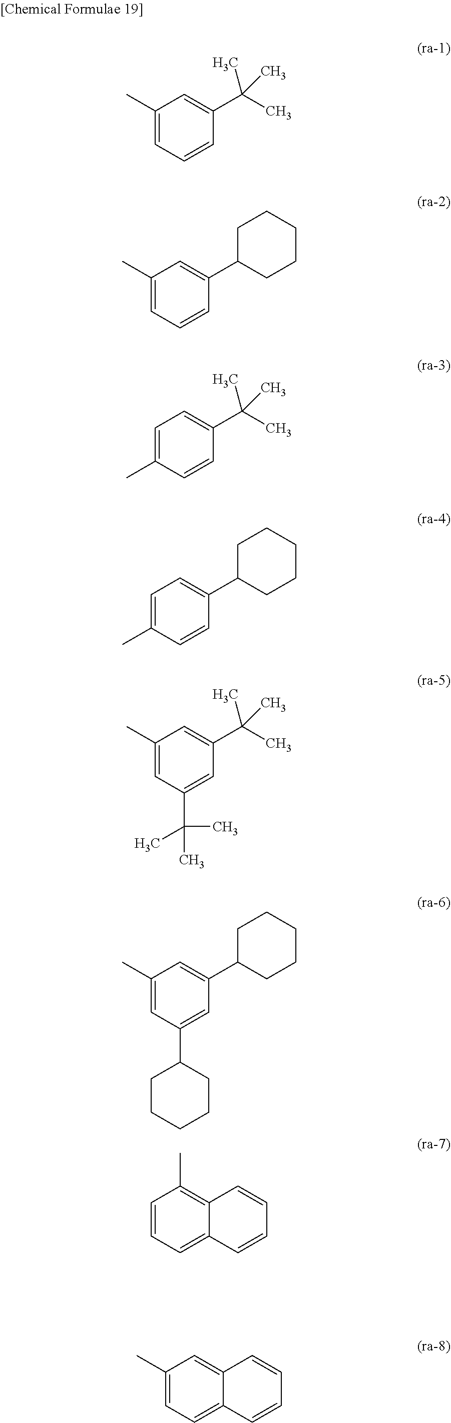

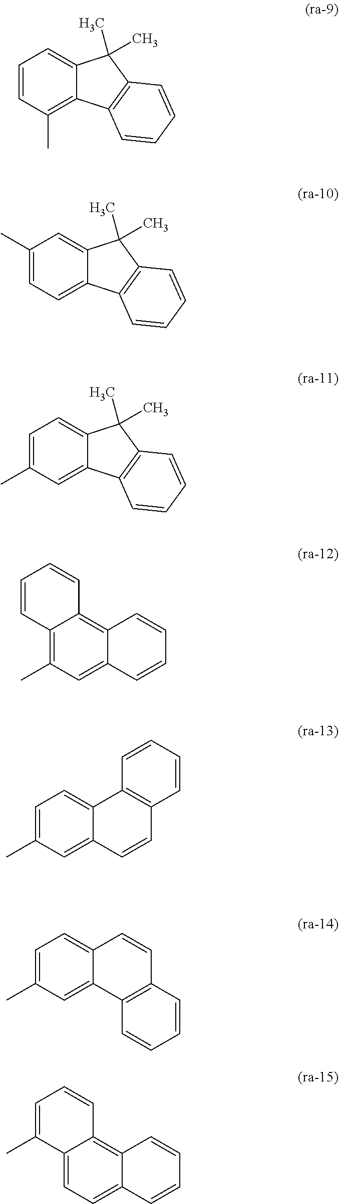

In the above structures, the substituted or unsubstituted pyridyl group in each of the organic compounds represented by General Formula (G1), General Formula (G2), General Formula (G3), and General Formula (G4) is preferably an unsubstituted pyridyl group or a pyridyl group to which one or more methyl groups are bonded.

Another embodiment of the present invention is an organic compound represented by General Formula (G4′).

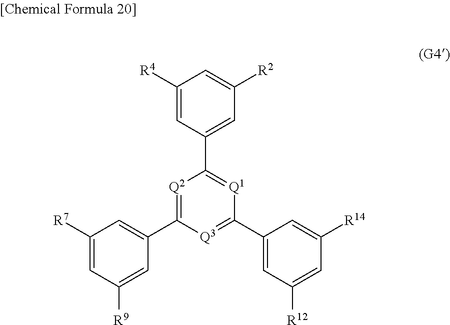

Two or three of Q¹ to Q³ represent N in General Formula (G4′). When two of Q¹ to Q³ represent N, the remaining one of Q¹ to Q³ represents CH. Furthermore, R² is represented by Formula (R²-1) below, and R⁴, R⁷, R⁹, R¹², and R¹⁴ each independently represent any one of Formulae (r-1) to (r-20). Note that in Formula (R²-1), β represents a substituted or unsubstituted phenylene group or a substituted or unsubstituted biphenyldiyl group, R²⁵ represents any one of Formulae (r-1) to (r-20), and n represents 1 or 2. Note that the organic compound represented by General Formula (G4′) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

Note that in the structure of the organic compound represented by General Formula (G4′), the proportion of carbon atoms forming a bond by the sp³ hybrid orbitals affects the refractive index of the organic compound. That is, an increase in the number of carbon atoms forming a bond by the sp³ hybrid orbitals lowers the refractive index; thus, the outcoupling efficiency of a light-emitting device containing the organic compound can be improved. Furthermore, the number of carbon atoms forming a bond by the sp³ hybrid orbitals is preferably large, in which case heat resistance such as a glass transition temperature is improved. However, when the number of carbon atoms forming a bond by the sp³ hybrid orbitals is too large, an overlap of LUMO between adjacent molecules in the organic compound is inhibited and thus the carrier-transport property (e.g., electron-transport and electron-injection properties) is lowered. Hence, the proportion of carbon atoms forming a bond by the sp³ hybrid orbitals in the total number of carbon atoms in a molecule of the organic compound is preferably higher than or equal to 10% and lower than or equal to 60%, further preferably higher than or equal to 20% and lower than or equal to 50%. Moreover, the proportion of carbon atoms forming a bond by the sp³ hybrid orbitals in the total number of carbon atoms in a molecule of the organic compound is preferably higher than or equal to 20% and lower than or equal to 40%.

The organic compound represented by General Formula (G4′) is preferably formed of only a six-membered heteroaromatic ring including Q¹ to Q³, a six-membered aromatic ring (i.e., a substituted or unsubstituted phenyl group), and a hydrocarbon group/hydrocarbon groups forming a bond/bonds by the sp³ hybrid orbitals (e.g., an alkyl group or an alicyclic group), that is, the organic compound preferably includes no condensed ring, in which case the refractive index is lowered and the transport property of carriers (electrons) is increased.

A pyridine ring or a triazine ring can be used as the six-membered ring including Q¹ to Q³ in the organic compound represented by General Formula (G4′). In the case where the organic compound represented by General Formula (G4′) is used for a light-emitting layer or a layer in contact with an active layer, a triazine ring, a pyrazine ring, or a pyrimidine ring, which easily injects electrons into these layers and has a high electron-transport property, is preferably used, and a triazine ring is particularly preferable.

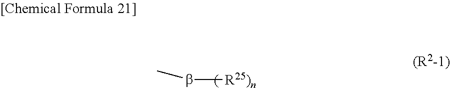

A substituted or unsubstituted phenylene group, biphenylene group, or benzenetriyl group can be used as β in Formula (R²-1). In the case where β has a substituent, an alkyl group having 1 to 6 carbon atoms or an alicyclic group having 3 to 10 carbon atoms can be used as the substituent.

Furthermore, R²⁵ in Formula (R²-1) that represents R² in the organic compound represented by General Formula (G4′) is represented by any one of Formulae (r-1) to (r-20), and β is represented by any one of Formulae (β-1) to (β-14).

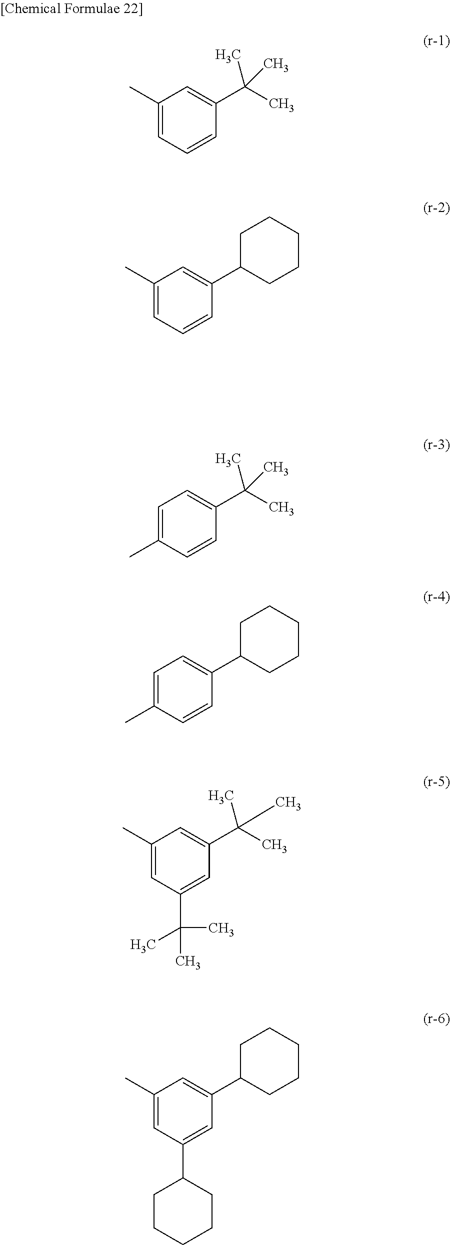

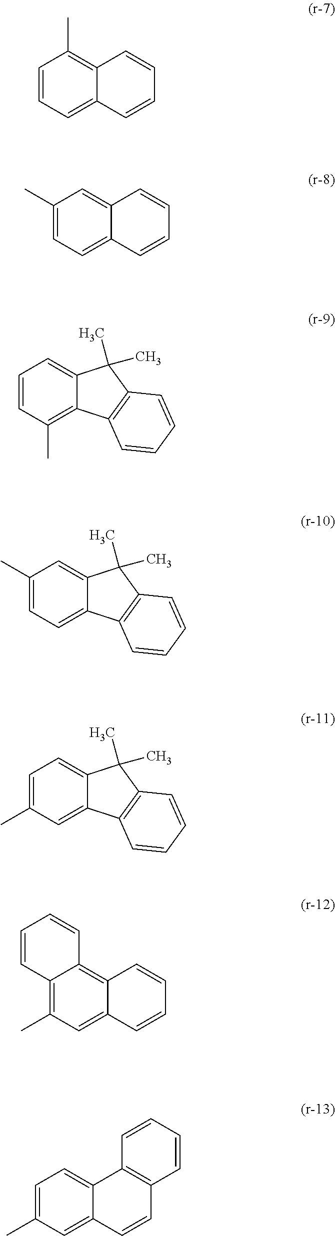

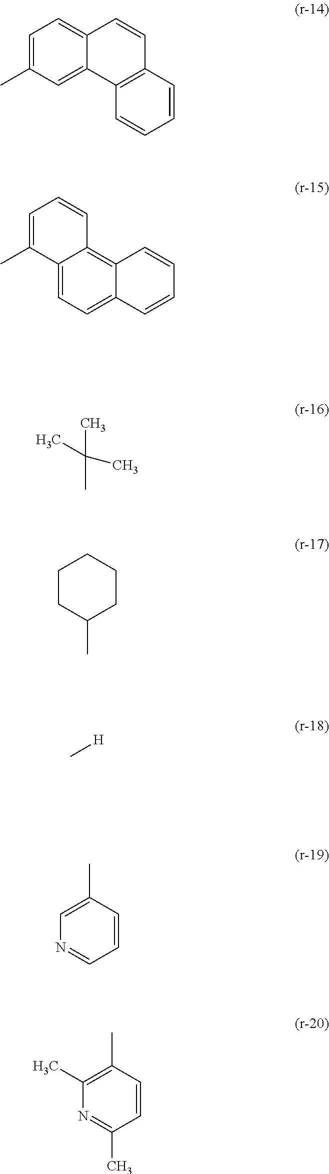

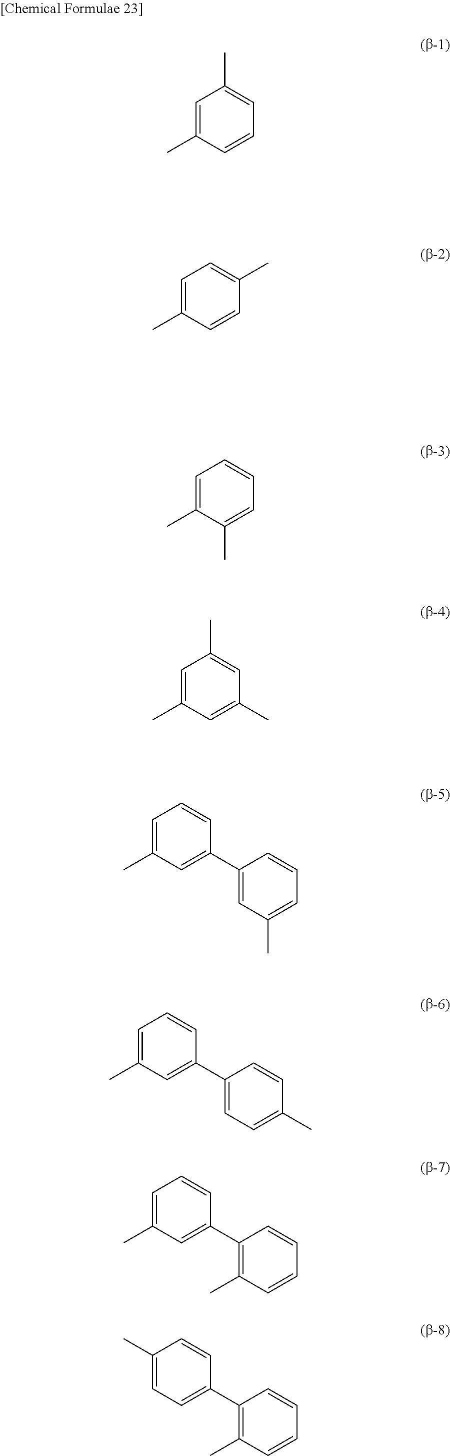

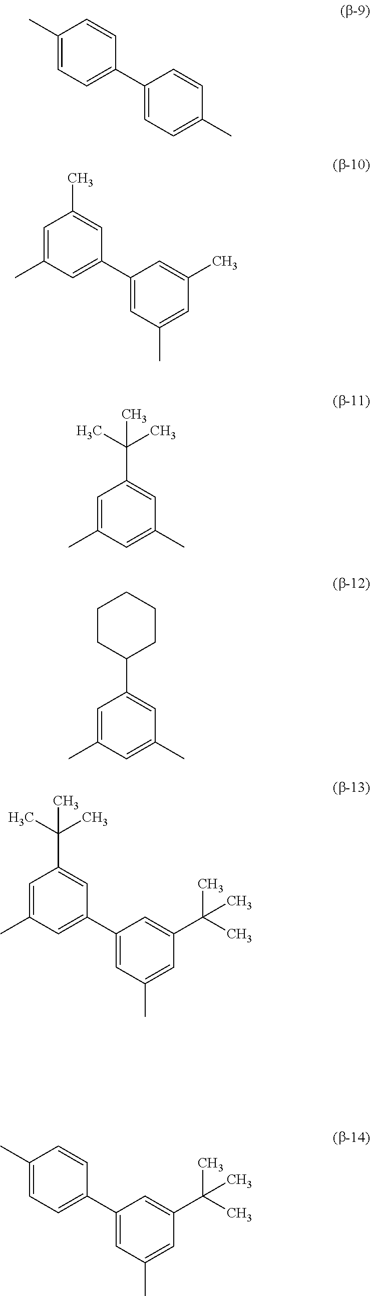

Next, specific examples of the organic compounds of one embodiment of the present invention having the above structures are shown below.

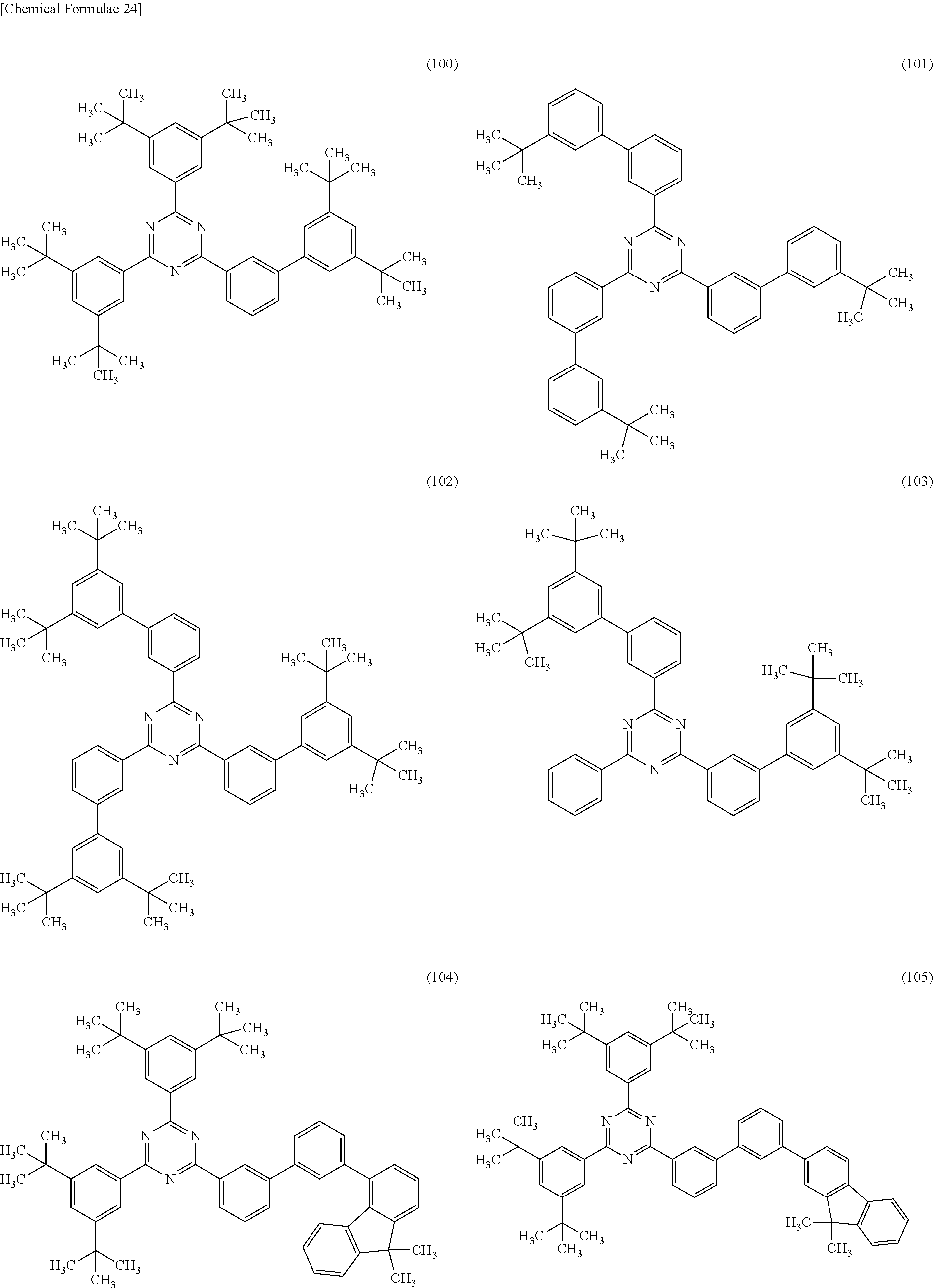

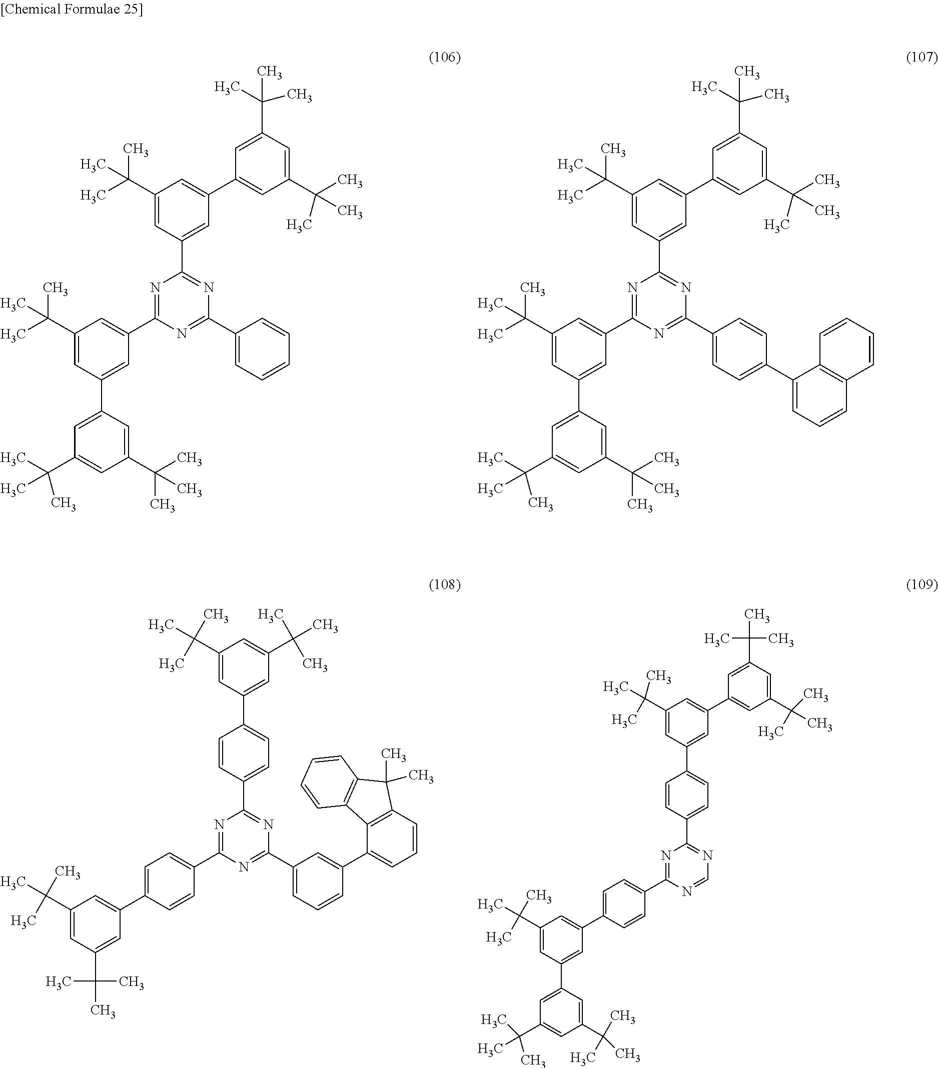

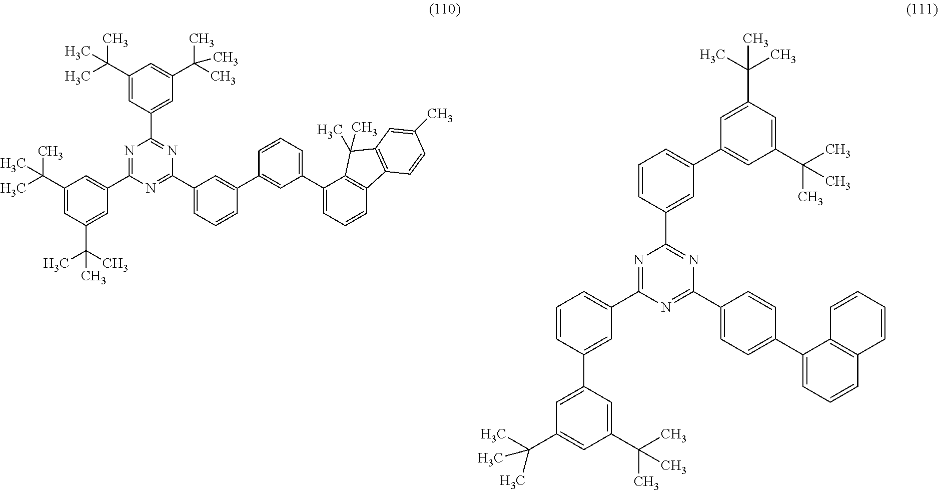 [SMILES: Cc1ccc2c(c1)C(C)(C)c1c(-c3cccc(-c4cccc(-c5nc(-c6cc(C(C)(C)C)cc(C(C)(C)C)c6)nc(-c6cc(C(C)(C)C)cc(C(C)(C)C)c6)n5)c4)c3)cccc1-2 ; CC(C)(C)c1cc(-c2cccc(-c3nc(-c4ccc(-c5cccc6ccccc56)cc4)nc(-c4cccc(-c5cc(C(C)(C)C)cc(C(C)(C)C)c5)c4)n3)c2)cc(C(C)(C)C)c1]

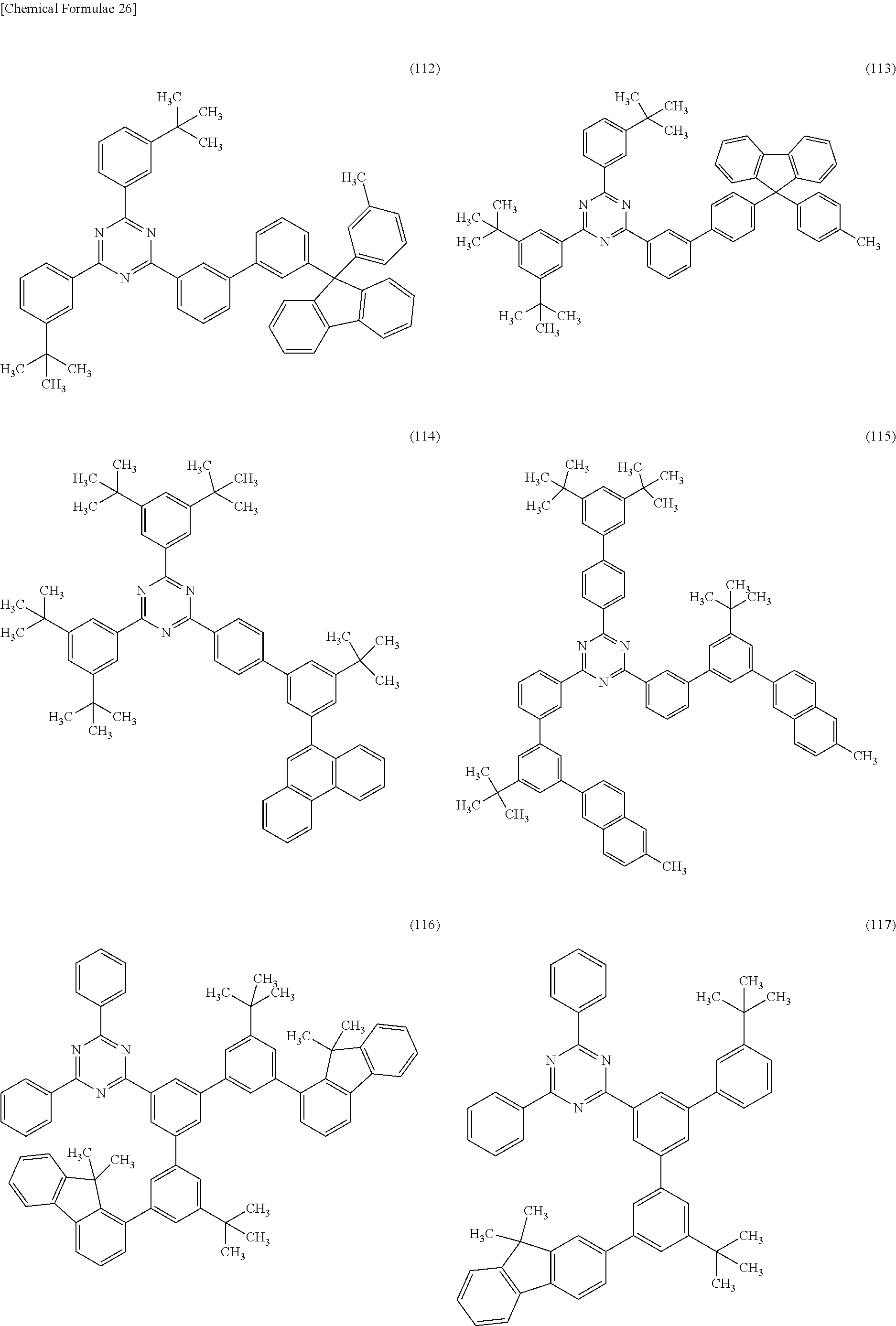

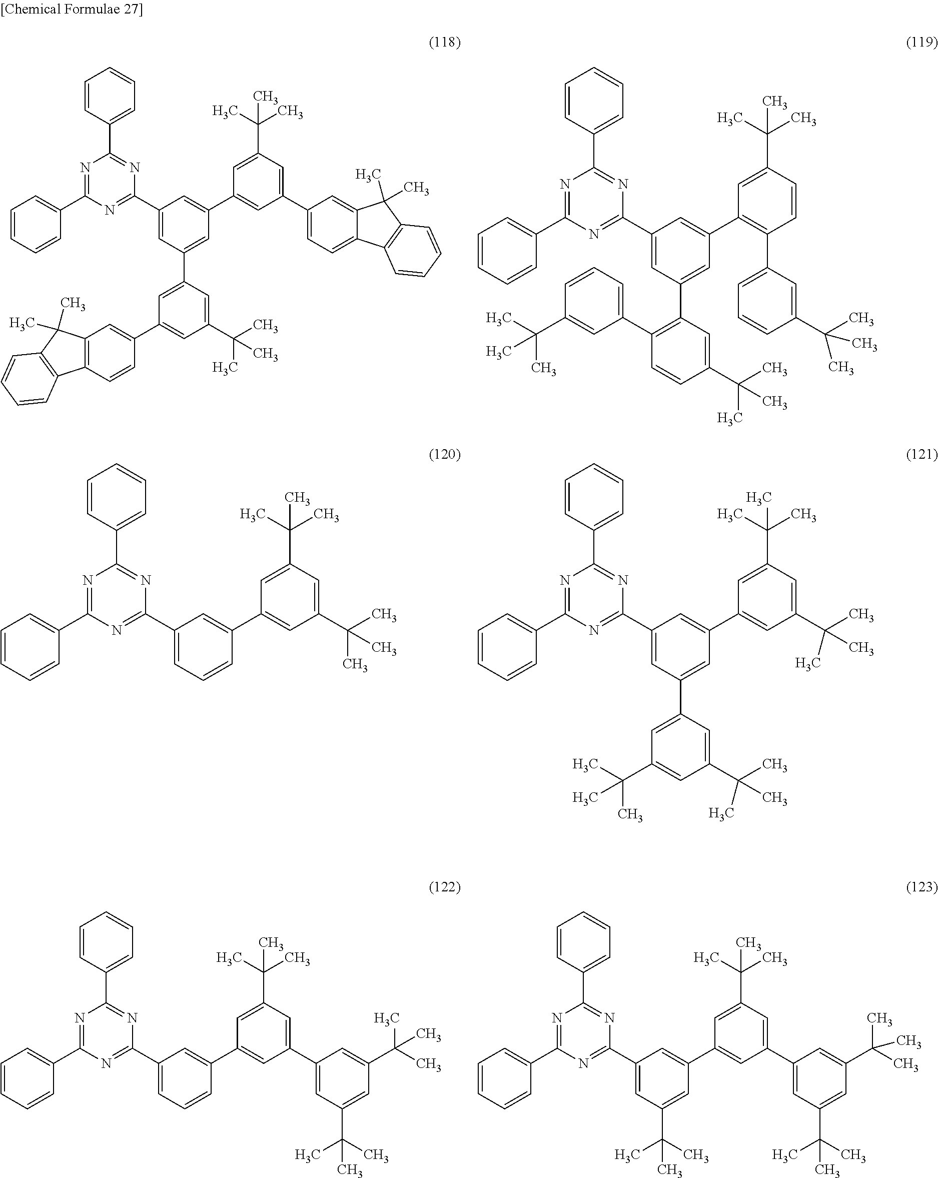

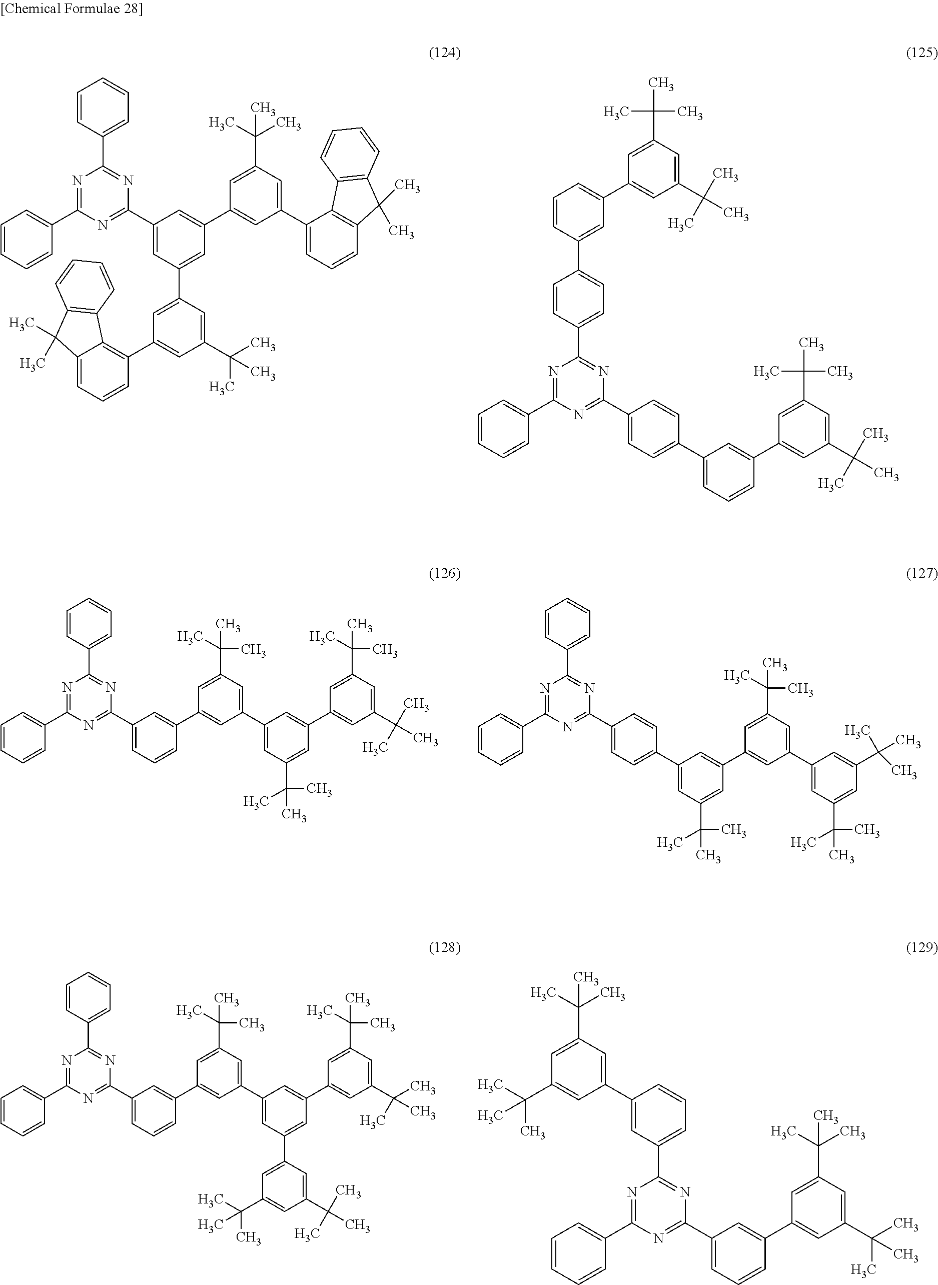

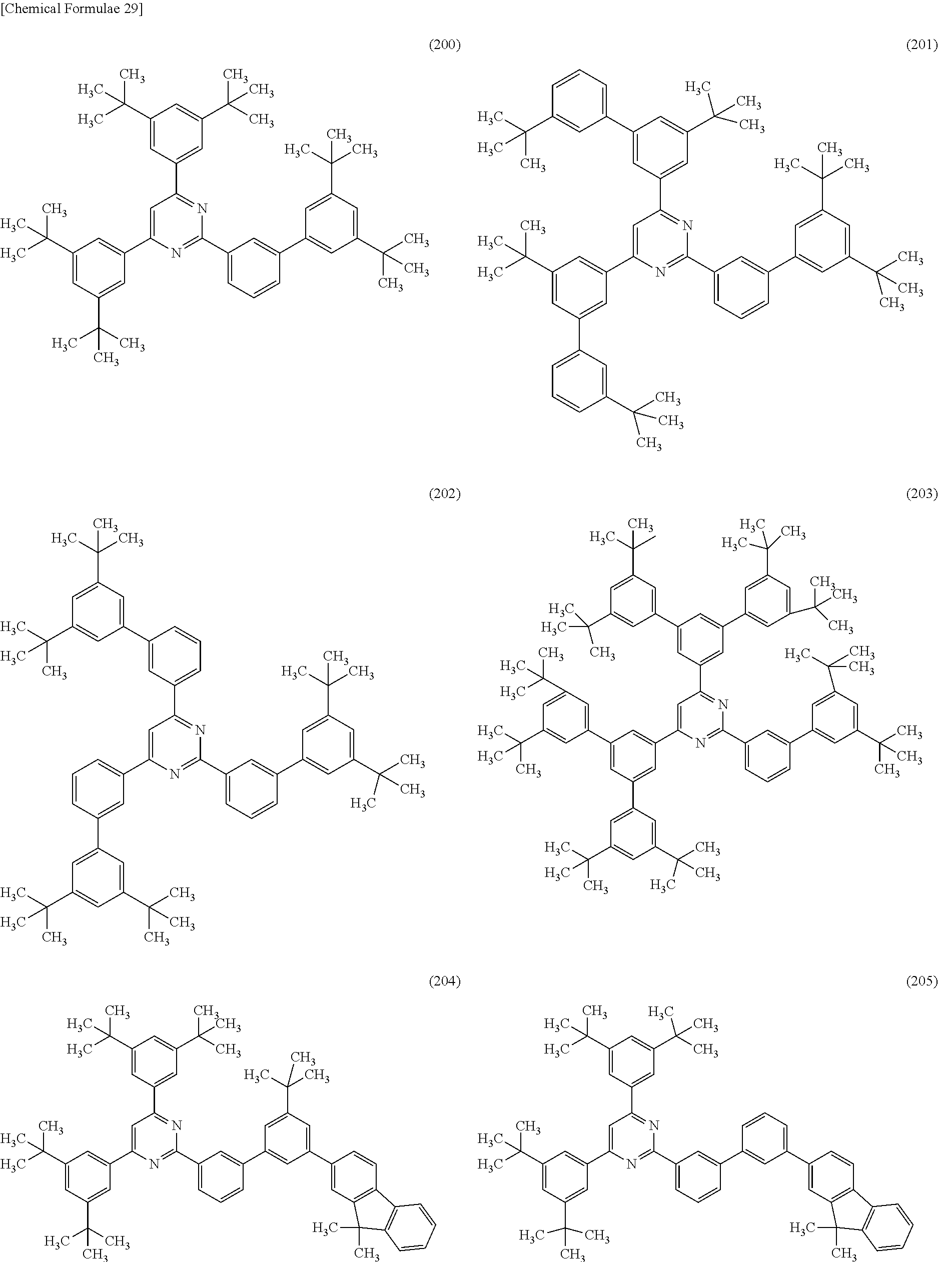

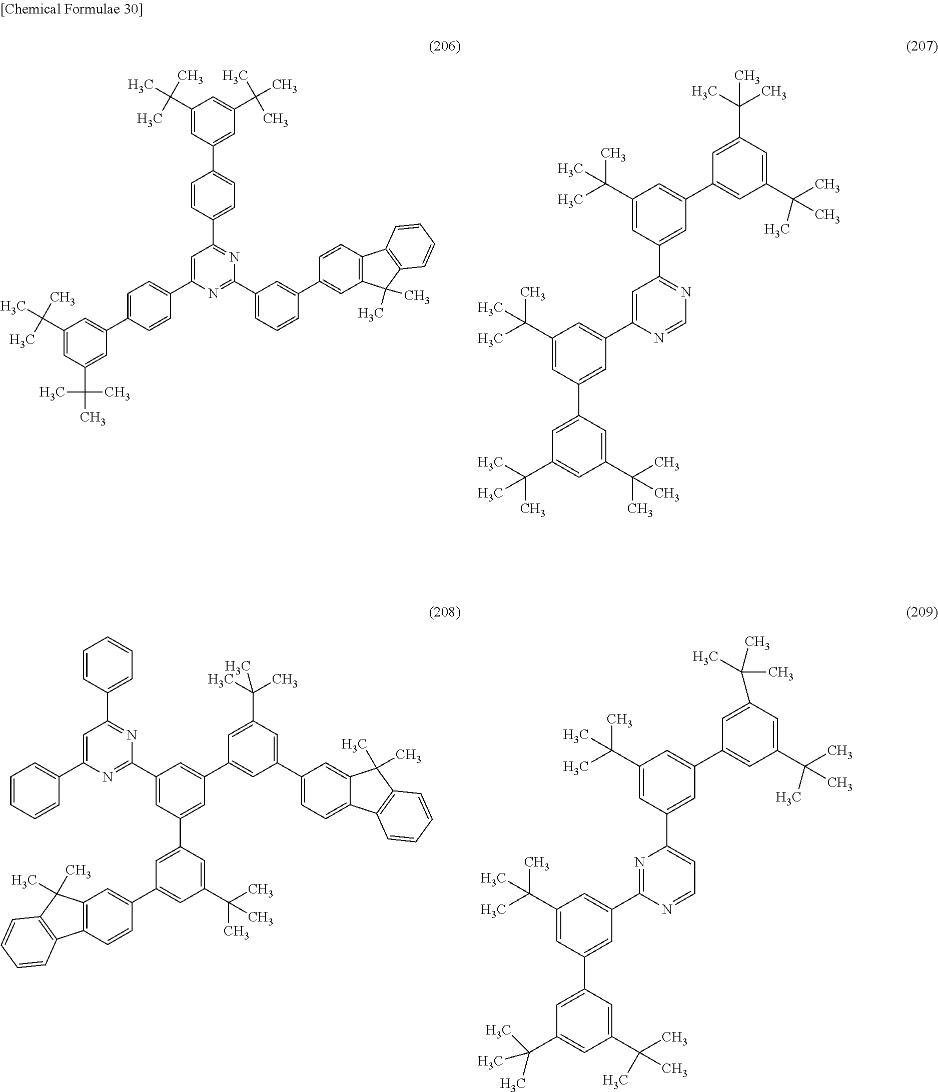

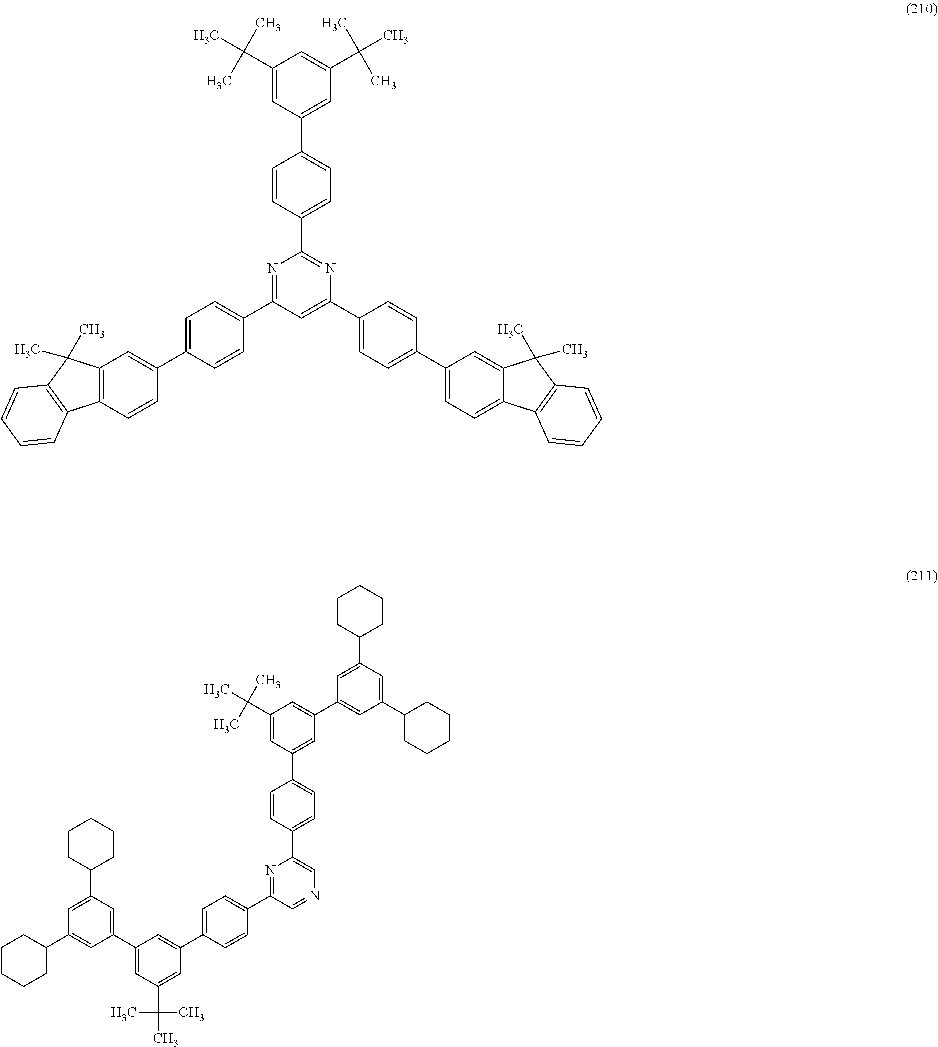 [SMILES: CC(C)(C)c1cc(-c2ccc(-c3nc(-c4ccc(-c5ccc6c(c5)C(C)(C)c5ccccc5-6)cc4)cc(-c4ccc(-c5ccc6c(c5)C(C)(C)c5ccccc5-6)cc4)n3)cc2)cc(C(C)(C)C)c1 ; CC(C)(C)c1cc(-c2ccc(-c3cncc(-c4ccc(-c5cc(-c6cc(C7CCCCC7)cc(C7CCCCC7)c6)cc(C(C)(C)C)c5)cc4)n3)cc2)cc(-c2cc(C3CCCCC3)cc(C3CCCCC3)c2)c1]

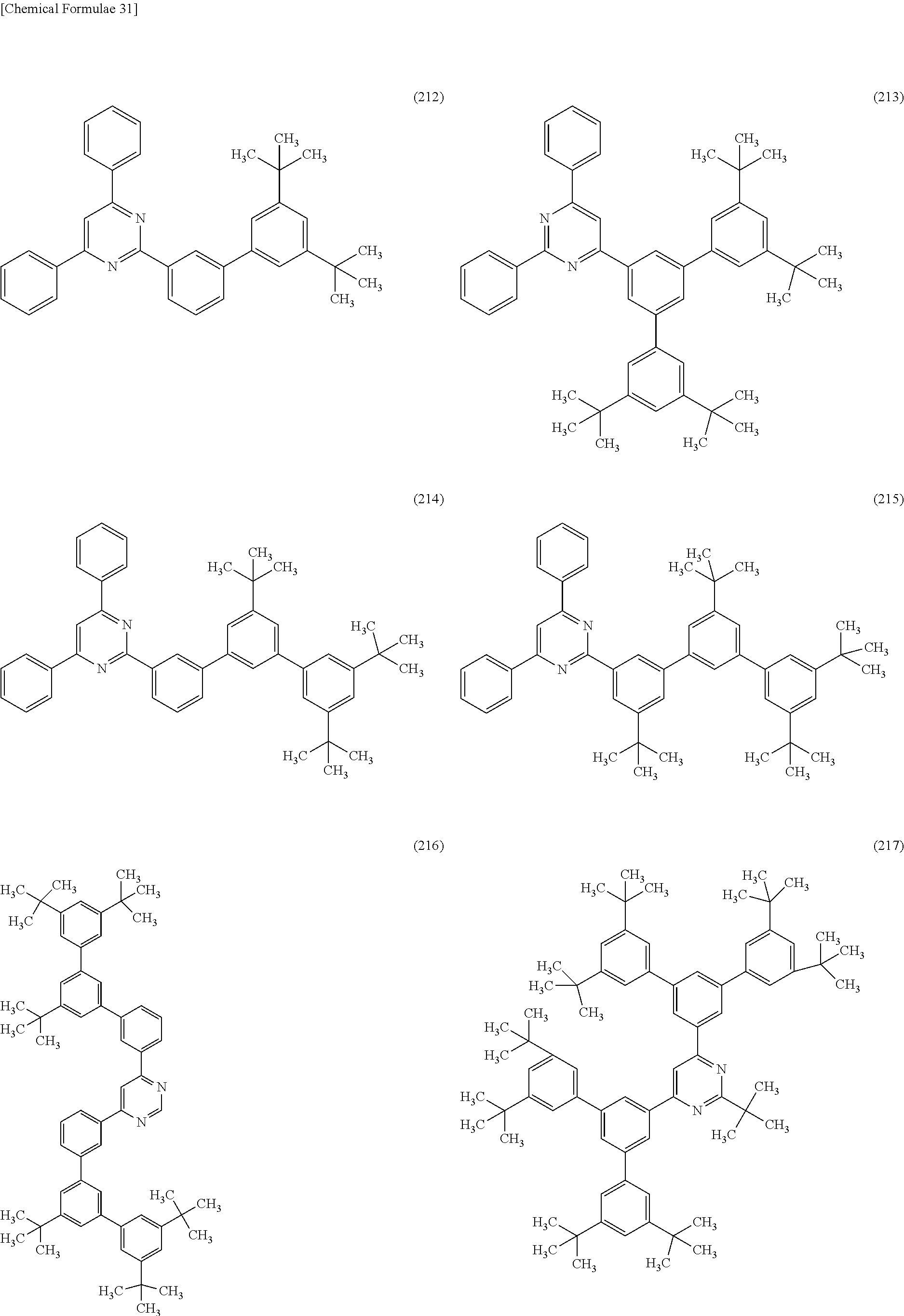

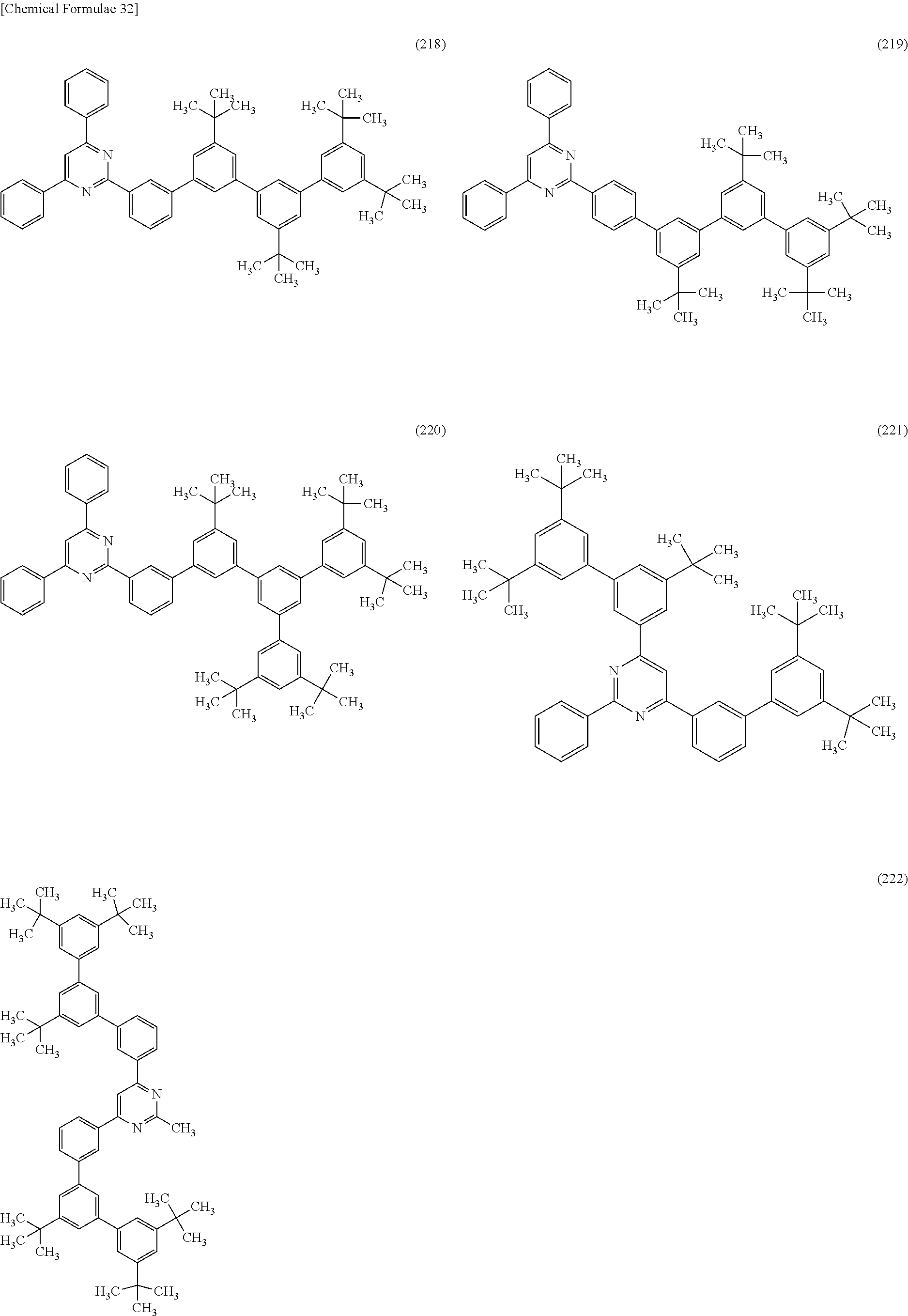

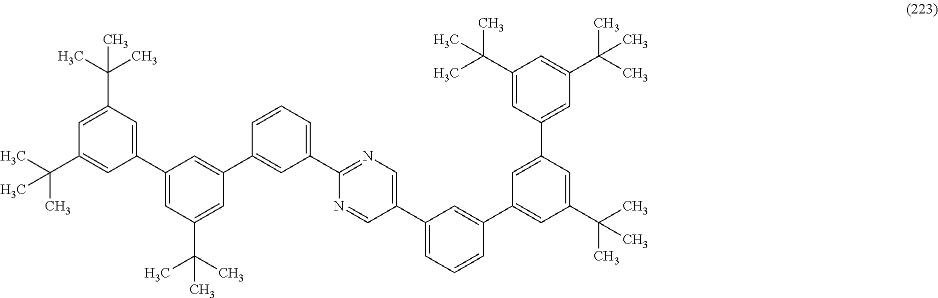 [SMILES: CC(C)(C)c1cc(-c2cccc(-c3cnc(-c4cccc(-c5cc(-c6cc(C(C)(C)C)cc(C(C)(C)C)c6)cc(C(C)(C)C)c5)c4)nc3)c2)cc(-c2cc(C(C)(C)C)cc(C(C)(C)C)c2)c1]

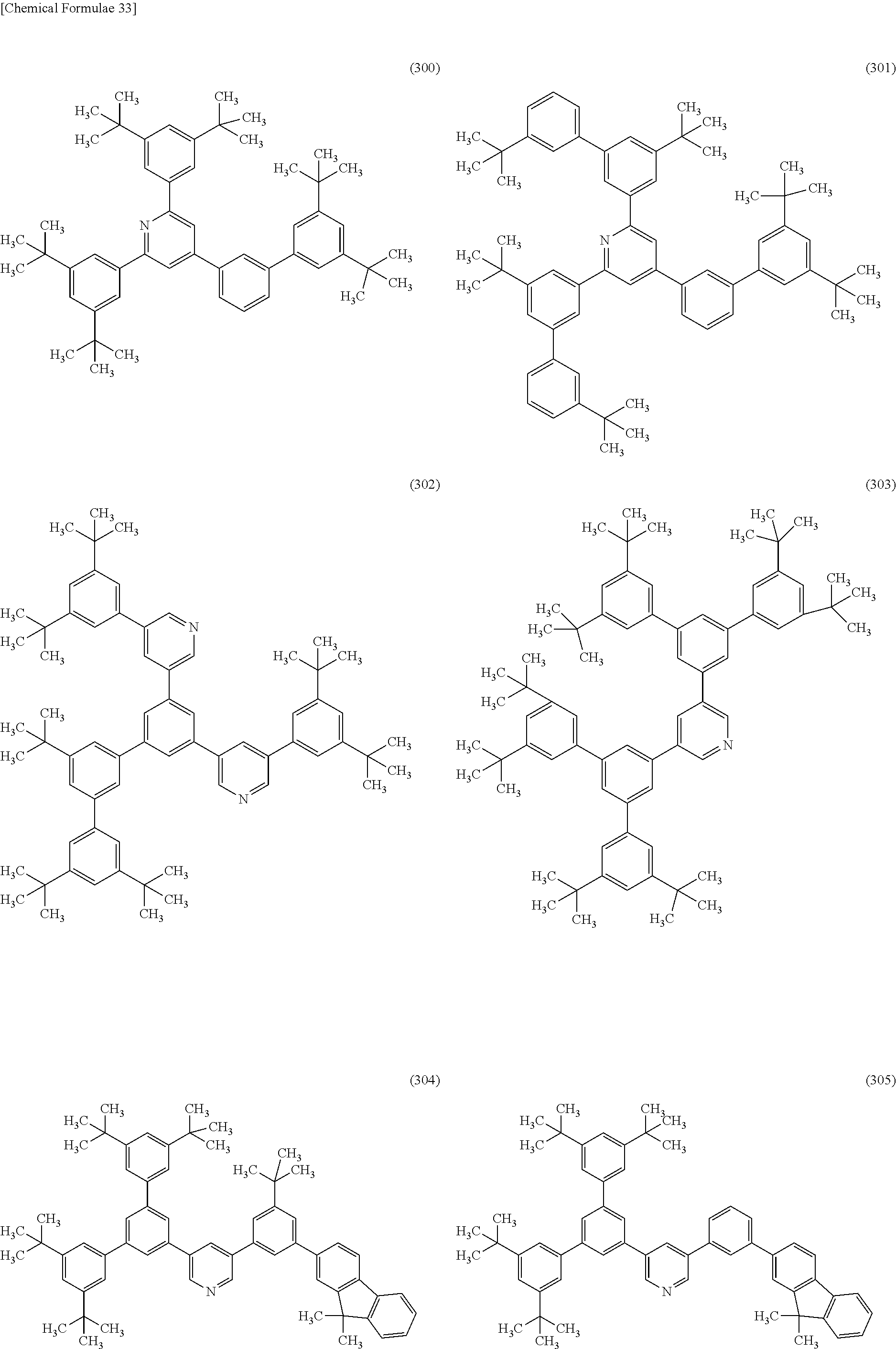

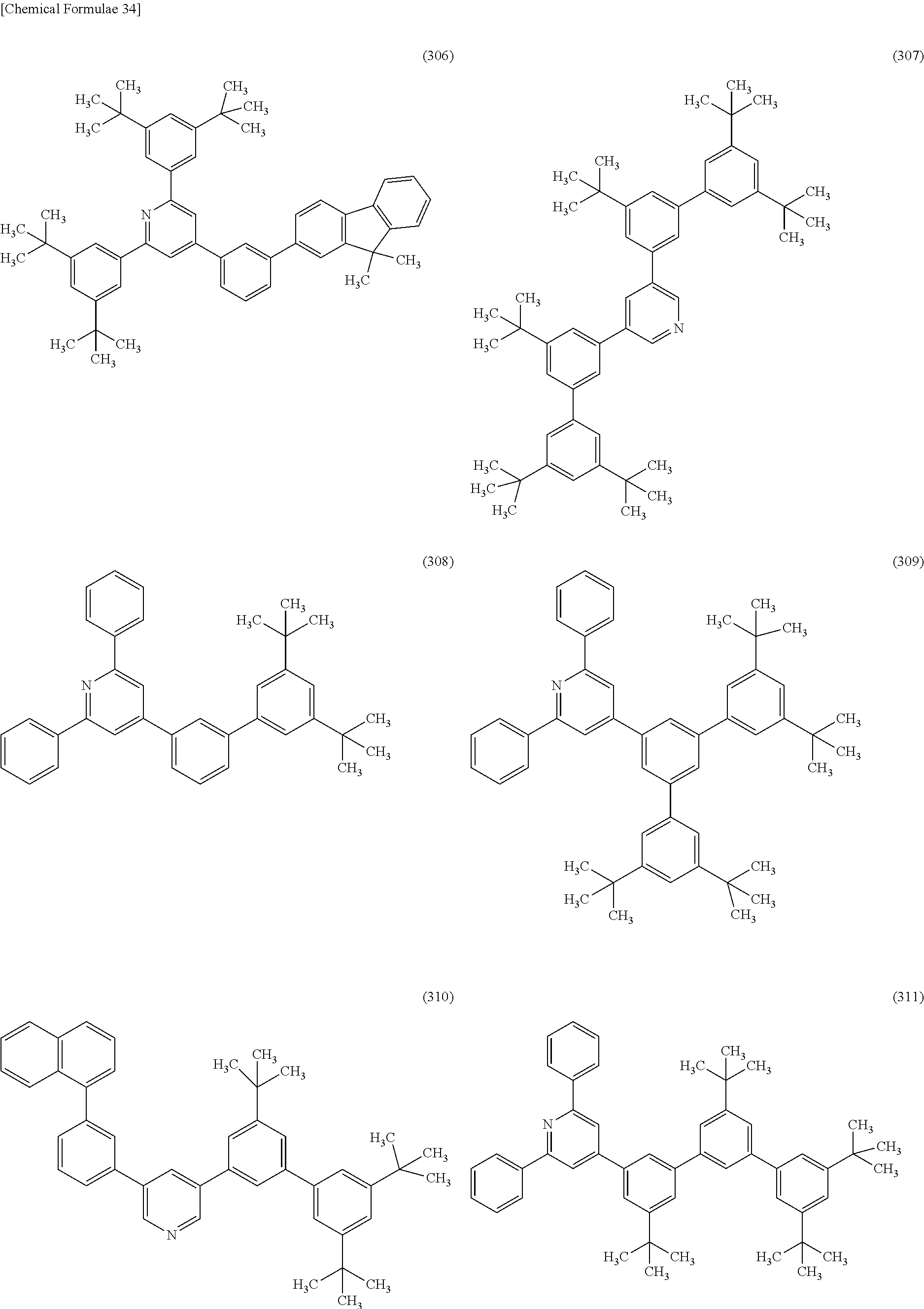

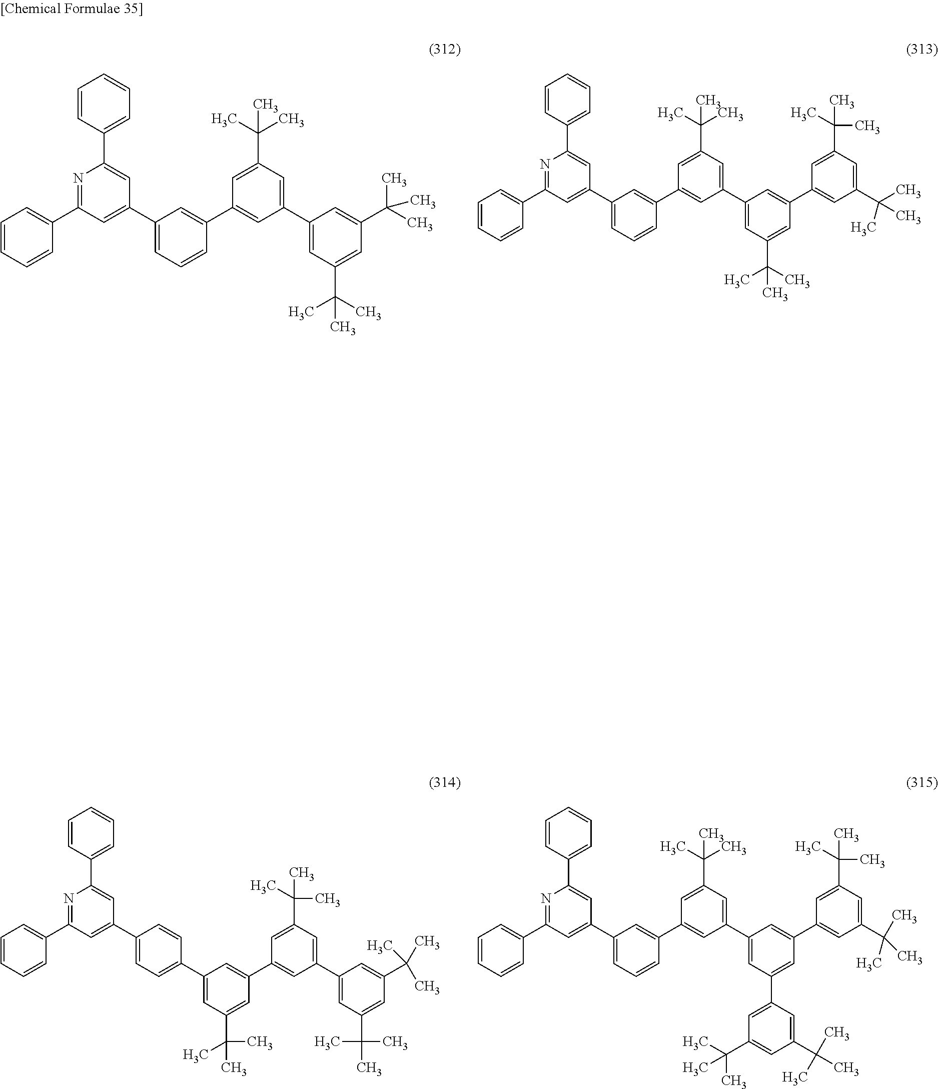 [SMILES: CC(C)(C)c1cc(-c2cccc(-c3cc(-c4ccccc4)nc(-c4ccccc4)c3)c2)cc(-c2cc(C(C)(C)C)cc(C(C)(C)C)c2)c1 ; CC(C)(C)c1cc(-c2cccc(-c3cc(-c4ccccc4)nc(-c4ccccc4)c3)c2)cc(-c2cc(-c3cc(C(C)(C)C)cc(C(C)(C)C)c3)cc(C(C)(C)C)c2)c1 ; CC(C)(C)c1cc(-c2ccc(-c3cc(-c4ccccc4)nc(-c4ccccc4)c3)cc2)cc(-c2cc(-c3cc(C(C)(C)C)cc(C(C)(C)C)c3)cc(C(C)(C)C)c2)c1 ; CC(C)(C)c1cc(-c2cccc(-c3cc(-c4ccccc4)nc(-c4ccccc4)c3)c2)cc(-c2cc(-c3cc(C(C)(C)C)cc(C(C)(C)C)c3)cc(-c3cc(C(C)(C)C)cc(C(C)(C)C)c3)c2)c1]

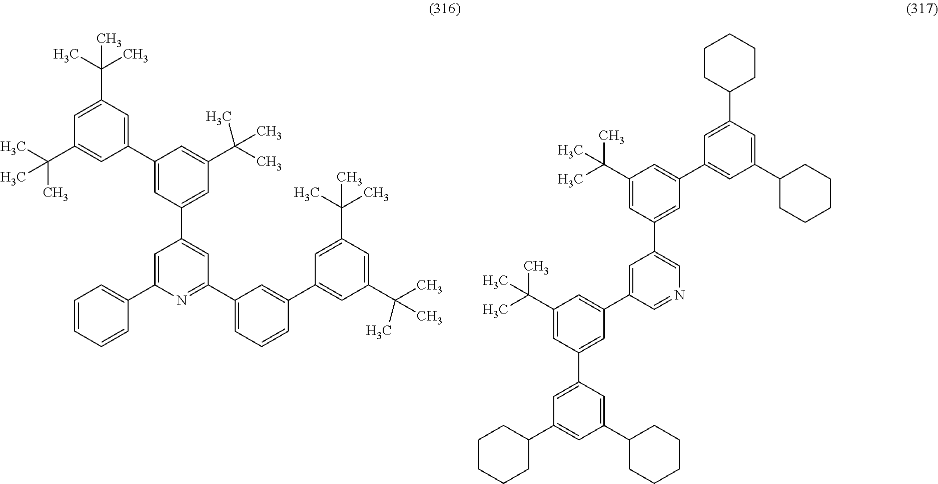 [SMILES: CC(C)(C)c1cc(-c2cc(C(C)(C)C)cc(C(C)(C)C)c2)cc(-c2cc(-c3ccccc3)nc(-c3cccc(-c4cc(C(C)(C)C)cc(C(C)(C)C)c4)c3)c2)c1 ; CC(C)(C)c1cc(-c2cncc(-c3cc(-c4cc(C5CCCCC5)cc(C5CCCCC5)c4)cc(C(C)(C)C)c3)c2)cc(-c2cc(C3CCCCC3)cc(C3CCCCC3)c2)c1]

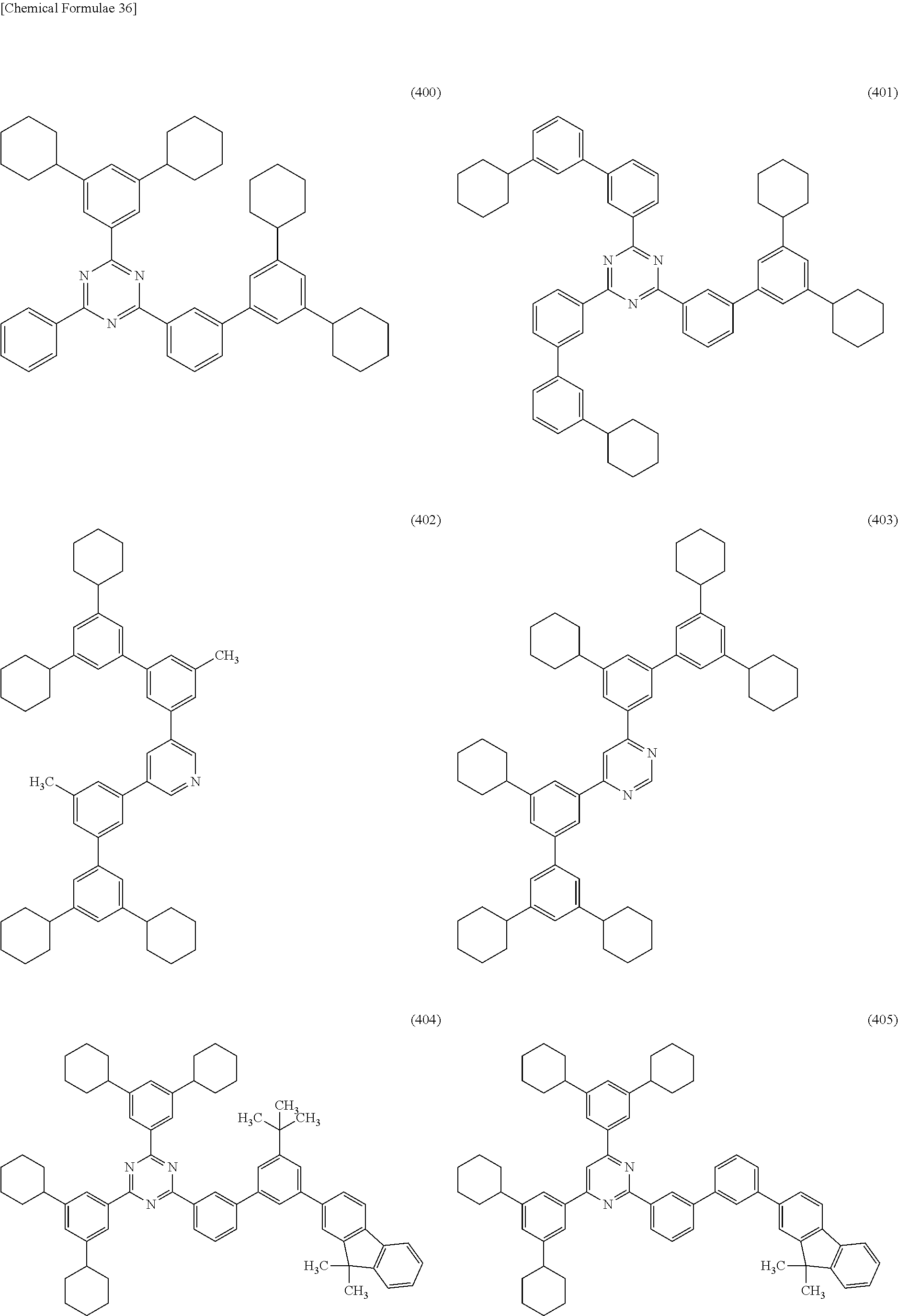

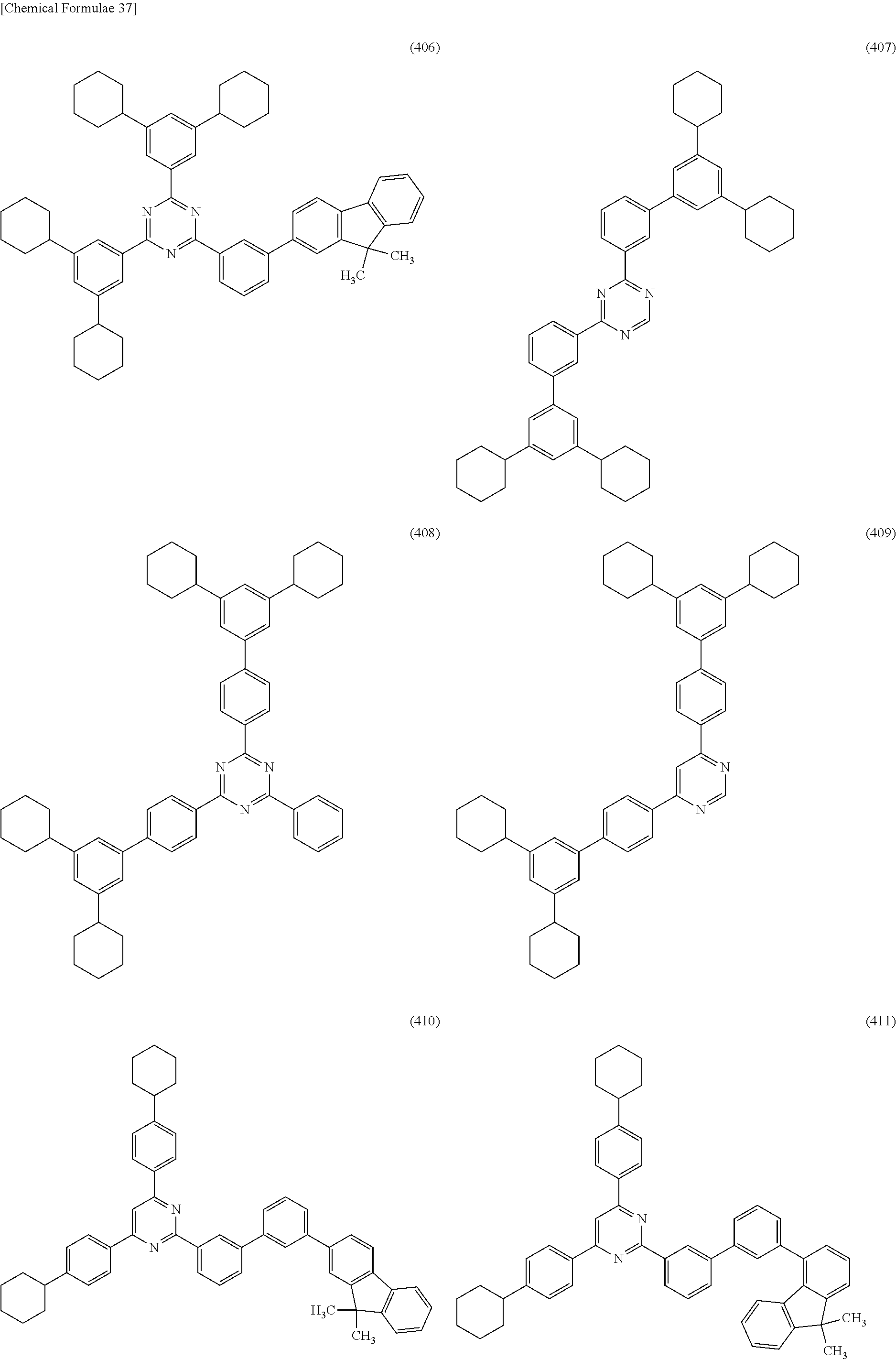

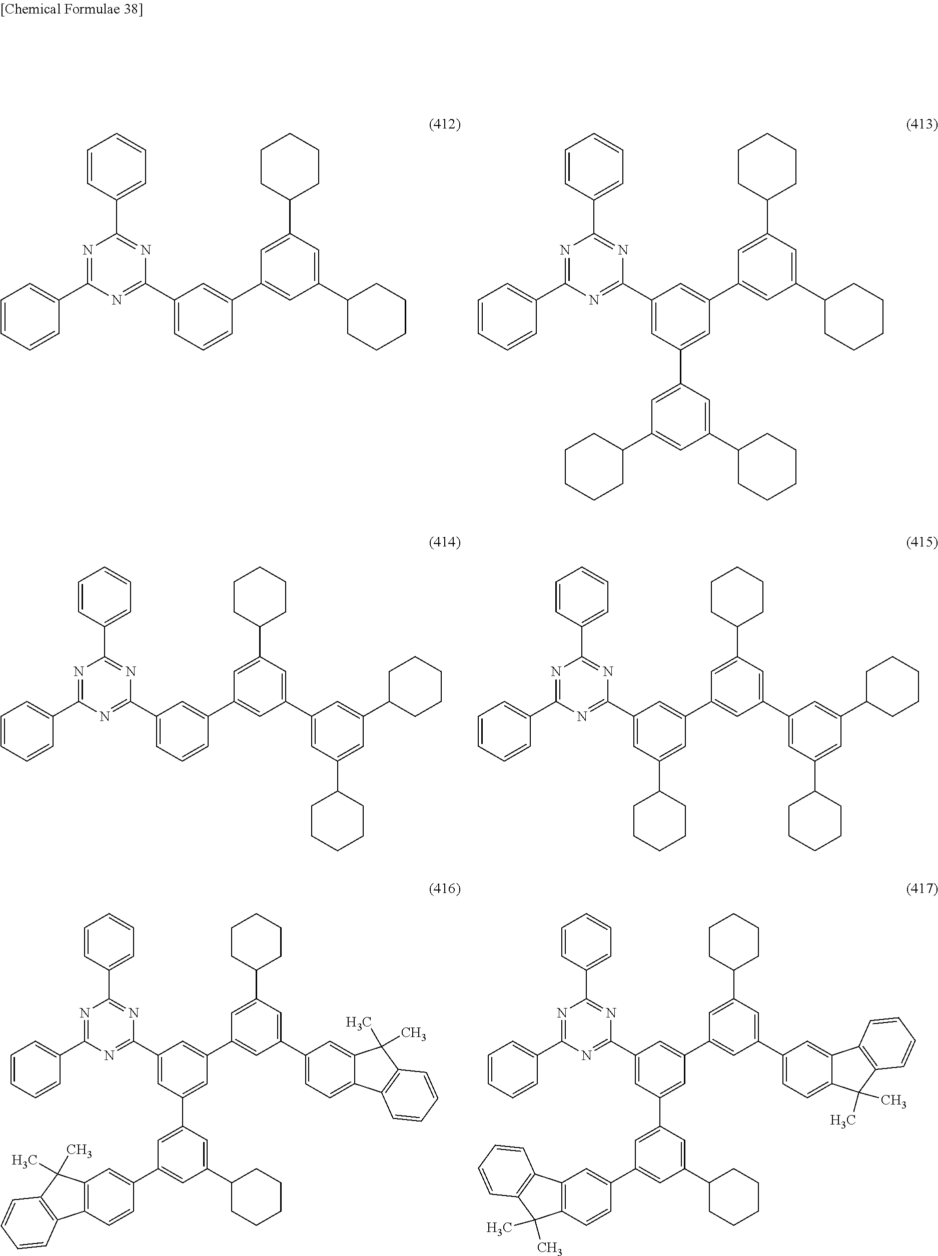

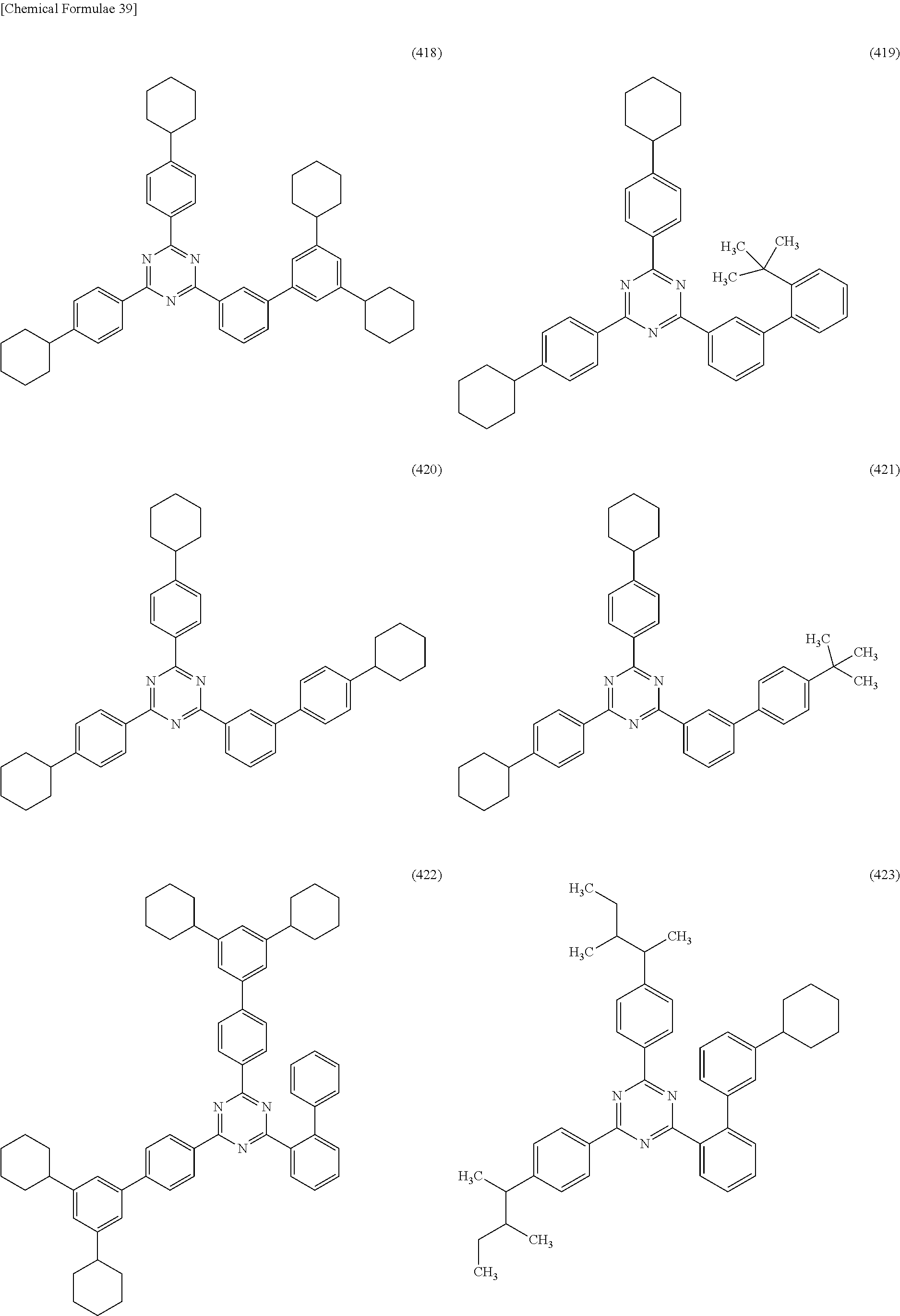

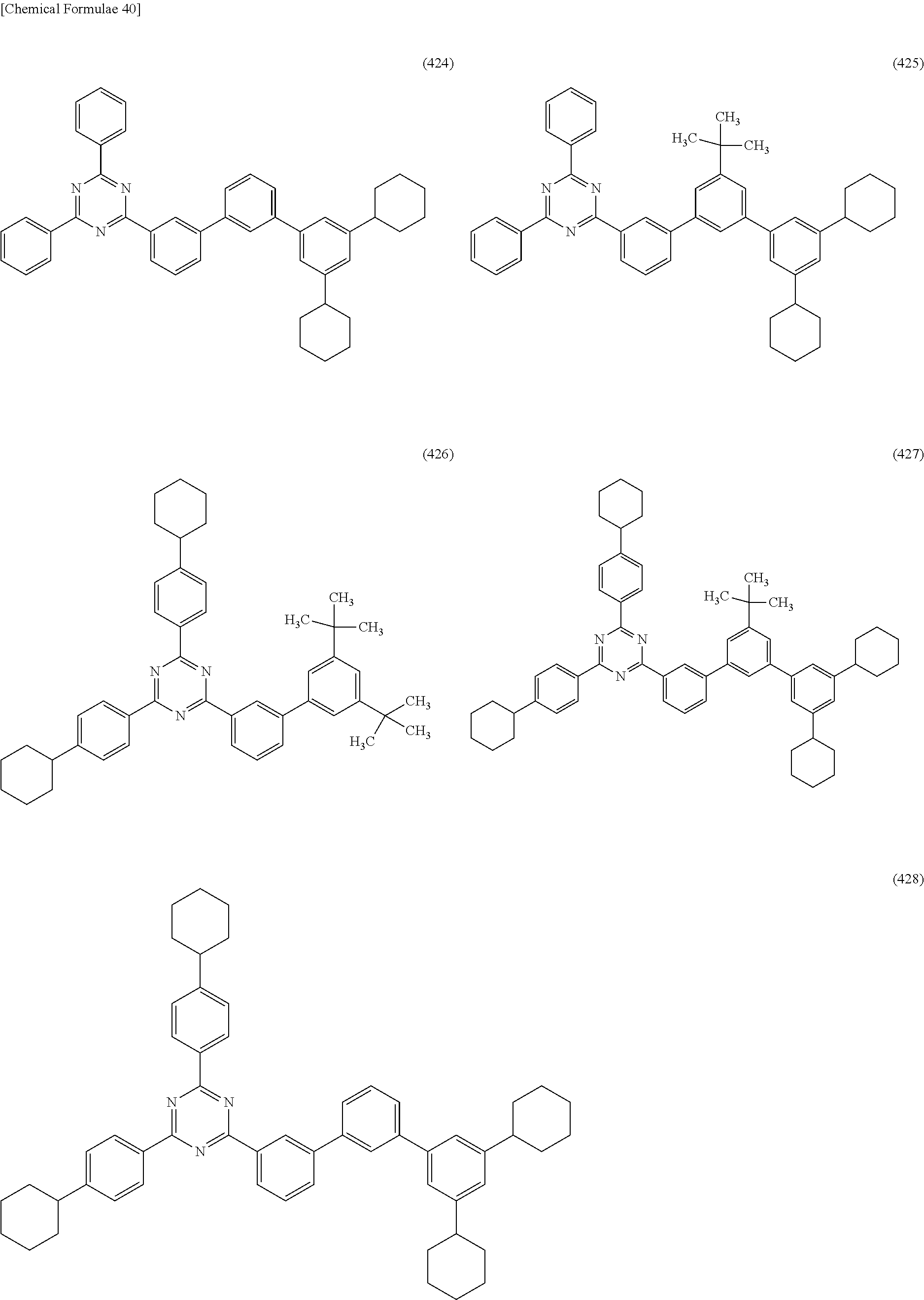 [SMILES: c1ccc(-c2nc(-c3ccccc3)nc(-c3cccc(-c4cccc(-c5cc(C6CCCCC6)cc(C6CCCCC6)c5)c4)c3)n2)cc1 ; CC(C)(C)c1cc(-c2cccc(-c3nc(-c4ccccc4)nc(-c4ccccc4)n3)c2)cc(-c2cc(C3CCCCC3)cc(C3CCCCC3)c2)c1 ; CC(C)(C)c1cc(-c2cccc(-c3nc(-c4ccc(C5CCCCC5)cc4)nc(-c4ccc(C5CCCCC5)cc4)n3)c2)cc(C(C)(C)C)c1 ; CC(C)(C)c1cc(-c2cccc(-c3nc(-c4ccc(C5CCCCC5)cc4)nc(-c4ccc(C5CCCCC5)cc4)n3)c2)cc(-c2cc(C3CCCCC3)cc(C3CCCCC3)c2)c1 ; c1cc(-c2cccc(-c3nc(-c4ccc(C5CCCCC5)cc4)nc(-c4ccc(C5CCCCC5)cc4)n3)c2)cc(-c2cc(C3CCCCC3)cc(C3CCCCC3)c2)c1]

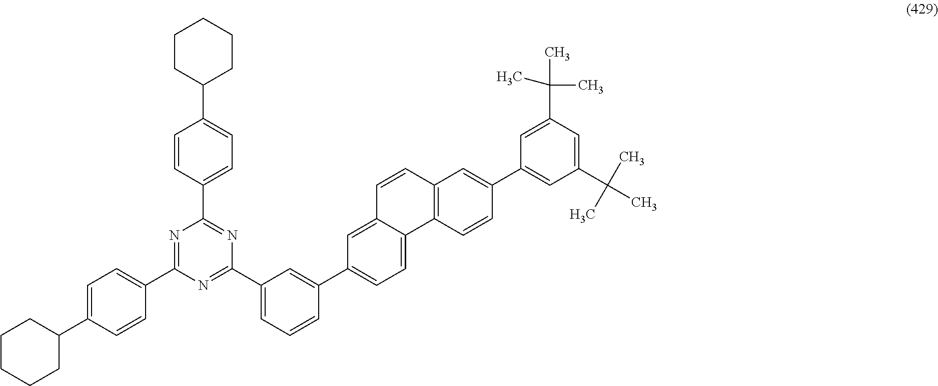 [SMILES: CC(C)(C)c1cc(-c2ccc3c(ccc4cc(-c5cccc(-c6nc(-c7ccc(C8CCCCC8)cc7)nc(-c7ccc(C8CCCCC8)cc7)n6)c5)ccc43)c2)cc(C(C)(C)C)c1]

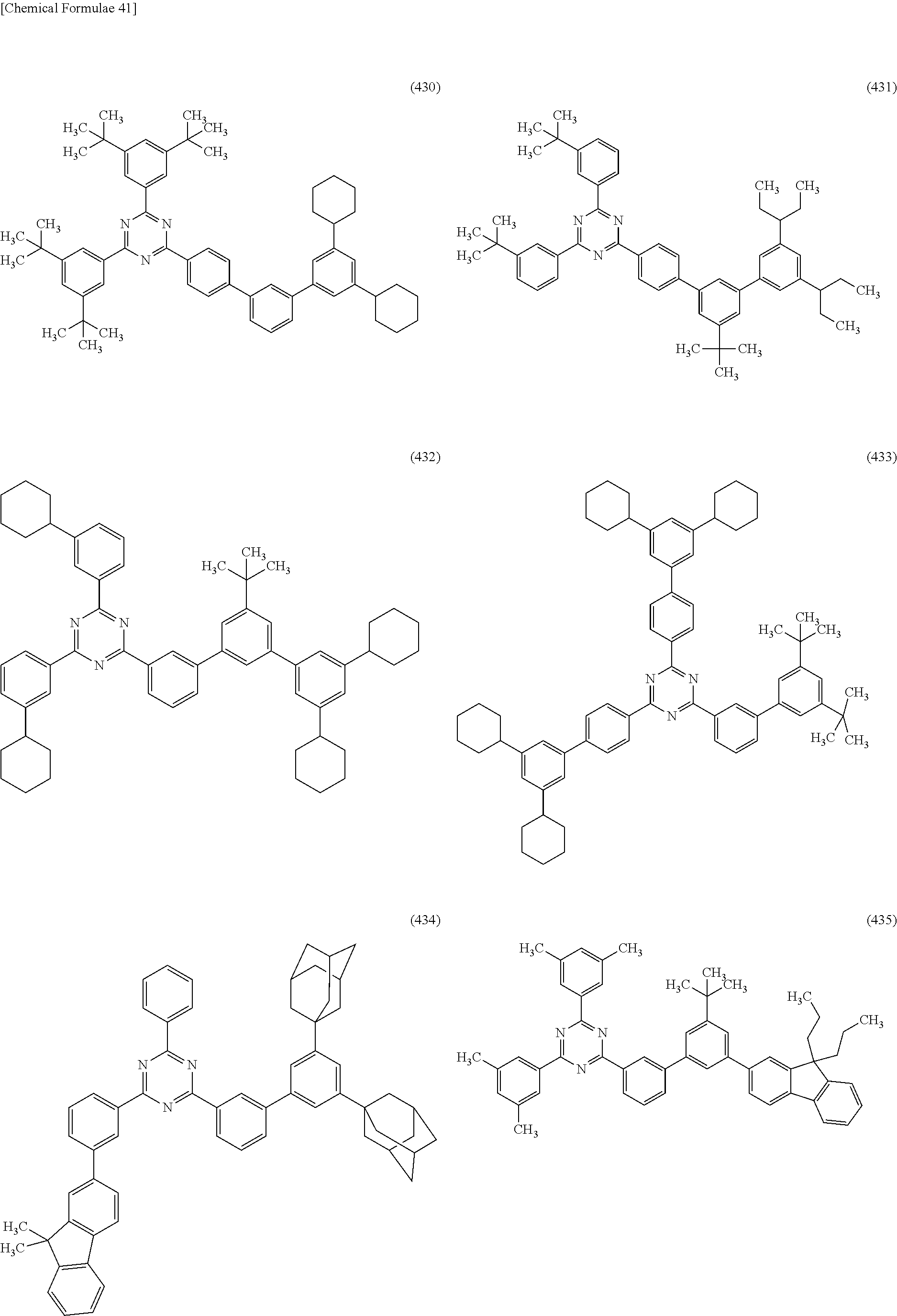

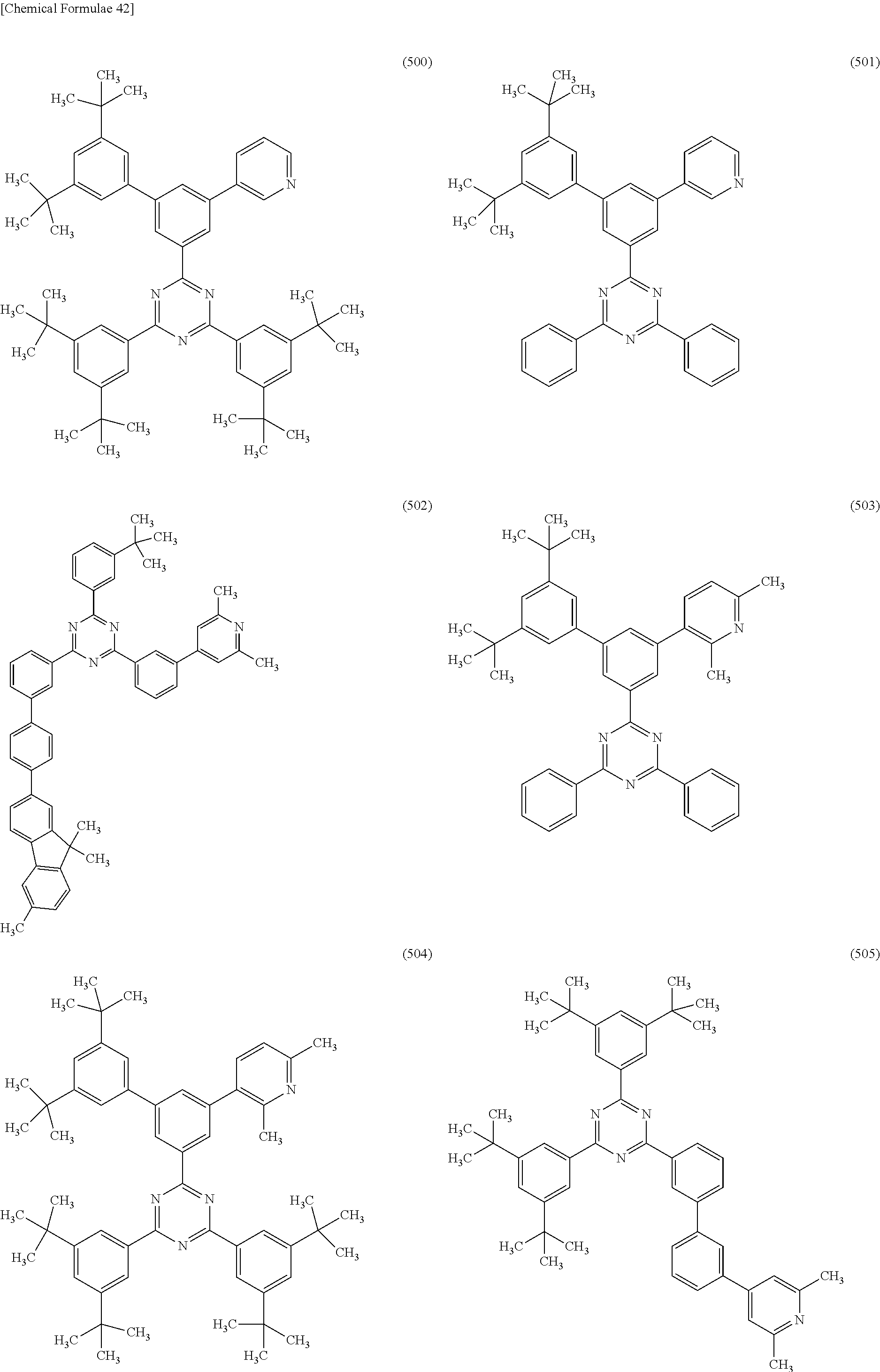

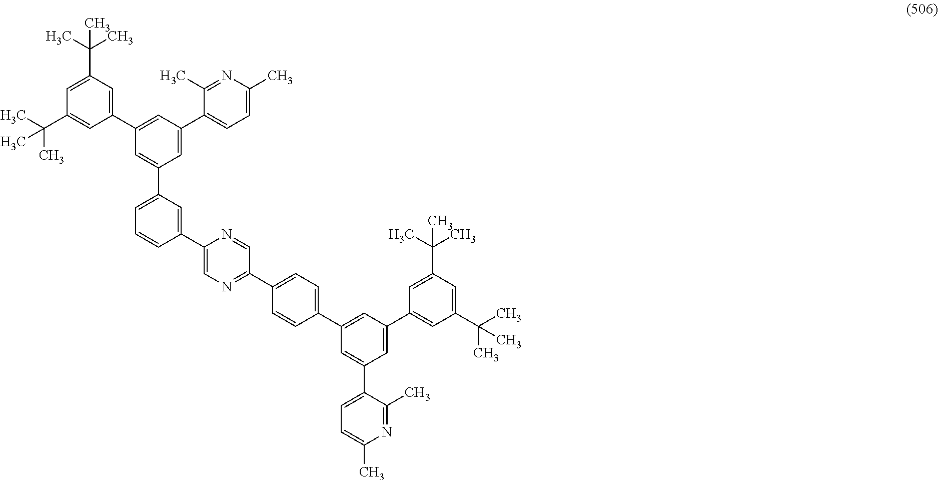 [SMILES: Cc1ccc(-c2cc(-c3ccc(-c4cnc(-c5cccc(-c6cc(-c7cc(C(C)(C)C)cc(C(C)(C)C)c7)cc(-c7ccc(C)nc7C)c6)c5)cn4)cc3)cc(-c3cc(C(C)(C)C)cc(C(C)(C)C)c3)c2)c(C)n1]

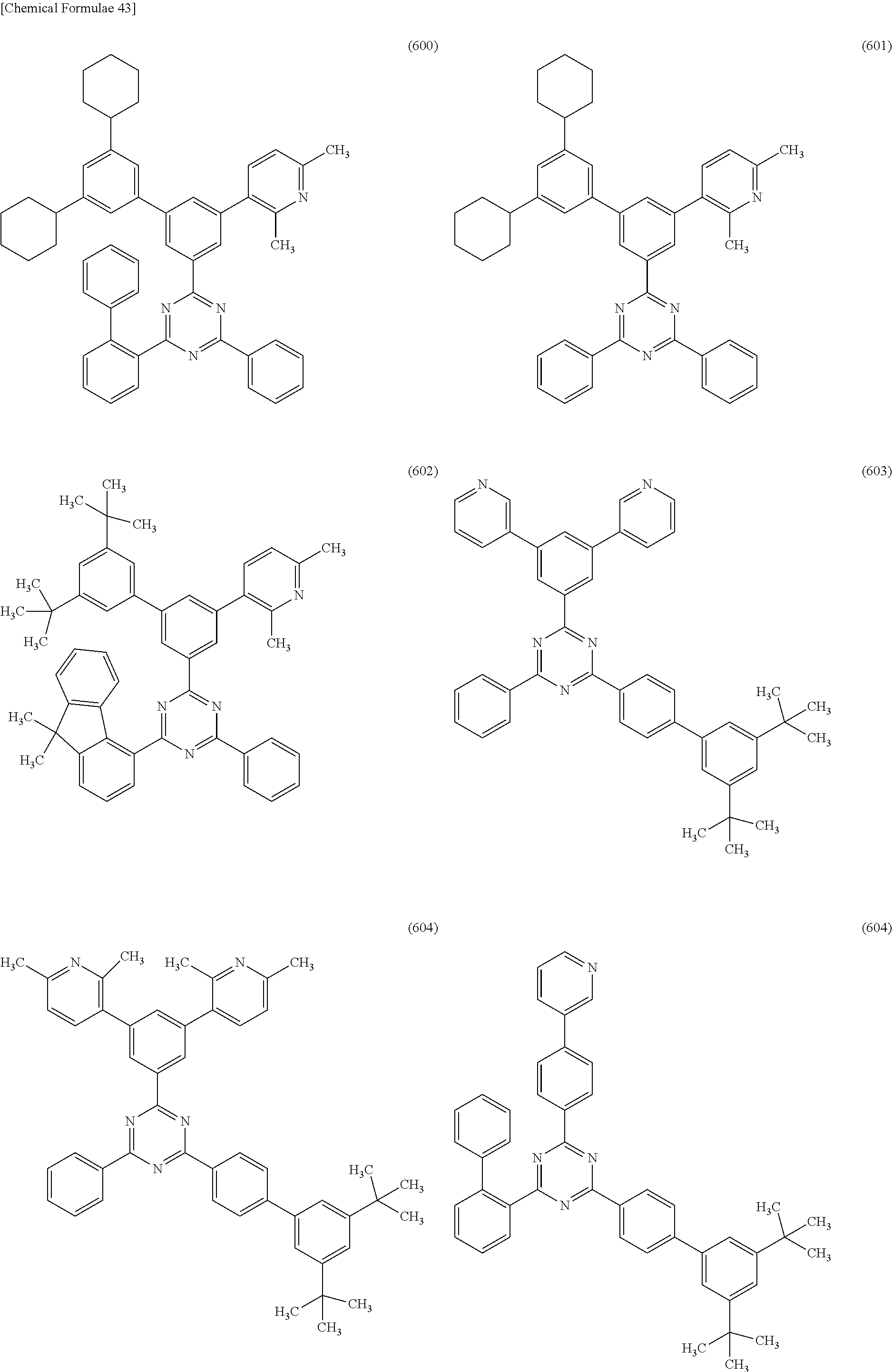

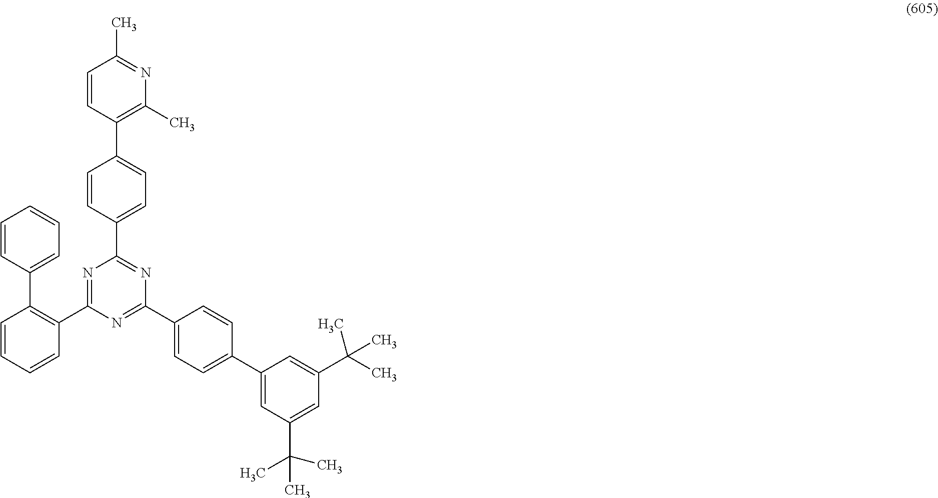 [SMILES: Cc1ccc(-c2ccc(-c3nc(-c4ccc(-c5cc(C(C)(C)C)cc(C(C)(C)C)c5)cc4)nc(-c4ccccc4-c4ccccc4)n3)cc2)c(C)n1]

The organic compound represented by General Formula (G1) is preferably formed of only a six-membered ring, an alkyl group, and an alicyclic group because the refractive index and the absorptance in the visible region can be made low; when such an organic compound is used as an electron-transport material of an optical device, the driving voltage can be kept low.

In the case where three substituted or unsubstituted phenyl groups are bonded to A in General Formula (G1), at least one of the phenyl groups preferably includes no substituent such as an alkyl group or an alicyclic group because, when the organic compound is used as an electron-transport material of an electronic device, the driving voltage can be kept low. Examples of the organic compound include organic compounds represented by Structural Formulae (103), (107), (111), (116) to (129), (212) to (215), (218) to (221), (311) to (316), (412) to (417), and (600) to (605).

The organic compounds represented by Structural Formulae (100) to (129), (200) to (223), (300) to (317), (400) to (435), (500) to (506), and (600) to (605) are examples of the organic compound represented by General Formula (G1). The organic compound of one embodiment of the present invention is not limited thereto.

Next, a method for synthesizing the organic compound of one embodiment of the present invention represented by General Formula (G1) will be described.

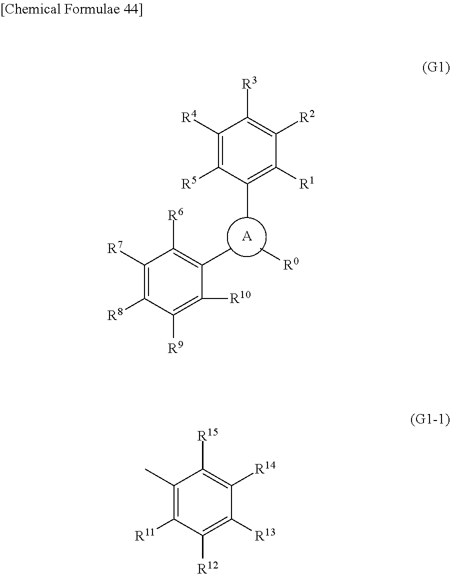

In General Formula (G1), A represents a six-membered heteroaromatic ring having 1 to 3 nitrogen atoms. R⁰ represents any of hydrogen, an alkyl group having 1 to 6 carbon atoms, an alicyclic group having 3 to 10 carbon atoms, and a substituent represented by Formula (G1-1). At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Note that the organic compound represented by General Formula (G1) includes a plurality of hydrocarbon groups selected from an alkyl group having 1 to 6 carbon atoms and an alicyclic group having 3 to 10 carbon atoms, and the proportion of carbon atoms forming a bond by the sp³ hybrid orbitals in the total number of carbon atoms in a molecule of the organic compound is higher than or equal to 10% and lower than or equal to 60%.

### <<Method for Synthesizing Organic Compound Represented by General Formula (G1)>>

An example of a synthesis method of the organic compound represented by General Formula (G1) is described below. A variety of reactions can be used for the synthesis of this organic compound. For example, as shown in Synthesis Scheme (A-1), a compound (a4) can be obtained by reaction between halogenated aryl (a1), halogenated aryl (a2), and halogenated aryl (a3). In the reaction, halogenated aryl (a2) and halogenated aryl (a3) are a Grignard reagent or a lithium compound and halogenated aryl (a1) is made to react in an ether solvent, whereby the compound (a4) can be obtained.

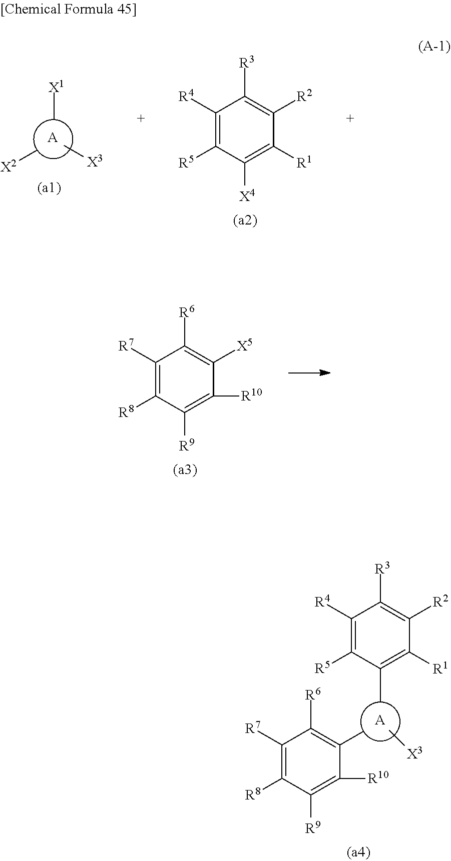

In the formula, A represents a six-membered heteroaromatic ring having 1 to 3 nitrogen atoms. At least one of R¹ to R¹⁰ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Furthermore, X¹ to X⁵ each independently represent any of chlorine, bromine, and iodine.

In the reaction, halogenated aryl (a2) and halogenated aryl (a3) preferably have the same structure because the yield of halogenated aryl (a4) can be increased. Note that in the case where halogenated aryl (a2) and halogenated aryl (a3) have different structures, the yield of the compound (a4) can be increased by performing the synthesis in two or more steps: a first step of reacting halogenated aryl (a1) with halogenated aryl (a2) and a second step of reacting halogenated aryl (a3) with the obtained product, for example.

In the case where R⁰ in General Formula (G1) is hydrogen, hydrogen is used as X³, whereby the organic compound represented by General Formula (G1) can be synthesized through Synthesis Scheme (A-1).

Next, as shown in Synthesis Scheme (A-2) below, halogenated aryl (a4) and an arylboron compound (a5) are coupled, whereby the target compound (G1) can be synthesized. In this reaction, a synthesis method in which a metal catalyst is used under the presence of a base, e.g., the Suzuki-Miyaura reaction, can be used.

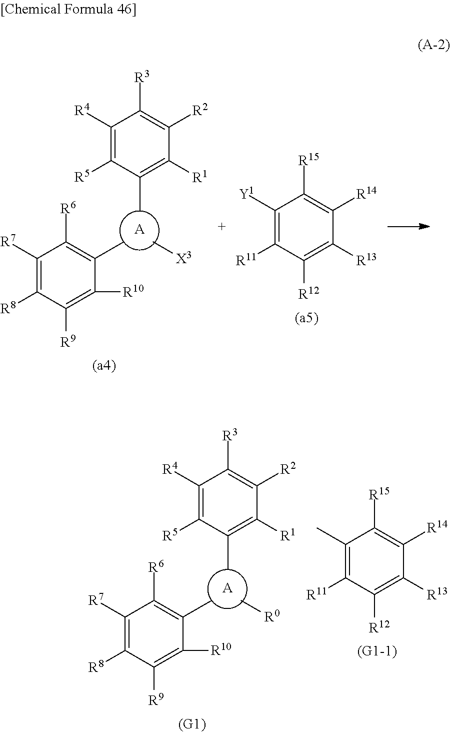

In the formula, A represents a six-membered heteroaromatic ring having 1 to 3 nitrogen atoms. R⁰ represents a substituent represented by Formula (G1-1). At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Y¹ represents a boronic acid or a boronic ester such as pinacol boron. X³ is any of chlorine, bromine, and iodine, and an element with a larger atomic number is preferably used because reactivity can be increased. Alternatively, X³ may be a boronic acid or a boronic ester such as pinacol boron and Y¹ may be halogen or a sulfonyloxy group in the reaction.

When R⁰ is an alkyl group having 1 to 6 carbon atoms or an alicyclic group having 3 to 10 carbon atoms, a Grignard reagent or a lithium compound of the alkyl group or the alicyclic group is made to react with halogenated aryl (a4) with the use of a metal catalyst in an ether solvent, whereby the compound (a4) can be obtained. For example, the Kumada-Tamao-Corriu coupling can be used.

Note that in Synthesis Scheme (A-1), halogenated aryl (a1) and the arylboron compound (a5) may be reacted with each other first, and then the obtained compound may be reacted with halogenated aryl (a2) and halogenated aryl (a3). Three substituents bonded to A of the target compound (G1) preferably have the same structure because coupling reaction caused by substitution of the three substituents by X¹ to X³ of halogenated aryl (a1) in the first step can reduce the synthesis cost.

Alternatively, as shown in Synthesis Scheme (B-1) below, halogenated aryl ((b1)+(b1-1)) and an arylboron compound (b2) are coupled, whereby the target compound (G1) can be synthesized. In this reaction, a synthesis method in which a metal catalyst is used under the presence of a base, e.g., the Suzuki-Miyaura reaction, can be used.

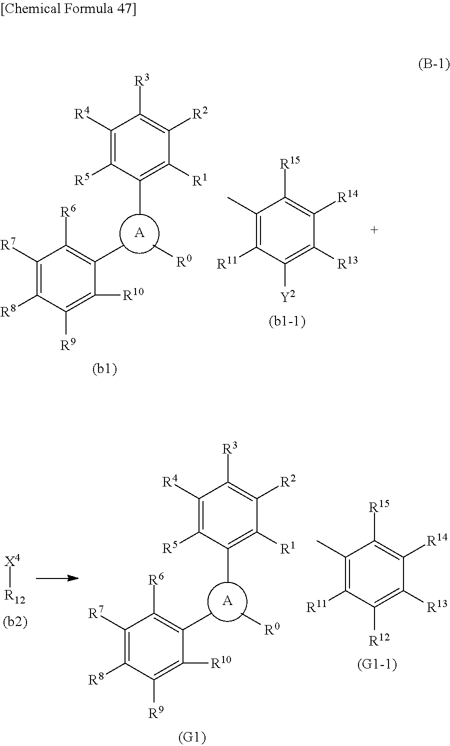

In the formula, A represents a six-membered heteroaromatic ring having 1 to 3 nitrogen atoms. R⁰ represents any of hydrogen, an alkyl group having 1 to 6 carbon atoms, an alicyclic group having 3 to 10 carbon atoms, and a substituent represented by Formula (G1-1). At least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. The phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring. Y^{Z} represents a boronic acid or a boronic ester such as pinacol boron. X⁴ is any of chlorine, bromine, iodine, and a sulfonyloxy group, and an element with a larger atomic number is preferably used because reactivity can be increased. Alternatively, X⁴ may be a boronic acid or a boronic ester such as pinacol boron and Y² may be halogen or a sulfonyloxy group in the reaction.

Although this synthesis scheme shows a reaction example in which R¹⁻² of the arylboron compound (b2) is substituted for Y² of the substituent (b1-1) of halogenated aryl ((b1)+(b1-1)), the site of substitution may be any of R¹ to R¹¹ and R¹³ to R¹⁵ of halogenated aryl ((b1)+(b1-1)).

Although an example of a method for synthesizing the organic compound of one embodiment of the present invention is described above, the present invention is not limited thereto and any other synthesis method may be employed.

The structures described in this embodiment can be used in combination with any of the structures described in the other embodiments, as appropriate.

### Embodiment 2

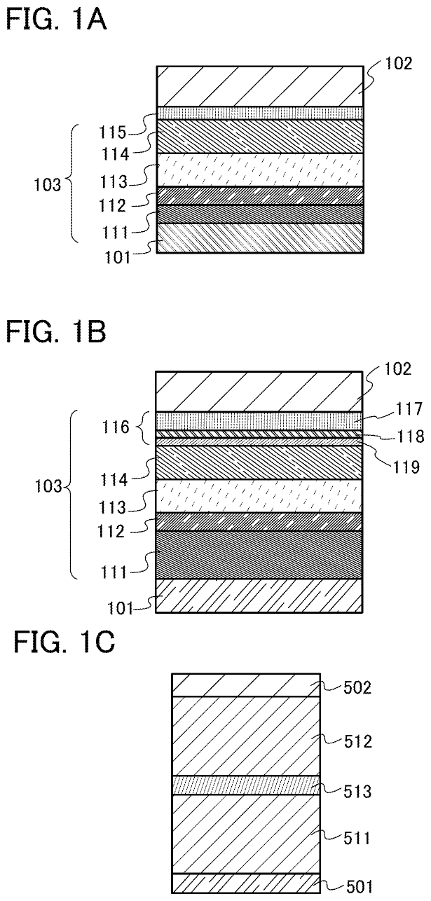

FIG. 1A illustrates a light-emitting device of one embodiment of the present invention. The light-emitting device of one embodiment of the present invention includes a first electrode 101, a second electrode 102, and an EL layer 103, and the electron-transport layer material or the organic compound described in Embodiment 1 is used for the EL layer.

The EL layer 103 includes a light-emitting layer 113 and may also include a hole-injection layer 111 and/or a hole-transport layer 112. The light-emitting layer 113 includes a light-emitting material, and light is emitted from the light-emitting material in the light-emitting device of one embodiment of the present invention. The light-emitting layer 113 may include a host material and other materials. The electron-transport layer material described in Embodiment 1 is included in an electron-transport layer 114 and/or an electron-injection layer 115. The organic compound described in Embodiment 1 may be included in the light-emitting layer 113, the electron-transport layer 114, the electron-injection layer 115, or all of them.

Note that FIG. 1A additionally illustrates the hole-injection layer 111 and the hole-transport layer 112; however, the structure of the light-emitting device is not limited thereto. For example, an electron-blocking layer may be provided between the hole-transport layer 112 and the light-emitting layer 113, or a hole-blocking layer may be provided between the light-emitting layer 113 and the electron-transport layer 114.

The use of the electron-transport layer material or the organic compound for the electron-transport layer 114 is effective because of its excellent electron-transport property. A mixture of the electron-transport layer material or the organic compound of one embodiment of the present invention and an organometallic complex of an alkali metal is preferably used for the electron-transport layer 114 and/or the electron-injection layer 115, in which case the driving voltage and the emission efficiency can be improved.

The organic compound of one embodiment of the present invention can also be used as a host material. In that case, the organic compound and a hole-transport material may be deposited by co-evaporation so that an exciplex can be formed of the organic compound and the hole-transport material. The exciplex having an appropriate emission wavelength allows effective energy transfer to the light-emitting material, achieving a light-emitting device with high efficiency and a long lifetime.

Since the organic compound of one embodiment of the present invention has a low refractive index, the light-emitting device using the organic compound in its EL layer can have high external quantum efficiency.

Next, examples of specific structures and materials of the above-described light-emitting device will be described. As described above, the light-emitting device of one embodiment of the present invention includes, between the pair of electrodes of the first electrode 101 and the second electrode 102, the EL layer 103 including a plurality of layers; the EL layer 103 includes the electron-transport layer material or the organic compound disclosed in Embodiment 1 in any of the layers.

The first electrode 101 is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide, indium oxide-zinc oxide, and indium oxide containing tungsten oxide and zinc oxide (IWZO). Such conductive metal oxide films are usually formed by a sputtering method, but may be formed by application of a sol-gel method or the like. In an example of the formation method, indium oxide-zinc oxide is deposited by a sputtering method using a target obtained by adding 1 wt % to 20 wt % of zinc oxide to indium oxide. Furthermore, a film of indium oxide containing tungsten oxide and zinc oxide (IWZO) can be formed by a sputtering method using a target in which tungsten oxide and zinc oxide are added to indium oxide at 0.5 wt % to 5 wt % and 0.1 wt % to 1 wt %, respectively. Alternatively, gold (Au), platinum (Pt), nickel (Ni), tungsten (W), chromium (Cr), molybdenum (Mo), iron (Fe), cobalt (Co), copper (Cu), palladium (Pd), nitride of a metal material (e.g., titanium nitride), or the like can be used. Graphene can also be used. Note that when a composite material described later is used for a layer in the EL layer 103 that is in contact with the first electrode 101, an electrode material can be selected regardless of its work function.

Although the EL layer 103 preferably has a stacked-layer structure, there is no particular limitation on the stacked-layer structure, and various layers such as a hole-injection layer, a hole-transport layer, an electron-transport layer, an electron-injection layer, a carrier-blocking layer (e.g., a hole-blocking layer and an electron-blocking layer), an exciton-blocking layer, and a charge-generation layer can be employed. This embodiment describes two kinds of structures: a structure illustrated in FIG. 1A including the hole-injection layer 111 and the hole-transport layer 112 in addition to the electron-transport layer 114, the electron-injection layer 115, and the light-emitting layer 113; and a structure illustrated in FIG. 1B including a charge-generation layer 116 in addition to the electron-transport layer 114, the electron-injection layer 115, the light-emitting layer 113, the hole-injection layer 111, and the hole-transport layer 112. Materials for the layers will be specifically described below.

The hole-injection layer 111 contains a substance having an acceptor property. Either an organic compound or an inorganic compound can be used as the substance having an acceptor property.

As the substance having an acceptor property, it is possible to use a compound having an electron-withdrawing group (a halogen group or a cyano group); for example, 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylene-1,3,4,5,6,8,9,10-octafluoro-7H-pyren-2-ylidene)malononitrile can be used. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group or a halogen group such as a fluoro group) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the sub stance having an acceptor property, molybdenum oxide, vanadium oxide, ruthenium oxide, tungsten oxide, manganese oxide, or the like can be used, other than the above-described organic compounds. Alternatively, the hole-injection layer 111 can be formed using a phthalocyanine-based complex compound such as phthalocyanine (abbreviation: H₂Pc) and copper phthalocyanine (CuPc), an aromatic amine compound such as 4,4′-bis[N-(4-diphenylaminophenyl)-N-phenylamino]biphenyl (abbreviation: DPAB) and N,N′-bis{4-[bis(3-methylphenyl)amino]phenyl}-N,N-diphenyl-(1,1′-biphenyl)-4,4′-diamine (abbreviation: DNTPD), or a high molecular compound such as poly(3,4-ethylenedioxythiophene)/poly(styrenesulfonic acid) (PEDOT/PSS). The sub stance having an acceptor property can extract electrons from an adjacent hole-transport layer (or hole-transport material) by the application of an electric field.

Alternatively, a composite material in which a material having a hole-transport property contains any of the aforementioned substances having an acceptor property can be used for the hole-injection layer 111. By using a composite material in which a material having a hole-transport property contains an acceptor substance, a material used to form an electrode can be selected regardless of its work function. In other words, besides a material having a high work function, a material having a low work function can be used for the first electrode 101.

As the material having a hole-transport property used for the composite material, any of a variety of organic compounds such as aromatic amine compounds, carbazole derivatives, aromatic hydrocarbons, and high molecular compounds (e.g., oligomers, dendrimers, or polymers) can be used. Note that the material having a hole-transport property used for the composite material preferably has a hole mobility of 1×10⁻⁶ cm²/Vs or higher. Organic compounds which can be used as the material having a hole-transport property in the composite material are specifically given below.

Examples of the aromatic amine compounds that can be used for the composite material include N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), {4-[bis(3-methylphenyl)amino]phenyl}-N,N-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B). Specific examples of the carbazole derivative include 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenylanthracen-9-yl)phenyl]-9H-carbazole (abbreviation: CzPA), and 1,4-bis[4-(N-carbazolyl)phenyl]-2,3,5,6-tetraphenylbenzene. Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon may have a vinyl skeleton. Examples of the aromatic hydrocarbon having a vinyl group include 4,4′-bis(2,2-diphenylvinyl)biphenyl (abbreviation: DPVBi) and 9,10-bis[4-(2,2-diphenylvinyl)phenyl]anthracene (abbreviation: DPVPA). Note that the organic compound of one embodiment of the present invention can also be used.

Other examples include high molecular compounds such as poly(N-vinylcarbazole) (abbreviation: PVK), poly(-vinyltriphenylamine) (abbreviation: PVTPA), poly [N-(4-{N-[4-(4-diphenylamino)phenyl]phenyl-N′-phenylamino}phenyl)methacrylamide] (abbreviation: PTPDMA), and poly[N,N-bis(4-butylphenyl)-N,N-bis(phenyl)benzidine] (abbreviation: poly-TPD).

The material having a hole-transport property used for the composite material further preferably has any of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that includes a naphthalene ring, or an aromatic monoamine in which a 9-fluorenyl group is bonded to nitrogen of amine through an arylene group may be used. Note that the organic compound having an N,N-bis(4-biphenyl)amino group is preferable because a light-emitting device having a long lifetime can be fabricated. Specific examples of the organic compound include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II) (4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi11BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis([1,1′-biphenyl]-4-yl)-9,9′-spirobi [9H-fluoren]-2-amine (abbreviation: BBASF), N,N-bis(1,1′-biphenyl-4-yl)-9,9′-spirobi [9H-fluoren]-4-amine (abbreviation: BBASF(4)), N-(1,1′-biphenyl-2-yl)-N-(9,9-dimethyl-9H-fluoren-2-yl)-9,9′-spirobi(9H-fluoren)-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)tri phenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(1,1′-biphenyl-4-yl)-9,9-dimethyl-N-[4-(9-phenyl-9H-carbazol-3-yl)phenyl]-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Note that it is further preferable that the material having a hole-transport property used for the composite material have a relatively deep HOMO (Highest Occupied Molecular Orbital) level of higher than or equal to −5.7 eV and lower than or equal to −5.4 eV. Using the hole-transport material with a relatively deep HOMO level in the composite material makes it easy to inject holes into the hole-transport layer 112 and to obtain a light-emitting device having a long lifetime.

Note that mixing the above composite material with a fluoride of an alkali metal or an alkaline earth metal (the proportion of fluorine atoms in a layer using the mixed material is preferably greater than or equal to 20%) can lower the refractive index of the layer. This also enables a layer with a low refractive index to be formed in the EL layer 103, leading to higher external quantum efficiency of the light-emitting device.

The formation of the hole-injection layer 111 can improve the hole-injection property, which allows the light-emitting device to be driven at a low voltage. In addition, the organic compound having an acceptor property is easy to use because it is easily deposited by vapor deposition.

The hole-transport layer 112 is formed using a material having a hole-transport property. The material having a hole-transport property preferably has a hole mobility higher than or equal to 1×10⁻⁶ cm²/Vs.

Examples of the material having a hole-transport property include compounds having an aromatic amine skeleton, such as 4,4′-bis[N-(1-naphthyl)-N-phenylamino]biphenyl (abbreviation: NPB), N,N-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi [9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 3,3′-bis(9-phenyl-9H-carbazole) (abbreviation: PCCP); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. Note that any of the substances given as examples of the material having a hole-transport property used for the composite material for the hole-injection layer 111 can also be suitably used as the material included in the hole-transport layer 112.

The light-emitting layer 113 includes a light-emitting substance and a host material. The light-emitting layer 113 may additionally include other materials. Alternatively, the light-emitting layer 113 may be a stack of two layers with different compositions.

The light-emitting substance may be a fluorescent substance, a phosphorescent substance, a substance exhibiting thermally activated delayed fluorescence (TADF), or any other light-emitting substance. Note that one embodiment of the present invention is more suitably used in the case where the light-emitting layer 113 emits fluorescence, specifically, blue fluorescence.

Examples of the material that can be used as a fluorescent substance in the light-emitting layer 113 are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N-diphenyl stilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N,N-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-c]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N-diphenyl-N,N′-(1,6-pyrene-diyl)bis[(6-phenylbenzo[b]naphtho[1,2-d]furan)-8-amine] (abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, and high reliability.

Examples of the material that can be used when a phosphorescent substance is used as the light-emitting substance in the light-emitting layer 113 are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5 sopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[(1-2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-j]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)). These compounds emit blue phosphorescence and have an emission peak at 440 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), and bis(2-phenylquinolinato-N,C^{2′})iridium(III) acetylacetonate (abbreviation: [Ir(pq)₂(acac)]); and a rare earth metal complex such as tris(acetylacetonato) (monophenanthroline)terbium(III) (abbreviation: [Tb(acac)₃(Phen)]). These are mainly compounds that emit green phosphorescence and have an emission peak at 500 nm to 600 nm. Note that organometallic iridium complexes having a pyrimidine skeleton have distinctively high reliability and emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]) and bis(1-phenylisoquinolinato-N,C^{2′})iridium(III) acetylacetonate (abbreviation: [Ir(piq)₂(acac)]); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato) (monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescence and have an emission peak at 600 nm to 700 nm. Furthermore, the organometallic iridium complexes having a pyrazine skeleton can provide red light emission with favorable chromaticity.

Besides the above phosphorescent compounds, known phosphorescent substances may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

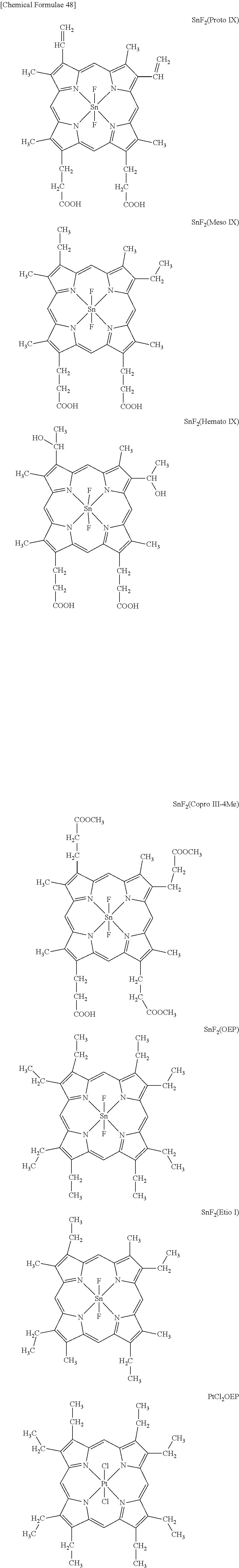

It is also possible to use a heterocyclic compound having one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), diphenyl-1,3,5-triazin-2-yl)-9′-phenyl-9H,9′H-3,3′-bicarbazole (abbreviation: PCCzTzn), 9-[4-(4,6-diphenyl-1,3,5-triazin-2-yl)phenyl]-9′-phenyl-9H,9′H-3,3′-bicarbazole (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having excellent electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferred because of their high stability and reliability. In particular, a benzofuropyrimidine skeleton, a benzothienopyrimidine skeleton, a benzofuropyrazine skeleton, and a benzothienopyrazine skeleton are preferred because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; therefore, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the S1 level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

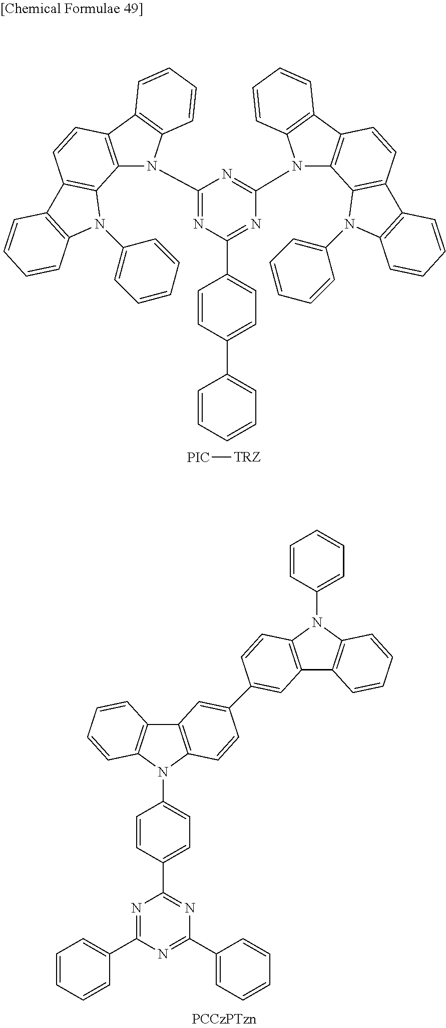

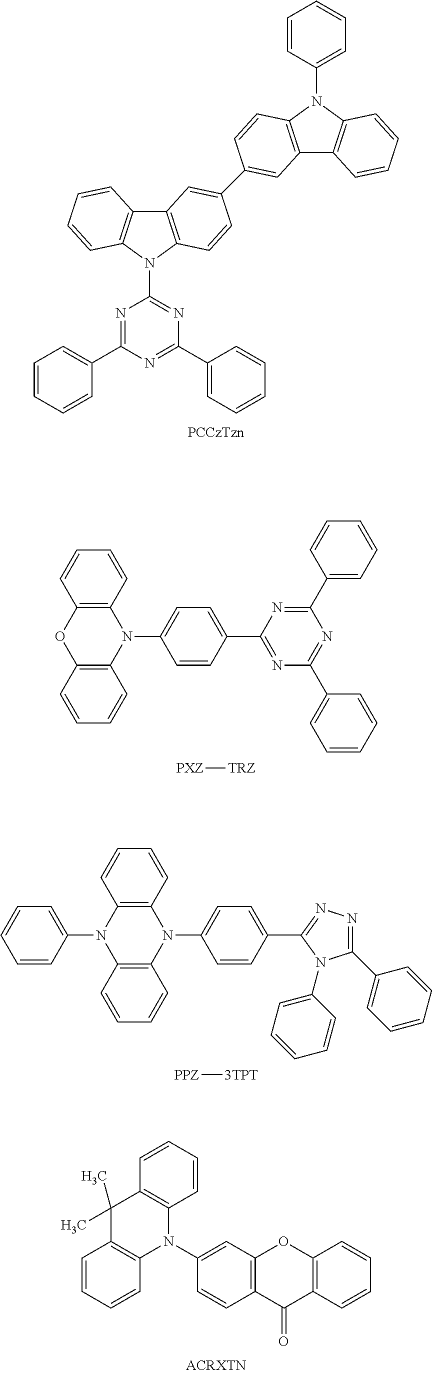

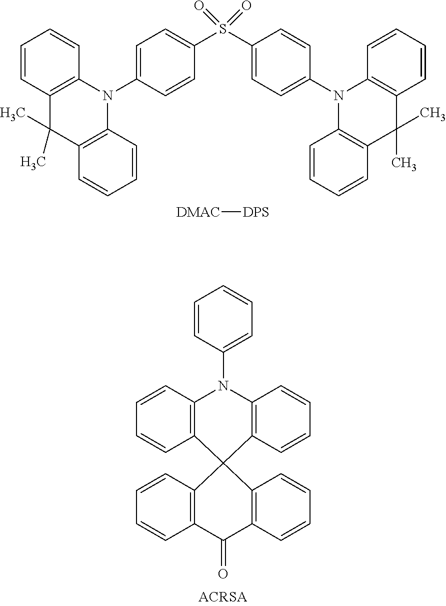

Note that the TADF material is a material having a small difference between the Si level and the Ti level and a function of converting triplet excitation energy into singlet excitation energy by reverse intersystem crossing. Thus, the TADF material can upconvert triplet excitation energy into singlet excitation energy (i.e., reverse intersystem crossing) using a small amount of thermal energy and efficiently generate a singlet excited state. In addition, the triplet excitation energy can be converted into luminescence.

An exciplex whose excited state is formed of two kinds of substances has an extremely small difference between the Si level and the Ti level and functions as a TADF material capable of converting triplet excitation energy into singlet excitation energy.

A phosphorescent spectrum observed at a low temperature (e.g., 77 K to 10 K) is used for an index of the T1 level. When the level of energy with a wavelength of the line obtained by extrapolating a tangent to the fluorescent spectrum at a tail on the short wavelength side is the Si level and the level of energy with a wavelength of the line obtained by extrapolating a tangent to the phosphorescent spectrum at a tail on the short wavelength side is the T1 level, the difference between the S1 level and the T1 level of the TADF material is preferably smaller than or equal to 0.3 eV, further preferably smaller than or equal to 0.2 eV.

When a TADF material is used as the light-emitting substance, the S1 level of the host material is preferably higher than that of the TADF material. In addition, the T1 level of the host material is preferably higher than that of the TADF material.

As the host material in the light-emitting layer, various carrier-transport materials such as materials having an electron-transport property, materials having a hole-transport property, and the TADF materials can be used.

The material having a hole-transport property is preferably an organic compound having an amine skeleton or a π-electron rich heteroaromatic ring skeleton. Examples of the material include compounds having an aromatic amine skeleton, such as 4,4′-bis[N-(1-naphthyl)-N-phenylamino]biphenyl (abbreviation: NPB), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 3,3′-bis(9-phenyl-9H-carbazole) (abbreviation: PCCP); compounds having a thiophene skeleton, such as 4,4′,4″-(benzene-1,3,5-triyl)tri(dibenzothiophene) (abbreviation: DB T3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. In addition, the organic compounds given as examples of the material having a hole-transport property in the hole-transport layer 112 can also be used.

As the material having an electron-transport property, metal complexes such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); or an organic compound having a π-electron deficient heteroaromatic ring skeleton is preferable. Examples of the organic compound having a π-electron deficient heteroaromatic ring skeleton include heterocyclic compounds having a polyazole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), and 2-[3-(dibenzothiophen-4-yl)phenyl]-1-phenyl-1H-benzimidazole (abbreviation: mDBTBIm-II); heterocyclic compounds having a diazine skeleton, such as 2-[3-(dibenzothiophen-4-yl)phenyl]dibenzo[fh]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDB q), 4,6-bis[3-(phenanthren-9-yl)phenyl]pyrimidine (abbreviation: 4,6mPnP2Pm), and 4,6-bis[3-(4-dibenzothienyl)phenyl]pyrimidine (abbreviation: 4,6mDBTP2Pm-II); and heterocyclic compounds having a pyridine skeleton, such as 3,5-bis[3-(9H-carbazol-9-yl)phenyl]pyridine (abbreviation: 35DCzPPy) and 1,3,5-tri[3-(3-pyridyl)phenyl]benzene (abbreviation: TmPyPB). Among the above materials, the heterocyclic compound having a diazine skeleton and the heterocyclic compound having a pyridine skeleton have high reliability and thus are preferable. In particular, the heterocyclic compound having a diazine (pyrimidine or pyrazine) skeleton has a high electron-transport property to contribute to a reduction in driving voltage. Note that the organic compounds described in Embodiment 1 have electron-transport properties and thus can be used as the host material. A layer with a low refractive index can be formed in the EL layer 103 with the use of the organic compound described in Embodiment 1, leading to higher external quantum efficiency of the light-emitting device.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S1 level of the TADF material is preferably higher than that of the fluorescent substance in order that high emission efficiency can be achieved. Furthermore, the T1 level of the TADF material is preferably higher than the S1 level of the fluorescent substance. Therefore, the T1 level of the TADF material is preferably higher than that of the fluorescent substance.

A TADF material that emits light whose wavelength overlaps with the wavelength on a lowest-energy-side absorption band of the fluorescent substance is preferably used, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferred because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to carbazole because the HOMO level thereof is shallower than that of carbazole by approximately 0.1 eV and thus holes enter the host material easily. In particular, the host material preferably has a dibenzocarbazole skeleton because the HOMO level thereof is shallower than that of carbazole by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), and 9-(1-naphthyl)-10-[4-(2-naphthyl)phenyl]anthracene (abbreviation: αN-βNPAnth). In particular, CzPA, cgDBCzPA, 2mBnfPPA, and PCzPA have excellent characteristics and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength on a lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to the LUMO level of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient PL lifetime of the mixed film has more long lifetime components or has a larger proportion of delayed components than that of each of the materials, observed by comparison of transient photoluminescence (PL) of the material having a hole-transport property, the material having an electron-transport property, and the mixed film of the materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of the materials.

The electron-transport layer 114 contains a substance having an electron-transport property. The thickness of the electron-transport layer 114 is greater than or equal to 10 nm and less than or equal to 50 nm, preferably greater than or equal to 15 nm and less than or equal to 35 nm, for example. As the substance having an electron-transport property, the electron-transport layer material or the organic compound described in Embodiment 1 is preferably used. The use of the electron-transport layer material or the organic compound described in Embodiment 1 for the electron-transport layer 114 enables a layer with a low refractive index to be formed in the EL layer 103, improving the external quantum efficiency of the light-emitting device.

When a material other than the electron-transport layer material and the organic compound described in Embodiment 1 is used for the electron-transport layer 114, any of the above-described substances having electron-transport properties that can be used as the host material can be used.

The electron-transport layer 114 preferably includes a material having an electron-transport property and an alkali metal, an alkaline earth metal, a compound thereof, or a complex thereof. In particular, an organometallic complex of an alkali metal is preferable, and an organometallic complex of lithium is further preferable. As a ligand of the organometallic complex, a ligand having a 8-quinolinolate structure is particularly preferable, and 8-quinolinolato-lithium or 6-methyl-8-quinolinolato-lithium is further preferable. Note that the electron-transport layer 114 having this structure also serves as the electron-injection layer 115 in some cases.

The electron mobility of the material included in the electron-transport layer 114 in the case where the square root of the electric field strength [V/cm] is 600 is preferably higher than or equal to 1×10⁻⁷ cm²/Vs and lower than or equal to 5×10⁻⁵ cm²/Vs. The amount of electrons injected into the light-emitting layer can be controlled by the reduction in the electron-transport property of the electron-transport layer 114, whereby the light-emitting layer can be prevented from having excess electrons. It is particularly preferable that this structure be employed when the hole-injection layer is formed using a composite material that includes a material having a hole-transport property with a relatively deep HOMO level of −5.7 eV or higher and −5.4 eV or lower, in which case the light-emitting device can have a long lifetime. In this case, the material having an electron-transport property preferably has a HOMO level of −6.0 eV or higher. The material having an electron-transport property is preferably an organic compound having an anthracene skeleton and further preferably an organic compound having both an anthracene skeleton and a heterocyclic skeleton. The heterocyclic skeleton is preferably a nitrogen-containing five-membered ring skeleton or a nitrogen-containing six-membered ring skeleton, and particularly preferably a nitrogen-containing five-membered ring skeleton or a nitrogen-containing six-membered ring skeleton including two heteroatoms in the ring, such as a pyrazole ring, an imidazole ring, an oxazole ring, a thiazole ring, a pyrazine ring, a pyrimidine ring, or a pyridazine ring. In addition, it is preferable that the alkali metal, the alkaline earth metal, the compound thereof, or the complex thereof have a 8-hydroxyquinolinato structure. Specific examples include 8-hydroxyquinolinato-lithium (abbreviation: Liq) and 8-hydroxyquinolinato-sodium (abbreviation: Naq). In particular, a complex of a monovalent metal ion, especially a complex of lithium is preferable, and Liq is further preferable. Note that in the case where the 8-hydroxyquinolinato structure is included, a methyl-substituted product (e.g., a 2-methyl-substituted product or a 5-methyl-substituted product) of the alkali metal, the alkaline earth metal, the compound, or the complex can also be used, for example. There is preferably a difference in the concentration (including 0) of the alkali metal, the alkaline earth metal, the compound thereof, or the complex thereof in the electron-transport layer in the thickness direction.

The electron-transport layer 114 is provided between the light-emitting layer 113 and the second electrode 102. A layer containing an alkali metal, an alkaline earth metal, or a compound thereof such as lithium fluoride (LiF), cesium fluoride (CsF), calcium fluoride (CaF₂), or 8-hydroxyquinolinato-lithium (Liq) may be provided as the electron-injection layer 115 between the electron-transport layer 114 and the second electrode 102. As the electron-injection layer 115, an electrode or a layer that is formed using a substance having an electron-transport property and that includes an alkali metal, an alkaline earth metal, or a compound thereof can be used. Examples of the electrode include a substance in which electrons are added at high concentration to calcium oxide-aluminum oxide.

A material in which a material having an electron-transport property and an alkali metal, an alkaline earth metal, a compound thereof, or a complex thereof are mixed may be used for the electron-injection layer 115. As the material having an electron-transport property, any of the above-described materials that can be used for the electron-transport layer 114 can also be used here. Note that the electron-injection layer 115 having this structure also serves as the electron-transport layer 114 in some cases.

Note that as the electron-injection layer 115, it is possible to use a layer that contains a substance having an electron-transport property (preferably an organic compound having a bipyridine skeleton) and contains a fluoride of the alkali metal or the alkaline earth metal at a concentration higher than that at which the electron-injection layer 115 becomes in a microcrystalline state (50 wt % or higher). Since the layer has a low refractive index, a light-emitting device including the layer can have high external quantum efficiency.

Instead of the electron-injection layer 115, the charge-generation layer 116 may be provided (FIG. 1B). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film containing the above-described acceptor material as a material included in the composite material and a film containing a hole-transport material. When a potential is applied to the p-type layer 117, electrons are injected into the electron-transport layer 114 and holes are injected into the second electrode 102 serving as a cathode; thus, the light-emitting device operates.

Note that the charge-generation layer 116 preferably includes an electron-relay layer 118 and/or an electron-injection buffer layer 119 in addition to the p-type layer 117.

The electron-relay layer 118 includes at least the substance having an electron-transport property and has a function of preventing an interaction between the electron-injection buffer layer 119 and the p-type layer 117 and smoothly transferring electrons. The LUMO level of the substance having an electron-transport property included in the electron-relay layer 118 is preferably between the LUMO level of the acceptor substance in the p-type layer 117 and the LUMO level of a substance included in a layer of the electron-transport layer 114 that is in contact with the charge-generation layer 116. As a specific value of the energy level, the LUMO level of the substance having an electron-transport property in the electron-relay layer 118 is preferably higher than or equal to −5.0 eV, further preferably higher than or equal to −5.0 eV and lower than or equal to −3.0 eV. Note that as the substance having an electron-transport property in the electron-relay layer 118, a phthalocyanine-based material or a metal complex having a metal-oxygen bond and an aromatic ligand is preferably used.

A substance having a high electron-injection property can be used for the electron-injection buffer layer 119; for example, an alkali metal, an alkaline earth metal, a rare earth metal, or a compound thereof (an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)) can be used.

In the case where the electron-injection buffer layer 119 contains the substance having an electron-transport property and a donor substance, an organic compound such as tetrathianaphthacene (abbreviation: TTN), nickelocene, or decamethylnickelocene can be used as the donor substance, as well as an alkali metal, an alkaline earth metal, a rare earth metal, or a compound thereof (e.g., an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used. Since the organic compound of one embodiment of the present invention has a low refractive index, using the organic compound for the electron-injection buffer layer 119 enables the light-emitting device to have high external quantum efficiency.

For the second electrode 102, a metal, an alloy, an electrically conductive compound, or a mixture thereof that has a low work function (specifically, lower than or equal to 3.8 eV) or the like can be used. Specific examples of such a cathode material include elements belonging to Group 1 or 2 of the periodic table, such as alkali metals (e.g., lithium (Li) and cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the second electrode 102 and the electron-transport layer, a variety of conductive materials such as Al, Ag, ITO, or indium oxide-tin oxide containing silicon or silicon oxide can be used for the second electrode 102 regardless of the work function. Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Furthermore, any of a variety of methods can be used for forming the EL layer 103, regardless of whether it is a dry method or a wet method. For example, a vacuum evaporation method, a gravure printing method, an offset printing method, a screen printing method, an ink-jet method, a spin coating method, or the like may be used.

Different methods may be used to form the electrodes or the layers described above.

The structure of the layers provided between the first electrode 101 and the second electrode 102 is not limited to the above-described structure. Preferably, a light-emitting region where holes and electrons recombine is positioned away from the first electrode 101 and the second electrode 102 so as to prevent quenching due to the proximity of the light-emitting region and a metal used for electrodes and carrier-injection layers.

Furthermore, in order that transfer of energy from an exciton generated in the light-emitting layer can be suppressed, preferably, the hole-transport layer and the electron-transport layer which are in contact with the light-emitting layer 113, particularly a carrier-transport layer closer to the recombination region in the light-emitting layer 113, are formed using a substance having a wider band gap than the light-emitting material of the light-emitting layer or the light-emitting material included in the light-emitting layer.

Next, an embodiment of a light-emitting device with a structure in which a plurality of light-emitting units are stacked (this type of light-emitting device is also referred to as a stacked or tandem light-emitting device) is described with reference to FIG. 1C. This light-emitting device includes a plurality of light-emitting units between an anode and a cathode. One light-emitting unit has substantially the same structure as the EL layer 103 illustrated in FIG. 1A. In other words, the light-emitting device illustrated in FIG. 1A or FIG. 1B includes a single light-emitting unit, and the light-emitting device illustrated in FIG. 1C includes a plurality of light-emitting units.

In FIG. 1C, a first light-emitting unit 511 and a second light-emitting unit 512 are stacked between an anode 501 and a cathode 502, and a charge-generation layer 513 is provided between the first light-emitting unit 511 and the second light-emitting unit 512. The anode 501 and the cathode 502 correspond, respectively, to the first electrode 101 and the second electrode 102 illustrated in FIG. 1A, and the materials given in the description for FIG. 1A can be used. Furthermore, the first light-emitting unit 511 and the second light-emitting unit 512 may have the same structure or different structures.

The charge-generation layer 513 has a function of injecting electrons into one of the light-emitting units and injecting holes into the other of the light-emitting units when a voltage is applied between the anode 501 and the cathode 502. That is, in FIG. 1C, the charge-generation layer 513 injects electrons into the first light-emitting unit 511 and holes into the second light-emitting unit 512 when a voltage is applied such that the potential of the anode becomes higher than the potential of the cathode.

The charge-generation layer 513 preferably has a structure similar to that of the charge-generation layer 116 described with reference to FIG. 1B. A composite material of an organic compound and a metal oxide has an excellent carrier-injection property and an excellent carrier-transport property; thus, low-voltage driving and low-current driving can be achieved. In the case where the anode-side surface of a light-emitting unit is in contact with the charge-generation layer 513, the charge-generation layer 513 can also function as a hole-injection layer of the light-emitting unit; therefore, a hole-injection layer is not necessarily provided in the light-emitting unit.

In the case where the charge-generation layer 513 includes the electron-injection buffer layer 119, the electron-injection buffer layer 119 functions as the electron-injection layer in the light-emitting unit on the anode side; thus, an electron-injection layer is not necessarily formed in the light-emitting unit on the anode side.

The light-emitting device having two light-emitting units is described with reference to FIG. 1C; however, one embodiment of the present invention can also be applied to a light-emitting device in which three or more light-emitting units are stacked. When a plurality of light-emitting units partitioned by the charge-generation layer 513 are provided between a pair of electrodes as in the light-emitting device of this embodiment, it is possible to provide a long-life device that can emit light with high luminance at a low current density. A light-emitting apparatus that can be driven at a low voltage and has low power consumption can be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as the whole.

The above-described layers and electrodes such as the EL layer 103, the first light-emitting unit 511, the second light-emitting unit 512, and the charge-generation layer can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the layers and electrodes.

### Embodiment 3

In this embodiment, a light-emitting apparatus including the light-emitting device described in Embodiment 2 is described.

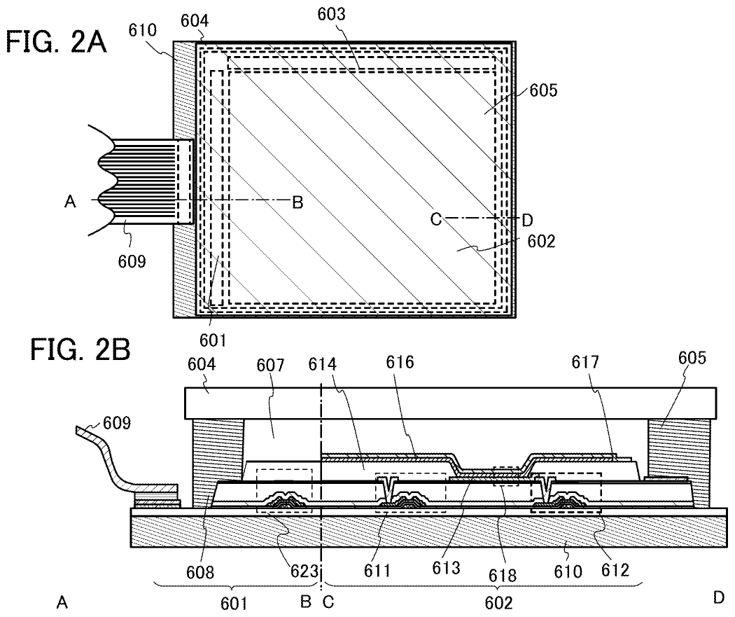

In this embodiment, the light-emitting apparatus manufactured using the light-emitting device described in Embodiment 2 is described with reference to FIGS. 2A and 2B. Note that FIG. 2A is a top view of the light-emitting apparatus and FIG. 2B is a cross-sectional view taken along the lines A-B and C-D in FIG. 2A. This light-emitting apparatus includes a driver circuit portion (source line driver circuit) 601, a pixel portion 602, and a driver circuit portion (gate line driver circuit) 603, which control light emission of a light-emitting device and are illustrated with dotted lines. Reference numeral 604 denotes a sealing substrate; 605, a sealing material; and 607, a space surrounded by the sealing material 605.

Reference numeral 608 denotes a lead wiring for transmitting signals to be input to the source line driver circuit 601 and the gate line driver circuit 603 and receiving signals such as a video signal, a clock signal, a start signal, and a reset signal from a flexible printed circuit (FPC) 609 serving as an external input terminal. Although only the FPC is illustrated here, a printed wiring board (PWB) may be attached to the FPC. The light-emitting apparatus in this specification includes, in its category, not only the light-emitting apparatus itself but also the light-emitting apparatus provided with the FPC or the PWB.

Next, a cross-sectional structure is described with reference to FIG. 2B. The driver circuit portions and the pixel portion are formed over an element substrate 610; here, the source line driver circuit 601, which is a driver circuit portion, and one pixel in the pixel portion 602 are illustrated.

The element substrate 610 may be a substrate containing glass, quartz, an organic resin, a metal, an alloy, or a semiconductor or a plastic substrate formed of fiber reinforced plastic (FRP), polyvinyl fluoride (PVF), polyester, an acrylic resin, or the like.

The structure of transistors used in pixels and driver circuits is not particularly limited. For example, inverted staggered transistors may be used, or staggered transistors may be used. Furthermore, top-gate transistors or bottom-gate transistors may be used. A semiconductor material used for the transistors is not particularly limited, and for example, silicon, germanium, silicon carbide, gallium nitride, or the like can be used. Alternatively, an oxide semiconductor containing at least one of indium, gallium, and zinc, such as an In—Ga—Zn-based metal oxide, may be used.

There is no particular limitation on the crystallinity of a semiconductor material used for the transistors, and an amorphous semiconductor or a semiconductor having crystallinity (a microcrystalline semiconductor, a polycrystalline semiconductor, a single crystal semiconductor, or a semiconductor partly including crystal regions) may be used. A semiconductor having crystallinity is preferably used because deterioration of the transistor characteristics can be suppressed.

Here, an oxide semiconductor is preferably used for semiconductor devices such as the transistors provided in the pixels and driver circuits and transistors used for touch sensors described later, and the like. In particular, an oxide semiconductor having a wider band gap than silicon is preferably used. When an oxide semiconductor having a wider band gap than silicon is used, off-state current of the transistors can be reduced.

The oxide semiconductor preferably contains at least indium (In) or zinc (Zn). Further preferably, the oxide semiconductor contains an oxide represented by an In-M-Zn-based oxide (M represents a metal such as Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and in which the adjacent crystal parts have no grain boundary.

The use of such materials for the semiconductor layer makes it possible to provide a highly reliable transistor in which a change in the electrical characteristics is suppressed.

Charge accumulated in a capacitor through a transistor including the above-described semiconductor layer can be held for a long time because of the low off-state current of the transistor. When such a transistor is used in a pixel, operation of a driver circuit can be stopped while a gray scale of an image displayed in each display region is maintained. As a result, an electronic device with extremely low power consumption can be obtained.

For stable characteristics of the transistor, a base film is preferably provided. The base film can be formed with a single layer or stacked layers using an inorganic insulating film such as a silicon oxide film, a silicon nitride film, a silicon oxynitride film, or a silicon nitride oxide film. The base film can be formed by a sputtering method, a chemical vapor deposition (CVD) method (e.g., a plasma CVD method, a thermal CVD method, or a metal organic CVD (MOCVD) method), an atomic layer deposition (ALD) method, a coating method, a printing method, or the like. Note that the base film is not necessarily provided.

Note that an FET 623 is illustrated as a transistor formed in the driver circuit portion 601. The driver circuit may be formed with any of a variety of circuits such as a CMOS circuit, a PMOS circuit, or an NMOS circuit. Although a driver integrated type in which the driver circuit is formed over the substrate is illustrated in this embodiment, the driver circuit is not necessarily formed over the substrate, and the driver circuit can be formed outside, not over the substrate.

The pixel portion 602 includes a plurality of pixels including a switching FET 611, a current controlling FET 612, and a first electrode 613 electrically connected to a drain of the current controlling FET 612. One embodiment of the present invention is not limited to the structure. The pixel portion 602 may include three or more FETs and a capacitor in combination.

Note that an insulator 614 is formed to cover an end portion of the first electrode 613. Here, the insulator 614 can be formed using a positive photosensitive acrylic resin film.

In order to improve the coverage with an EL layer or the like which is formed later, the insulator 614 is formed to have a curved surface with curvature at its upper or lower end portion. For example, in the case where a positive photosensitive acrylic resin is used as a material of the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a second electrode 617 are formed over the first electrode 613. Here, as a material used for the first electrode 613 functioning as an anode, a material having a high work function is preferably used. For example, a single-layer film of an ITO film, an indium tin oxide film containing silicon, an indium oxide film containing zinc oxide at 2 wt % to 20 wt %, a titanium nitride film, a chromium film, a tungsten film, a Zn film, a Pt film, or the like, a stack of a titanium nitride film and a film containing aluminum as its main component, a stack of three layers of a titanium nitride film, a film containing aluminum as its main component, and a titanium nitride film, or the like can be used. The stacked-layer structure enables low wiring resistance, favorable ohmic contact, and a function as an anode.

The EL layer 616 is formed by any of a variety of methods such as an evaporation method using an evaporation mask, an ink-jet method, and a spin coating method. The EL layer 616 has the structure described in Embodiment 2. As another material included in the EL layer 616, a low molecular compound or a high molecular compound (including an oligomer or a dendrimer) may be used.

As a material used for the second electrode 617, which is formed over the EL layer 616 and functions as a cathode, a material having a low work function (e.g., Al, Mg, Li, and Ca, or an alloy or a compound thereof, such as MgAg, MgIn, and AlLi) is preferably used. In the case where light generated in the EL layer 616 is transmitted through the second electrode 617, a stack of a thin metal film and a transparent conductive film (e.g., ITO, indium oxide containing zinc oxide at 2 wt % to 20 wt %, indium tin oxide containing silicon, or zinc oxide (ZnO)) is preferably used for the second electrode 617.

Note that the light-emitting device is formed with the first electrode 613, the EL layer 616, and the second electrode 617. The light-emitting device is the light-emitting device described in Embodiment 2. In the light-emitting apparatus of this embodiment, the pixel portion, which includes a plurality of light-emitting devices, may include both the light-emitting device described in Embodiment 2 and a light-emitting device having a different structure.

The sealing substrate 604 is attached to the element substrate 610 with the sealing material 605, so that a light-emitting device 618 is provided in the space 607 surrounded by the element substrate 610, the sealing substrate 604, and the sealing material 605. The space 607 may be filled with a filler, or may be filled with an inert gas (such as nitrogen or argon), or the sealing material. It is preferable that the sealing substrate be provided with a recessed portion and a drying agent be provided in the recessed portion, in which case deterioration due to influence of moisture can be suppressed.

An epoxy-based resin or glass frit is preferably used for the sealing material 605. It is preferable that such a material transmit moisture or oxygen as little as possible. As the sealing substrate 604, a glass substrate, a quartz substrate, or a plastic substrate formed of fiber reinforced plastic (FRP), polyvinyl fluoride (PVF), polyester, an acrylic resin, or the like can be used.

Although not illustrated in FIGS. 2A and 2B, a protective film may be provided over the second electrode. As the protective film, an organic resin film or an inorganic insulating film may be formed. The protective film may be formed so as to cover an exposed portion of the sealing material 605. The protective film may be provided so as to cover surfaces and side surfaces of the pair of substrates and exposed side surfaces of a sealing layer, an insulating layer, and the like.

The protective film can be formed using a material that does not easily transmit an impurity such as water. Thus, diffusion of an impurity such as water from the outside into the inside can be effectively suppressed.

As a material of the protective film, an oxide, a nitride, a fluoride, a sulfide, a ternary compound, a metal, a polymer, or the like can be used. For example, the material may contain aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, aluminum nitride, hafnium nitride, silicon nitride, tantalum nitride, titanium nitride, niobium nitride, molybdenum nitride, zirconium nitride, gallium nitride, a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like.

The protective film is preferably formed using a deposition method with favorable step coverage. One such method is an atomic layer deposition (ALD) method. A material that can be deposited by an ALD method is preferably used for the protective film. A dense protective film having reduced defects such as cracks or pinholes or a uniform thickness can be formed by an ALD method. Furthermore, damage to a process member in forming the protective film can be reduced.

By an ALD method, a uniform protective film with few defects can be formed even on, for example, a surface with a complex uneven shape or upper, side, and lower surfaces of a touch panel.

As described above, the light-emitting apparatus manufactured using the light-emitting device described in Embodiment 2 can be obtained.

The light-emitting apparatus in this embodiment is manufactured using the light-emitting device described in Embodiment 2 and thus can have favorable characteristics. Specifically, since the light-emitting device described in Embodiment 2 has high emission efficiency, the light-emitting apparatus can achieve low power consumption.

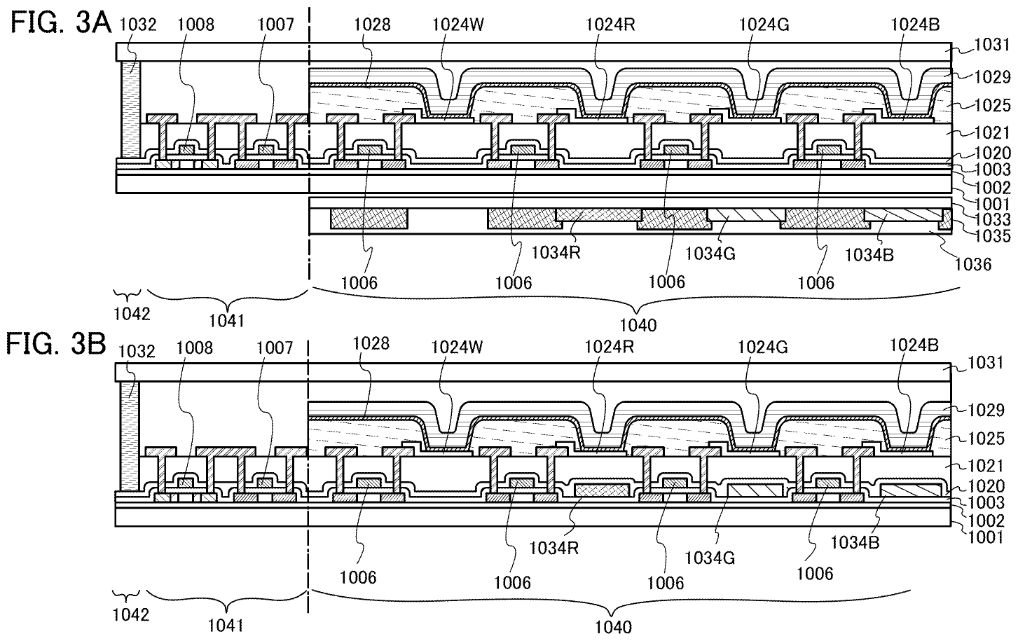

FIGS. 3A and 3B each illustrate an example of a light-emitting apparatus in which full color display is achieved by formation of a light-emitting device exhibiting white light emission and with the use of coloring layers (color filters) and the like. FIG. 3A illustrates a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, first electrodes 1024W, 1024R, 1024G, and 1024B of light-emitting devices, a partition 1025, an EL layer 1028, a second electrode 1029 of the light-emitting devices, a sealing substrate 1031, a sealing material 1032, and the like.

In FIG. 3A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix is aligned and fixed to the substrate 1001. Note that the coloring layers and the black matrix 1035 are covered with an overcoat layer 1036. In FIG. 3A, light emitted from part of the light-emitting layer does not pass through the coloring layers, while light emitted from the other part of the light-emitting layer passes through the coloring layers. Since the light that does not pass through the coloring layers is white and the light that passes through any one of the coloring layers is red, green, or blue, an image can be displayed using pixels of the four colors.

FIG. 3B illustrates an example in which the coloring layers (the red coloring layer 1034R, the green coloring layer 1034G, and the blue coloring layer 1034B) are provided between the gate insulating film 1003 and the first interlayer insulating film 1020. As in the structure, the coloring layers may be provided between the substrate 1001 and the sealing substrate 1031.

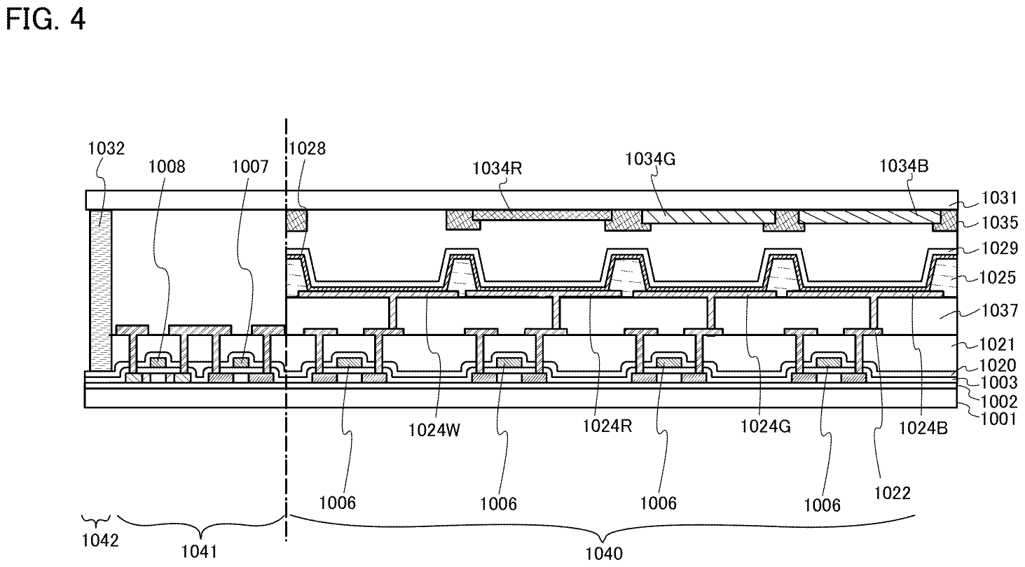

The above-described light-emitting apparatus is a light-emitting apparatus having a structure in which light is extracted from the substrate 1001 side where FETs are formed (a bottom emission structure), but may be a light-emitting apparatus having a structure in which light is extracted from the sealing substrate 1031 side (a top emission structure). FIG. 4 is a cross-sectional view of a light-emitting apparatus having a top emission structure. In this case, a substrate that does not transmit light can be used as the substrate 1001. The process up to the step of forming a connection electrode that connects the FET and the anode of the light-emitting device is performed in a manner similar to that of the light-emitting apparatus having a bottom emission structure. Then, a third interlayer insulating film 1037 is formed to cover an electrode 1022. This insulating film may have a planarization function. The third interlayer insulating film 1037 can be formed using a material similar to that of the second interlayer insulating film, and can alternatively be formed using any of other known materials.

The first electrodes 1024W, 1024R, 1024G, and 1024B of the light-emitting devices each serve as an anode here, but may serve as a cathode. Furthermore, in the case of a light-emitting apparatus having a top emission structure as illustrated in FIG. 4, the first electrodes are preferably reflective electrodes. The EL layer 1028 is formed to have a structure similar to the structure of the EL layer 103, which is described in Embodiment 2, with which white light emission can be obtained.

In the case of a top emission structure as illustrated in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 which is positioned between pixels. The coloring layers (the red coloring layer 1034R, the green coloring layer 1034G, and the blue coloring layer 1034B) and the black matrix may be covered with the overcoat layer 1036. Note that a light-transmitting substrate is used as the sealing substrate 1031. Although an example in which full color display is performed using four colors of red, green, blue, and white is shown here, there is no particular limitation and full color display using four colors of red, yellow, green, and blue or three colors of red, green, and blue may be performed.

In the light-emitting apparatus having a top emission structure, a microcavity structure can be suitably employed. A light-emitting device with a microcavity structure is formed with the use of a reflective electrode as the first electrode and a transflective electrode as the second electrode. The light-emitting device with a microcavity structure includes at least an EL layer between the reflective electrode and the transflective electrode, which includes at least a light-emitting layer serving as a light-emitting region.

Note that the reflective electrode has a visible light reflectivity of 40% to 100%, preferably 70% to 100%, and a resistivity of 1×10⁻² Ωcm or lower. In addition, the transflective electrode has a visible light reflectivity of 20% to 80%, preferably 40% to 70%, and a resistivity of 1×10⁻² Ωcm or lower.

Light emitted from the light-emitting layer included in the EL layer is reflected and resonated by the reflective electrode and the transflective electrode.

In the light-emitting device, by changing thicknesses of the transparent conductive film, the composite material, the carrier-transport material, and the like, the optical path length between the reflective electrode and the transflective electrode can be changed. Thus, light with a wavelength that is resonated between the reflective electrode and the transflective electrode can be intensified while light with a wavelength that is not resonated therebetween can be attenuated.

Note that light that is reflected back by the reflective electrode (first reflected light) considerably interferes with light that directly enters the transflective electrode from the light-emitting layer (first incident light). For this reason, the optical path length between the reflective electrode and the light-emitting layer is preferably adjusted to (2n−1)λ/4 (n is a natural number of 1 or larger and λ is a wavelength of light to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer. The tandem light-emitting device described above may be combined with a plurality of EL layers; for example, a light-emitting device may have a structure in which a plurality of EL layers are provided, a charge-generation layer is provided between the EL layers, and each EL layer includes a plurality of light-emitting layers or a single light-emitting layer.

With the microcavity structure, emission intensity with a specific wavelength in the front direction can be increased, whereby power consumption can be reduced. Note that in the case of a light-emitting apparatus that displays images with subpixels of four colors, red, yellow, green, and blue, the light-emitting apparatus can have favorable characteristics because the luminance can be increased owing to yellow light emission and each subpixel can employ a microcavity structure suitable for wavelengths of the corresponding color.

The light-emitting apparatus in this embodiment is manufactured using the light-emitting device described in Embodiment 2 and thus can have favorable characteristics. Specifically, since the light-emitting device described in Embodiment 2 has high emission efficiency, the light-emitting apparatus can achieve low power consumption.

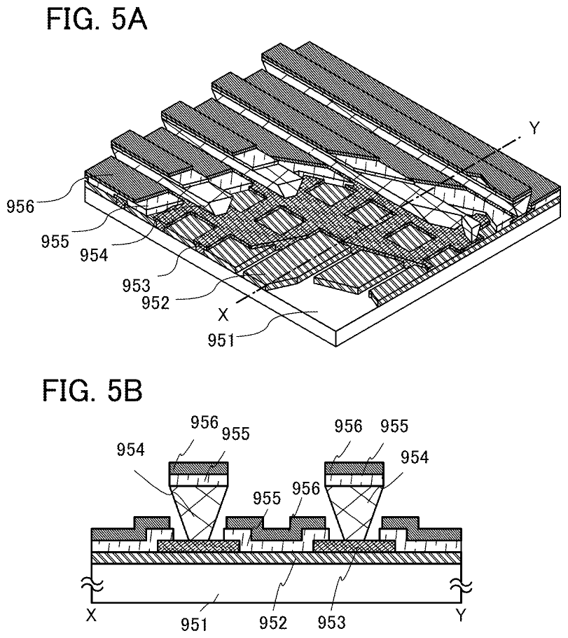

An active matrix light-emitting apparatus is described above, whereas a passive matrix light-emitting apparatus is described below. FIGS. 5A and 5B illustrate a passive matrix light-emitting apparatus manufactured using the present invention. Note that FIG. 5A is a perspective view of the light-emitting apparatus, and FIG. 5B is a cross-sectional view taken along the line X-Y in FIG. 5A. In FIGS. 5A and 5B, over a substrate 951, an EL layer 955 is provided between an electrode 952 and an electrode 956. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section taken along the direction of the short side of the partition layer 954 is trapezoidal, and the lower side (a side of the trapezoid that is parallel to the surface of the insulating layer 953 and is in contact with the insulating layer 953) is shorter than the upper side (a side of the trapezoid that is parallel to the surface of the insulating layer 953 and is not in contact with the insulating layer 953). The partition layer 954 provided in this manner can prevent defects in the light-emitting device due to static electricity or others. The passive matrix light-emitting apparatus also includes the light-emitting device described in Embodiment 2; thus, the light-emitting apparatus can have high reliability or low power consumption.

In the light-emitting apparatus described above, many minute light-emitting devices arranged in a matrix can each be controlled; thus, the light-emitting apparatus can be suitably used as a display device for displaying images.

This embodiment can be freely combined with any of the other embodiments.

### Embodiment 4

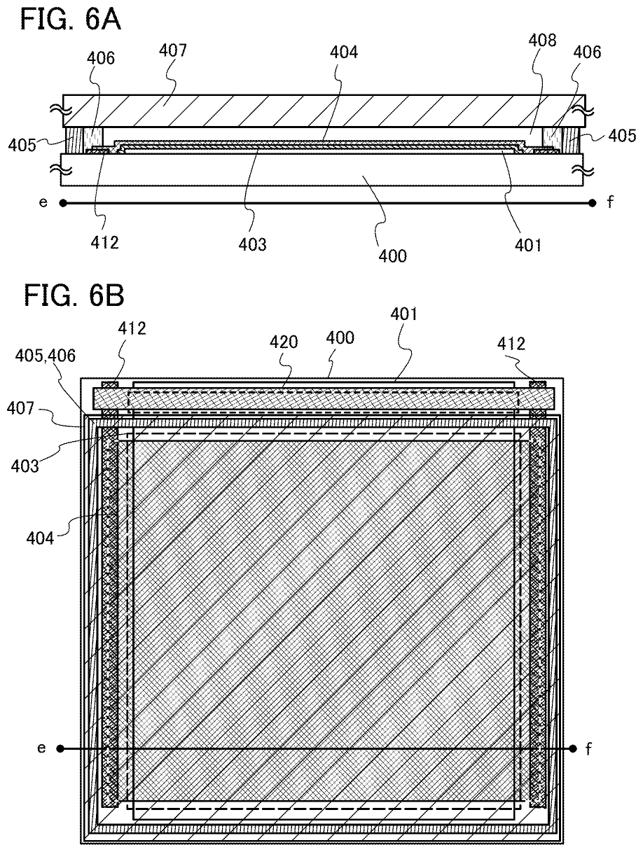

In this embodiment, an example in which the light-emitting device described in Embodiment 2 is used for a lighting device will be described with reference to FIGS. 6A and 6B. FIG. 6B is a top view of the lighting device, and FIG. 6A is a cross-sectional view taken along the line e-f in FIG. 6B.

In the lighting device in this embodiment, a first electrode 401 is formed over a substrate 400 which is a support with a light-transmitting property. The first electrode 401 corresponds to the first electrode 101 in Embodiment 2. When light is extracted through the first electrode 401 side, the first electrode 401 is formed using a material having a light-transmitting property.

A pad 412 for applying voltage to a second electrode 404 is provided over the substrate 400.

An EL layer 403 is formed over the first electrode 401. The structure of the EL layer 403 corresponds to, for example, the structure of the EL layer 103 in Embodiment 2, or the structure in which the light-emitting units 511 and 512 and the charge-generation layer 513 are combined. Refer to the description for the structure.

The second electrode 404 is formed to cover the EL layer 403. The second electrode 404 corresponds to the second electrode 102 in Embodiment 2. The second electrode 404 is formed using a material having high reflectance when light is extracted through the first electrode 401 side. The second electrode 404 is connected to the pad 412, whereby voltage is applied.

As described above, the lighting device described in this embodiment includes a light-emitting device including the first electrode 401, the EL layer 403, and the second electrode 404. Since the light-emitting device is a light-emitting device with high emission efficiency, the lighting device in this embodiment can have low power consumption.

The substrate 400 provided with the light-emitting device having the above structure is fixed to a sealing substrate 407 with sealing materials 405 and 406 and sealing is performed, whereby the lighting device is completed. It is possible to use only either the sealing material 405 or the sealing material 406. The inner sealing material 406 (not shown in FIG. 6B) can be mixed with a desiccant which enables moisture to be adsorbed, increasing reliability.

When parts of the pad 412 and the first electrode 401 are extended to the outside of the sealing materials 405 and 406, the extended parts can serve as external input terminals. An IC chip 420 mounted with a converter or the like may be provided over the external input terminals.

The lighting device described in this embodiment includes as an EL device the light-emitting device described in Embodiment 2; thus, the light-emitting apparatus can have low power consumption.

### Embodiment 5

In this embodiment, examples of electronic devices each including the light-emitting device described in Embodiment 2 will be described. The light-emitting device described in Embodiment 2 has high emission efficiency and low power consumption. As a result, the electronic devices described in this embodiment can each include a light-emitting portion having low power consumption.

Examples of the electronic device including the above light-emitting device include television devices (also referred to as TV or television receivers), monitors for computers and the like, digital cameras, digital video cameras, digital photo frames, cellular phones (also referred to as mobile phones or mobile phone devices), portable game machines, portable information terminals, audio playback devices, and large game machines such as pachinko machines. Specific examples of these electronic devices are shown below.

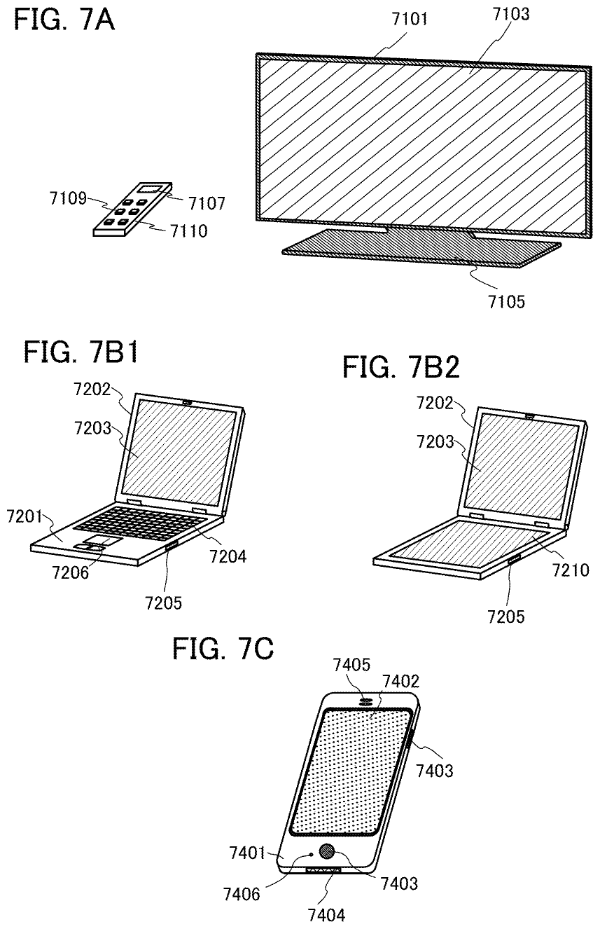

FIG. 7A illustrates an example of a television device. In the television device, a display portion 7103 is incorporated in a housing 7101. Here, the housing 7101 is supported by a stand 7105. Images can be displayed on the display portion 7103, and in the display portion 7103, the light-emitting devices described in Embodiment 2 are arranged in a matrix.

The television device can be operated with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device is provided with a receiver, a modem, and the like. With the use of the receiver, a general television broadcast can be received. Moreover, when the television device is connected to a communication network with or without wires via the modem, one-way (from a sender to a receiver) or two-way (between a sender and a receiver or between receivers, for example) data communication can be performed.

FIG. 7B1 illustrates a computer, which includes a main body 7201, a housing 7202, a display portion 7203, a keyboard 7204, an external connection port 7205, a pointing device 7206, and the like. Note that this computer is manufactured using the light-emitting devices described in Embodiment 2 and arranged in a matrix in the display portion 7203. The computer illustrated in FIG. 7B1 may have a structure illustrated in FIG. 7B2. A computer illustrated in FIG. 7B2 is provided with a second display portion 7210 instead of the keyboard 7204 and the pointing device 7206. The second display portion 7210 is a touch panel, and input operation can be performed by touching display for input on the second display portion 7210 with a finger or a dedicated pen. The second display portion 7210 can also display images other than the display for input. The display portion 7203 may also be a touch panel. Connecting the two screens with a hinge can prevent troubles; for example, the screens can be prevented from being cracked or broken while the computer is being stored or carried.

FIG. 7C illustrates an example of a portable terminal. A cellular phone is provided with a display portion 7402 incorporated in a housing 7401, operation buttons 7403, an external connection port 7404, a speaker 7405, a microphone 7406, and the like. Note that the cellular phone has the display portion 7402 including the light-emitting devices described in Embodiment 2 and arranged in a matrix.

When the display portion 7402 of the portable terminal illustrated in FIG. 7C is touched with a finger or the like, data can be input into the portable terminal. In this case, operations such as making a call and creating an e-mail can be performed by touching the display portion 7402 with a finger or the like.

The display portion 7402 has mainly three screen modes. The first mode is a display mode mainly for displaying images. The second mode is an input mode mainly for inputting data such as text. The third mode is a display-and-input mode in which the two modes, the display mode and the input mode, are combined.

For example, in the case of making a call or creating an e-mail, a text input mode mainly for inputting text is selected for the display portion 7402 so that text displayed on the screen can be input. In this case, it is preferable to display a keyboard or number buttons on almost the entire screen of the display portion 7402.

When a sensing device including a sensor such as a gyroscope sensor or an acceleration sensor for detecting inclination is provided inside the portable terminal, display on the screen of the display portion 7402 can be automatically changed in direction by determining the orientation of the portable terminal (whether the portable terminal is placed horizontally or vertically).

The screen modes are switched by touching the display portion 7402 or operating the operation buttons 7403 of the housing 7401. Alternatively, the screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal sensed by an optical sensor in the display portion 7402 is sensed, the screen mode may be controlled so as to be switched from the input mode to the display mode.

The display portion 7402 may also function as an image sensor. For example, an image of a palm print, a fingerprint, or the like is taken when the display portion 7402 is touched with the palm or the finger, whereby personal authentication can be performed. Furthermore, by providing a backlight or a sensing light source that emits near-infrared light in the display portion, an image of a finger vein, a palm vein, or the like can be taken.

Note that the structure described in this embodiment can be combined with any of the structures described in Embodiments 1 to 4 as appropriate.

As described above, the application range of the light-emitting apparatus including the light-emitting device described in Embodiment 2 is extremely wide, so that this light-emitting apparatus can be used for electronic devices in a variety of fields. By using the light-emitting device described in Embodiment 2, an electronic device with low power consumption can be obtained.

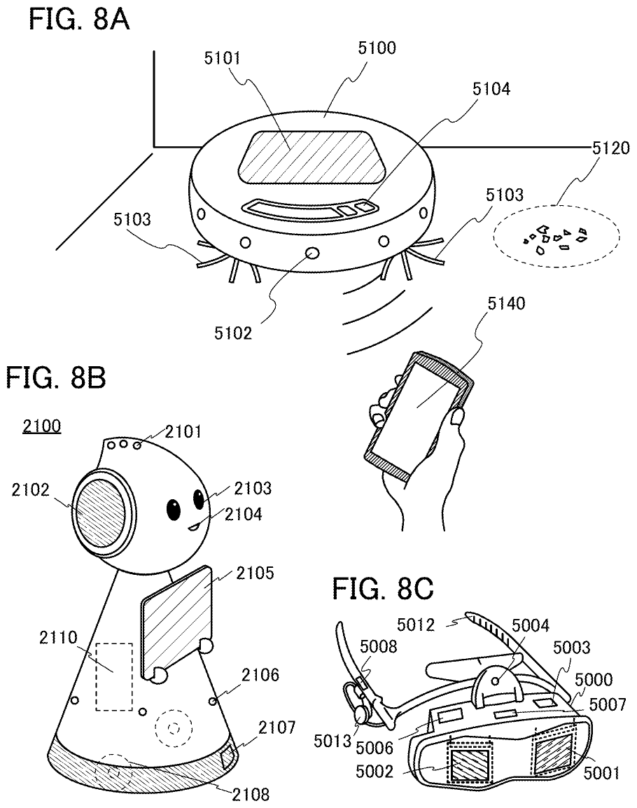

FIG. 8A is a schematic view illustrating an example of a cleaning robot.

A cleaning robot 5100 includes a display 5101 on its top surface, a plurality of cameras 5102 on its side surface, a brush 5103, and operation buttons 5104. Although not illustrated, the bottom surface of the cleaning robot 5100 is provided with a tire, an inlet, and the like. Furthermore, the cleaning robot 5100 includes various sensors such as an infrared sensor, an ultrasonic sensor, an acceleration sensor, a piezoelectric sensor, an optical sensor, and a gyroscope sensor. The cleaning robot 5100 has a wireless communication means.

The cleaning robot 5100 is self-propelled, detects dust 5120, and sucks up the dust through the inlet provided on the bottom surface.

The cleaning robot 5100 can determine whether there is an obstacle such as a wall, furniture, or a step by analyzing images taken by the cameras 5102. When the cleaning robot 5100 detects an object that is likely to be caught in the brush 5103 (e.g., a wire) by image analysis, the rotation of the brush 5103 can be stopped.

The display 5101 can display the remaining capacity of a battery, the amount of collected dust, and the like. The display 5101 may display a path on which the cleaning robot 5100 has run. The display 5101 may be a touch panel, and the operation buttons 5104 may be provided on the display 5101.

The cleaning robot 5100 can communicate with a portable electronic device 5140 such as a smartphone. Images taken by the cameras 5102 can be displayed on the portable electronic device 5140. Accordingly, an owner of the cleaning robot 5100 can monitor his/her room even when the owner is not at home. The owner can also check the display on the display 5101 by the portable electronic device such as a smartphone.

The light-emitting apparatus of one embodiment of the present invention can be used for the display 5101.

A robot 2100 illustrated in FIG. 8B includes an arithmetic device 2110, an illuminance sensor 2101, a microphone 2102, an upper camera 2103, a speaker 2104, a display 2105, a lower camera 2106, an obstacle sensor 2107, and a moving mechanism 2108.

The microphone 2102 has a function of detecting a speaking voice of a user, an environmental sound, and the like. The speaker 2104 has a function of outputting sound. The robot 2100 can communicate with a user using the microphone 2102 and the speaker 2104.

The display 2105 has a function of displaying various kinds of information. The robot 2100 can display information desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 8C illustrates an example of a goggle-type display. The goggle-type display includes, for example, a housing 5000, a display portion 5001, a speaker 5003, an LED lamp 5004, a connection terminal 5006, a sensor 5007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared ray), a microphone 5008, a second display portion 5002, a support 5012, and an earphone 5013.

The light-emitting apparatus of one embodiment of the present invention can be used for the display portion 5001 and the second display portion 5002.

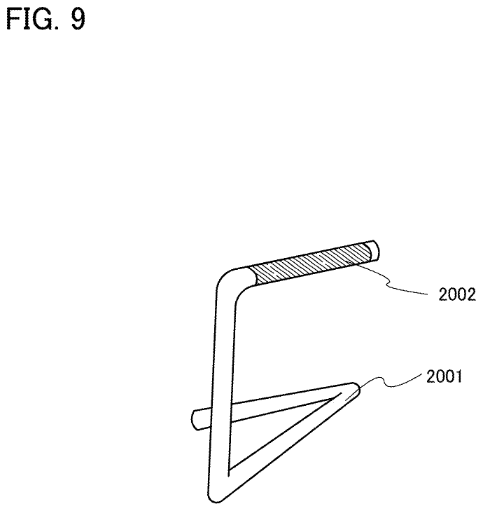

FIG. 9 illustrates an example in which the light-emitting device described in Embodiment 2 is used for a table lamp which is a lighting device. The table lamp illustrated in FIG. 9 includes a housing 2001 and a light source 2002, and the lighting device described in Embodiment 3 may be used for the light source 2002.

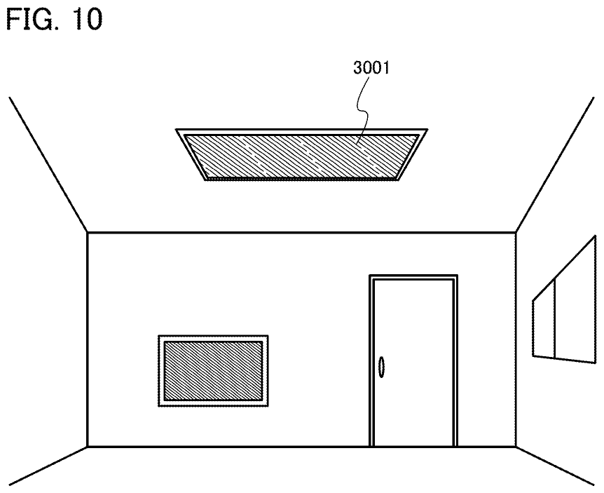

FIG. 10 illustrates an example in which the light-emitting device described in Embodiment 2 is used for an indoor lighting device 3001. Since the light-emitting device described in Embodiment 2 has high emission efficiency, the lighting device can have low power consumption. Furthermore, since the light-emitting device described in Embodiment 2 can have a large area, the light-emitting device can be used for a large-area lighting device. Furthermore, since the light-emitting device described in Embodiment 2 is thin, the light-emitting device can be used for a lighting device having a reduced thickness.

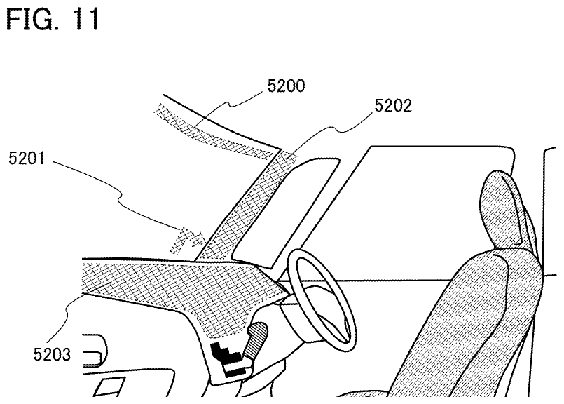

The light-emitting device described in Embodiment 2 can also be used for an automobile windshield or an automobile dashboard. FIG. 11 illustrates one mode in which the light-emitting devices described in Embodiment 2 are used for an automobile windshield and an automobile dashboard. Display regions 5200 to 5203 each include the light-emitting device described in Embodiment 2.

The display regions 5200 and 5201 are display devices which are provided in the automobile windshield and include the light-emitting device described in Embodiment 2. The light-emitting device described in Embodiment 2 can be formed into what is called a see-through display device, through which the opposite side can be seen, by including a first electrode and a second electrode formed of light-transmitting electrodes. Such see-through display devices can be provided even in the automobile windshield without hindering the view. In the case where a driving transistor or the like is provided, a transistor having a light-transmitting property, such as an organic transistor including an organic semiconductor material or a transistor including an oxide semiconductor, is preferably used.

The display region 5202 is a display device which is provided in a pillar portion and includes the light-emitting device described in Embodiment 2. The display region 5202 can compensate for the view hindered by the pillar by displaying an image taken by an imaging unit provided in the car body. Similarly, the display region 5203 provided in the dashboard portion can compensate for the view hindered by the car body by displaying an image taken by an imaging unit provided on the outside of the automobile. Thus, blind areas can be eliminated to enhance the safety. Images that compensate for the areas which a driver cannot see enable the driver to ensure safety easily and comfortably.

The display region 5203 can provide a variety of kinds of information by displaying navigation data, a speedometer, a tachometer, air-condition setting, and the like. The content or layout of the display can be changed freely by a user as appropriate. Note that such information can also be displayed on the display regions 5200 to 5202. The display regions 5200 to 5203 can also be used as lighting devices.

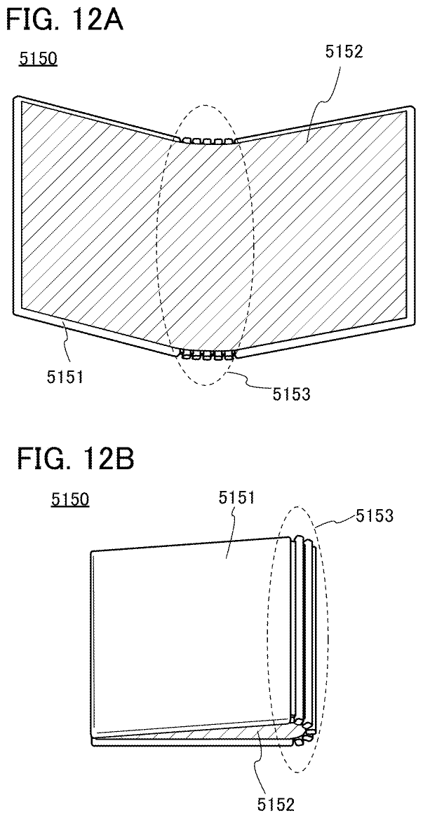

FIGS. 12A and 12B illustrate a foldable portable information terminal 5150. The foldable portable information terminal 5150 includes a housing 5151, a display region 5152, and a bend portion 5153. FIG. 12A illustrates the portable information terminal 5150 that is opened. FIG. 12B illustrates the portable information terminal 5150 that is folded. Despite its large display region 5152, the portable information terminal 5150 is compact in size and has excellent portability when folded.

The display region 5152 can be folded in half with the bend portion 5153. The bend portion 5153 includes a flexible member and a plurality of supporting members. When the display region is folded, the flexible member expands and the bend portion 5153 has a radius of curvature of greater than or equal to 2 mm, preferably greater than or equal to 3 mm.

Note that the display region 5152 may be a touch panel (an input/output device) including a touch sensor (an input device). The light-emitting apparatus of one embodiment of the present invention can be used for the display region 5152.

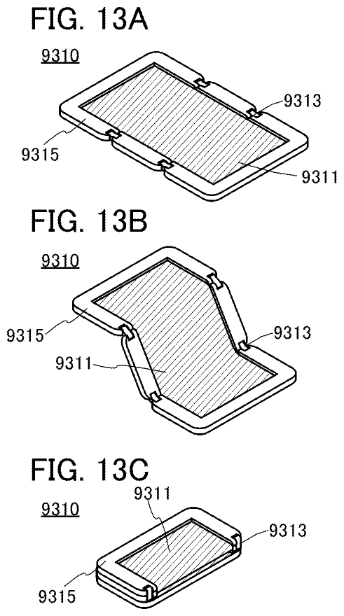

FIGS. 13A to 13C illustrate a foldable portable information terminal 9310. FIG. 13A illustrates the portable information terminal 9310 that is opened. FIG. 13B illustrates the portable information terminal 9310 that is being opened or being folded. FIG. 13C illustrates the portable information terminal 9310 that is folded. The portable information terminal 9310 is highly portable when folded. The portable information terminal 9310 is highly browsable when opened because of a seamless large display region.

A display panel 9311 is supported by three housings 9315 joined together by hinges 9313. Note that the display panel 9311 may be a touch panel (an input/output device) including a touch sensor (an input device). By folding the display panel 9311 at the hinges 9313 between two housings 9315, the portable information terminal 9310 can be reversibly changed in shape from the opened state to the folded state. The light-emitting apparatus of one embodiment of the present invention can be used for the display panel 9311.

### Example 1

### Synthesis Example 1

In this example, a method for synthesizing 2-{(3′,5′-di-tert-butyl)-1,1′-biphenyl-3-yl}-4,6-bis(3,5-di-tert-butylphenyl)-1,3,5-triazine (abbreviation: mmtBumBP-dmmtBuPTzn), which is the organic compound represented by Structural Formula (100) in Embodiment 1, is described. The structural formula of mmtBumBP-dmmtBuPTzn is shown below.

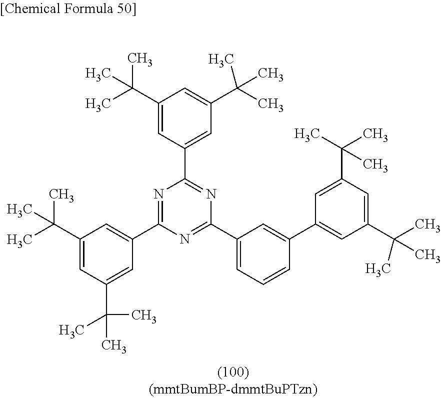 [SMILES: CC(C)(C)c1cc(-c2cccc(-c3nc(-c4cc(C(C)(C)C)cc(C(C)(C)C)c4)nc(-c4cc(C(C)(C)C)cc(C(C)(C)C)c4)n3)c2)cc(C(C)(C)C)c1]

### Step 1: Synthesis of 3-bromo-3′,5′-di-tert-butylbiphenyl

Into a three-neck flask were put 1.0 g (4.3 mmol) of 3,5-di-t-butylphenylboronic acid, 1.5 g (5.2 mmol) of 1-bromo-3-iodobenzene, 4.5 mL of an aqueous solution of potassium carbonate (2 mol/L), 20 mL of toluene, and 3 mL of ethanol, and the mixture was degassed by being stirred under reduced pressure. To this mixture were added 52 mg (0.17 mmol) of tris(2-methylphenyl)phosphine and 10 mg (0.043 mmol) of palladium(II) acetate, and reaction was caused under a nitrogen atmosphere at 80° C. for 14 hours. After the reaction, extraction was performed with toluene and the obtained organic layer was dried with magnesium sulfate. This mixture was gravity-filtered, and the obtained filtrate was purified by silica gel column chromatography with a developing solvent of hexane to give 1.0 g of a target white solid in a yield of 68%. The synthesis scheme of Step 1 is shown in (a-1) below.

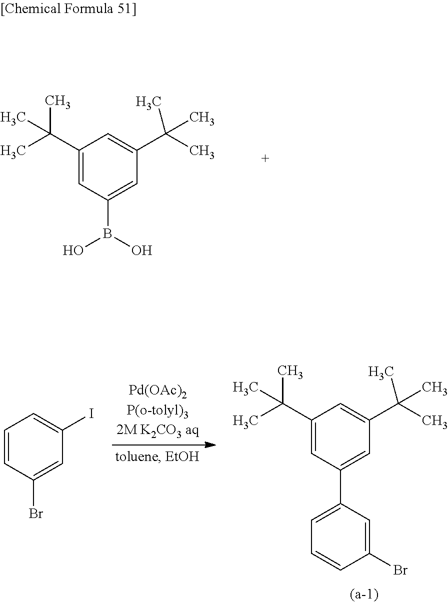 [SMILES: CC(C)(C)c1cc(B(O)O)cc(C(C)(C)C)c1 ; Brc1cccc(I)c1 ; CC(C)(C)c1cc(-c2cccc(Br)c2)cc(C(C)(C)C)c1]

### Step 2: Synthesis of 2-(3′,5′-di-tert-butylbiphenyl-3-yl)-4,4,5,5-tetramethyl-1,3,2-dioxaborolane

Into a three-neck flask were put 1.0 g (2.9 mmol) of 3-bromo-3′,5′-di-tert-butylbiphenyl, 0.96 g (3.8 mmol) of bis(pinacolato)diboron, 0.94 g (9.6 mmol) of potassium acetate, and 30 mL of 1,4-dioxane, and the mixture was degassed by being stirred under reduced pressure. To this mixture were added 0.12 g (0.30 mmol) of 2-dicyclohexylphosphino-2′,6′-dimethoxybiphenyl and 0.12 g (0.15 mmol) of [1,1′-bis(diphenylphosphino)ferrocene]palladium(II) dichloride dichloromethane adduct, and reaction was caused under a nitrogen atmosphere at 110° C. for 24 hours. After the reaction, extraction was performed with toluene and the obtained organic layer was dried with magnesium sulfate. This mixture was gravity-filtered, and the obtained filtrate was purified by silica gel column chromatography with a developing solvent of toluene to give 0.89 g of a target yellow oil in a yield of 78%. The synthesis scheme of Step 2 is shown in (a-2) below.

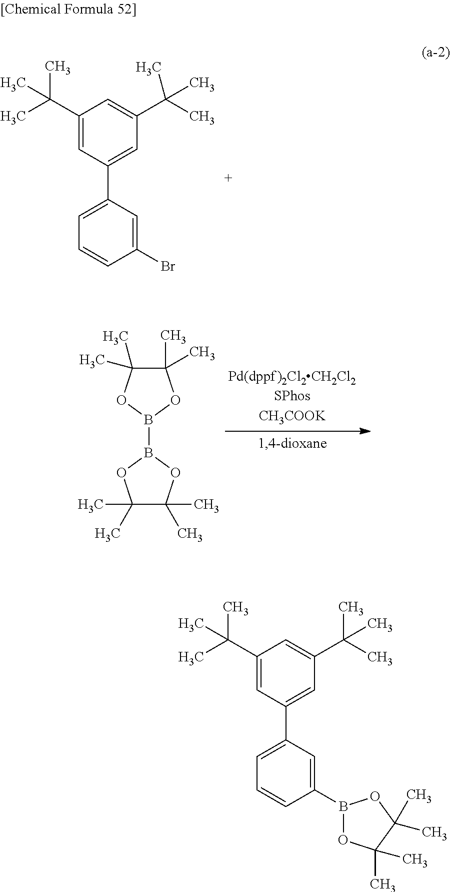

### Step 3: Synthesis of mmtBumBP-dmmtBuPTzn

Into a three-neck flask were put 0.8 g (1.6 mmol) of 4,6-bis(3,5-di-tert-butyl-phenyl)-2-chloro-1,3,5-triazine, 0.89 g (2.3 mmol) of 2-(3′,5′-di-tert-butylbiphenyl-3-yl)-4,4,5,5-tetramethyl-1,3,2-dioxaborolane, 0.68 g (3.2 mmol) of tripotassium phosphate, 3 mL of water, 8 mL of toluene, and 3 mL of 1,4-dioxane, and the mixture was degassed by being stirred under reduced pressure. To this mixture were added 3.5 mg (0.016 mmol) of palladium(II) acetate and 10 mg (0.032 mmol) of tris(2-methylphenyl)phosphine, and the mixture was heated and refluxed under a nitrogen atmosphere for 12 hours. After the reaction, extraction was performed with ethyl acetate and the obtained organic layer was dried with magnesium sulfate. This mixture was gravity-filtered, and the obtained filtrate was concentrated, which was then purified by silica gel column chromatography with a developing solvent of ethyl acetate and hexane in a ratio of 1:20 to give a solid. This solid was purified by silica gel column chromatography with a developing solvent of chloroform and hexane in a ratio of 5:1, which was then changed to 1:0. The obtained solid was recrystallized with hexane to give 0.88 g of a target white solid in a yield of 76%. The synthesis scheme of Step 3 is shown in (a-3) below.

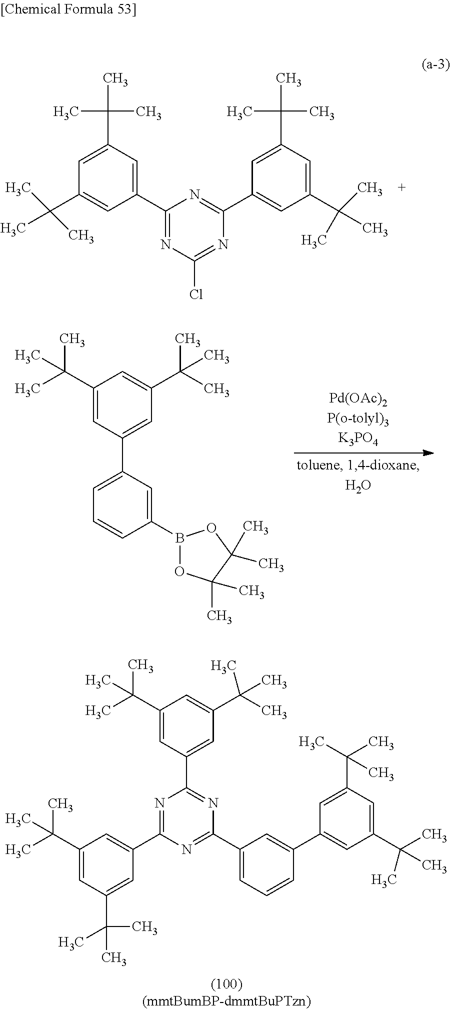

Then, 0.87 g of the obtained white solid was purified by a train sublimation method at 230° C. under a pressure of 5.8 Pa while an argon gas was made to flow. After the purification by sublimation, 0.82 g of a target white solid was obtained at a collection rate of 95%.

Analysis results by nuclear magnetic resonance (¹H-NMR) spectroscopy of the white solid obtained in Step 3 are shown below. These results reveal that the organic compound of one embodiment of the present invention, mmtBumBP-dmmtBuPTzn represented by Structural Formula (100), was obtained in this example.

¹H NMR (CDCl₃, 300 MHz): δ=1.42-1.49 (m, 54H), 7.50 (s, 1H), 7.61-7.70 (m, 5H), 7.87 (d, 1H), 8.68-8.69 (m, 4H), 8.78 (d, 1H), 9.06 (s, 1H).

The absorption spectrum of mmtBumBP-dmmtBuPTzn was measured with an ultraviolet-visible light spectrophotometer (V550, manufactured by JASCO Corporation). The absorption spectrum of mmtBumBP-dmmtBuPTzn in the dichloromethane solution was obtained by subtracting the spectrum of dichloromethane alone in a quartz cell from the absorption spectrum of mmtBumBP-dmmtBuPTzn in the dichloromethane solution in the quartz cell. As a result, the absorption peak was observed at 267 nm, revealing that no absorption was shown in the visible region range from 440 nm to 700 nm.

Next, mmtBumBP-dmmtBuPTzn obtained in this example was analyzed by liquid chromatography mass spectrometry (LC/MS).

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumBP-dmmtBuPTzn in an organic solvent at an arbitrary concentration, and the injection amount was 5.0 μL.

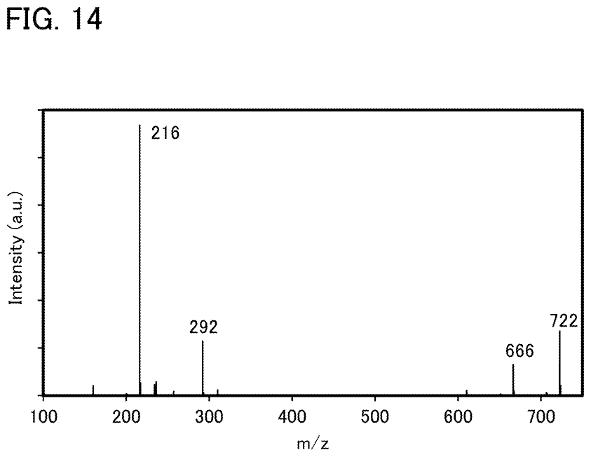

The MS/MS measurement of m/z=721.53 corresponding to the exact mass of mmtBumBP-dmmtBuPTzn was performed by a PRM method. For setting of the PRM, the mass range of a target ion was set to m/z=721.53±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 14.

Fragment ions at m/z of 216.17 and m/z of 292.21 were observed. These fragment ions are each probably a fragment formed of one substituent bonded to triazine and carbon and nitrogen derived from triazine. For example, the fragment ion at m/z of 216.17 is probably a fragment in which one carbon atom and one nitrogen atom each derived from triazine are bonded to a phenyl group to which two tertiary butyl groups are bonded. In addition, the fragment ion at m/z of 292.21 is probably a fragment in which one carbon atom and one nitrogen atom each derived from triazine are bonded to a biphenyl group to which two tertiary butyl groups are bonded. These fragments can each be regarded as a feature of a compound having a triazine skeleton.

Next, the glass transition temperature was measured with a differential scanning calorimeter (Pyris 1 DSC, manufactured by PerkinElmer, Inc.). The measurement results show that the glass transition temperature is 112° C. Thus, the organic compound of one embodiment of the present invention has a high glass transition temperature and favorable heat resistance.

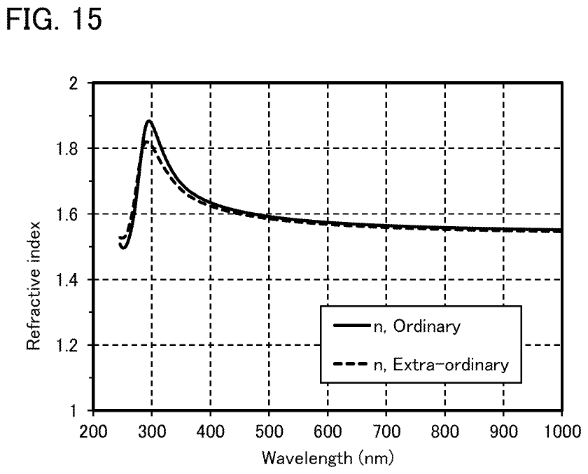

FIG. 15 shows the results of measuring the refractive index of mmtBumBP-dmmtBuPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 15.

FIG. 15 shows that mmtBumBP-dmmtBuPTzn is a material with a low refractive index: the ordinary refractive index of mmtBumBP-dmmtBuPTzn in the entire blue emission region (from 455 nm to 465 nm) is in the range of 1.50 to 1.75, and the ordinary refractive index of mmtBumBP-dmmtBuPTzn at a wavelength of 633 nm is in the range of 1.45 to 1.70.

Note that favorable light emission can be obtained from a light-emitting device fabricated using the organic compound as an electron-transport material.

### Example 2

### Synthesis Example 2

In this example, a method for synthesizing 2-{(3′,5′-di-tert-butyl)-1,1′-biphenyl-3-yl}-4,6-diphenyl-1,3,5-triazine (abbreviation: mmtBumBPTzn), which is the organic compound represented by Structural Formula (120) in Embodiment 1, is described. The structure of mmtBumBPTzn is shown below.

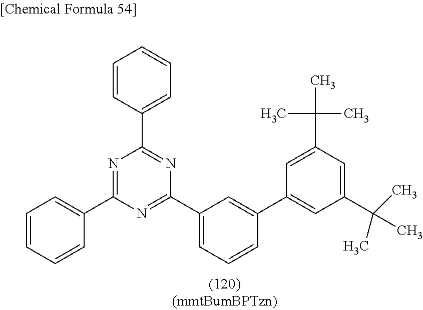 [SMILES: CC(C)(C)c1cc(-c2cccc(-c3nc(-c4ccccc4)nc(-c4ccccc4)n3)c2)cc(C(C)(C)C)c1]

### Step 1: Synthesis of 3-bromo-3′,5′-di-tert-butylbiphenyl

This synthesis step is similar to Step 1 in Synthesis example 1.

### Step 2: Synthesis of 2-(3 5′-di-tert-butylbiphenyl-3-yl)-4,4,5,5-tetramethyl-1,3,2-dioxaborolane

This synthesis step is similar to Step 2 in Synthesis example 1.

### Step 3: Synthesis of mmtBumBPTzn

Into a three-neck flask were put 1.5 g (5.6 mmol) of 4,6-diphenyl-2-chloro-1,3,5-triazine, 2.4 g (6.2 mmol) of 2-(3′,5′-di-tert-butylbiphenyl-3-yl)-4,4,5,5-tetramethyl-1,3,2-dioxaborolane, 2.4 g (11 mmol) of tripotassium phosphate, 10 mL of water, 28 mL of toluene, and 10 mL of 1,4-dioxane, and the mixture was degassed by being stirred under reduced pressure. To this mixture were added 13 mg (0.056 mmol) of palladium(II) acetate and 34 mg (0.11 mmol) of tris(2-methylphenyl)phosphine, and the mixture was heated and refluxed under a nitrogen atmosphere for 14 hours to cause reaction. After the reaction, extraction was performed with ethyl acetate and water in the obtained organic layer was removed with magnesium sulfate. This mixture was gravity-filtered, and the obtained filtrate was purified by silica gel column chromatography with a developing solvent of chloroform and hexane in a ratio of 1:5, which was then changed to 1:3, and then recrystallized with hexane to give 2.0 g of a target white solid in a yield of 51%. The synthesis scheme of Step 3 is shown in (b-1) below.

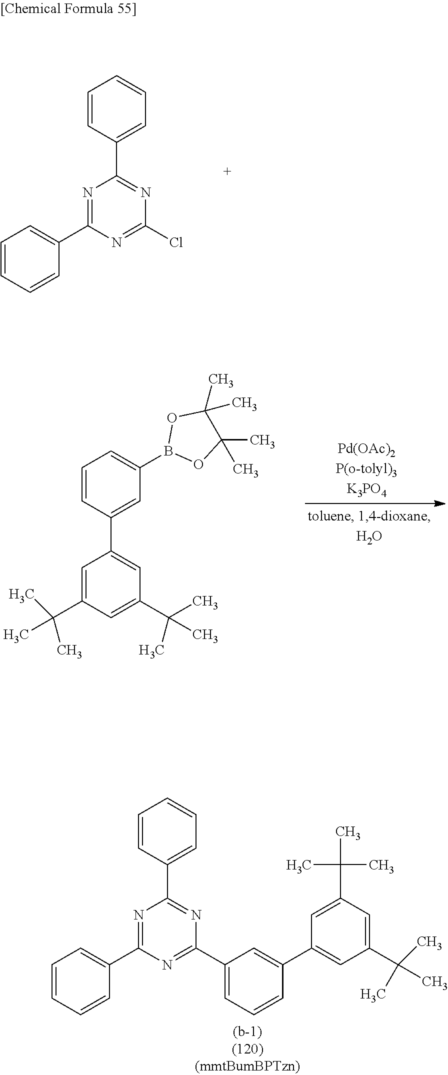 [SMILES: Clc1nc(-c2ccccc2)nc(-c2ccccc2)n1 ; CC(C)(C)c1cc(-c2cccc(B3OC(C)(C)C(C)(C)O3)c2)cc(C(C)(C)C)c1 ; CC(C)(C)c1cc(-c2cccc(-c3nc(-c4ccccc4)nc(-c4ccccc4)n3)c2)cc(C(C)(C)C)c1]

Then, 2.0 g of the obtained white solid was purified by a train sublimation method under an argon gas stream at 220° C. under a pressure of 3.4 Pa. The solid was heated. After the purification by sublimation, 1.8 g of a target white solid was obtained at a collection rate of 80%.

Analysis results by nuclear magnetic resonance (¹H-NMR) spectroscopy of the white solid obtained in Step 3 are shown below. These results reveal that the organic compound of one embodiment of the present invention, mmtBumBPTzn represented by Structural Formula (120), was obtained in this example.

¹H NMR (CDCl₃, 300 MHz): δ=1.44 (s, 18H), 7.51-7.68 (m, 10H), 7.83 (d, 1H), 8.73-8.81 (m, 5H), 9.01 (s, 1H).

Next, the absorption spectrum of mmtBumBPTzn was measured with an ultraviolet-visible light spectrophotometer (V550, manufactured by JASCO Corporation). The absorption spectrum of mmtBumBPTzn in the dichloromethane solution was obtained by subtracting the spectrum of dichloromethane alone in a quartz cell from the absorption spectrum of mmtBumBPTzn in the dichloromethane solution in the quartz cell. As a result, mmtBumBPTzn in the dichloromethane solution had an absorption peak at 271 nm. Since no absorption was shown in the visible region range from 440 nm to 700 nm, mmtBumBPTzn had excellent absorption characteristics as a display material.

Next, mmtBumBPTzn obtained in this example was analyzed by liquid chromatography mass spectrometry (LC/MS).

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumBPTzn in an organic solvent at an arbitrary concentration, and the injection amount was 5.0 μL.

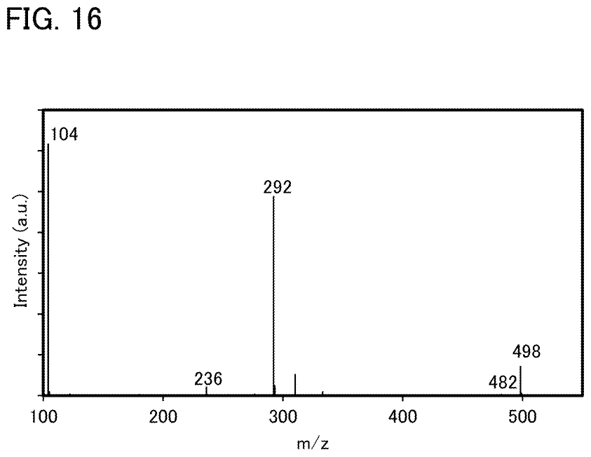

The MS/MS measurement of m/z=497.28 corresponding to the exact mass of mmtBumBPTzn was performed by a PRM method. For setting of the PRM, the mass range of a target ion was set to m/z=497.28±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 16.

Fragment ions at m/z of 104.05 and m/z of 292.21 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 292.21 is probably a fragment in which one carbon atom and one nitrogen atom each derived from triazine are bonded to a substituent other than a phenyl group. These fragments can each be regarded as a feature of a compound having a triazine skeleton.

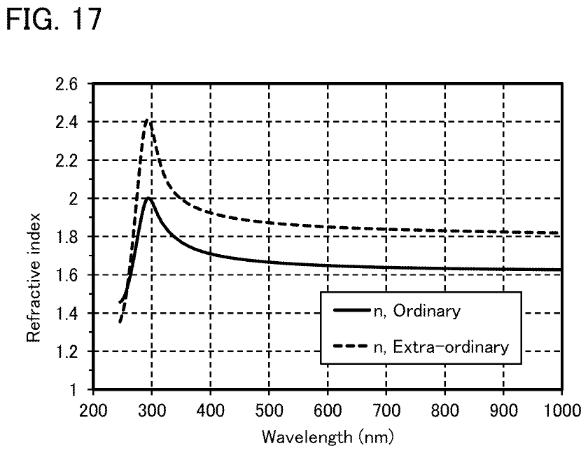

FIG. 17 shows the results of measuring the refractive index of mmtBumBPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 17.

FIG. 17 shows that mmtBumBPTzn is a material with a low refractive index: the ordinary refractive index of mmtBumBPTzn in the entire blue emission region (from 455 nm to 465 nm) is in the range of 1.50 to 1.75, and the ordinary refractive index of mmtBumBPTzn at a wavelength of 633 nm is in the range of 1.45 to 1.70.

Note that favorable light emission can be obtained from a light-emitting device fabricated using the organic compound as an electron-transport material.

### Example 3

### Synthesis Example 3

In this example, a method for synthesizing 2-(3,3″,5,5″-tetra-tert-butyl-1,1′:3′,1″-terphenyl-5′-yl)-4,6-diphenyl-1,3,5-triazine (abbreviation: mmtBumTPTzn), which is the organic compound represented by Structural Formula (121) in Embodiment 1, is described. The structure of mmtBumTPTzn is shown below.

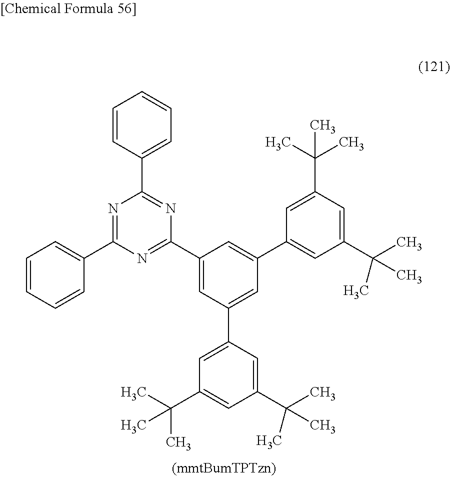 [SMILES: CC(C)(C)c1cc(-c2cc(-c3cc(C(C)(C)C)cc(C(C)(C)C)c3)cc(-c3nc(-c4ccccc4)nc(-c4ccccc4)n3)c2)cc(C(C)(C)C)c1]

### Step 1: Synthesis of mmtBumTPTzn

Into a three-neck flask were put 0.67 g (2.5 mmol) of 4,6-diphenyl-2-chloro-1,3,5-triazine, 1.6 g (2.8 mmol) of 2-3,5-bis(3,5-di-tert-butylphenyl)benzen-1-yl-4,4,5,5-tetramethyl-1,3,2-dioxaborolane, 1.1 g (5.0 mmol) of tripotassium phosphate, 5 mL of water, 14 mL of toluene, and 5 mL of 1,4-dioxane, and the mixture was degassed by being stirred under reduced pressure. To this mixture were added 5.6 mg (0.025 mmol) of palladium(II) acetate and 15 mg (0.050 mmol) of tris(2-methylphenyl)phosphine, and the mixture was heated and refluxed under a nitrogen atmosphere for 19 hours. After the reaction, the reaction solution was filtered, and a filtrate and a residue (1) were separated. The obtained filtrate was subjected to extraction with ethyl acetate and the obtained organic layer was dried with magnesium sulfate. This mixture was filtered, and the obtained filtrate was concentrated and filtered to give a residue (2).

The residue (1) and the residue (2) were collectively purified by silica gel column chromatography with a developing solvent of chloroform and hexane in a ratio of 1:5 and then recrystallized with toluene to give 1.2 g of a target white solid in a yield of 71%. The synthesis scheme of Step 1 is shown in (c-1) below.

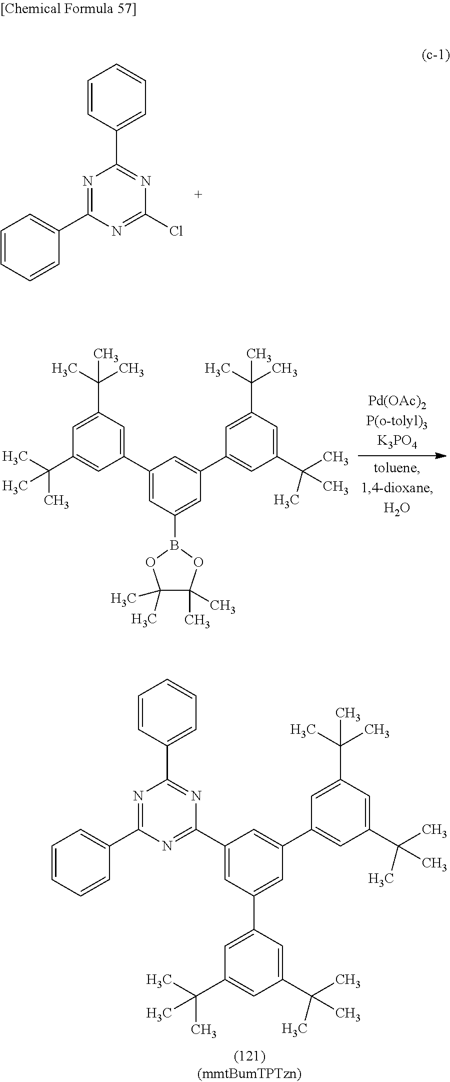 [SMILES: Clc1nc(-c2ccccc2)nc(-c2ccccc2)n1 ; CC(C)(C)c1cc(-c2cc(B3OC(C)(C)C(C)(C)O3)cc(-c3cc(C(C)(C)C)cc(C(C)(C)C)c3)c2)cc(C(C)(C)C)c1 ; CC(C)(C)c1cc(-c2cc(-c3cc(C(C)(C)C)cc(C(C)(C)C)c3)cc(-c3nc(-c4ccccc4)nc(-c4ccccc4)n3)c2)cc(C(C)(C)C)c1]

Then, 1.2 g of the obtained white solid was purified by a train sublimation method under an argon gas stream at 285° C. under a pressure of 3.4 Pa. After the purification by sublimation, 1.1 g of a target white solid was obtained at a collection rate of 89%.

Analysis results by nuclear magnetic resonance (¹H-NMR) spectroscopy of the white solid obtained in Step 1 are shown below. These results reveal that the organic compound of one embodiment of the present invention, mmtBumTPTzn represented by Structural Formula (121), was obtained in this example.

¹H NMR (CDCl₃, 300 MHz): δ=1.44 (s, 36H), 7.54-7.62 (m, 12H), 7.99 (t, 1H), 8.79 (d, 4H), 8.92 (d, 2H).

Next, the absorption spectrum of mmtBumTPTzn was measured with an ultraviolet-visible light spectrophotometer (V550, manufactured by JASCO Corporation). The absorption spectrum of mmtBumTPTzn in the dichloromethane solution was obtained by subtracting the spectrum of dichloromethane alone in a quartz cell from the absorption spectrum of mmtBumTPTzn in the dichloromethane solution in the quartz cell. As a result, the absorption peak was observed at 265 nm, revealing that no absorption was shown in the visible region range from 440 nm to 700 nm.

Next, mmtBumTPTzn obtained in this example was analyzed by liquid chromatography mass spectrometry (LC/MS).

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumTPTzn in an organic solvent at an arbitrary concentration, and the injection amount was 5.0 μL.

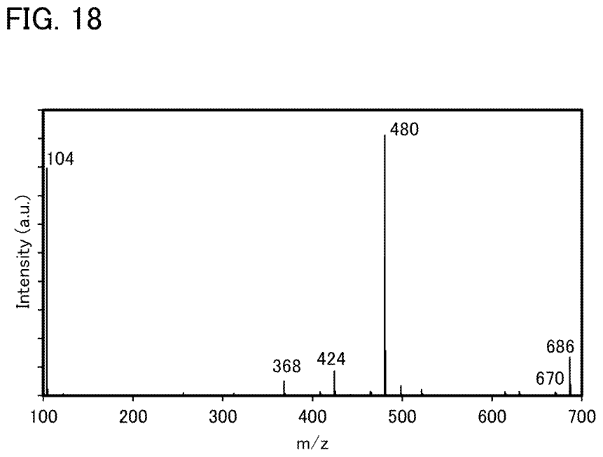

The MS/MS measurement of m/z=685.44 corresponding to the exact mass of mmtBumTPTzn was performed by a PRM method. For setting of the PRM, the mass range of a target ion was set to m/z=685.44±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 18.

Fragment ions at m/z of 104.05 and m/z of 480.36 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 480.36 is probably a fragment in which one carbon atom and one nitrogen atom each derived from triazine are bonded to a substituent other than a phenyl group. These fragments can each be regarded as a feature of a compound having a triazine skeleton.

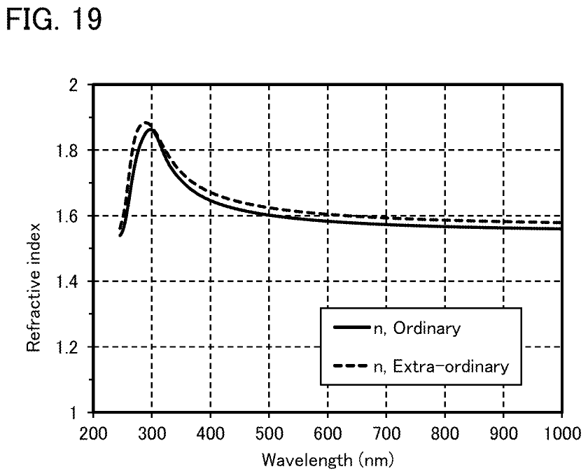

FIG. 19 shows the results of measuring the refractive index of mmtBumTPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 19.

FIG. 19 shows that mmtBumTPTzn is a material with a low refractive index: the ordinary refractive index of mmtBumTPTzn in the entire blue emission region (from 455 nm to 465 nm) is in the range of 1.50 to 1.75, and the ordinary refractive index of mmtBumTPTzn at a wavelength of 633 nm is in the range of 1.45 to 1.70.

Note that favorable light emission can be obtained from a light-emitting device fabricated using the organic compound as an electron-transport material.

### Example 4

### Synthesis Example 4

In this example, a method for synthesizing 2-(3′,5′-di-tert-butylbiphenyl-3-yl)-4,6-bis(3,5-di-tert-butylphenyl)pyrimidine (abbreviation: mmtBumBP-dmmtBuPPm), which is the organic compound represented by Structural Formula (200) in Embodiment 1, is described. The structure of mmtBumBP-dmmtBuPPm is shown below.

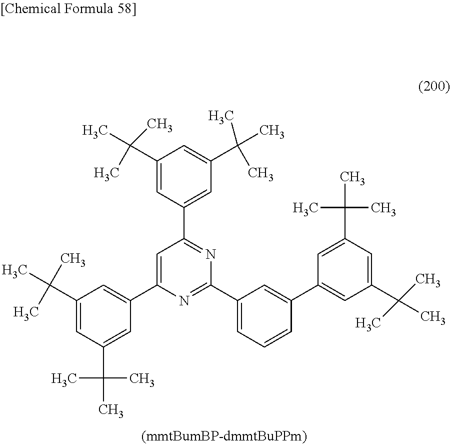 [SMILES: CC(C)(C)c1cc(-c2cccc(-c3nc(-c4cc(C(C)(C)C)cc(C(C)(C)C)c4)cc(-c4cc(C(C)(C)C)cc(C(C)(C)C)c4)n3)c2)cc(C(C)(C)C)c1]

### Step 1: Synthesis of 4,6-bis(3,5-di-tert-butylphenyl)-2-chloro-1,3-pyrimidine

Into a three-neck flask were put 1.4 g (7.8 mmol) of 2,4,6-trichloropyrimidine, 40 mL of acetonitrile, 16 mL of water, 3.8 g (16 mmol) of 3,5-di-tert-butylphenylboronic acid, and 4.3 g (31 mmol) of potassium carbonate, and the mixture was degassed by being stirred under reduced pressure. To this mixture was added 0.22 g (0.31 mmol) of bis(triphenylphosphine)palladium(II) dichloride, and the resulting mixture was stirred under a nitrogen atmosphere at 50° C. for 4 hours. After the reaction, toluene was added to the reaction mixture, the mixture was washed with water and saturated saline, and water in the obtained organic layer was removed with magnesium sulfate. This mixture was gravity-filtered, and the obtained filtrate was concentrated, which was then purified by silica gel column chromatography with a developing solvent of hexane and toluene in a ratio of 1:1 to give 2.7 g of a target white solid in a yield of 71%. The synthesis scheme of Step 1 is shown in (d-1) below.

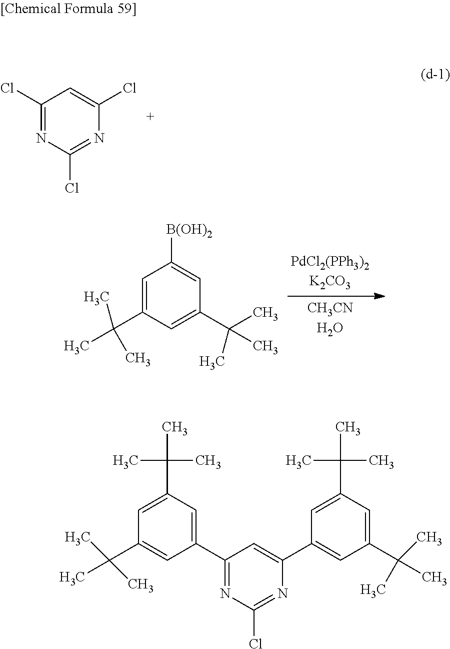 [SMILES: Clc1cc(Cl)nc(Cl)n1 ; CC(C)(C)c1cc(B(O)O)cc(C(C)(C)C)c1 ; CC(C)(C)c1cc(-c2cc(-c3cc(C(C)(C)C)cc(C(C)(C)C)c3)nc(Cl)n2)cc(C(C)(C)C)c1]

### Step 2: Synthesis of 3-bromo-3′,5′-di-tert-butylbiphenyl

This synthesis step is similar to Step 1 in Synthesis example 1.

### Step 3: Synthesis of 2-(3′,5′-di-tert-butylbiphenyl-3-yl)-4,4,5,5-tetramethyl-1,3,2-dioxaborolane

This synthesis step is similar to Step 2 in Synthesis example 1.

### Step 4: Synthesis of mmtBumBP-dmmtBuPPm

Into a three-neck flask were put 0.93 g (1.9 mmol) of 4,6-bis(3,5-di-tert-butylphenyl)-2-chloro-1,3-pyrimidine synthesized in Step 1, 0.92 g (2.3 mmol) of 2-(3′,5′-di-tert-butylbiphenyl-3-yl)-4,4,5,5-tetramethyl-1,3,2-dioxaborolane synthesized in Step 3, 0.53 g (3.8 mmol) of potassium carbonate, 20 mL of tetrahydrofuran (abbreviation: THF), and 4 mL of water, and the mixture was degassed by being stirred under reduced pressure. To this mixture were added 17 mg (0.057 mmol) of tri-tert-butylphosphoniumtetrafluoroborate and 17 mg (0.019 mmol) of tris(dibenzylideneacetone)dipalladium(0), and this solution was stirred under a nitrogen atmosphere at 80° C. for 17 hours. After the reaction, toluene was added to the reaction mixture, the mixture was washed with water and saturated saline, and water in the obtained organic layer was removed with magnesium sulfate. This mixture was gravity-filtered, and the obtained filtrate was purified by silica gel column chromatography with a developing solvent of hexane and toluene in a ratio of 4:1 to give approximately 1.3 g of a target white solid in a yield of approximately 95%. The synthesis scheme of Step 4 is shown in (d-2) below.

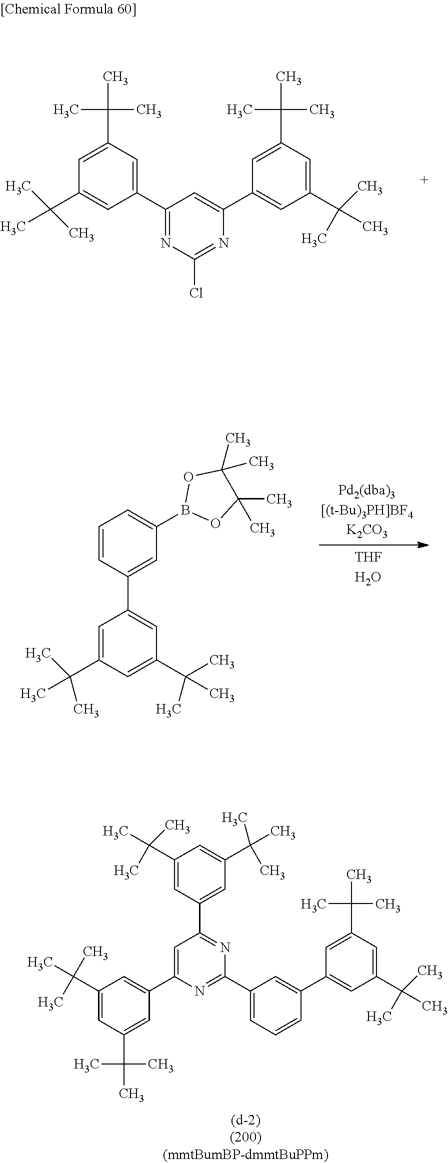

Analysis results by nuclear magnetic resonance (¹H-NMR) spectroscopy of the white solid obtained in Step 4 are shown below. These results reveal that the organic compound of one embodiment of the present invention, mmtBumBP-dmmtBuPPm represented by Structural Formula (200), was obtained in this example.

¹H NMR (CDCl₃, 300 MHz): δ=1.39-1.45 (m, 54H), 7.47 (t, 1H), 7.59-7.65 (m, 5H), 7.76 (d, 1H), 7.95 (s, 1H), 8.06 (d, 4H), 8.73 (d, 1H), 8.99 (s, 1H).

Next, the absorption spectrum of mmtBumBP-dmmtBuPPm was measured with an ultraviolet-visible light spectrophotometer (V550, manufactured by JASCO Corporation). The absorption spectrum of mmtBumBP-dmmtBuPPm in the dichloromethane solution was obtained by subtracting the spectrum of dichloromethane alone in a quartz cell from the absorption spectrum of mmtBumBP-dmmtBuPPm in the dichloromethane solution in the quartz cell. As a result, the absorption peak was observed at 267 nm, revealing that no absorption was shown in the visible region range from 440 nm to 700 nm.

Next, mmtBumBP-dmmtBuPPm obtained in this example was analyzed by liquid chromatography mass spectrometry (LC/MS).

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumBP-dmmtBuPPm in an organic solvent at an arbitrary concentration, and the injection amount was 5.0 μL.

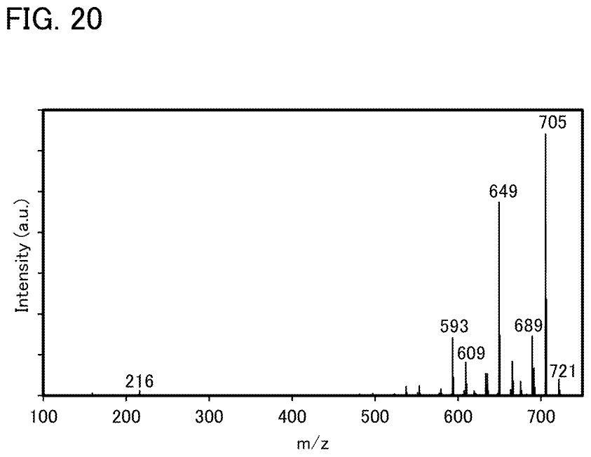

The MS/MS measurement of m/z=720.54 corresponding to the exact mass of mmtBumBP-dmmtBuPPm was performed by a PRM method. For setting of the PRM, the mass range of a target ion was set to m/z=720.54±2.0 (isolation window=4) and detection was performed in a positive mode. The measurement was performed with energy (normalized collision energy: NCE) for accelerating a target ion in a collision cell set to 70. The MS spectrum obtained by the MS/MS measurement is shown in FIG. 20.

A fragment ion at m/z of 216.17 was observed. The fragment ion is probably a fragment formed of one substituent bonded to triazine and carbon and nitrogen derived from triazine. For example, the fragment ion at m/z of 216.17 is probably a fragment in which one carbon atom and one nitrogen atom each derived from pyrimidine are bonded to a phenyl group. The fragment can be regarded as a feature of a compound having a pyrimidine skeleton.

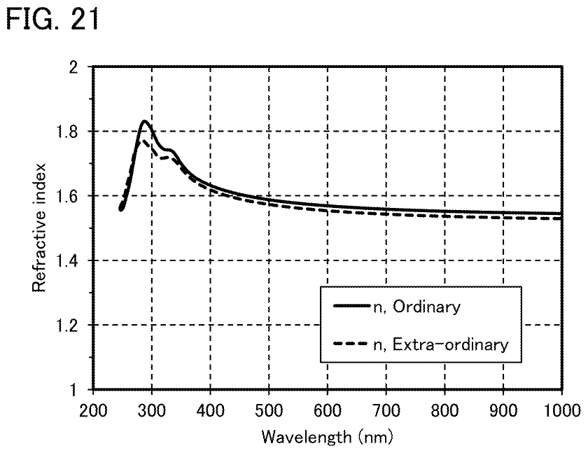

FIG. 21 shows the results of measuring the refractive index of mmtBumBP-dmmtBuPPm with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 21.

FIG. 21 shows that mmtBumBP-dmmtBuPPm is a material with a low refractive index: the ordinary refractive index of mmtBumBP-dmmtBuPPm in the entire blue emission region (from 455 nm to 465 nm) is in the range of 1.50 to 1.75, and the ordinary refractive index of mmtBumBP-dmmtBuPPm at a wavelength of 633 nm is in the range of 1.45 to 1.70.

Note that favorable light emission can be obtained from a light-emitting device fabricated using the organic compound as an electron-transport material.

### Example 5

In this example, a light-emitting device 1 of one embodiment of the present invention described in the above embodiments and a comparative light-emitting device 1 are described. Structural formulae of organic compounds used in this example are shown below.

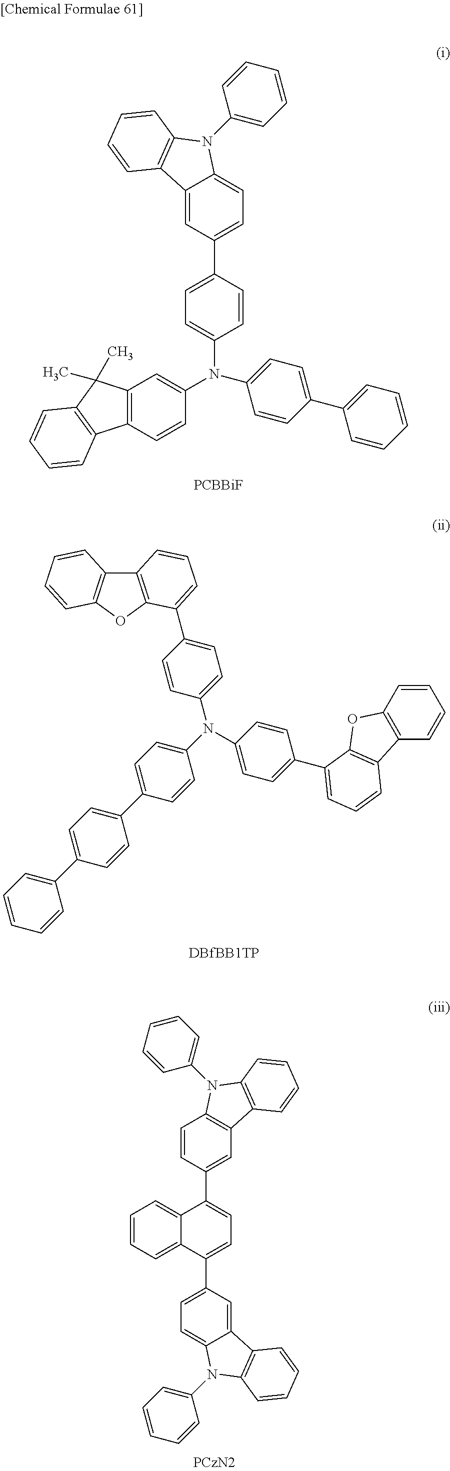

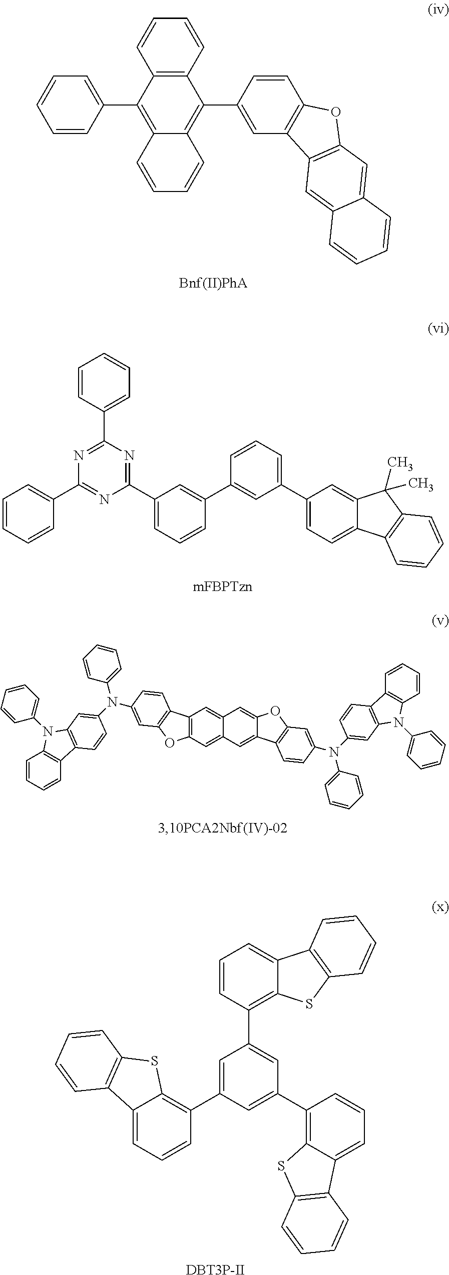 [SMILES: c1ccc(-c2c3ccccc3c(-c3ccc4oc5cc6ccccc6cc5c4c3)c3ccccc23)cc1 ; CC1(C)c2ccccc2-c2ccc(-c3cccc(-c4cccc(-c5nc(-c6ccccc6)nc(-c6ccccc6)n5)c4)c3)cc21 ; c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1 ; c1ccc2c(c1)sc1c(-c3cc(-c4cccc5c4sc4ccccc45)cc(-c4cccc5c4sc4ccccc45)c3)cccc12 ; "DBT3P-II"]

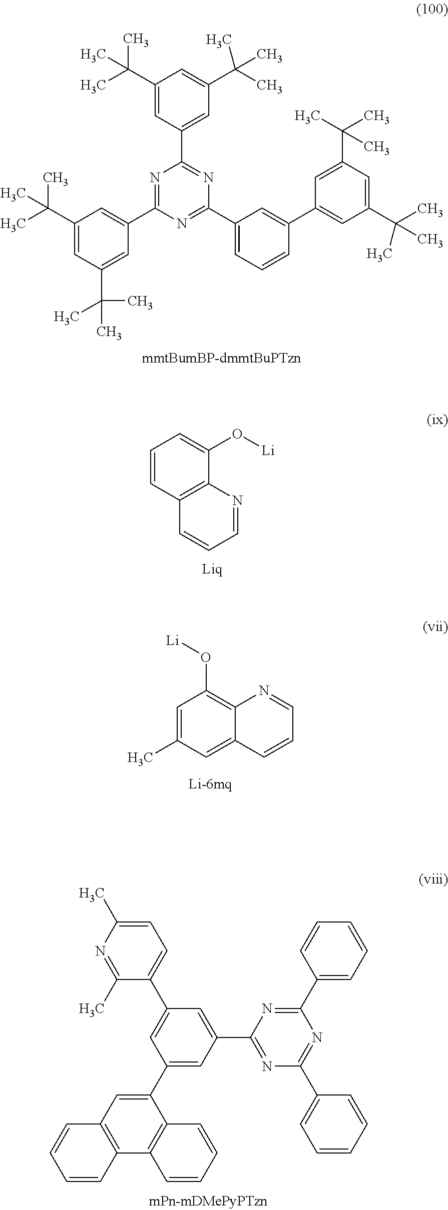

### (Fabrication Method of Light-Emitting Device 1)

First, as a reflective electrode, an alloy film of silver (Ag), palladium (Pd), and copper (Cu), i.e., an Ag—Pd—Cu (APC) film, was formed over a glass substrate to a thickness of 100 nm by a sputtering method, and then, as a transparent electrode, indium tin oxide containing silicon oxide (ITSO) was formed to a thickness of 85 nm by a sputtering method, whereby the first electrode 101 was formed. The electrode area was 4 mm² (2 mm×2 mm).

Next, in pretreatment for forming the light-emitting device over a substrate, a surface of the substrate was washed with water and baked at 200° C. for one hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structure Formula (i) and an electron acceptor material (OCHD-001) were deposited by co-evaporation to a thickness of 10 nm such that the weight ratio of PCBBiF to OCHD-001 was 1:0.05, whereby the hole-injection layer 111 was formed.

Over the hole-injection layer 111, PCBBiF was deposited by evaporation to a thickness of 20 nm, and then N,N-bis[4-(dibenzofuran-4-yl)phenyl]-4-amino-p-terphenyl (abbreviation: DBfBB1TP) represented by Structural Formula (ii) was deposited by evaporation to a thickness of 10 nm, whereby the hole-transport layer 112 was formed.

Subsequently, over the hole-transport layer 112, 3,3′-(naphthalene-1,4-diyl)bis(9-phenyl-9H-carbazole) (abbreviation: PCzN2) represented by Structural Formula (iii) was deposited by evaporation to a thickness of 10 nm, whereby an electron-blocking layer was formed.

Then, 2-(10-phenyl-9-anthracenyl)-benzo[b]naphtho[2,3-d]furan (abbreviation: Bnf(II)PhA) represented by Structural Formula (iv) and 3,10-bis[N-(9-phenyl-9H-carbazol-2-yl)-N-phenylamino]naphtho[2,3-b;6,7-b]bisbenzofuran (abbreviation: 3,10PCA2Nbf(IV)-02) represented by Structural Formula (v) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, 2-{(3′,5′-di-tert-butyl)-1,1′-biphenyl-3-yl}-4,6-bis(3,5-di-tert-butylphenyl)-1,3,5-triazine (abbreviation: mmtBumBP-dmmtBuPTzn), which is the low refractive index material of one embodiment of the present invention described in Example 1 and is represented by Structural Formula (100), was deposited by evaporation to a thickness of 10 nm to form a hole-blocking layer. Then, mmtBumBP-dmmtBuPTzn and 6-methyl-8-quinolinolato-lithium (abbreviation: Li-6mq) represented by Structural Formula (vii) were deposited by co-evaporation to a thickness of 20 nm such that the weight ratio of mmtBumBP-dmmtBuPTzn to Li-6mq was 1:1, whereby the electron-transport layer 114 was formed.

After the electron-transport layer 114 was formed, Li-6mq was deposited by evaporation to a thickness of 1 nm to form the electron-injection layer 115, and lastly silver (Ag) and magnesium (Mg) were deposited by co-evaporation to a thickness of 15 nm such that the volume ratio of Ag to Mg was 1:0.1 to form the second electrode 102, whereby the light-emitting device 1 was fabricated. The second electrode 102 is a transflective electrode having a function of reflecting light and a function of transmitting light; thus, the light-emitting device of this example is a top-emission device in which light is extracted through the second electrode 102. Over the second electrode 102, 1,3,5-tri(dibenzothiophen-4-yl)-benzene (abbreviation: DBT3P-II) represented by Structural Formula (x) was deposited by evaporation to a thickness of 70 nm so that outcoupling efficiency can be improved.

### (Fabrication Method of Comparative Light-Emitting Device 1)

The comparative light-emitting device 1 was fabricated in the same manner as the light-emitting device 1 except that the thickness of the hole-transport layer 112 was nm; 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (vi) was used instead of mmtBumBP-dmmtBuPTzn for the hole-blocking layer; 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (viii) was used instead of mmtBumBP-dmmtBuPTzn for the electron-transport layer 114; and 8-quinolinolato-lithium (abbreviation: Liq) represented by Structural Formula (ix) was used instead of Li-6mq for the electron-transport layer 114 and the electron-injection layer 115.

The structures of the light-emitting device 1 and the comparative light-emitting device 1 are listed in Table 1 below.

TABLE 1

| | film | Light- | Comparative light- |
|---|---|---|---|
| | thickness | emitting device 1 | emitting device 1 |
| electron-injection layer | 1 nm | Li-6mq | Liq |
| electron-transport layer | 20 nm | mmtBumBP-dmmtBuPTzn:Li-6mq | mPn-mDMePyPTzn:Liq |
| | | (1:1) | (1:1) |
| hole-blocking layer | 10 nm | mmtBumBP-dmmtBuPTzn | mFBPTzn |

| | | |
|---|---|---|
| light-emitting layer | 25 nm | Bnf(II)PhA:3,10PCA2Nbf(IV)-02 |
| | | (1:0.015) |
| electron-blocking layer | 10 nm | PCzN2 |
| hole-transport layer | 10 nm | DBfBB1TP |
| | *1 | PCBBiF |
| hole-injection layer | 10 nm | PCBBiF:OCHD-001 |
| | | (1:0.05) |

*1 Light-emitting device 1: 20 nm, Comparative light-emitting device 1: 15 nm

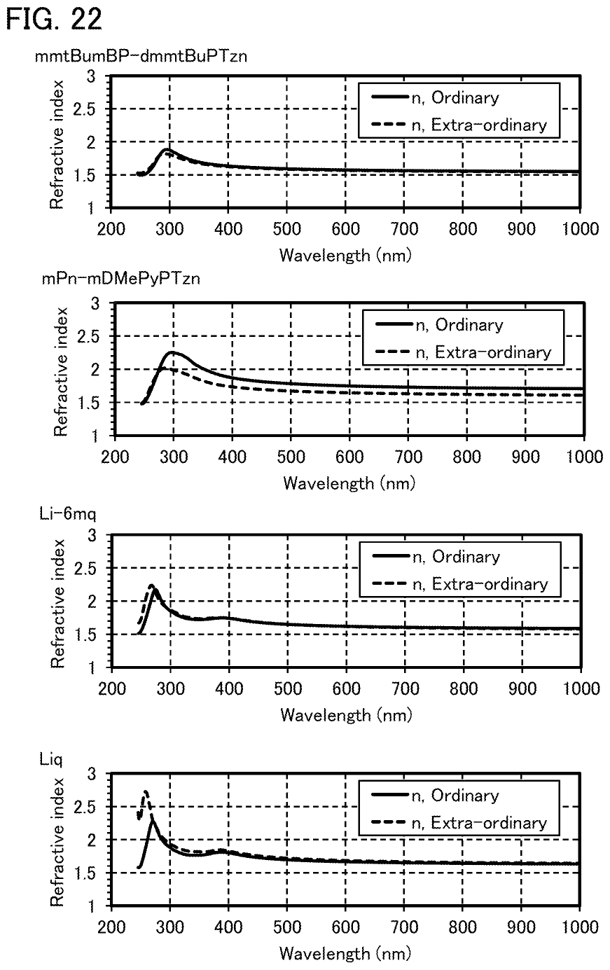

FIG. 22 shows the refractive indices of mmtBumBP-dmmtBuPTzn, mPn-mDMePyPTzn, Li-6mq, and Liq, and Table 2 shows the refractive indices at a wavelength of 456 nm. The refractive indices were measured with a spectroscopic ellipsometer (M-2000U, produced by J.A. Woollam Japan Corp.). A sample used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 22.

TABLE 2

| | ordinary refractive index (n, Ordinary) |
|---|---|
| | at 456 nm |
| mmtBumBP-dmmtBuPTzn | 1.61 |
| mPn-mDMePyPTzn | 1.81 |
| mFBPTzn | 1.80 |
| Li-6mq | 1.67 |
| Liq | 1.72 |

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

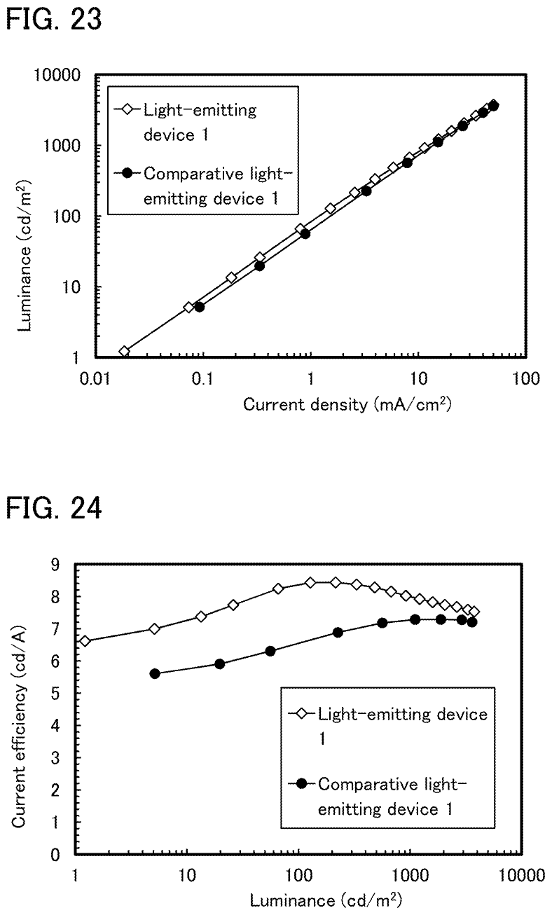

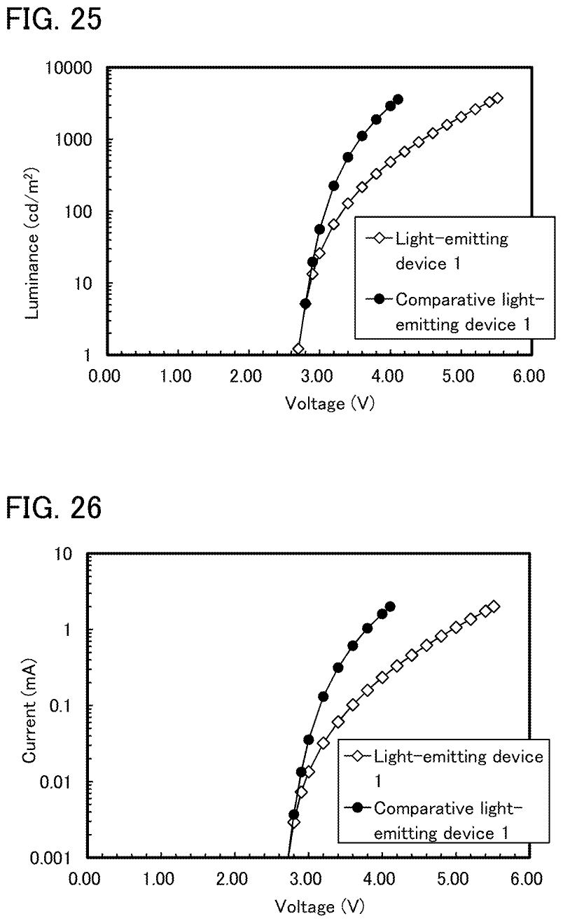

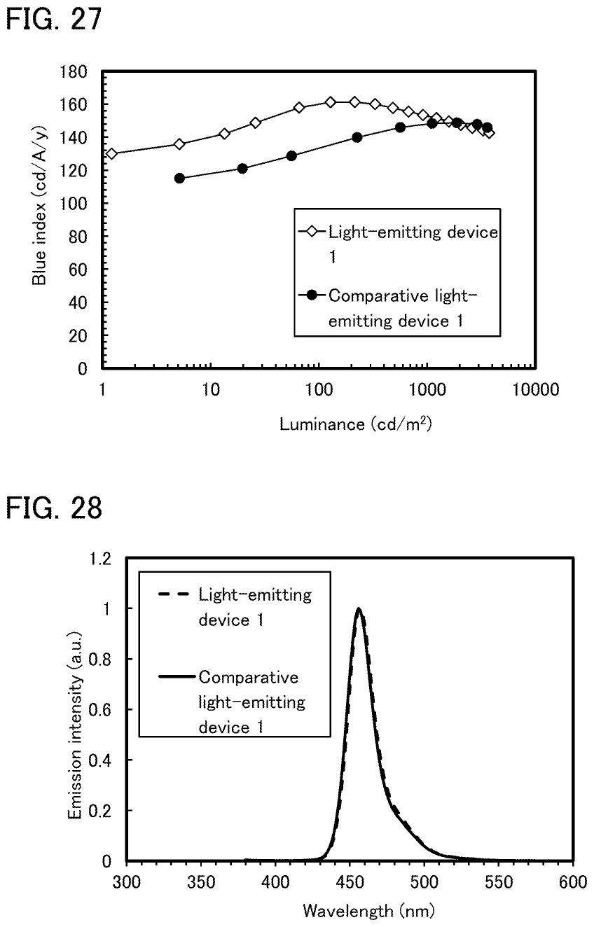

FIG. 23 shows the luminance-current density characteristics of the light-emitting device 1 and the comparative light-emitting device 1. FIG. 24 shows the current efficiency-luminance characteristics thereof. FIG. 25 shows the luminance-voltage characteristics thereof. FIG. 26 shows the current-voltage characteristics thereof. FIG. 27 shows the blue index-luminance characteristics thereof. FIG. 28 shows the emission spectra thereof. Table 3 shows the main characteristics of the light-emitting device 1 and the comparative light-emitting device 1 at a luminance of about 1000 cd/m². Luminance, CIE chromaticity, and emission spectra were measured at normal temperature with a spectroradiometer (SR-UL1R manufactured by TOPCON TECHNOHOUSE CORPORATION).

Note that the blue index (BI) is a value obtained by dividing current efficiency (cd/A) by chromaticity y, and is one of the indicators of characteristics of blue light emission. As the chromaticity y is smaller, the color purity of blue light emission tends to be higher. With high color purity, a wide range of blue can be expressed even with a small number of luminance components; thus, using blue light emission with high color purity reduces the luminance needed for expressing blue, leading to lower power consumption. Thus, BI that is based on chromaticity y, which is one of the indicators of color purity of blue, is suitably used as a means for showing efficiency of blue light emission. The light-emitting device with higher BI can be regarded as a blue light-emitting device having higher efficiency for a display.

TABLE 3

| | | | | | | current | |
|---|---|---|---|---|---|---|---|
| | voltage | current | current density | | | efficiency | BI |
| | (V) | (mA) | (mA/cm²) | chromaticity x | chromaticity y | (cd/A) | (cd/A/y) |
| Light-emitting device 1 | 4.4 | 0.46 | 11.4 | 0.14 | 0.05 | 8.0 | 153 |
| Comparative light-emitting device 1 | 3.6 | 0.61 | 15.3 | 0.14 | 0.05 | 7.3 | 148 |

The results in FIG. 23 to FIG. 28 and Table 3 reveal that the light-emitting device 1 fabricated using the low refractive index material of one embodiment of the present invention is an EL device having substantially the same emission spectrum as the comparative light-emitting device 1 and having higher current efficiency than the comparative light-emitting device 1.

The blue indices (BI) of the light-emitting device 1 and the comparative light-emitting device 1 at around 1000 cd/m² were 153 (cd/A/y) and 148 (cd/A/y), respectively, and the maximum values of the BI of the light-emitting device 1 and the comparative light-emitting device 1 were 161 (cd/A/y) and 149 (cd/A/y), respectively. Thus, the light-emitting device 1 can be regarded as having especially favorable BI. Accordingly, one embodiment of the present invention is suitable for a light-emitting device used for a display.

### Example 6

In this example, a light-emitting device 2 of one embodiment of the present invention and a comparative light-emitting device 2 are described. Structural formulae of organic compounds used in this example are shown below.

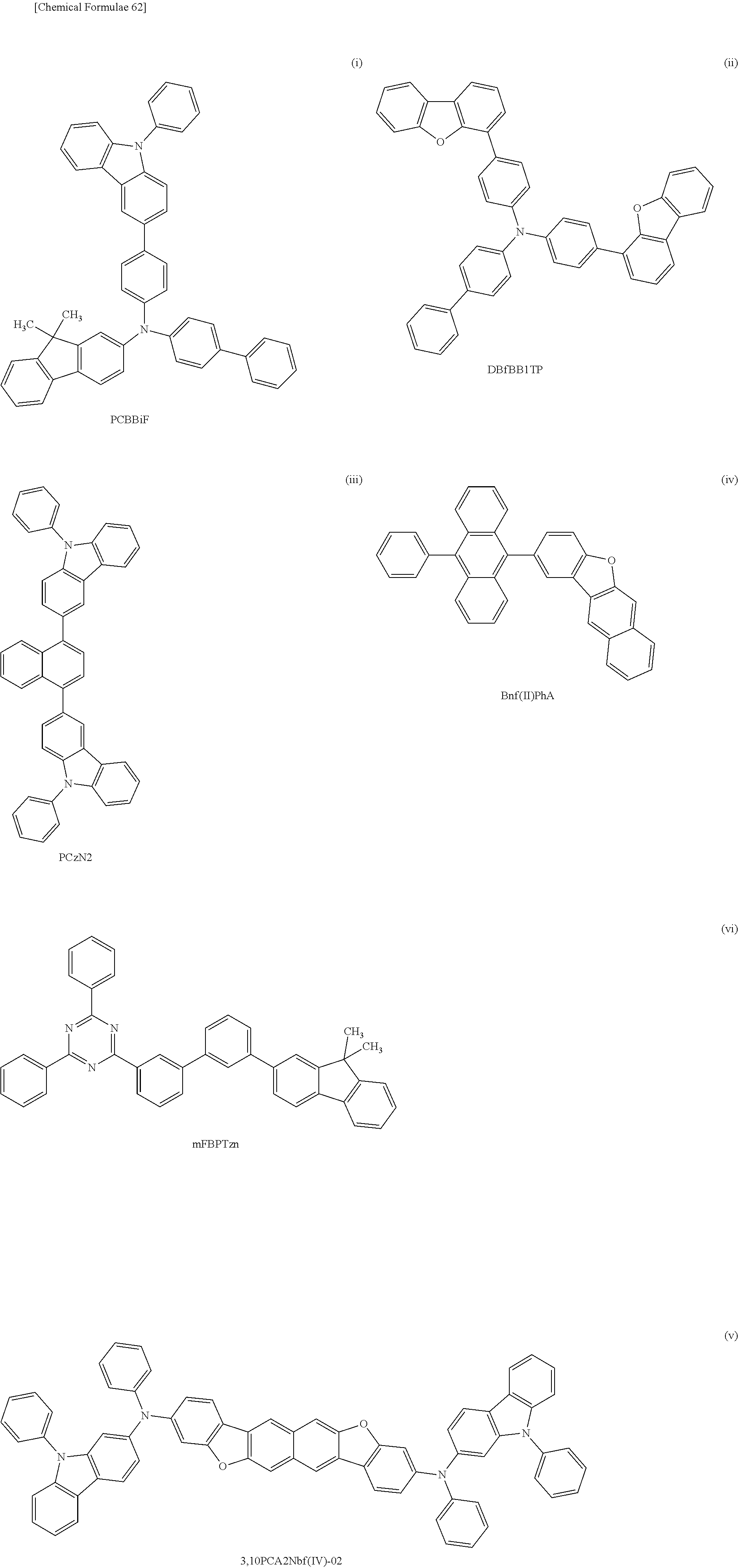

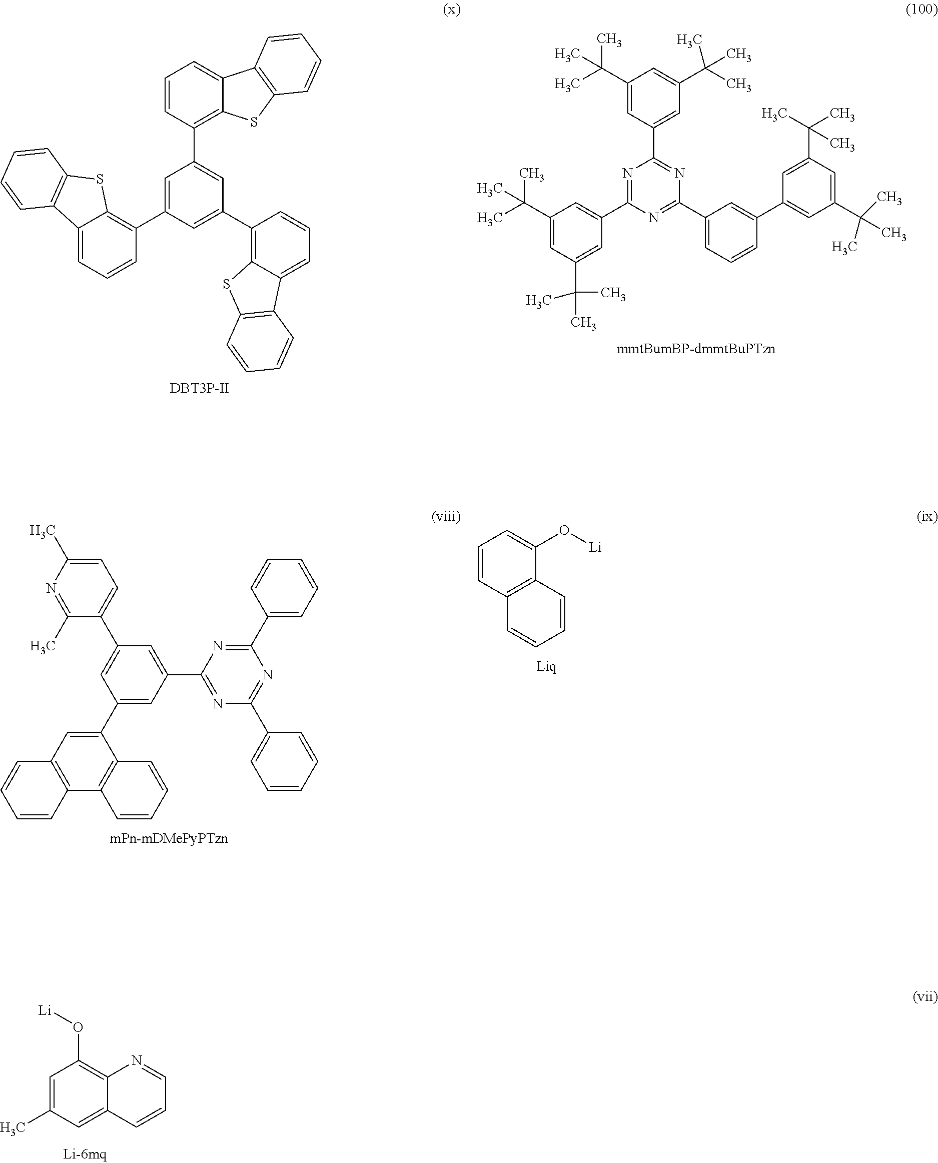

### (Fabrication Method of Light-Emitting Device 2)

First, as a reflective electrode, an alloy film of silver (Ag), palladium (Pd), and copper (Cu), i.e., an Ag—Pd—Cu (APC) film, was formed over a glass substrate to a thickness of 100 nm by a sputtering method, and then, as a transparent electrode, indium tin oxide containing silicon oxide (ITSO) was formed to a thickness of 85 nm by a sputtering method, whereby the first electrode 101 was formed. The electrode area was 4 mm² (2 mm×2 mm).

Next, in pretreatment for forming the light-emitting device over a substrate, a surface of the substrate was washed with water and baked at 200° C. for one hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structure Formula (i) and an electron acceptor material (OCHD-001) were deposited by co-evaporation to a thickness of 10 nm such that the weight ratio of PCBBiF to OCHD-001 was 1:0.05, whereby the hole-injection layer 111 was formed.

Over the hole-injection layer 111, PCBBiF was deposited by evaporation to a thickness of 20 nm, and then N,N-bis[4-(dibenzofuran-4-yl)phenyl]-4-amino-p-terphenyl (abbreviation: DBfBB1TP) represented by Structural Formula (ii) was deposited by evaporation to a thickness of 10 nm, whereby the hole-transport layer 112 was formed.

Subsequently, over the hole-transport layer 112, 3,3′-(naphthalene-1,4-diyl)bis(9-phenyl-9H-carbazole) (abbreviation: PCzN2) represented by Structural Formula (iii) was deposited by evaporation to a thickness of 10 nm, whereby the electron-blocking layer was formed.

Then, 2-(10-phenyl-9-anthracenyl)-benzo[b]naphtho[2,3-d]furan (abbreviation: Bnf(II)PhA) represented by Structural Formula (iv) and 3,10-bis[N-(9-phenyl-9H-carbazol-2-yl)-N-phenylamino]naphtho[2,3-b;6,7-b]bisbenzofuran (abbreviation: 3,10PCA2Nbf(IV)-02) represented by Structural Formula (v) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, 2-{(3′,5′-di-tert-butyl)-1,1′-biphenyl-3-yl}-4,6-diphenyl-1,3,5-triazine (abbreviation: mmtBumBPTzn), which is the low refractive index material of one embodiment of the present invention described in Example 2 and is represented by Structural Formula (120), was deposited by evaporation to a thickness of 10 nm to form the hole-blocking layer. Then, mmtBumBPTzn and 8-quinolinolato-lithium (abbreviation: Liq) represented by Structural Formula (ix) were deposited by co-evaporation to a thickness of 20 nm such that the weight ratio of mmtBumBPTzn to Liq was 1:1, whereby the electron-transport layer 114 was formed.

After the electron-transport layer 114 was formed, Liq was deposited by evaporation to a thickness of 1 nm to form the electron-injection layer 115, and lastly silver (Ag) and magnesium (Mg) were deposited by co-evaporation to a thickness of 15 nm such that the volume ratio of Ag to Mg was 1:0.1 to form the second electrode 102, whereby the light-emitting device 2 was fabricated. The second electrode 102 is a transflective electrode having a function of reflecting light and a function of transmitting light; thus, the light-emitting device of this example is a top-emission device in which light is extracted through the second electrode 102. Over the second electrode 102, 1,3,5-tri(dibenzothiophen-4-yl)-benzene (abbreviation: DBT3P-II) represented by Structural Formula (x) was deposited by evaporation to a thickness of 70 nm so that outcoupling efficiency can be improved.

### (Fabrication Method of Comparative Light-Emitting Device 2)

The comparative light-emitting device 2 was fabricated in the same manner as the light-emitting device 2 except that the thickness of the hole-transport layer 112 was 15 nm; 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (vi) was used instead of mmtBumBPTzn for the hole-blocking layer; and 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (viii) was used instead of mmtBumBPTzn for the electron-transport layer 114.

The structures of the light-emitting device 2 and the comparative light-emitting device 2 are listed in Table 4 below.

TABLE 4

| | film | Light- | Comparative light- |
|---|---|---|---|
| | thickness | emitting device 2 | emitting device 2 |
| electron-injection layer | 1 nm | Liq | |
| electron-transport layer | 20 nm | mmtBumBPTzn:Liq | mPn-mDMePyPTzn:Liq |
| | | (1:1) | (1:1) |
| hole-blocking layer | 10 nm | mmtBumBPTzn | mFBPTzn |

| | | |
|---|---|---|
| light-emitting layer | 25 nm | Bnf(II)PhA:3,10PCA2Nbf(IV)-02 |
| | | (1:0.015) |
| electron-blocking layer | 10 nm | PCzN2 |
| hole-transport layer | 10 nm | DBfBB1TP |
| | *2 | PCBBiF |
| hole-injection layer | 10 nm | PCBBiF:OCHD-001 |
| | | (1:0.05) |

*2 Light-emitting device 2: 20 nm, Comparative light-emitting device 2: 15 nm

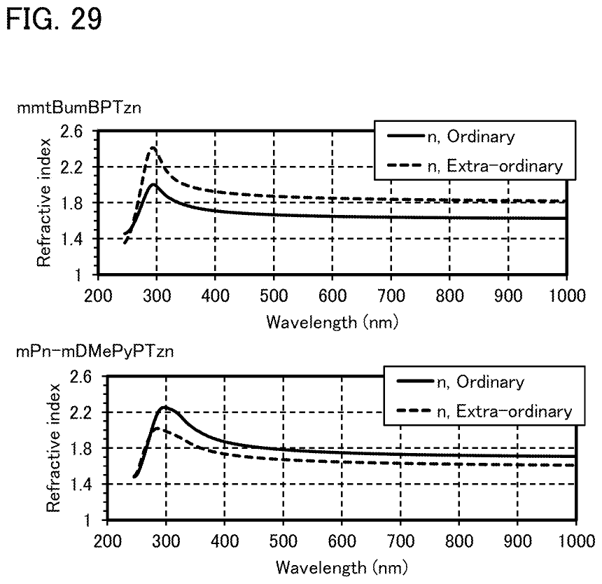

FIG. 29 shows the refractive indices of mmtBumBPTzn and mPn-mDMePyPTzn, and Table 5 shows the refractive indices at a wavelength of 456 nm. The refractive indices were measured with a spectroscopic ellipsometer (M-2000U, produced by J.A. Woollam Japan Corp.). A sample used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 29.

TABLE 5

| | ordinary refractive index (n, Ordinary) |
|---|---|
| | at 456 nm |
| mmtBumBPTzn | 1.68 |
| mFBPTzn | 1.80 |
| mPn-mDMePyPTzn | 1.81 |

The light-emitting device and the comparative light-emitting device were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

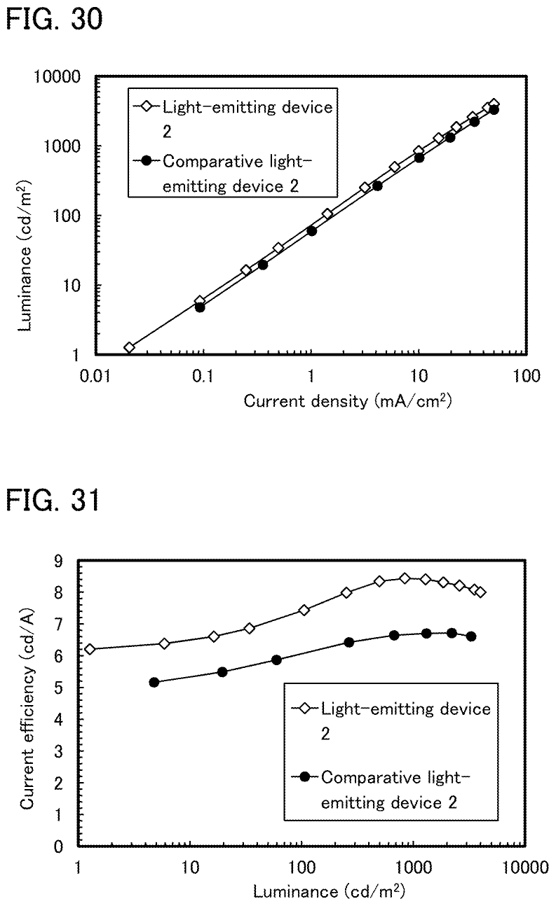

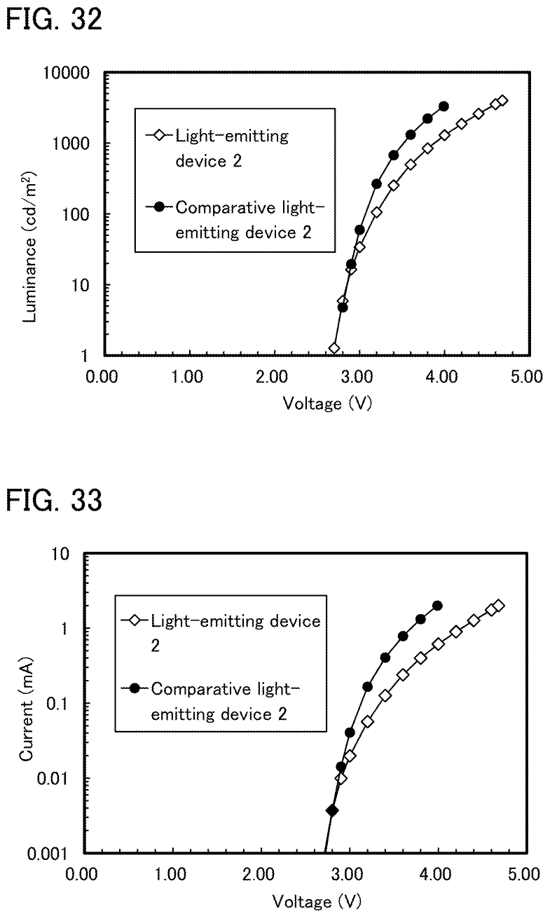

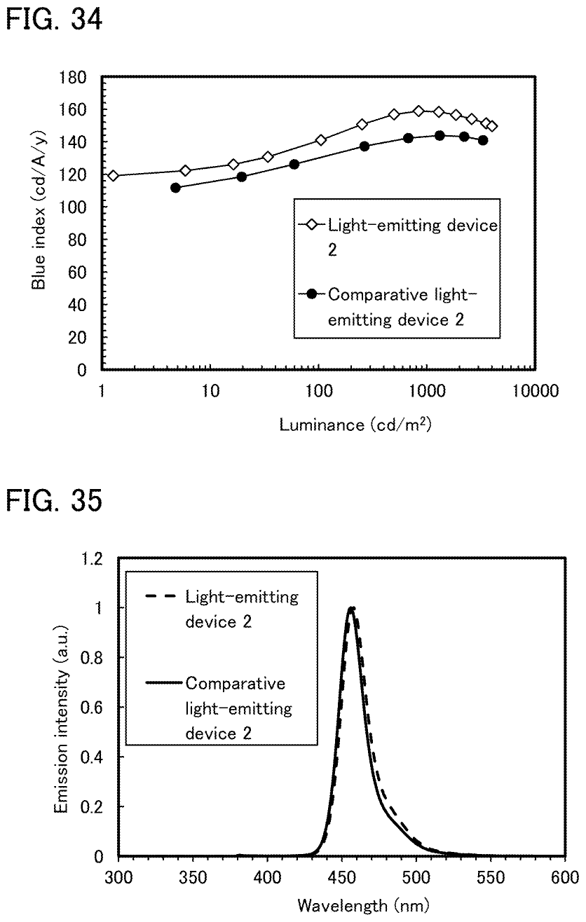

FIG. 30 shows the luminance-current density characteristics of the light-emitting device 2 and the comparative light-emitting device 2. FIG. 31 shows the current efficiency-luminance characteristics thereof. FIG. 32 shows the luminance-voltage characteristics thereof. FIG. 33 shows the current-voltage characteristics thereof. FIG. 34 shows the blue index-luminance characteristics thereof. FIG. 35 shows the emission spectra thereof. Table 6 shows the main characteristics of the light-emitting device 2 and the comparative light-emitting device 2 at a luminance of about 1000 cd/m². Luminance, CIE chromaticity, and emission spectra were measured at normal temperature with a spectroradiometer (SR-UL1R manufactured by TOPCON TECHNOHOUSE CORPORATION).

TABLE 6

| | | | | | | current | |
|---|---|---|---|---|---|---|---|
| | voltage | current | current density | | | efficiency | BI |
| | (V) | (mA) | (mA/cm²) | chromaticity x | chromaticity y | (cd/A) | (cd/A/y) |
| Light-emitting device 2 | 3.8 | 0.40 | 10.0 | 0.14 | 0.05 | 8.4 | 159 |
| Comparative light-emitting device 2 | 3.6 | 0.78 | 19.6 | 0.14 | 0.05 | 6.7 | 144 |

FIG. 30 to FIG. 35 and Table 6 reveal that the light-emitting device 2 fabricated using the low refractive index material of one embodiment of the present invention is an EL device having substantially the same emission spectrum as the comparative light-emitting device 2 and having higher current efficiency than the comparative light-emitting device 2.

The maximum values of the BI of the light-emitting device 2 and the comparative light-emitting device 2 were 159 (cd/A/y) and 144 (cd/A/y), respectively. Thus, the light-emitting device 2 can be regarded as having especially favorable BI. Accordingly, one embodiment of the present invention is suitable for a light-emitting device used for a display.

### Example 7

### Synthesis Example 5

In this example, a method for synthesizing 2-(3,3″,5′,5″-tetra-tert-butyl-1,1′:3′,1″-terphenyl-5-yl)-4,6-diphenyl-1,3,5-triazine (abbreviation: mmtBumTPTzn-02), which is the organic compound represented by Structural Formula (123) in Embodiment 1, is described. The structure of mmtBumTPTzn-02 is shown below.

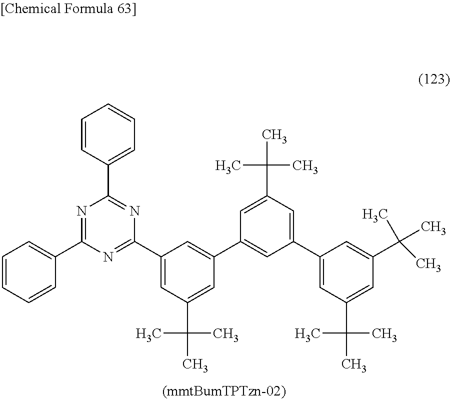 [SMILES: CC(C)(C)c1cc(-c2cc(-c3nc(-c4ccccc4)nc(-c4ccccc4)n3)cc(C(C)(C)C)c2)cc(-c2cc(C(C)(C)C)cc(C(C)(C)C)c2)c1]

### Step 1: Synthesis of 3,3″,5′,5″-tetra-t-butyl-1,1′:3′,1″-terphenyl-5-yl-4,4,5,5-tetramethyl-1,3,2-dioxaborolane

Into a three-neck flask were put 1.0 g (1.9 mmol) of 5-bromo-3,3″,5′,5″-tetra-t-butyl-1,1′:3′,1″-terphenyl, 0.62 g (2.4 mmol) of bis(pinacolato)diboron, 0.61 g (6.2 mmol) of potassium acetate, and 18 mL of 1,4-dioxane, and the mixture was degassed. To this mixture was added 0.077 g (0.094 mmol) of [1,1′-bis(diphenylphosphino)ferrocene]palladium(II) dichloride dichloromethane adduct, and reaction was caused under a nitrogen stream at 110° C. for 24 hours.

After the reaction, extraction was performed with toluene and the obtained organic layer was dried with magnesium sulfate. This mixture was gravity-filtered, and the obtained filtrate was concentrated, which was then purified by silica gel column chromatography with a developing solvent of toluene and hexane in a ratio of 2:1, which was then changed to only toluene, to give 0.78 g of a target white solid in a yield of 72%. The synthesis scheme of Step 1 is shown in (e-1) below.

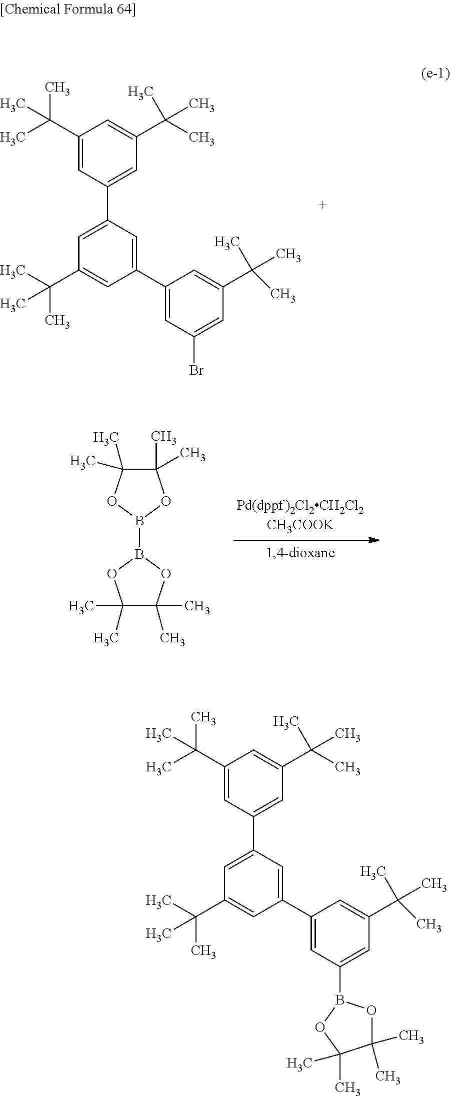

### Step 2: Synthesis of mmtBumTPTzn-02

Into a three-neck flask were put 0.33 g (1.2 mmol) of 4,6-diphenyl-2-chloro-1,3,5-triazine, 0.78 g (1.3 mmol) of 3,3″,5′,5″-tetra-t-butyl-1,1′:3′,1″-terphenyl-5-yl-4,4,5,5-tetramethyl-1,3,2-dioxaborolane, 0.57 g (2.7 mmol) of tripotassium phosphate, 3 mL of water, 7 mL of toluene, and 3 mL of 1,4-dioxane, and the mixture was degassed. To this mixture were added 3 mg (0.013 mmol) of palladium(II) acetate and 8 mg (0.027 mmol) of tris(2-methylphenyl)phosphine, and the mixture was heated and refluxed under a nitrogen stream for 20 hours. After the reaction, extraction was performed with toluene and the obtained organic layer was dried with magnesium sulfate. This mixture was gravity-filtered, and the obtained filtrate was concentrated to give a yellow solid. This solid was purified by silica gel column chromatography with a developing solvent of chloroform and hexane in a ratio of 5:1, which was then changed to 3:1, to give 0.70 g of a white solid. This solid was recrystallized with ethanol and hexane to give 0.64 g of a target white solid in a yield of 76%. The synthesis scheme of Step 2 is shown in (e-2) below.

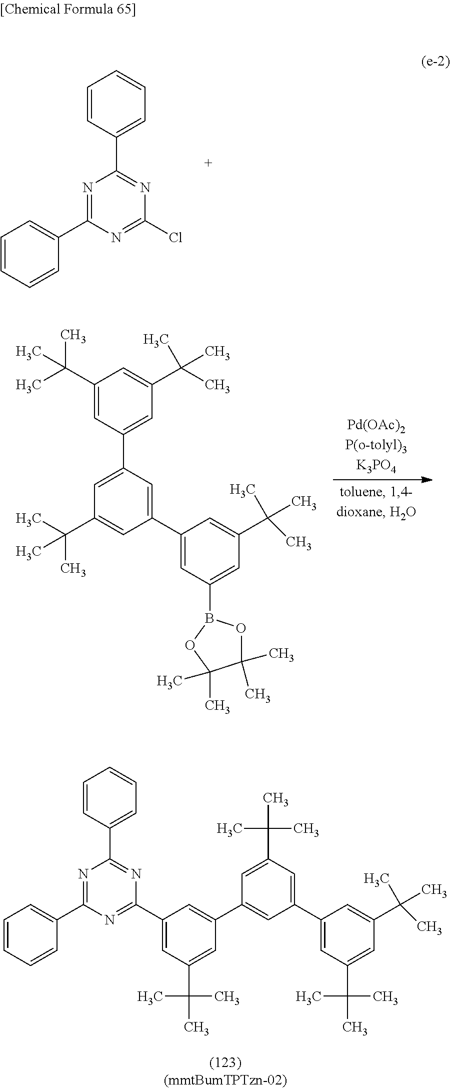 [SMILES: Clc1nc(-c2ccccc2)nc(-c2ccccc2)n1 ; CC(C)(C)c1cc(B2OC(C)(C)C(C)(C)O2)cc(-c2cc(-c3cc(C(C)(C)C)cc(C(C)(C)C)c3)cc(C(C)(C)C)c2)c1 ; CC(C)(C)c1cc(-c2cc(-c3nc(-c4ccccc4)nc(-c4ccccc4)n3)cc(C(C)(C)C)c2)cc(-c2cc(C(C)(C)C)cc(C(C)(C)C)c2)c1]

Analysis results by nuclear magnetic resonance (¹H-NMR) spectroscopy of the white solid obtained in Step 2 are shown below. These results reveal that the organic compound of one embodiment of the present invention, mmtBumTPTzn-02 represented by Structural Formula (123), was obtained in this example.

¹H NMR (CDCl₃, 300 MHz): δ=1.41 (s, 18H), 1.49 (s, 9H), 1.52 (s, 9H), 7.49 (s, 3H), 7.58-7.63 (m, 7H), 7.69-7.70 (m, 2H), 7.88 (t, 1H), 8.77-8.83 (m, 6H).

Next, the absorption spectrum of mmtBumTPTzn-02 was measured with an ultraviolet-visible light spectrophotometer (V550, manufactured by JASCO Corporation). The absorption spectrum of mmtBumTPTzn-02 in the dichloromethane solution was obtained by subtracting the spectrum of dichloromethane alone in a quartz cell from the absorption spectrum of mmtBumTPTzn-02 in the dichloromethane solution in the quartz cell. As a result, the absorption peak was observed at 267 nm, revealing that no absorption was shown in the visible region range from 440 nm to 700 nm.

Next, mmtBumTPTzn-02 obtained in this example was analyzed by liquid chromatography mass spectrometry (LC/MS).

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumTPTzn-02 in an organic solvent at an arbitrary concentration, and the injection amount was 5.0 μL.

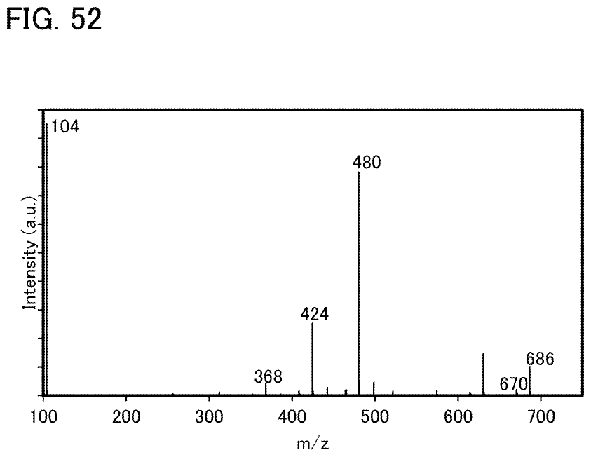

The MS/MS measurement of m/z=685.44 corresponding to the exact mass of mmtBumTPTzn-02 was performed by a PRM method. For setting of the PRM, the mass range of a target ion was set to m/z=685.44±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 52.

Fragment ions at m/z of 104.05 and m/z of 670.42 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 670.42 is probably a fragment in which one carbon atom and one nitrogen atom each derived from triazine are bonded to a substituent other than a phenyl group. These fragments can each be regarded as a feature of a compound having a triazine skeleton.

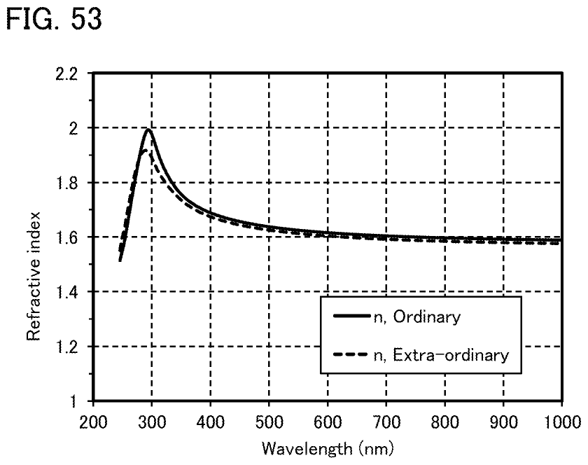

FIG. 53 shows the results of measuring the refractive index of mmtBumTPTzn-02 with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 53.

FIG. 53 shows that mmtBumTPTzn-02 is a material with a low refractive index: the ordinary refractive index of mmtBumTPTzn-02 in the entire blue emission region (from 455 nm to 465 nm) is 1.65, which is in the range of 1.50 to 1.75, and the ordinary refractive index of mmtBumTPTzn-02 at a wavelength of 633 nm is 1.61, which is in the range of 1.45 to 1.70.

Note that favorable light emission can be obtained from a light-emitting device fabricated using the organic compound as an electron-transport material.

### Example 8

In this example, light-emitting devices 3, 4, and 5 of one embodiment of the present invention are described. Structural formulae of organic compounds used in this example are shown below.

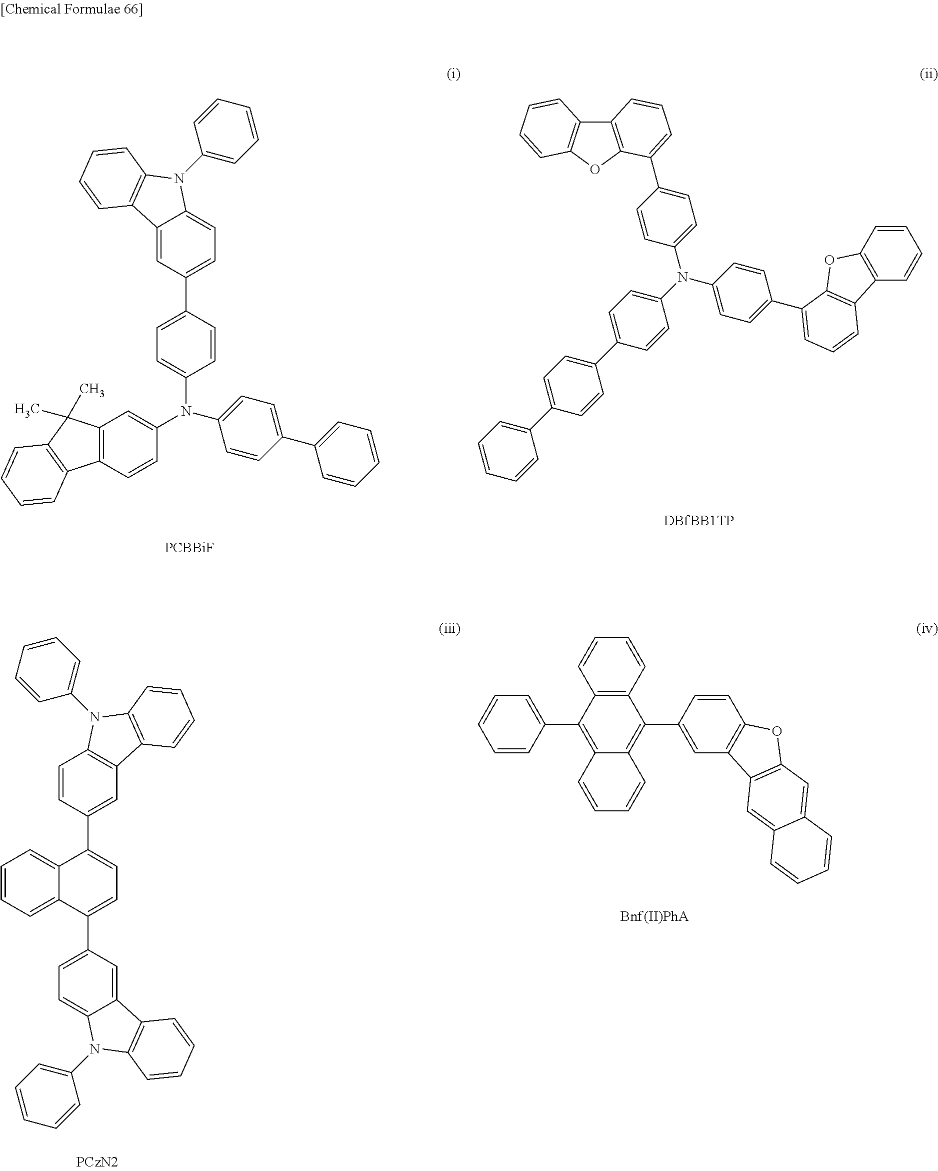

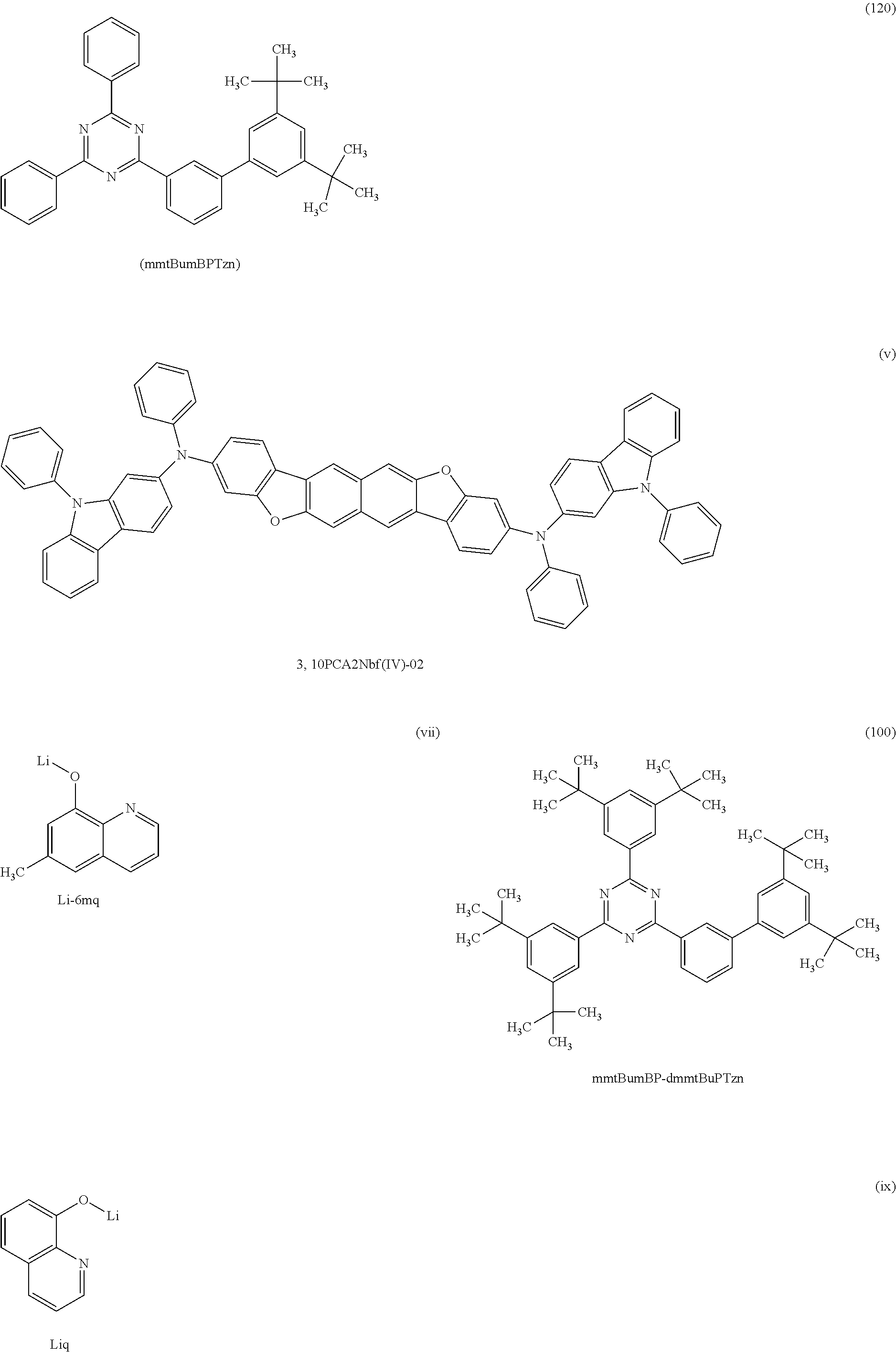

### (Fabrication Method of Light-Emitting Devices)

First, as a transparent electrode, indium tin oxide containing silicon oxide (ITSO) was formed over a glass substrate to a thickness of 110 nm by a sputtering method, whereby the first electrode 101 was formed. The electrode area was 4 mm² (2 mm×2 mm).

Next, in pretreatment for forming the light-emitting device over a substrate, a surface of the substrate was washed with water and baked at 200° C. for one hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structure Formula (i) and an electron acceptor material (OCHD-001) were deposited by co-evaporation to a thickness of 10 nm such that the weight ratio of PCBBiF to OCHD-001 was 1:0.05, whereby the hole-injection layer 111 was formed.

Over the hole-injection layer 111, PCBBiF was deposited by evaporation to a thickness of 80 nm, and then N,N-bis[4-(dibenzofuran-4-yl)phenyl]-4-amino-p-terphenyl (abbreviation: DBfBB1TP) represented by Structural Formula (ii) was deposited by evaporation to a thickness of 10 nm, whereby the hole-transport layer 112 was formed.

Subsequently, over the hole-transport layer 112, 3,3′-(naphthalene-1,4-diyl)bis(9-phenyl-9H-carbazole) (abbreviation: PCzN2) represented by Structural Formula (iii) was deposited by evaporation to a thickness of 10 nm, whereby the electron-blocking layer was formed.

Then, 2-(10-phenyl-9-anthracenyl)-benzo[b]naphtho[2,3-d]furan (abbreviation: Bnf(II)PhA) represented by Structural Formula (iv) and 3,10-bis[N-(9-phenyl-9H-carbazol-2-yl)-N-phenylamino]naphtho[2,3-b;6,7-b]bisbenzofuran (abbreviation: 3,10PCA2Nbf(IV)-02) represented by Structural Formula (v) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, in the light-emitting device 3 and the light-emitting device 4, 2-{(3′,5′-di-tert-butyl)-1,1′-biphenyl-3-yl}-4,6-bis(3,5-di-tert-butylphenyl)-1,3,5-triazine (abbreviation: mmtBumBP-dmmtBuPTzn), which is the low refractive index material of one embodiment of the present invention described in Example 1 and is represented by Structural Formula (100), was deposited by evaporation to a thickness of 10 nm to form the hole-blocking layer. Then, in the light-emitting device 3, mmtBumBP-dmmtBuPTzn and 8-quinolinolato-lithium (abbreviation: Liq) represented by Structural Formula (ix) were deposited by co-evaporation to a thickness of 20 nm such that the weight ratio of mmtBumBP-dmmtBuPTzn to Liq was 1:1, whereby the electron-transport layer 114 was formed. In the light-emitting device 4, mmtBumBP-dmmtBuPTzn and 6-methyl-8-quinolinolato-lithium (abbreviation: Li-6mq) represented by Structural Formula (vii) were deposited by co-evaporation to a thickness of 20 nm such that the weight ratio of mmtBumBP-dmmtBuPTzn to Li-6mq was 1:1, whereby the electron-transport layer 114 was formed. After the electron-transport layer 114 was formed, Liq was deposited by evaporation to a thickness of 1 nm to form the electron-injection layer 115 in the light-emitting device 3, and Li-6mq was deposited by evaporation to a thickness of 1 nm to form the electron-injection layer 115 in the light-emitting device 4.

In the light-emitting device 5, 2-{(3′,5′-di-tert-butyl)-1,1′-biphenyl-3-yl}-4,6-diphenyl-1,3,5-triazine (abbreviation: mmtBumBPTzn), which is the low refractive index material of one embodiment of the present invention described in Example 2 and is represented by Structural Formula (120), was deposited by evaporation to a thickness of 10 nm to form the hole-blocking layer. Then, mmtBumBPTzn and 8-quinolinolato-lithium (abbreviation: Liq) represented by Structural Formula (ix) were deposited by co-evaporation to a thickness of 20 nm such that the weight ratio of mmtBumBPTzn to Liq was 1:1, whereby the electron-transport layer 114 was formed. After the electron-transport layer 114 was formed, Liq was deposited by evaporation to a thickness of 1 nm to form the electron-injection layer 115.

Lastly, aluminum was deposited by evaporation to a thickness of 200 nm to form the second electrode 102. In the above manner, the light-emitting devices were fabricated. The second electrode 102 is a reflective electrode having a function of reflecting light; thus, the light-emitting devices of this example are each a bottom-emission device in which light is extracted through the first electrode 101.

The structures of the light-emitting devices 3, 4, and 5 are listed in Table 7 below.

TABLE 7

| | film | Light- | Light- | Light- |
|---|---|---|---|---|
| | thickness | emitting device 3 | emitting device 4 | emitting device 5 |
| electron-injection layer | 1 nm | Liq | Li-6mq | Liq |
| electron-transport layer | 20 nm | mmtBumBP- | mmtBumBP- | mmtBumBPTzn:Liq |
| | | dmmtBuPTzn:Liq | dmmtBuPTzn:Li-6mq | (1:1) |
| | | (1:1) | (1:1) | |

| | | | |
|---|---|---|---|
| hole-blocking layer | 10 nm | mmtBumBP-dmmtBuPTzn | mmtBumBPTzn |

| | | |
|---|---|---|
| light-emitting layer | 25 nm | Bnf(II)PhA:3,10PCA2Nbf(IV)-02 |
| | | (1:0.015) |
| electron-blocking layer | 10 nm | PCzN2 |
| hole-transport layer | 10 nm | DBfBB1TP |
| | 80 nm | PCBBiF |
| hole-injection layer | 10 nm | PCBBiF:OCHD-001 |
| | | (1:0.05) |

Table 8 shows the refractive indices of mmtBumBP-dmmtBuPTzn, mmtBumBPTzn, Li-6mq, and Liq at a wavelength of 456 nm. The refractive indices were measured with a spectroscopic ellipsometer (M-2000U, produced by J.A. Woollam Japan Corp.). A sample used for the measurement was formed to a thickness of approximately 50 nm with the material of each layer over a quartz substrate by a vacuum evaporation method.

TABLE 8

| | ordinary refractive index (n, Ordinary) |
|---|---|
| | at 456 nm |
| mmtBumBP-dmmtBuPTzn | 1.61 |
| mmtBumBPTzn | 1.68 |
| Li-6mq | 1.67 |
| Liq | 1.72 |

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

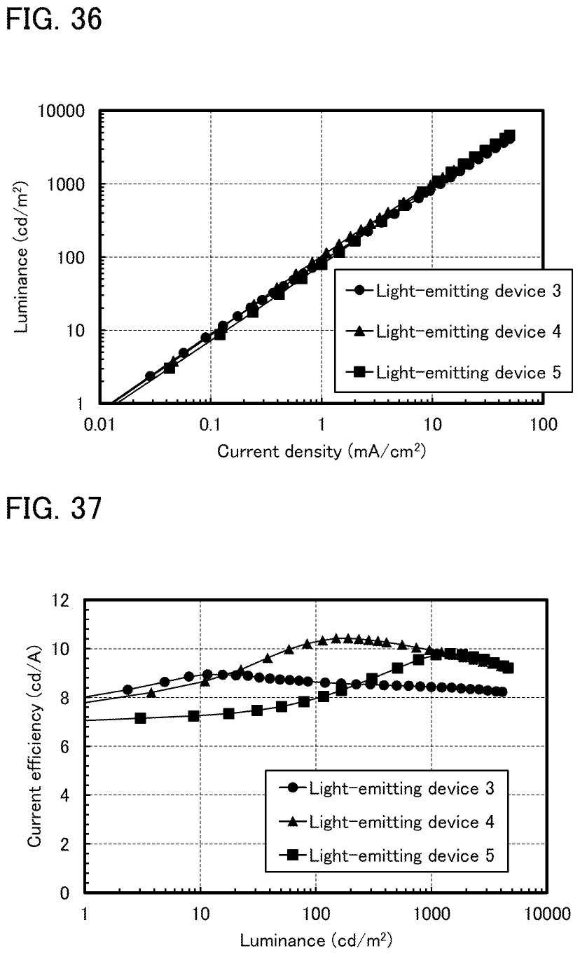

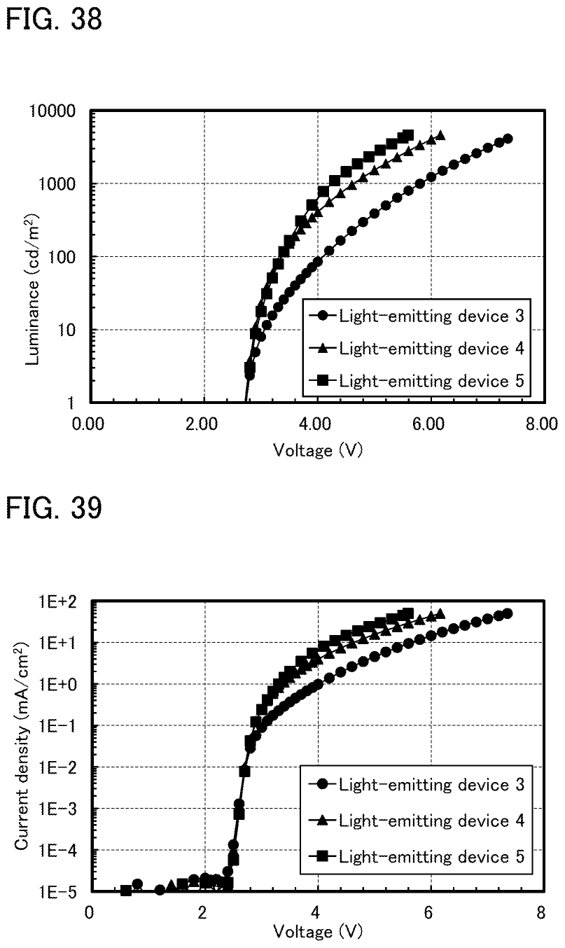

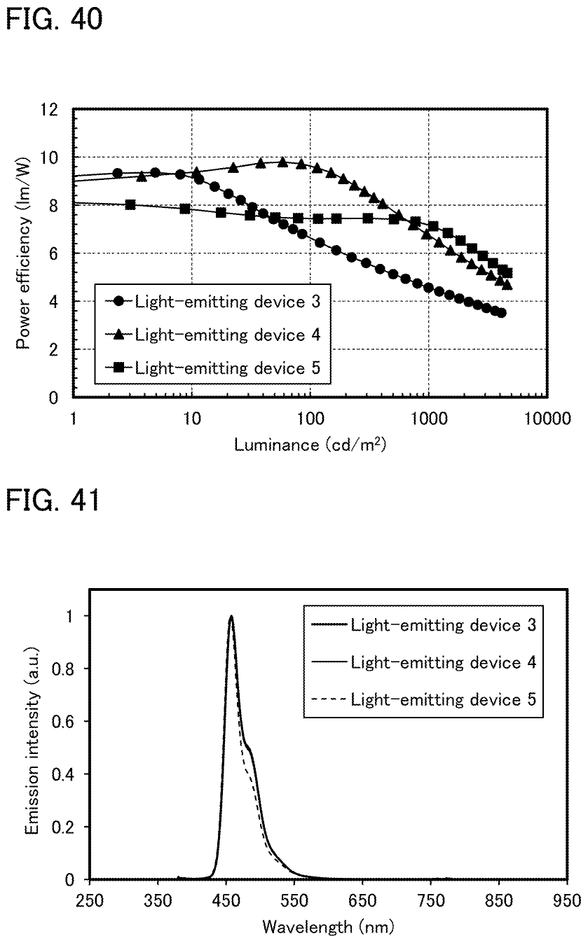

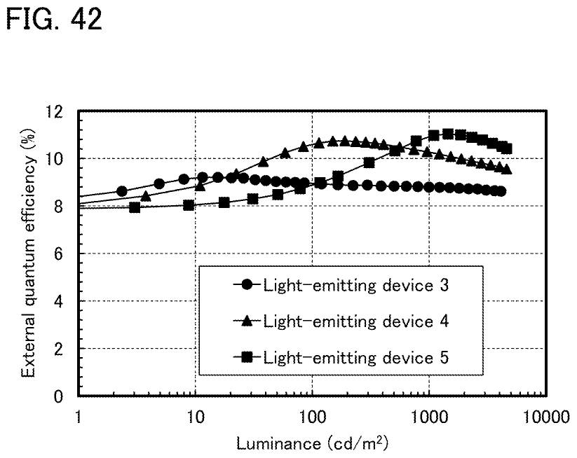

FIG. 36 shows the luminance-current density characteristics of the light-emitting devices 3, 4, and 5. FIG. 37 shows the current efficiency-luminance characteristics thereof. FIG. 38 shows the luminance-voltage characteristics thereof. FIG. 39 shows the current density-voltage characteristics thereof. FIG. 40 shows the power efficiency-luminance characteristics thereof. FIG. 41 shows the emission spectra thereof. FIG. 42 shows the external quantum efficiency-luminance characteristics thereof. Table 9 shows the main characteristics of the light-emitting devices 3, 4, and 5 at a luminance of about 1000 cd/m². Luminance, CIE chromaticity, and emission spectra were measured at normal temperature with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 9

| | | | | | | current | external |
|---|---|---|---|---|---|---|---|
| | voltage | current | current density | | | efficiency | quantum |
| | (V) | (mA) | (mA/cm²) | chromaticity x | chromaticity y | (cd/A) | efficiency (%) |
| Light-emitting device 3 | 5.8 | 0.47 | 12 | 0.14 | 0.11 | 8.4 | 8.8 |
| Light-emitting device 4 | 4.6 | 0.39 | 9.6 | 0.14 | 0.11 | 9.9 | 10 |
| Light-emitting device 5 | 4.3 | 0.45 | 11 | 0.14 | 0.099 | 9.8 | 11 |

The results in FIG. 36 to FIG. 42 and Table 9 reveal that the light-emitting devices 3, 4, and 5 fabricated using the low refractive index material of one embodiment of the present invention are each an EL device having high current efficiency and external quantum efficiency.

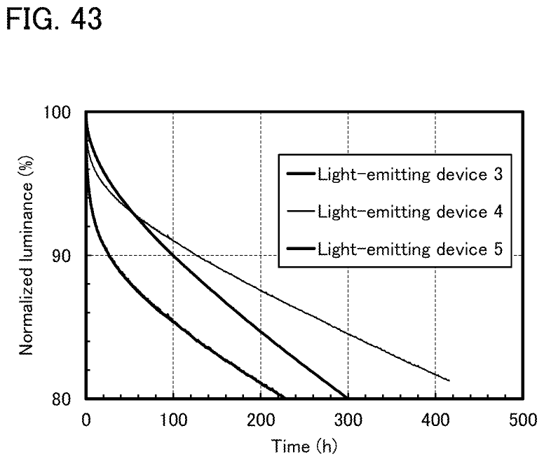

Next, reliability tests were performed on the light-emitting devices. FIG. 43 shows the reliability test results of the light-emitting devices 3, 4, and 5. In FIG. 43 showing the reliability, the vertical axis represents normalized luminance (%) with an initial luminance of 100%, and the horizontal axis represents driving time (h) of the devices. As the reliability tests, driving tests at a constant current density of 50 mA/cm² were performed on the light-emitting devices.

The above results demonstrate that the light-emitting devices 3, 4, and 5 in this example containing mmtBumBP-dmmtBuPTzn or mmtBumBPTzn, which is the low refractive index material of one embodiment of the present invention, in their EL layers each have a normalized luminance of approximately 80% or higher after 200 hours have elapsed, revealing that the light-emitting devices have high reliability.

### Example 9

### Synthesis Example 6

In this example, a method for synthesizing 2-{3-(3,5-dicyclohexylphenyl)phenyl}-4,6-diphenyl-1,3,5-triazine (abbreviation: mmchmBPTzn), which is the organic compound represented by Structural Formula (412) in Embodiment 1, is described. The structure of mmchmBPTzn is shown below.

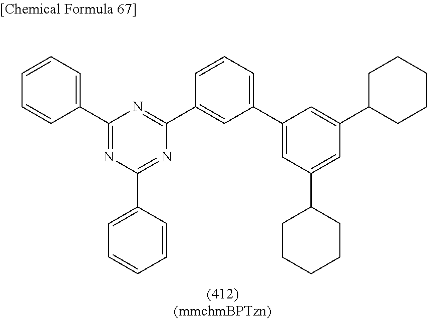 [SMILES: c1ccc(-c2nc(-c3ccccc3)nc(-c3cccc(-c4cc(C5CCCCC5)cc(C5CCCCC5)c4)c3)n2)cc1]

### Step 1: Synthesis of mmchmBPTzn

Into a three-neck flask were put 2.7 g (6.1 mmol) of 2,4-diphenyl-6-[3-(4,4,5,5-tetramethyl-1,3,2-dioxaborolan-2-yl)phenyl]-1,3,5-triazine, 1.7 g (4.3 mmol) of 3,5-dicyclohexyl-1-phenyl trifluoromethanesulfonate, and 1.7 g (12 mmol) of potassium carbonate. To this mixture were added 60 mL of toluene, 12 mL of ethanol, and 6 mL of water, and the mixture was degassed by being stirred under reduced pressure. Then, 0.25 g (0.61 mmol) of 2-dicyclohexylphosphino-2′,6′-dimethoxybiphenyl (SPhos) and 0.0275 g (0.123 mmol) of palladium(II) acetate were added to this mixture, and the mixture was stirred under a nitrogen atmosphere at 80° C. for 9.5 hours.

After the reaction, the reaction solution was filtered and extraction was performed with ethyl acetate. The solution of the extract was filtered through Celite and dehydrated with magnesium sulfate, which was then concentrated to give a brown solid containing a target substance. The obtained solid was purified by silica gel column chromatography with a developing solvent of chloroform and hexane in a ratio of 1:5, which was then changed to 1:3, to give a target white solid. The synthesis scheme of Step 1 is shown below.

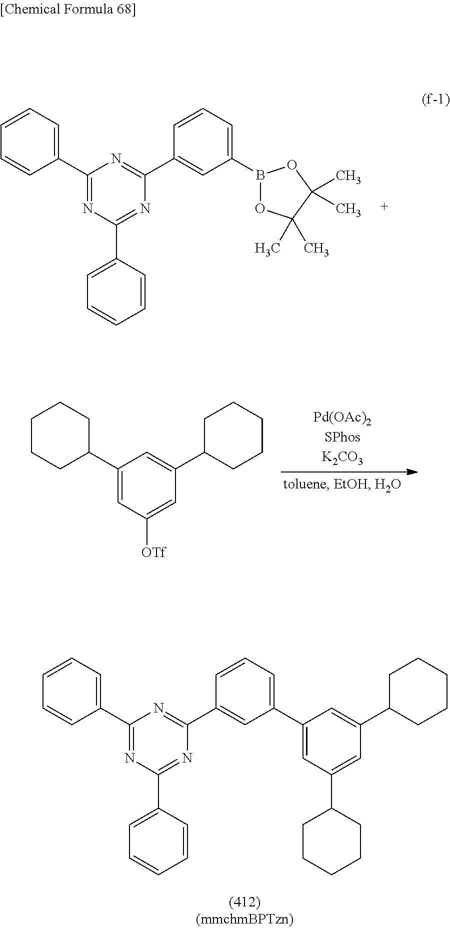 [SMILES: CC1(C)OB(c2cccc(-c3nc(-c4ccccc4)nc(-c4ccccc4)n3)c2)OC1(C)C ; O=S(=O)(Oc1cc(C2CCCCC2)cc(C2CCCCC2)c1)C(F)(F)F ; c1ccc(-c2nc(-c3ccccc3)nc(-c3cccc(-c4cc(C5CCCCC5)cc(C5CCCCC5)c4)c3)n2)cc1]

The resulting solid was further purified by high performance liquid column chromatography. The high performance liquid column chromatography was performed using chloroform as a developing solvent. The obtained fraction was concentrated to give a target oily substance. The obtained oil was recrystallized with hexane and toluene to give 1.5 g of a target white solid in a yield of 65%.

Then, 1.5 g of the obtained white solid was purified by a train sublimation method. In the purification by sublimation, the solid was heated under a pressure of 6.5 Pa at 265° C. for 19 hours while an argon gas was made to flow. After the purification by sublimation, 1.1 g of a target white solid was obtained at a collection rate of 73%.

The molecular weight of the target substance obtained by the above-described synthesis method was measured with a GC/MS detector (ITQ1100 ion trap GC/MS system, manufactured by Thermo Fisher Scientific K.K.). Accordingly, a main peak with a mass number of 549 (mode: EI+) was detected. The result reveals that the organic compound of one embodiment of the present invention, mmchmBPTzn represented by Structural Formula (412), was obtained in this example.

Next, the absorption spectrum of mmchmBPTzn was measured with an ultraviolet-visible light spectrophotometer (V550, manufactured by JASCO Corporation). The absorption spectrum of mmchmBPTzn in the dichloromethane solution was obtained by subtracting the spectrum of dichloromethane alone in a quartz cell from the absorption spectrum of mmchmBPTzn in the dichloromethane solution in the quartz cell. As a result, the absorption peak was observed at 271 nm, revealing that no absorption was shown in the visible region range from 440 nm to 700 nm.

Next, mmchmBPTzn obtained in this example was subjected to LC/MS analysis.

In the LC/MS analysis, LC separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmchmBPTzn in an organic solvent at an arbitrary concentration, and the injection amount was 5.0 μL.

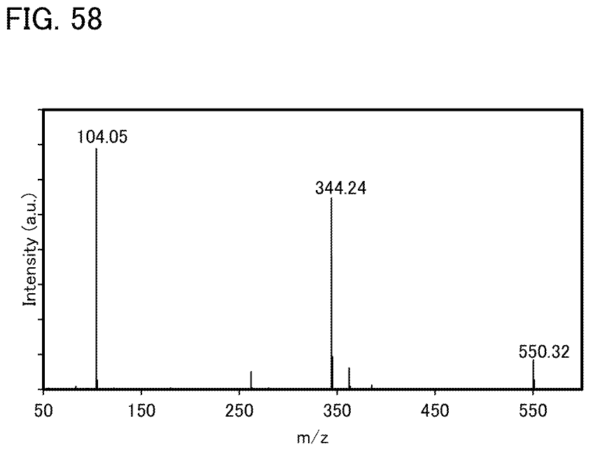

The MS/MS measurement of m/z=549.31 corresponding to the exact mass of mmchmBPTzn was performed by a PRM method. For setting of the PRM, the mass range of a target ion was set to m/z=549.31±2.0 (isolation window=4) and detection was performed in a positive mode. The measurement was performed with energy (NCE) for accelerating a target ion in a collision cell set to 50. The MS spectrum obtained by the MS/MS measurement is shown in FIG. 58.

Fragment ions at m/z of 104.05 and m/z of 344.24 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 344.24 is probably a fragment in which one carbon atom and one nitrogen atom each derived from triazine are bonded to a substituent other than a phenyl group. These fragments can each be regarded as a feature of a compound having a triazine skeleton.

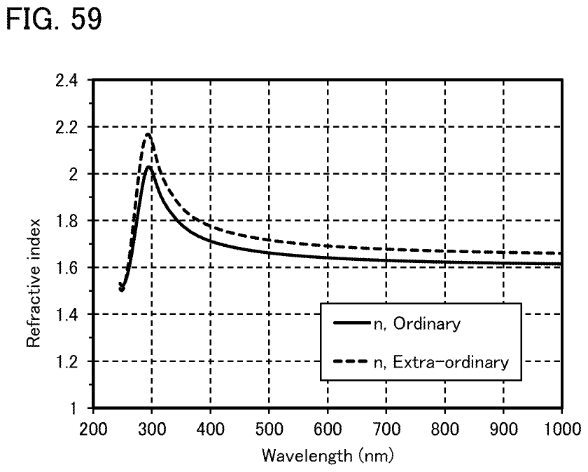

FIG. 59 shows the results of measuring the refractive index of mmchmBPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 59.

FIG. 59 shows that mmchmBPTzn is a material with a low refractive index: the ordinary refractive index of mmchmBPTzn in the entire blue emission region (from 455 nm to 465 nm) is 1.68, which is in the range of 1.50 to 1.75, and the ordinary refractive index of mmchmBPTzn at a wavelength of 633 nm is 1.64, which is in the range of 1.45 to 1.70.

Note that favorable light emission can be obtained from a light-emitting device fabricated using the organic compound as an electron-transport material.

### Example 10

In this example, light-emitting devices 6 and 7 of one embodiment of the present invention and a comparative light-emitting device 3 are described. Structural formulae of organic compounds used in this example are shown below.

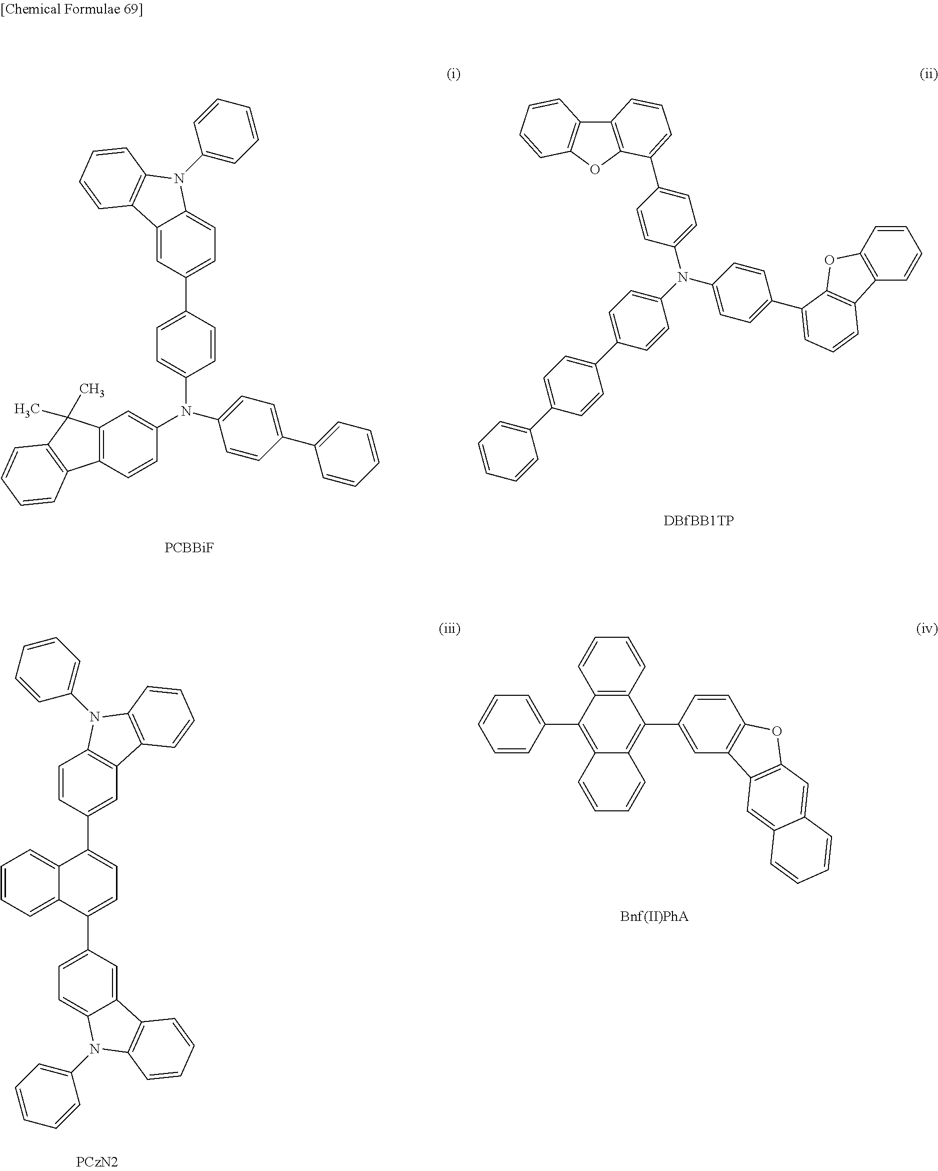

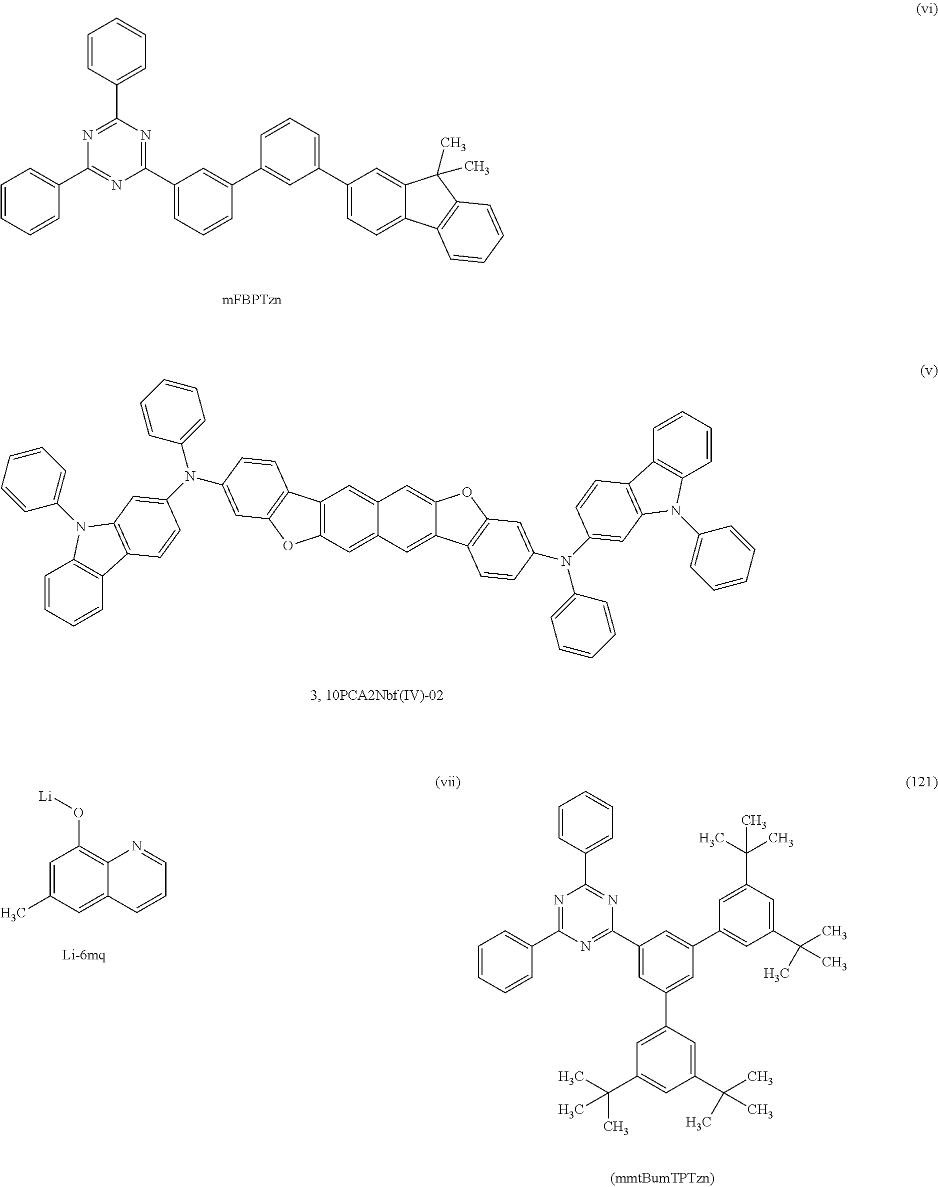

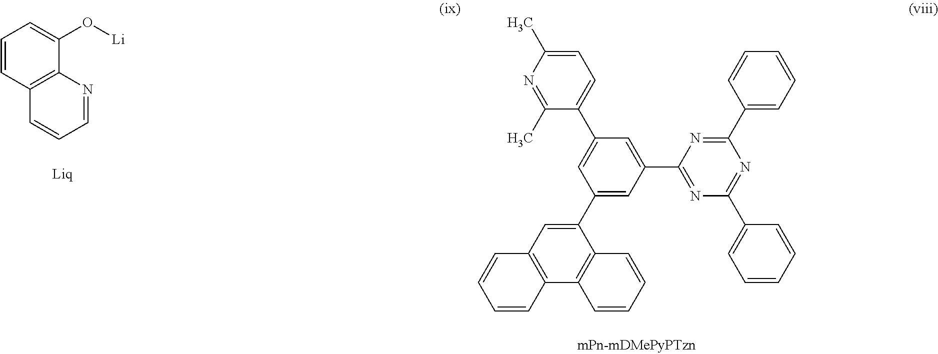

### (Fabrication Method of Light-Emitting Devices)

First, as a transparent electrode, indium tin oxide containing silicon oxide (ITSO) was formed over a glass substrate to a thickness of 110 nm by a sputtering method, whereby the first electrode 101 was formed. The electrode area was 4 mm² (2 mm×2 mm).

Next, in pretreatment for forming the light-emitting device over a substrate, a surface of the substrate was washed with water and baked at 200° C. for one hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structure Formula (i) and an electron acceptor material (OCHD-001) were deposited by co-evaporation to a thickness of 10 nm such that the weight ratio of PCBBiF to OCHD-001 was 1:0.05, whereby the hole-injection layer 111 was formed.

Over the hole-injection layer 111, PCBBiF was deposited by evaporation to a thickness of 20 nm, and then N,N-bis[4-(dibenzofuran-4-yl)phenyl]-4-amino-p-terphenyl (abbreviation: DBfBB1TP) represented by Structural Formula (ii) was deposited by evaporation to a thickness of 10 nm, whereby the hole-transport layer 112 was formed.

Subsequently, over the hole-transport layer 112, 3,3′-(naphthalene-1,4-diyl)bis(9-phenyl-9H-carbazole) (abbreviation: PCzN2) represented by Structural Formula (iii) was deposited by evaporation to a thickness of 10 nm, whereby the electron-blocking layer was formed.

Then, 2-(10-phenyl-9-anthracenyl)-benzo[b]naphtho[2,3-d]furan (abbreviation: Bnf(II)PhA) represented by Structural Formula (iv) and 3,10-bis[N-(9-phenyl-9H-carbazol-2-yl)-N-phenylamino]naphtho[2,3-b;6,7-b]bisbenzofuran (abbreviation: 3,10PCA2Nbf(IV)-02) represented by Structural Formula (v) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, in the light-emitting device 6 and the comparative light-emitting device 3, 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (vi) was deposited by evaporation to a thickness of 10 nm to form the hole-blocking layer. Then, in the light-emitting device 6, 2-(3,3″,5,5″-tetra-tert-butyl-1,1′: 3′,1″-terphenyl-5′-yl)-4,6-diphenyl-1,3,5-triazine (abbreviation: mmtBumTPTzn), which is the low refractive index material of one embodiment of the present invention and is represented by Structural Formula (121) in Embodiment 1, and 6-methyl-8-quinolinolato-lithium (abbreviation: Li-6mq) represented by Structural Formula (vii) were deposited by co-evaporation to a thickness of 20 nm such that the weight ratio of mmtBumTPTzn to Li-6mq was 1:1, whereby the electron-transport layer 114 was formed. In the comparative light-emitting device 3, 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (viii) and 8-quinolinolato-lithium (abbreviation: Liq) represented by Structural Formula (ix) were deposited by co-evaporation to a thickness of 20 nm such that the weight ratio of mPn-mDMePyPTzn to Liq was 1:1, whereby the electron-transport layer 114 was formed. After the light-emitting layer was formed in the light-emitting device 7, mmtBumTPTzn was deposited by evaporation to a thickness of 10 nm to form the hole-blocking layer, and then, mmtBumTPTzn and Li-6mq were deposited by co-evaporation to a thickness of 20 nm such that the weight ratio of mmtBumTPTzn to Li-6mq was 1:1 to form the electron-transport layer 114.

After the electron-transport layer 114 was formed, Liq was deposited by evaporation to a thickness of 1 nm to form the electron-injection layer 115. Lastly, aluminum was deposited by evaporation to a thickness of 200 nm to form the second electrode 102. In the above manner, the light-emitting devices were fabricated. The second electrode 102 is a reflective electrode having a function of reflecting light; thus, the light-emitting devices of this example are each a bottom-emission device in which light is extracted through the first electrode 101.

The structures of the light-emitting devices 6 and 7 and the comparative light-emitting device 3 are listed in Table 10.

TABLE 10

| | film | Light- | Light- | Comparative light- |
|---|---|---|---|---|
| | thickness | emitting device 6 | emitting device 7 | emitting device 3 |
| electron-injection layer | 1 nm | Li-6mq | | Liq |
| electron-transport layer | 20 nm | mmtBumTPTzn:Li-6mq | | mPn-mDMePyPTzn:Liq |
| | | (1:1) | | (1:1) |
| hole-blocking layer | 10 nm | mFBPTzn | mmtBumTPTzn | mFBPTzn |

| | | |
|---|---|---|
| light-emitting layer | 25 nm | Bnf(II)PhA:3,10PCA2Nbf(IV)-02 |
| | | (1:0.015) |
| electron-blocking layer | 10 nm | PCzN2 |
| hole-injection layer | 10 nm | DBfBB1TP |
| | 20 nm | PCBBiF |
| hole-transport layer | 10 nm | PCBBiF:OCHD-001 |
| | | (1:0.05) |

Table 11 shows the refractive indices of mmtBumTPTzn, mPn-mDMePyPTzn, Li-6mq, and Liq at a wavelength of 456 nm. The refractive indices were measured with a spectroscopic ellipsometer (M-2000U, produced by J.A. Woollam Japan Corp.). A sample used for the measurement was formed to a thickness of approximately 50 nm with the material of each layer over a quartz substrate by a vacuum evaporation method.

TABLE 11

| | ordinary refractive index (n, Ordinary) |
|---|---|
| | at 456 nm |
| mmtBumTPTzn | 1.62 |
| mPn-mDMePyPTzn | 1.81 |
| mFBPTzn | 1.80 |
| Li-6mq | 1.67 |
| Liq | 1.72 |

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

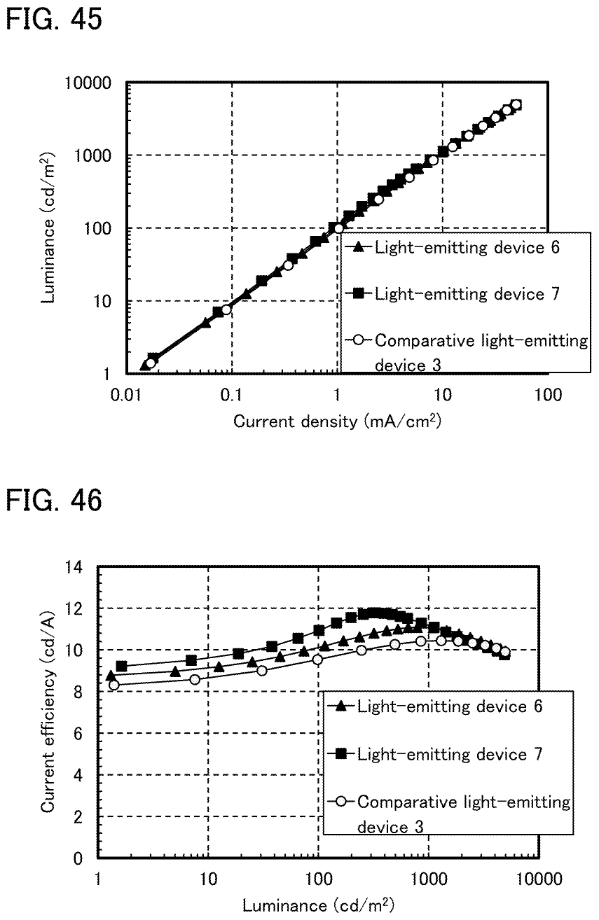

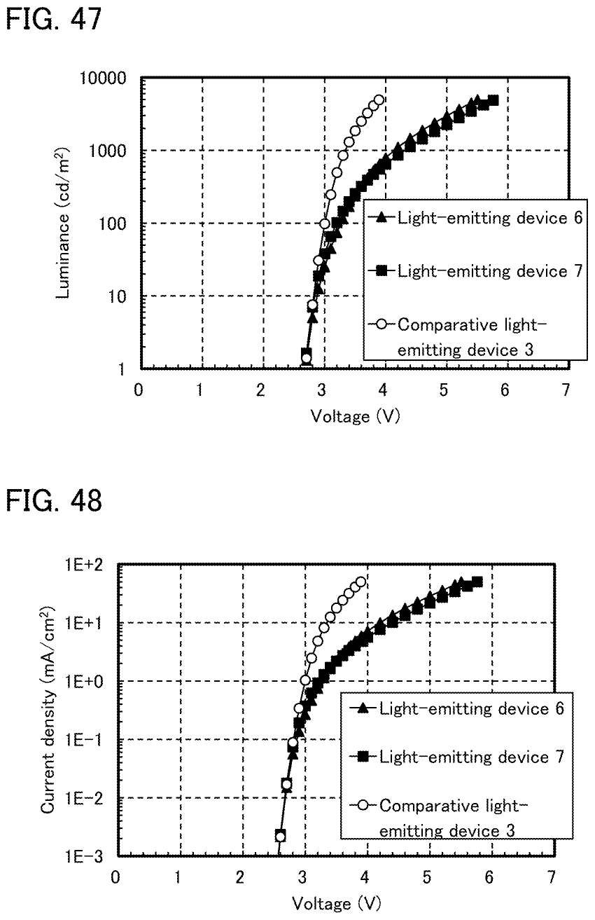

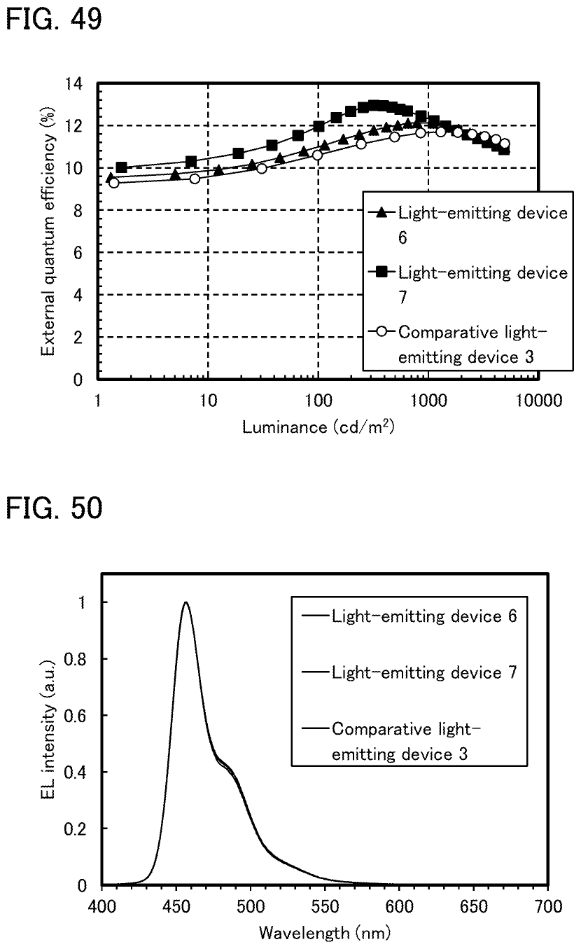

FIG. 45 shows the luminance-current density characteristics of the light-emitting devices 6 and 7 and the comparative light-emitting device 3. FIG. 46 shows the current efficiency-luminance characteristics thereof. FIG. 47 shows the luminance-voltage characteristics thereof. FIG. 48 shows the current density-voltage characteristics thereof. FIG. 49 shows the external quantum efficiency-luminance characteristics thereof. FIG. 50 shows the emission spectra thereof. Table 12 shows the main characteristics of the light-emitting devices 6 and 7 and the comparative light-emitting device 3 at a luminance of about 1000 cd/m². Luminance, CIE chromaticity, and emission spectra were measured at normal temperature with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 12

| | | | | | | current | external |
|---|---|---|---|---|---|---|---|
| | voltage | current | current density | | | efficiency | quantum |
| | (V) | (mA) | (mA/cm²) | chromaticity x | chromaticity y | (cd/A) | efficiency (%) |
| Light-emitting device 6 | 4.2 | 0.40 | 10 | 0.14 | 0.10 | 11.1 | 12 |
| Light-emitting device 7 | 4.4 | 0.41 | 10 | 0.14 | 0.10 | 11.1 | 12 |
| Comparative light-emitting device 3 | 3.3 | 0.33 | 8.2 | 0.14 | 0.10 | 10.4 | 12 |

The results in FIG. 45 to FIG. 50 and Table 12 reveal that these light-emitting devices are each an EL device having high current efficiency and external quantum efficiency.

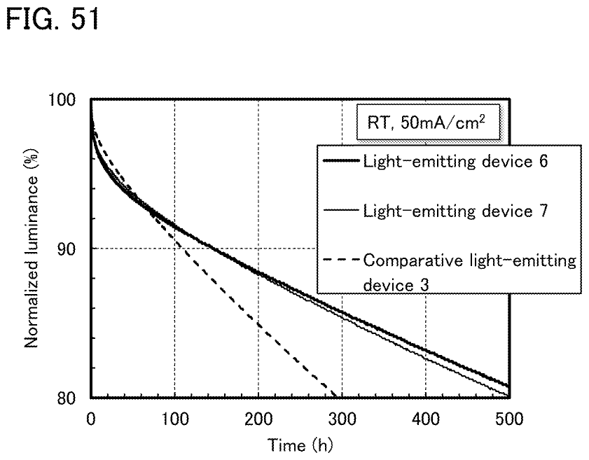

Next, reliability tests were performed on the light-emitting devices. FIG. 51 shows the reliability test results of the light-emitting devices 6 and 7. In FIG. 51 showing the reliability, the vertical axis represents normalized luminance (%) with an initial luminance of 100%, and the horizontal axis represents driving time (h) of the devices. As the reliability tests, driving tests at a constant current density of 50 mA/cm² were performed on the light-emitting devices.

The above results demonstrate that the light-emitting devices 6 and 7 containing the low refractive index material of one embodiment of the present invention each have a normalized luminance of approximately 80% or higher after 500 hours have elapsed, revealing that the light-emitting devices have high reliability.

Note that favorable light emission can be obtained from a light-emitting device fabricated using the organic compound as an electron-transport material.

### Example 11

### Synthesis Example 7

In this example, a method for synthesizing 2-(3″,5′,5″-tri-tert-butyl-1,1′:3′,1″-terphenyl-4-yl)-4,6-diphenyl-1,3,5-triazine (abbreviation: mmtBumTPTzn-04), which is the organic compound of one embodiment of the present invention, is described. The structure of mmtBumTPTzn-04 is shown below.

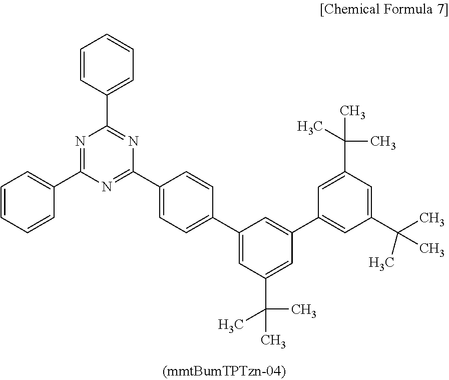 [SMILES: CC(C)(C)c1cc(-c2ccc(-c3nc(-c4ccccc4)nc(-c4ccccc4)n3)cc2)cc(-c2cc(C(C)(C)C)cc(C(C)(C)C)c2)c1]

### Step 1: Synthesis of mmtBumTPTzn-04

Into a three-neck flask were put 2.40 g (6.1 mmol) of 2-(4-bromophenyl)-4,6-diphenyl-1,3,5-triazine, 2.4 g (7.5 mmol) of 3′,5,5′-tri-tert-butyl-1,1′-biphenyl-3-yl-4,4,5,5-tetramethyl-1,3,2-dioxaborolane, 2.2 g (16 mmol) of potassium carbonate, 8 mL of H₂O, 31 mL of toluene, and 8 mL of ethanol, and the mixture was degassed. To this mixture were added 33 mg (0.15 mmol) of palladium(II) acetate and 81 mg (0.27 mmol) of tris(2-methylphenyl)phosphine, and the mixture was heated and refluxed under a nitrogen stream for 7 hours. After the reaction, extraction was performed with toluene and the obtained organic layer was dried with magnesium sulfate. This mixture was gravity-filtered, and the obtained filtrate was concentrated to give a white solid. This solid was purified by silica gel column chromatography with a developing solvent of chloroform and hexane in a ratio of 20:1, which was then changed to 4:1, to give a white solid. This solid was recrystallized with ethanol and hexane. After that, purification was performed by high performance liquid chromatography using chloroform as a mobile phase to give 2.2 g of a white solid in a yield of 57%. The synthesis scheme of Step 1 is shown below.

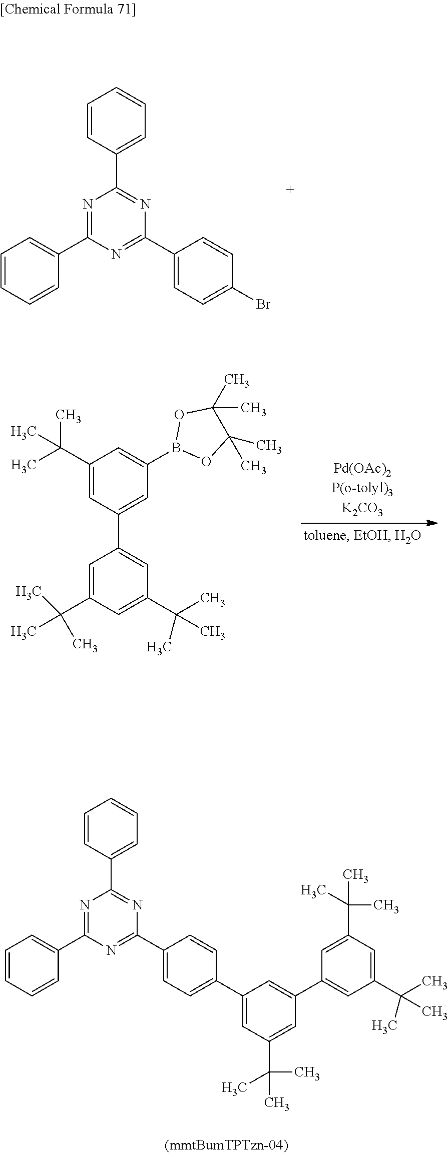 [SMILES: Brc1ccc(-c2nc(-c3ccccc3)nc(-c3ccccc3)n2)cc1 ; CC(C)(C)c1cc(B2OC(C)(C)C(C)(C)O2)cc(-c2cc(C(C)(C)C)cc(C(C)(C)C)c2)c1 ; CC(C)(C)c1cc(-c2ccc(-c3nc(-c4ccccc4)nc(-c4ccccc4)n3)cc2)cc(-c2cc(C(C)(C)C)cc(C(C)(C)C)c2)c1]

Then, 1.995 g of the obtained white solid was purified by a train sublimation method at 235° C. under a pressure of 3.4 Pa while an argon gas was made to flow. After the purification by sublimation, 700 mg of a target white solid was obtained at a collection rate of 35%.

Analysis results by nuclear magnetic resonance (¹H-NMR) spectroscopy of the white solid obtained in Step 1 are shown below. These results reveal that mmtBumTPTzn-04, which is the organic compound of one embodiment of the present invention, was obtained in this synthesis example.

¹H NMR (CDCl₃, 300 MHz): δ=1.41 (s, 18H), 1.49 (s, 9H), 7.47 (m, 2H), 7.49 (d, 1H), 7.56-7.63 (m, 7H), 7.68-7.70 (m, 2H), 7.87 (d, 2H), 8.79-8.83 (dd, 4H), 8.88 (d, 2H).

Next, the absorption spectrum of mmtBumTPTzn-04 was measured with an ultraviolet-visible light spectrophotometer (V550, manufactured by JASCO Corporation). The absorption spectrum of mmtBumTPTzn-04 in the dichloromethane solution was obtained by subtracting the spectrum of dichloromethane alone in a quartz cell from the absorption spectrum of mmtBumTPTzn-04 in the dichloromethane solution in the quartz cell. As a result, the absorption peak was observed at 270 nm, revealing that no absorption was shown in the visible region range from 440 nm to 700 nm.

Next, mmtBumTPTzn-04 obtained in this example was subjected to LC/MS analysis.

In the LC/MS analysis, LC separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumTPTzn-04 in an organic solvent at an arbitrary concentration, and the injection amount was 5.0 μL.

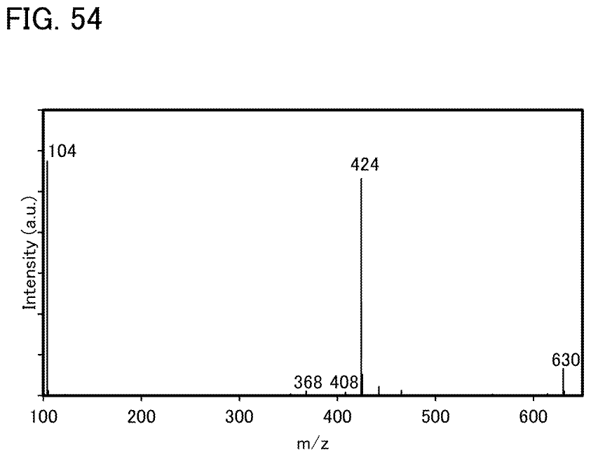

The MS/MS measurement of m/z=629.38 corresponding to the exact mass of mmtBumTPTzn-04 was performed by a PRM method. For setting of the PRM, the mass range of a target ion was set to m/z=629.38±2.0 (isolation window=4) and detection was performed in a positive mode. The measurement was performed with energy (NCE) for accelerating a target ion in a collision cell set to 50. The MS spectrum obtained by the MS/MS measurement is shown in FIG. 54.

Fragment ions at m/z of 104.05 and m/z of 424.30 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 424.30 is probably a fragment in which one carbon atom and one nitrogen atom each derived from triazine are bonded to a substituent other than a phenyl group. These fragments can each be regarded as a feature of a compound having a triazine skeleton.

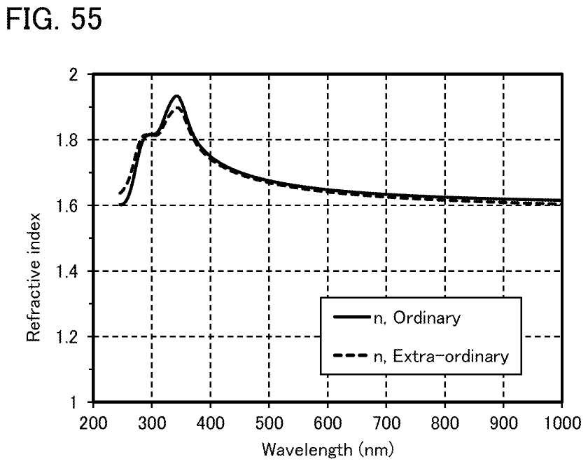

FIG. 55 shows the results of measuring the refractive index of mmtBumTPTzn-04 with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 55.

FIG. 55 shows that mmtBumTPTzn-04 is a material with a low refractive index: the ordinary refractive index of mmtBumTPTzn-04 in the entire blue emission region (from 455 nm to 465 nm) is in the range of 1.50 to 1.75, and the ordinary refractive index of mmtBumTPTzn-04 at a wavelength of 633 nm is in the range of 1.45 to 1.70.

Note that favorable light emission can be obtained from a light-emitting device fabricated using the organic compound as an electron-transport material.

### Example 12

### Synthesis Example 8

In this example, a method for synthesizing 2-[3-(2,6-dimethylpyridin-3-yl)-5-{(3,5-di-tert-butyl)phenyl}phenyl]-4,6-diphenyl-1,3,5-triazine (abbreviation: mmtBuPh-mDMePyPTzn), which is the organic compound of one embodiment of the present invention represented by Structural Formula (503) in Embodiment 1, is described. The structural formula of mmtBuPh-mDMePyPTzn is shown below.

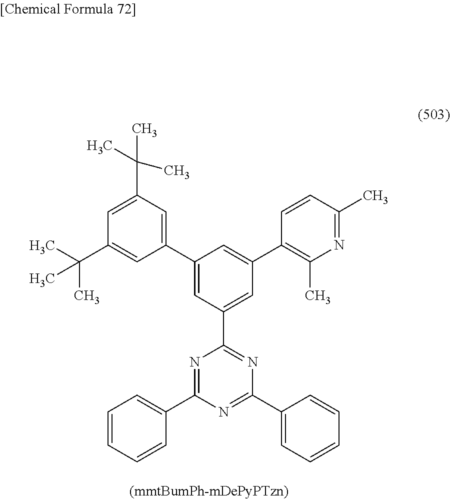 [SMILES: Cc1ccc(-c2cc(-c3cc(C(C)(C)C)cc(C(C)(C)C)c3)cc(-c3nc(-c4ccccc4)nc(-c4ccccc4)n3)c2)c(C)n1]

### Step 1: Synthesis of 2-{3-chloro-5-(3,5-di-tert-butylphenyl)phenyl}-4,6-diphenyl-1,3,5-triazine

Into a three-neck flask were put 8.0 g (19 mmol) of 2-(3-bromo-5-chlorophenyl)-4,6-diphenyl-1,3,5-triazine, 4.9 g (21 mmol) of 3,5-di-tert-butylphenylboronic acid, 19 mL of an aqueous solution of potassium carbonate (2 mol/L), 72 mL of toluene, and 36 mL of ethanol, and the mixture was degassed. To this mixture were added 0.23 g (0.76 mmol) of tris(2-methylphenyl)phosphine and 42 mg (0.19 mmol) of palladium(II) acetate, and the mixture was stirred at 80° C. for 14 hours. After the reaction, the reaction solution was filtered, and a residue and a filtrate (1) were obtained.

The obtained residue was dissolved in toluene by heating and then filtered to obtain a filtrate (2). The obtained filtrate (2) was concentrated and then recrystallized with toluene and ethanol to give 4.7 g of a target white solid.

The filtrate (1) was subjected to extraction with toluene, and the organic layer was dried with magnesium sulfate. This mixture was gravity-filtrated, and the obtained filtrate (3) was concentrated to give an orange solid. Ultrasonic cleaning was performed using ethanol, whereby a white solid was obtained. This solid was recrystallized with toluene and ethanol to give 4.9 g of a target white solid.

The white solid obtained from the residue and the white solid obtained from the filtrate (2) were mixed, so that 9.5 g of a target white solid was obtained in a yield of 95%. The synthesis scheme of Step 1 is shown below.

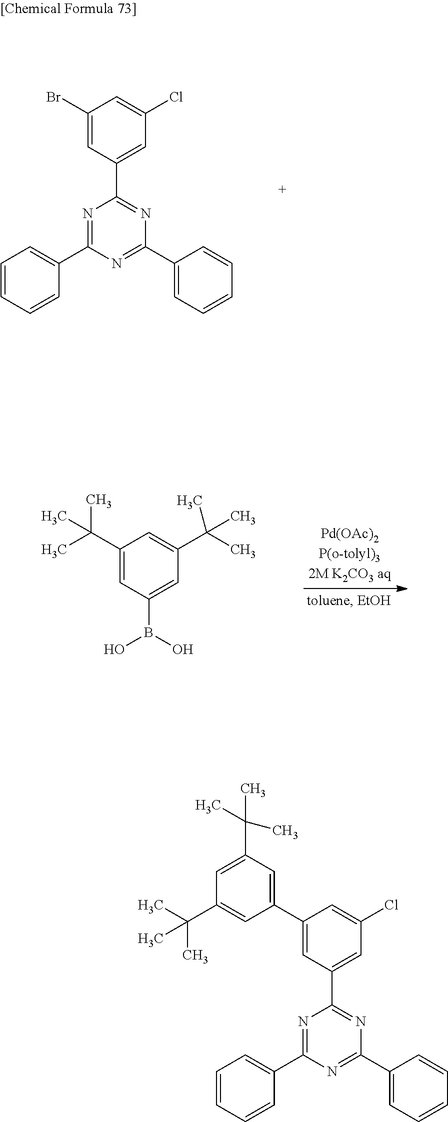 [SMILES: Clc1cc(Br)cc(-c2nc(-c3ccccc3)nc(-c3ccccc3)n2)c1 ; CC(C)(C)c1cc(B(O)O)cc(C(C)(C)C)c1 ; CC(C)(C)c1cc(-c2cc(Cl)cc(-c3nc(-c4ccccc4)nc(-c4ccccc4)n3)c2)cc(C(C)(C)C)c1]

### Step 2: Synthesis of 2-{3-(3,5-di-tert-butylphenyl)-5-(4,4,5,5-tetramethyl-1,3,2-dioxaborolan-2-yl)phenyl}-4,6-diphenyl-1,3,5-triazine

Into a three-neck flask were put 4.5 g (8.5 mmol) of 2-{3-chloro-5-(3,5-di-tert-butylphenyl)phenyl}-4,6-diphenyl-1,3,5-triazine, 3.2 g (13 mmol) of bis(pinacolato)diboron, 2.5 g (25 mmol) of potassium acetate, and 160 mL of 1,4-dioxane, and the mixture was degassed. To this mixture were added 81 mg (0.17 mmol) of 2-dicyclohexylphosphino-2′,4′,6′-triisopropylbiphenyl and 19 mg (0.085 mmol) of palladium(II) acetate, and the mixture was stirred at 100° C. for 11.5 hours. After the reaction, extraction was performed with toluene and the organic layer was dried with magnesium sulfate. This mixture was gravity-filtered, and the obtained filtrate was concentrated to give a pale yellow oil. This oil was purified by silica gel column chromatography with a developing solvent of toluene and hexane in a ratio of 2:1, which was then changed to only toluene, to give 5.1 g of a target white solid in a yield of 96%. The synthesis scheme of Step 2 is shown below.

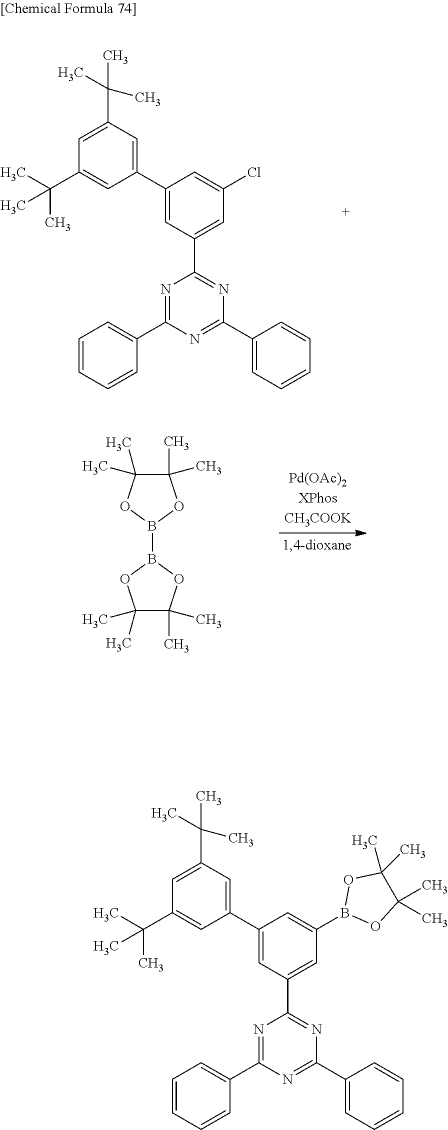

### Step 3: Synthesis of mmtBuPh-mDMePyPTzn

Into a three-neck flask were put 5.0 g (8.0 mmol) of 2-{3-(3,5-di-tert-butylphenyl)-5-(4,4,5,5-tetramethyl-1,3,2-dioxaborolan-2-yl)phenyl}-4,6-diphenyl-1,3,5-triazine, 1.4 g (7.3 mmol) of 3-bromo-2,6-dimethylpyridine, 40 mL of tetrahydrofuran, and 10 mL (4.6 g, 22 mmol) of an aqueous solution of tripotassium phosphate (2 mol/L), and the mixture was degassed. To this mixture were added 16 mg (0.073 mmol) of palladium(II) acetate and 70 mg (0.15 mmol) of 2-dicyclohexylphosphino-2′,4′,6′-triisopropylbiphenyl, and the mixture was stirred at 65° C. for 13.5 hours. After the reaction, extraction was performed with ethyl acetate and the organic layer was dried with magnesium sulfate. This mixture was gravity-filtered, and the obtained filtrate was concentrated to give a yellow solid. This solid was purified by silica gel column chromatography with a developing solvent of ethyl acetate and hexane in a ratio of 1:5 to give a pale yellow solid. The collected solid was recrystallized with toluene and ethanol to give 4.0 g of a target white solid in a yield of 90%. The synthesis scheme of Step 3 is shown below.

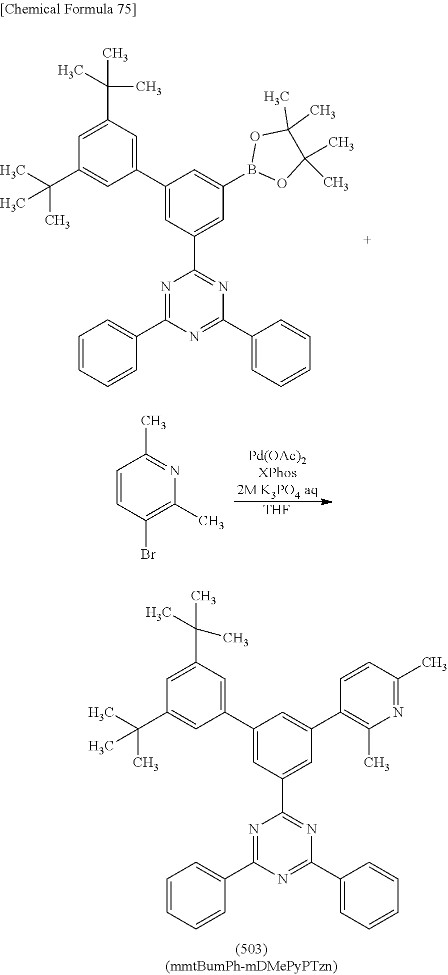

Then, 4.0 g of the obtained white solid was purified by a train sublimation method. In the purification by sublimation, the solid was heated under a pressure of 6.4 Pa at 255° C. for 22 hours while an argon gas was made to flow. After the purification by sublimation, 3.7 g of a target white solid was obtained at a collection rate of 94%.

Analysis results by nuclear magnetic resonance (¹H-NMR) spectroscopy of the white solid obtained above are shown below. These results reveal that the organic compound of one embodiment of the present invention, mmtBuPh-mDMePyPTzn represented by Structural Formula (503), was obtained in this example.

¹H NMR (CDCl₃, 300 MHz): δ=1.44 (s, 18H), 2.63 (s, 3H), 2.64 (s, 3H), 7.15 (d, 1H), 7.53-7.63 (m, 10H), 7.74 (t, 1H), 8.68 (t, 1H), 8.76-8.80 (d, 4H), 9.00 (t, 1H).

Next, the absorption spectrum of mmtBuPh-mDMePyPTzn was measured with an ultraviolet-visible light spectrophotometer (V550, manufactured by JASCO Corporation). The absorption spectrum of mmtBuPh-mDMePyPTzn in the dichloromethane solution was obtained by subtracting the spectrum of dichloromethane alone in a quartz cell from the absorption spectrum of mmtBuPh-mDMePyPTzn in the dichloromethane solution in the quartz cell. As a result, the absorption peak was observed at 270 nm, revealing that no absorption was shown in the visible region range from 440 nm to 700 nm.

Next, mmtBuPh-mDMePyPTzn obtained in this example was subjected to LC/MS analysis.

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBuPh-mDMePyPTzn in an organic solvent at an arbitrary concentration, and the injection amount was 5.0 μL.

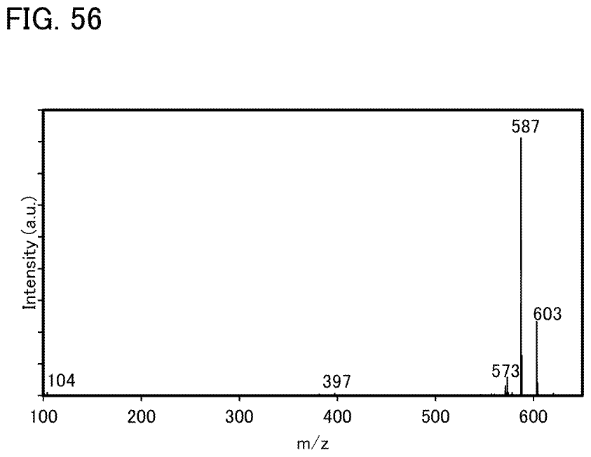

The MS/MS measurement of m/z=602.34 corresponding to the exact mass of mmtBuPh-mDMePyPTzn was performed by a PRM method. For setting of the PRM, the mass range of a target ion was set to m/z=602.34±2.0 (isolation window=4) and detection was performed in a positive mode. The measurement was performed with energy (NCE) for accelerating a target ion in a collision cell set to 60. The MS spectrum obtained by the MS/MS measurement is shown in FIG. 56.

Fragment ions at m/z of 104.05 and m/z of 397.26 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 397.26 is probably a fragment in which one carbon atom and one nitrogen atom each derived from triazine are bonded to a substituent other than a phenyl group. These fragments can each be regarded as a feature of a compound having a triazine skeleton.

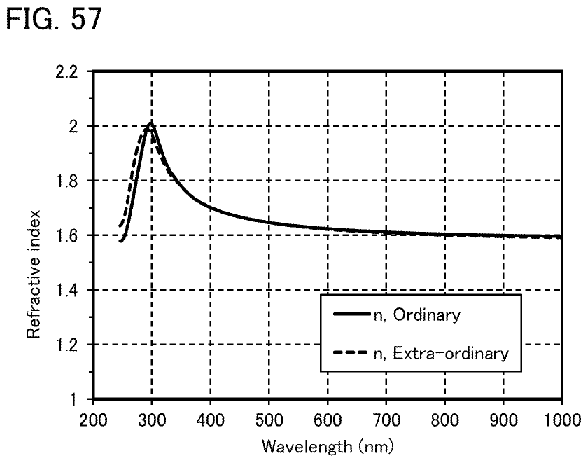

FIG. 57 shows the results of measuring the refractive index of mmtBuPh-mDMePyPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 57.

FIG. 57 shows that mmtBuPh-mDMePyPTzn is a material with a low refractive index: the ordinary refractive index of mmtBuPh-mDMePyPTzn in the entire blue emission region (from 455 nm to 465 nm) is 1.66, which is in the range of 1.50 to 1.75, and the ordinary refractive index of mmtBuPh-mDMePyPTzn at a wavelength of 633 nm is 1.62, which is in the range of 1.45 to 1.70.

Note that favorable light emission can be obtained from a light-emitting device fabricated using the organic compound as an electron-transport material.

### Example 13

### Synthesis Example 9

In this example, a method for synthesizing 2-(3″,5′,5″-tri-tert-butyl-1,1′:3′,1″-terphenyl-5-yl)-4,6-diphenyl-1,3,5-triazine (abbreviation: mmtBumTPTzn-03), which is the organic compound of one embodiment of the present invention represented by Structural Formula (122) in Embodiment 1, is described. The structural formula of mmtBumTPTzn-03 is shown below.

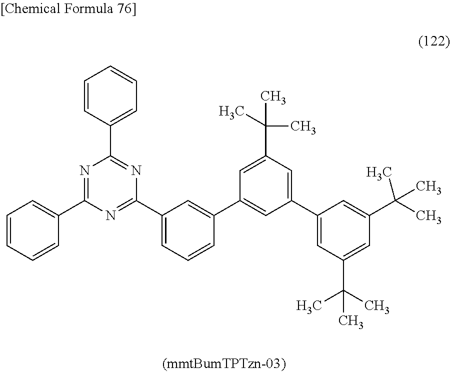 [SMILES: CC(C)(C)c1cc(-c2cccc(-c3nc(-c4ccccc4)nc(-c4ccccc4)n3)c2)cc(-c2cc(C(C)(C)C)cc(C(C)(C)C)c2)c1]

### Step 1: Synthesis of mmtBumTPTzn-03

Into a three-neck flask were put 1.9 g (4.8 mmol) of 2-(3-bromophenyl)-4,6-diphenyl-1,3,5-triazine, 2.4 g (5.4 mmol) of 2-(3′,5,5′,tri-tert-butyl[1,1′-biphenyl]-3-yl)-4,4,5,5-tetramethyl-1,3,2-dioxaborolane, 32 mL of toluene, 8 mL of ethanol (EtOH), and 8 mL of an aqueous solution of potassium carbonate (2 mol/L), and the mixture was degassed. To this mixture were added 14 mg (0.062 mmol) of palladium(II) acetate and 76 mg (0.25 mmol) of tris(2-methylphenyl)phosphine, and the mixture was heated under a nitrogen atmosphere at 80° C. for 16 hours. After the reaction, extraction was performed with ethyl acetate and the obtained organic layer was dried with magnesium sulfate. This mixture was gravity-filtered, and the obtained filtrate was concentrated. The obtained solid was purified by silica gel column chromatography with a developing solvent of chloroform and hexane in a ratio of 1:5. After that, purification was performed by high performance liquid chromatography using chloroform as a mobile phase. The obtained solid was recrystallized with ethanol and hexane to give 2.3 g of a target white solid in a yield of 63%. The synthesis scheme of Step 1 is shown below.

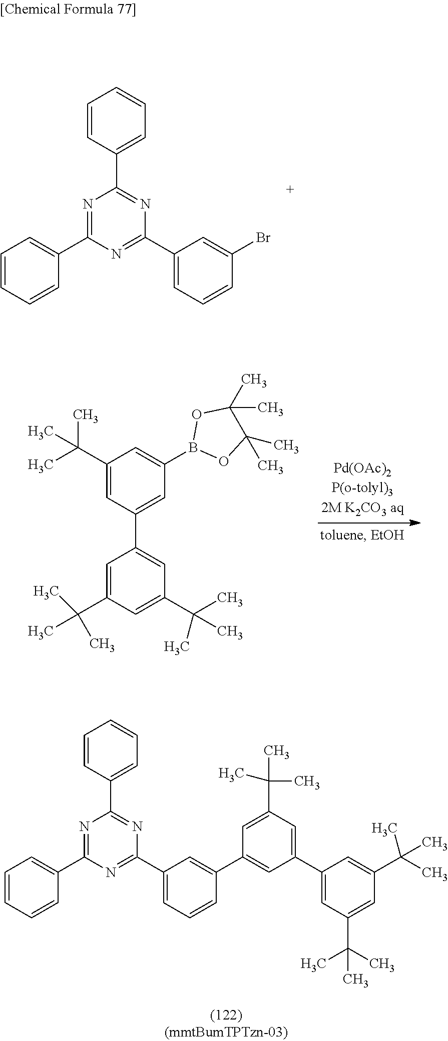 [SMILES: Brc1cccc(-c2nc(-c3ccccc3)nc(-c3ccccc3)n2)c1 ; CC(C)(C)c1cc(B2OC(C)(C)C(C)(C)O2)cc(-c2cc(C(C)(C)C)cc(C(C)(C)C)c2)c1 ; CC(C)(C)c1cc(-c2cccc(-c3nc(-c4ccccc4)nc(-c4ccccc4)n3)c2)cc(-c2cc(C(C)(C)C)cc(C(C)(C)C)c2)c1]

Then, 2.3 g of the obtained white solid was purified by a train sublimation method. In the purification by sublimation, the solid was heated under a pressure of 6.3 Pa at 260° C. for 19 hours while an argon gas was made to flow. After the purification by sublimation, 2.1 g of a target white solid was obtained at a collection rate of 93%.

Analysis results by nuclear magnetic resonance (¹H-NMR) spectroscopy of the white solid obtained above are shown below. These results reveal that the organic compound of one embodiment of the present invention, mmtBumTPTzn-03 represented by Structural Formula (122), was obtained in this example.

¹H NMR (CDCl₃, 300 MHz): δ=1.42 (s, 18H), 1.48 (s, 9H), 7.48-7.50 (m, 3H), 7.55-7.73 (m, 10H), 7.89 (d, 1H), 8.77-8.82 (m, 5H), 9.05 (s, 1H).

The absorption spectrum of mmtBumTPTzn-03 was measured with an ultraviolet-visible light spectrophotometer (V550, manufactured by JASCO Corporation). The absorption spectrum of mmtBumTPTzn-03 in the dichloromethane solution was obtained by subtracting the spectrum of dichloromethane alone in a quartz cell from the absorption spectrum of mmtBumTPTzn-03 in the dichloromethane solution in the quartz cell. As a result, the absorption peak was observed at 267 nm, revealing that no absorption was shown in the visible region range from 440 nm to 700 nm.

Next, mmtBumTPTzn-03 obtained in this example was subjected to LC/MS analysis.

In the LC/MS analysis, liquid chromatography (LC) separation was performed with UltiMate 3000 produced by Thermo Fisher Scientific K.K., and mass spectrometry (MS) was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumTPTzn-03 in an organic solvent at an arbitrary concentration, and the injection amount was 5.0 μL.

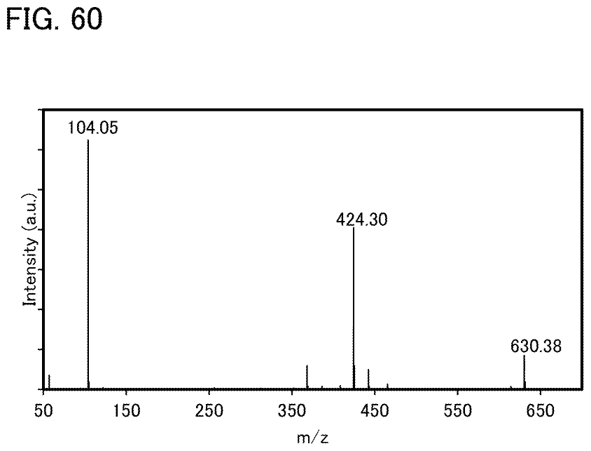

The MS/MS measurement of m/z=629.38 corresponding to the exact mass of mmtBumTPTzn-03 was performed by a PRM method. For setting of the PRM, the mass range of a target ion was set to m/z=629.38±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 60.

Fragment ions at m/z of 104.05 and m/z of 424.30 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 424.30 is probably a fragment in which one carbon atom and one nitrogen atom each derived from triazine are bonded to a substituent other than a phenyl group. These fragments can each be regarded as a feature of a compound having a triazine skeleton.

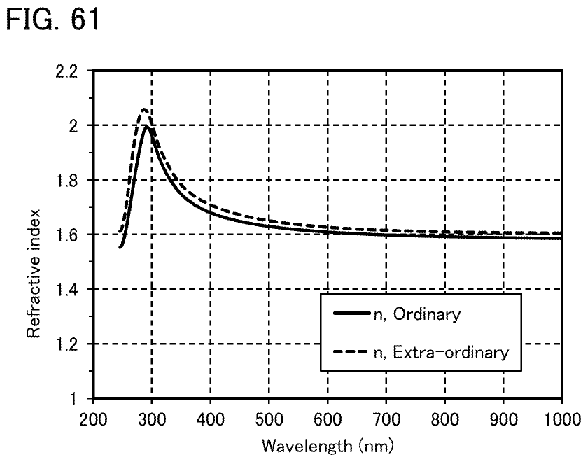

FIG. 61 shows the results of measuring the refractive index of mmtBumTPTzn-03 with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 61.

FIG. 61 shows that mmtBumTPTzn-03 is a material with a low refractive index: the ordinary refractive index of mmtBumTPTzn-03 in the entire blue emission region (from 455 nm to 465 nm) is 1.64, which is in the range of 1.50 to 1.75, and the ordinary refractive index of mmtBumTPTzn-03 at a wavelength of 633 nm is 1.60, which is in the range of 1.45 to 1.70.

Note that favorable light emission can be obtained from a light-emitting device fabricated using the organic compound as an electron-transport material.

### Example 14

### Synthesis Example 10

In this example, a method for synthesizing 2,4-bis[(3′,5′-di-tert-butyl)-1,1′-biphenyl-3-yl]-6-phenyl-1,3,5-triazine (abbreviation: mmtBumBP2Tzn), which is the organic compound represented by Structural Formula (103) in Embodiment 1, is described. The structural formula of mmtBumBP2Tzn is shown below.

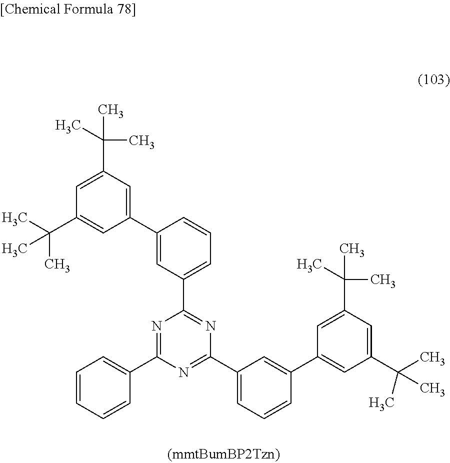 [SMILES: CC(C)(C)c1cc(-c2cccc(-c3nc(-c4ccccc4)nc(-c4cccc(-c5cc(C(C)(C)C)cc(C(C)(C)C)c5)c4)n3)c2)cc(C(C)(C)C)c1]

### Step 1: Synthesis of 2-(3 5′-di-tert-butylbiphenyl-3-yl)-4,4,5,5-tetramethyl-1,3,2-dioxaborolane

This synthesis step is similar to Step 2 in Synthesis example 1.

### Step 2: Synthesis of mmtBumBP2Tzn

Into a three-neck flask were put 1.3 g (5.8 mmol) of 2,4-dichloro-6-phenyl-1,3,5-triazine, 5.0 g (13 mmol) of 2-(3′,5′-di-tert-butylbiphenyl-3-yl)-4,4,5,5-tetramethyl-1,3,2-dioxaborolane, 2.4 g (17 mmol) of potassium carbonate, 8.6 mL of water, 38 mL of toluene, and 12 mL of ethanol, and the mixture was degassed. To this mixture were added 0.026 g (0.12 mmol) of palladium(II) acetate and 0.070 g (0.23 mmol) of tris(2-methylphenyl)phosphine, and reaction was caused at 85° C. for 2.5 hours. Then, reaction was caused at 95° C. for 24 hours. After the reaction, extraction was performed with toluene and the organic layer was dried with magnesium sulfate. This mixture was gravity-filtered, and the obtained filtrate was concentrated to give 4.4 g of a black solid. This solid was purified by silica gel column chromatography with a developing solvent of hexane and toluene in a ratio of 3:1, which was then changed to 1;1, to give 3.2 g of a white solid. After that, purification was performed by high performance liquid chromatography using chloroform as a mobile phase. The obtained solid was recrystallized with hexane and ethanol to give 1.8 g of a white solid in a yield of 47%. The synthesis scheme of Step 2 is shown below.

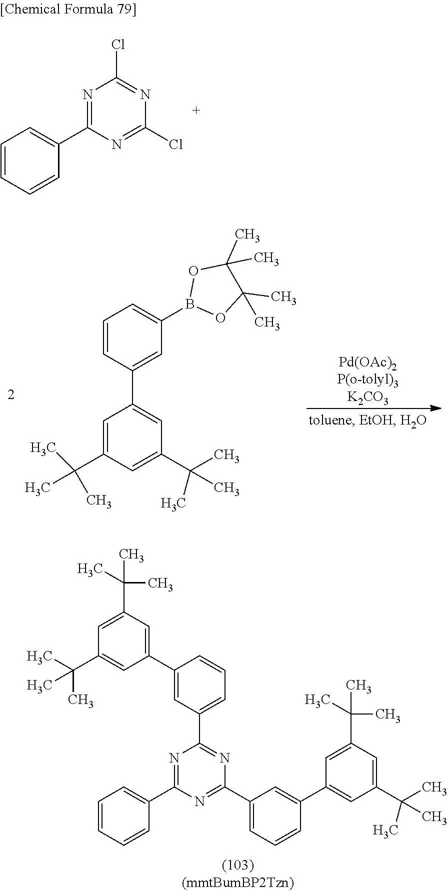 [SMILES: Clc1nc(Cl)nc(-c2ccccc2)n1 ; CC(C)(C)c1cc(-c2cccc(B3OC(C)(C)C(C)(C)O3)c2)cc(C(C)(C)C)c1 ; CC(C)(C)c1cc(-c2cccc(-c3nc(-c4ccccc4)nc(-c4cccc(-c5cc(C(C)(C)C)cc(C(C)(C)C)c5)c4)n3)c2)cc(C(C)(C)C)c1]

Then, 1.8 g of the obtained white solid was purified by a train sublimation method. In the purification by sublimation, the solid was heated under a pressure of 5.8 Pa at 260° C. while an argon gas was made to flow. After the purification by sublimation, 1.6 g of a target white solid was obtained at a collection rate of 88%.

Analysis results by nuclear magnetic resonance (¹H-NMR) spectroscopy of the white solid obtained above are shown below. These results reveal that the organic compound of one embodiment of the present invention, mmtBumBP2Tzn represented by Structural Formula (103), was obtained in this example.

¹H NMR (CDCl₃, 300 MHz): δ=1.43 (s, 36H), 7.51-7.67 (m, 11H), 7.82 (d, 2H), 8.73-8.81 (m, 4H), 9.00 (s, 2H).

The absorption spectrum of mmtBumBP2Tzn was measured with an ultraviolet-visible light spectrophotometer (V550, manufactured by JASCO Corporation). The absorption spectrum of mmtBumBP2Tzn in the dichloromethane solution was obtained by subtracting the spectrum of dichloromethane alone in a quartz cell from the absorption spectrum of mmtBumBP2Tzn in the dichloromethane solution in the quartz cell. As a result, the absorption peak was observed at 274 nm, revealing that no absorption was shown in the visible region range from 440 nm to 700 nm.

Next, mmtBumBP2Tzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBumBP2Tzn in an organic solvent at an arbitrary concentration, and the injection amount was 5.0 μL.

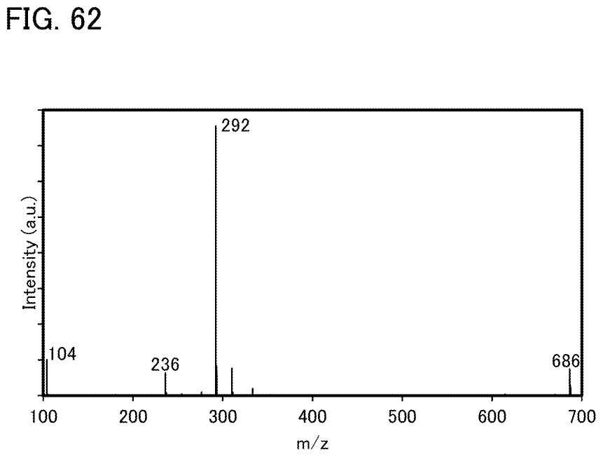

The MS/MS measurement of m/z=685.44 corresponding to the exact mass of mmtBumBP2Tzn was performed by a PRM method. For setting of the PRM, the mass range of a target ion was set to m/z=685.44±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 62.

Fragment ions at m/z of 104.05 and m/z of 292.21 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. In addition, the fragment ion at m/z of 292.21 is probably a fragment in which one carbon atom and one nitrogen atom each derived from triazine are bonded to a biphenyl group to which two tertiary butyl groups are bonded. These fragments can each be regarded as a feature of a compound having a triazine skeleton.

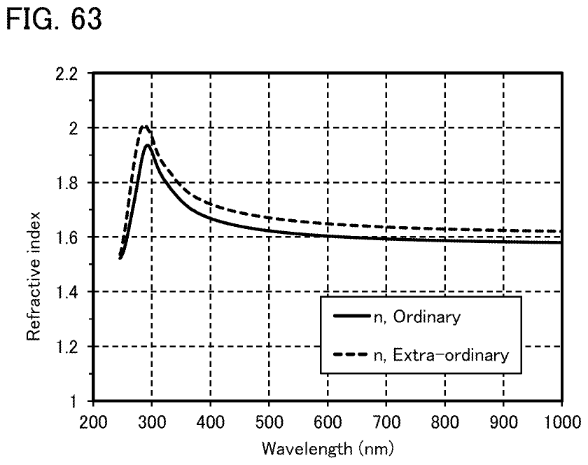

FIG. 63 shows the results of measuring the refractive index of mmtBumBP2Tzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 63.

FIG. 63 shows that mmtBumBP2Tzn is a material with a low refractive index: the ordinary refractive index of mmtBumBP2Tzn in the entire blue emission region (from 455 nm to 465 nm) is 1.64, which is in the range of 1.50 to 1.75, and the ordinary refractive index of mmtBumBP2Tzn at a wavelength of 633 nm is 1.60, which is in the range of 1.45 to 1.70.

Note that favorable light emission can be obtained from a light-emitting device fabricated using the organic compound as an electron-transport material.

### Example 15

### Synthesis Example 11

In this example, a method for synthesizing 2-{(1,1′-biphenyl)-2-yl}-4-{(3′,5′-di-tert-butyl)-1,1′-biphenyl-3-yl}-6-phenyl-1,3,5-triazine (abbreviation: oBP-mmtBumBPTzn), which is the organic compound of one embodiment of the present invention, is described. The structural formula of oBP-mmtBumBPTzn is shown below.

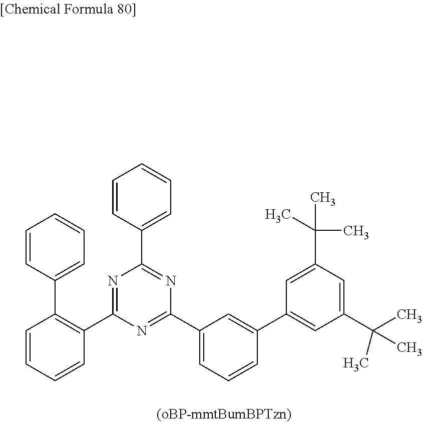 [SMILES: CC(C)(C)c1cc(-c2cccc(-c3nc(-c4ccccc4)nc(-c4ccccc4-c4ccccc4)n3)c2)cc(C(C)(C)C)c1]

### Step 1: Synthesis of 2-(3 5′-di-tert-butylbiphenyl-3-yl)-4,4,5,5-tetramethyl-1,3,2-dioxaborolane

This synthesis step is similar to Step 2 in Synthesis example 1.

### Step 2: Synthesis of oBP-mmtBumBPTzn

Into a three-neck flask were put 2.7 g (7.7 mmol) of 2-{(1,1′-biphenyl)-2-yl}-4-chloro-6-phenyl-1,3,5-triazine, 3.3 g (8.5 mmol) of 2-(3′,5′-di-tert-butylbiphenyl-3-yl)-4,4,5,5-tetramethyl-1,3,2-dioxaborolane, 3.3 g (15 mmol) of tripotassium phosphate, 15 mL of water, 40 mL of toluene, and 15 mL of 1,4-dioxane, and the mixture was degassed. To this mixture were added 17 mg (0.077 mmol) of palladium(II) acetate and 46 mg (0.15 mmol) of tris(2-methylphenyl)phosphine, and the mixture was heated and refluxed for 11 hours. After the reaction, extraction was performed with toluene and the organic layer was dried with magnesium sulfate. This mixture was gravity-filtered, and the obtained filtrate was concentrated to give a yellow oil. This oil was purified by silica gel column chromatography with a developing solvent of toluene and hexane in a ratio of 1:2 to give a white solid. After that, purification was performed by high performance liquid chromatography using chloroform as a mobile phase. The obtained solid was recrystallized with toluene and ethanol to give 3.0 g of a target white solid in a yield of 68%. The synthesis scheme of Step 2 is shown below.

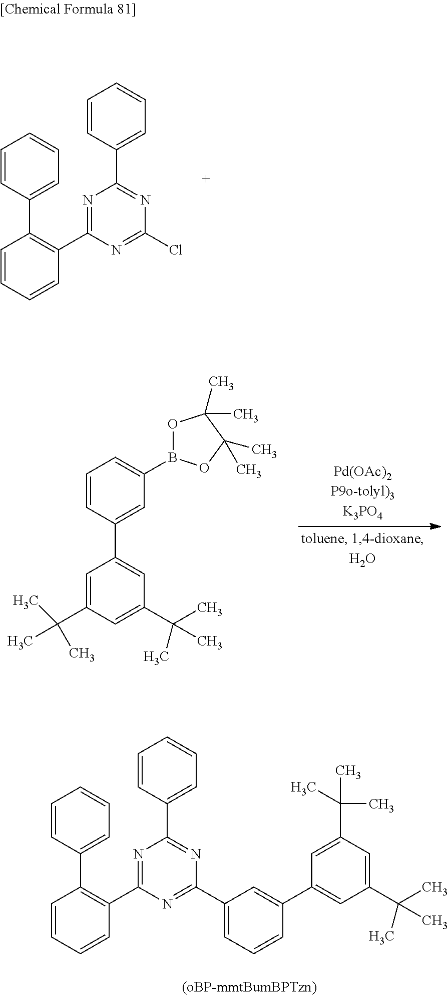 [SMILES: Clc1nc(-c2ccccc2)nc(-c2ccccc2-c2ccccc2)n1 ; CC(C)(C)c1cc(-c2cccc(B3OC(C)(C)C(C)(C)O3)c2)cc(C(C)(C)C)c1 ; CC(C)(C)c1cc(-c2cccc(-c3nc(-c4ccccc4)nc(-c4ccccc4-c4ccccc4)n3)c2)cc(C(C)(C)C)c1]

Then, 3.0 g of the obtained white solid was purified by a train sublimation method. In the purification by sublimation, the solid was heated under a pressure of 6.3 Pa at 220° C. for 17 hours while an argon gas was made to flow. After the purification by sublimation, 3.1 g of a target white solid was obtained at a collection rate of 77%.

Analysis results by nuclear magnetic resonance (¹H-NMR) spectroscopy of the white solid obtained above are shown below. These results reveal that the organic compound of one embodiment of the present invention, oBP-mmtBumBPTzn represented by the above structural formula, was obtained in this example.

¹H NMR (CDCl₃, 300 MHz): δ=1.43 (s, 18H), 7.14-7.20 (m, 1H), 7.28-7.35 (m, 3H), 7.40-7.64 (m, 11H), 7.74 (d, 1H), 8.26-8.37 (m, 4H), 8.67 (s, 1H).

The absorption spectrum of oBP-mmtBumBPTzn was measured with an ultraviolet-visible light spectrophotometer (V550, manufactured by JASCO Corporation). The absorption spectrum of oBP-mmtBumBPTzn in the dichloromethane solution was obtained by subtracting the spectrum of dichloromethane alone in a quartz cell from the absorption spectrum of oBP-mmtBumBPTzn in the dichloromethane solution in the quartz cell. As a result, the absorption peak was observed at 272 nm, revealing that no absorption was shown in the visible region range from 440 nm to 700 nm.

Next, oBP-mmtBumBPTzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving oBP-mmtBumBPTzn in an organic solvent at an arbitrary concentration, and the injection amount was 5.0 μL.

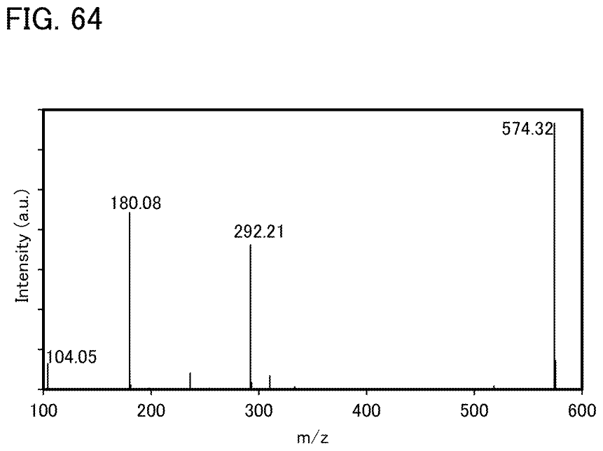

The MS/MS measurement of m/z=573.31 corresponding to the exact mass of oBP-mmtBumBPTzn was performed by a PRM method. For setting of the PRM, the mass range of a target ion was set to m/z=573.31±2.0 (isolation window=4) and detection was performed in a positive mode. The measurement was performed with energy (normalized collision energy: NCE) for accelerating a target ion in a collision cell set to 40. The MS spectrum obtained by the MS/MS measurement is shown in FIG. 64.

Fragment ions at m/z of 104.05, m/z of 180.08, and m/z of 292.21 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. In addition, the fragment ion at m/z of 180.08 is probably a fragment in which one carbon atom and one nitrogen atom each derived from triazine are bonded to a biphenyl group. Furthermore, the fragment ion at m/z of 292.21 is probably a fragment in which one carbon atom and one nitrogen atom each derived from triazine are bonded to a substituent other than a phenyl group and a biphenyl group. These fragments can each be regarded as a feature of a compound having a triazine skeleton.

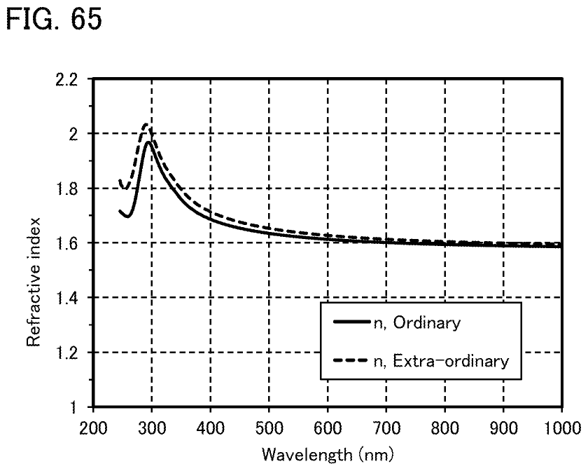

FIG. 65 shows the results of measuring the refractive index of oBP-mmtBumBPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 65.

FIG. 65 shows that oBP-mmtBumBPTzn is a material with a low refractive index: the ordinary refractive index of oBP-mmtBumBPTzn in the entire blue emission region (from 455 nm to 465 nm) is 1.65, which is in the range of 1.50 to 1.75, and the ordinary refractive index of oBP-mmtBumBPTzn at a wavelength of 633 nm is 1.62, which is in the range of 1.45 to 1.70.

Note that favorable light emission can be obtained from a light-emitting device fabricated using the organic compound as an electron-transport material.

### Example 16

### Synthesis Example 12

In this example, a method for synthesizing 2-[(1,1′-biphenyl)-2-yl]-4-{(3′,5′-di-tert-butyl)-1,1′-biphenyl-4-yl}-6-phenyl-1,3,5-triazine (abbreviation: oBP-mmtBuBPTzn), which is the organic compound of one embodiment of the present invention, is described. The structural formula of oBP-mmtBuBPTzn is shown below.

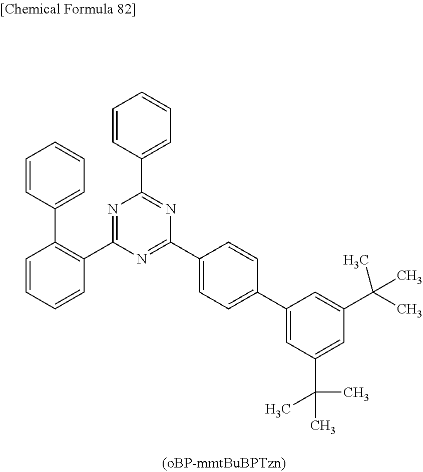 [SMILES: CC(C)(C)c1cc(-c2ccc(-c3nc(-c4ccccc4)nc(-c4ccccc4-c4ccccc4)n3)cc2)cc(C(C)(C)C)c1]

### Step 1: Synthesis of 4-bromo-3′,5′-di-tert-butylbiphenyl

Into a three-neck flask were put 18 g (77 mmol) of 3,5-di-t-butylphenylboronic acid, 26 g (92 mmol) of 1-bromo-4-iodobenzene, 77 mL of an aqueous solution of potassium carbonate (2 mol/L), 360 mL of toluene, and 54 mL of ethanol, and the mixture was degassed. To this mixture were added 0.94 g (3.1 mmol) of tris(2-methylphenyl)phosphine and 0.17 g (0.77 mmol) of palladium(II) acetate, and reaction was caused at 80° C. for 10 hours. After the reaction, extraction was performed with toluene and the organic layer was dried with magnesium sulfate. This mixture was gravity-filtered, and the obtained filtrate was concentrated to give a brownish black solid. This solid was purified by silica gel column chromatography with a developing solvent of hexane to give 19 g of a target white solid in a yield of 70%. The synthesis scheme of Step 1 is shown below.

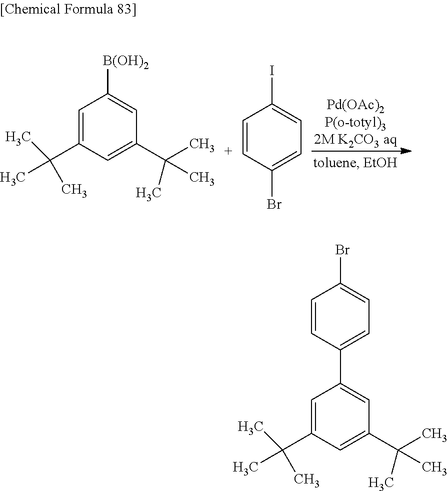 [SMILES: CC(C)(C)c1cc(B(O)O)cc(C(C)(C)C)c1 ; Brc1ccc(I)cc1 ; CC(C)(C)c1cc(-c2ccc(Br)cc2)cc(C(C)(C)C)c1]

### Step 2: Synthesis of 2-(3 5′-di-tert-butylbiphenyl-4-yl)-4,4,5,5-tetramethyl-1,3,2-dioxaborolane

Into a three-neck flask were put 18 g (54 mmol) of 4-bromo-3′,5′-di-tert-butylbiphenyl, 15 g (59 mmol) of bis(pinacolato)diboron, 16 g (160 mmol) of potassium acetate, and 180 mL of N,N-dimethylformamide, and the mixture was degassed. To this mixture was added 2.2 g (2.7 mmol) of {1,1′-bis(diphenylphosphino)ferrocene}palladium(II) dichloride dichloromethane adduct, and reaction was caused at 100° C. for 14 hours. After the reaction, extraction was performed with toluene and the organic layer was dried with magnesium sulfate. This mixture was gravity-filtered, and the obtained filtrate was concentrated to give a black solid. This solid was purified by silica gel column chromatography with a developing solvent of toluene and hexane in a ratio of 2:1, which was then changed to only toluene, to give 17 g of a target white solid in a yield of 80%. The synthesis scheme of Step 2 is shown below.

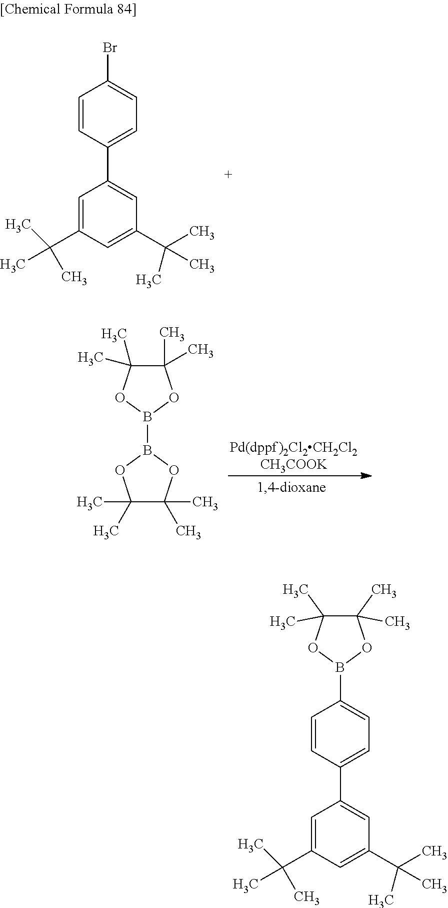

### Step 3: Synthesis method of oBP-mmtBuBPTzn

Into a three-neck flask were put 3.5 g (10 mmol) of 2-{(1,1′-biphenyl)-2-yl}-4-chloro-6-phenyl-1,3,5-triazine, 4.4 g (11 mmol) of 2-(3′,5′-di-tert-butylbiphenyl-4-yl)-4,4,5,5-tetramethyl-1,3,2-dioxaborolane, 4.3 g (20 mmol) of tripotassium phosphate, 55 mL of toluene, 20 mL of 1,4-dioxane, and 20 mL of water, and the mixture was degassed. To this mixture were added 23 mg (0.10 mmol) of palladium(II) acetate and 62 mg (0.20 mmol) of tris(2-methylphenyl)phosphine, and the mixture was heated for 13.5 hours. After the reaction, extraction was performed with toluene and the organic layer was dried with magnesium sulfate. This mixture was gravity-filtered, and the obtained filtrate was concentrated to give a yellow oil. This oil was purified by silica gel column chromatography with a developing solvent of toluene and hexane in a ratio of 1:2. The obtained solid was recrystallized with toluene and ethanol to give 4.2 g of a target white solid in a yield of 72%. The synthesis scheme of Step 3 is shown below.

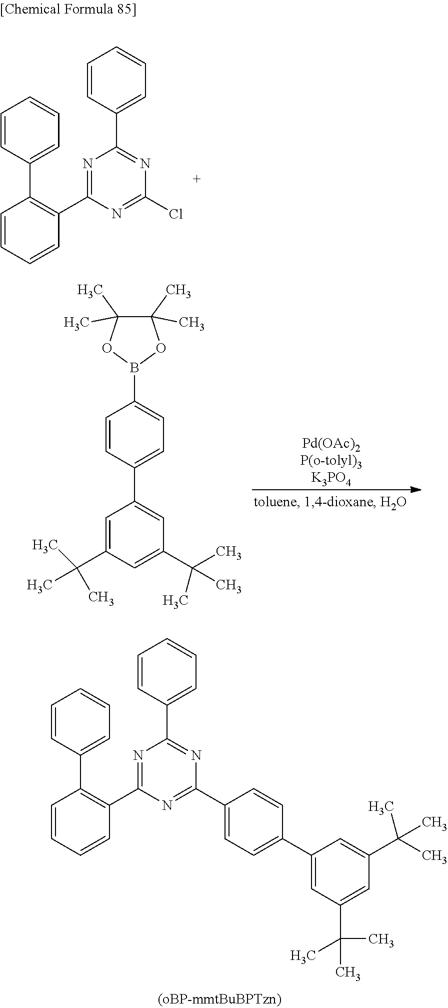

Then, 4.0 g of the obtained white solid was purified by a train sublimation method. In the purification by sublimation, the solid was heated at 230° C. for 19 hours and then at 255° C. for 24 hours under a pressure of 5.5 Pa while an argon gas was made to flow. After the purification by sublimation, 2.6 g of a target white solid was obtained at a collection rate of 86%.

Analysis results by nuclear magnetic resonance (¹H-NMR) spectroscopy of the white solid obtained above are shown below. These results reveal that the organic compound of one embodiment of the present invention, oBP-mmtBuBPTzn, was obtained in this example.

¹H NMR (CDCl₃, 300 MHz): δ=1.41 (s, 18H), 7.28-7.30 (m, 1H), 7.35-7.36 (m, 4H), 7.43-7.69 (m, 11H), 8.32-8.42 (m, 5H).

The absorption spectrum of oBP-mmtBuBPTzn was measured with an ultraviolet-visible light spectrophotometer (V550, manufactured by JASCO Corporation). The absorption spectrum of oBP-mmtBuBPTzn in the dichloromethane solution was obtained by subtracting the spectrum of dichloromethane alone in a quartz cell from the absorption spectrum of oBP-mmtBuBPTzn in the dichloromethane solution in the quartz cell. As a result, the absorption peaks were observed at 278 nm and 311 nm, revealing that no absorption was shown in the visible region range from 440 nm to 700 nm.

Next, oBP-mmtBuBPTzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving oBP-mmtBuBPTzn in an organic solvent at an arbitrary concentration, and the injection amount was 5.0 μL.

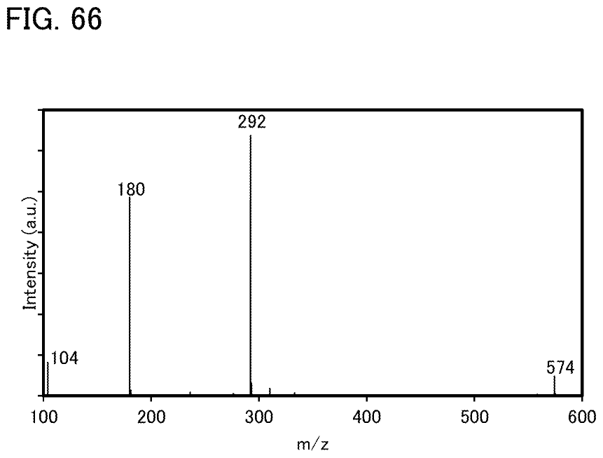

The MS/MS measurement of m/z=573.31 corresponding to an ion derived from oBP-mmtBuBPTzn was performed by a PRM method. For setting of the PRM, the mass range of a target ion was set to m/z=573.31±2.0 (isolation window=4) and detection was performed in a positive mode. The measurement was performed with energy (normalized collision energy: NCE) for accelerating a target ion in a collision cell set to 50. The MS spectrum obtained by the MS/MS measurement is shown in FIG. 66.

Fragment ions at m/z of 104.05, m/z of 180.08, and m/z of 292.21 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. In addition, the fragment ion at m/z of 180.08 is probably a fragment in which one carbon atom and one nitrogen atom each derived from triazine are bonded to a biphenyl group. Furthermore, the fragment ion at m/z of 292.21 is probably a fragment in which one carbon atom and one nitrogen atom each derived from triazine are bonded to a substituent other than a phenyl group and a biphenyl group. These fragments can each be regarded as a feature of a compound having a triazine skeleton.

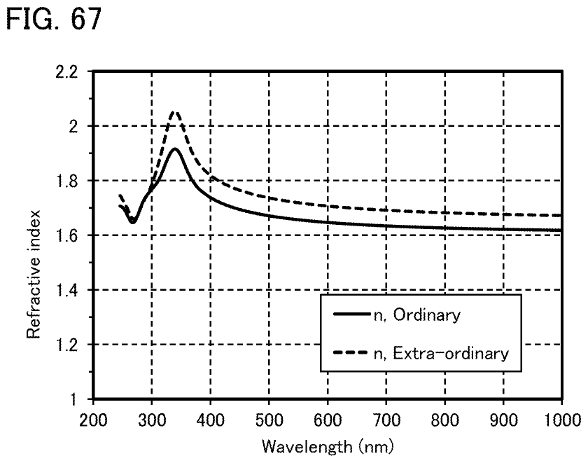

FIG. 67 shows the results of measuring the refractive index of oBP-mmtBuBPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 67.

FIG. 67 shows that oBP-mmtBuBPTzn is a material with a low refractive index: the ordinary refractive index of oBP-mmtBuBPTzn in the entire blue emission region (from 455 nm to 465 nm) is 1.69, which is in the range of 1.50 to 1.75, and the ordinary refractive index of oBP-mmtBuBPTzn at a wavelength of 633 nm is 1.64, which is in the range of 1.45 to 1.70.

Note that favorable light emission can be obtained from a light-emitting device fabricated using the organic compound as an electron-transport material.

### Example 17

### Synthesis Example 13

In this example, a method for synthesizing 2-[3-{(3,5-di-tert-butyl)phenyl}-5-(3-pyridyl)phenyl]-4,6-diphenyl-1,3,5-triazine (abbreviation: mmtBuPh-mPyPTzn), which is the organic compound represented by Structural Formula (501) in Embodiment 1, is described. The structural formula of mmtBuPh-mPyPTzn is shown below.

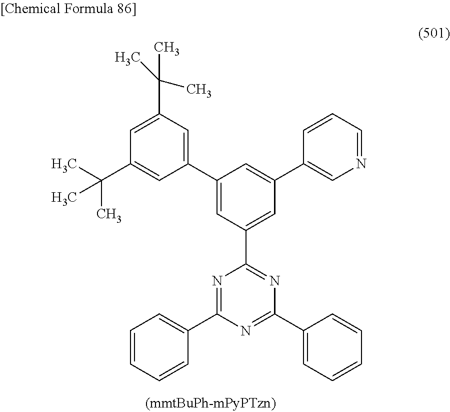 [SMILES: CC(C)(C)c1cc(-c2cc(-c3cccnc3)cc(-c3nc(-c4ccccc4)nc(-c4ccccc4)n3)c2)cc(C(C)(C)C)c1]

### Step 1: Synthesis of mmtBuPh-mPyPTzn

Into a three-neck flask were put 3.5 g (6.6 mmol) of 2-{3-chlorophenyl-5-(3,5-di-tert-butylphenyl)}-4,6-diphenyl-1,3,5-triazine, 1.1 g (8.6 mmol) of 3-pyridylboronic acid, 10 mL of an aqueous solution of potassium carbonate (2 mol/L), and 35 mL of tetrahydrofuran, and the mixture was degassed and then stirred at 50° C. for 30 minutes. To this reaction solution were added 30 mg (0.13 mmol) of palladium(II) acetate and 125 mg (0.26 mmol) of 2-dicyclohexylphosphino-2′,4′,6′-triisopropylbiphenyl, and reaction was caused at 65° C. for 20 hours. After the reaction, extraction was performed with ethyl acetate and the organic layer was dried with magnesium sulfate. This mixture was gravity-filtered, and the obtained filtrate was concentrated to give a gray solid. This solid was purified by silica gel column chromatography with a developing solvent of ethyl acetate and hexane in a ratio of 1:2. The obtained solid was recrystallized with a mixed solvent of toluene and ethanol to give 3.2 g of a target white solid in a yield of 85%. The synthesis scheme of Step 1 is shown below.

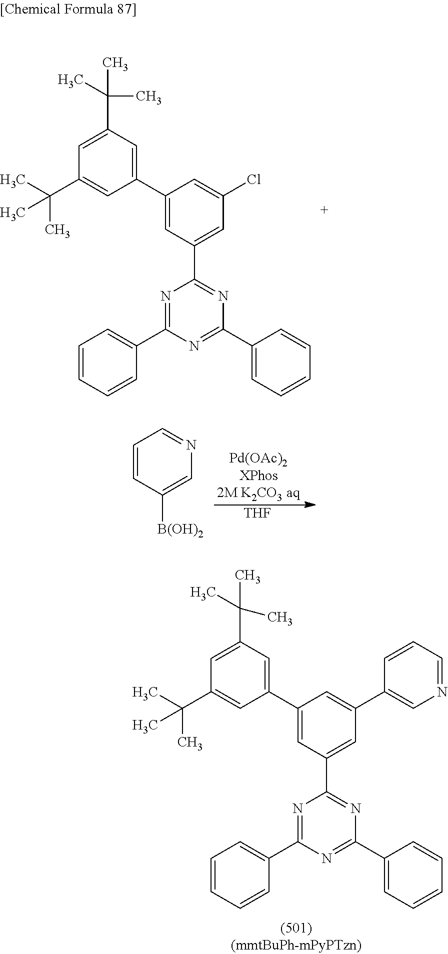 [SMILES: CC(C)(C)c1cc(-c2cc(Cl)cc(-c3nc(-c4ccccc4)nc(-c4ccccc4)n3)c2)cc(C(C)(C)C)c1 ; OB(O)c1cccnc1 ; CC(C)(C)c1cc(-c2cc(-c3cccnc3)cc(-c3nc(-c4ccccc4)nc(-c4ccccc4)n3)c2)cc(C(C)(C)C)c1 ; "THF"]

Then, 3.2 g of the obtained white solid was purified by a train sublimation method. In the purification by sublimation, the solid was heated at 235° C. for 29 hours and then at 240° C. for 24 hours under a pressure of 6.5 Pa while an argon gas was made to flow. After the purification by sublimation, 3.0 g of a target white solid was obtained at a collection rate of 95%.

Analysis results by nuclear magnetic resonance (¹H-NMR) spectroscopy of the white solid obtained above are shown below. These results reveal that the organic compound of one embodiment of the present invention, mmtBuPh-mPyPTzn represented by Structural Formula (501), was obtained in this example.

¹H NMR (CDCl₃, 300 MHz): δ=1.45 (s, 18H), 7.46-7.51 (m, 1H), 7.55-7.67 (m, 9H), 8.00 (s, 1H), 8.10 (d, 1H), 8.69 (d, 1H), 8.80 (d, 4H), 8.96 (s, 1H), 9.03 (s, 1H), 9.09 (d, 1H).

The absorption spectrum of mmtBuPh-mPyPTzn was measured with an ultraviolet-visible light spectrophotometer (V550, manufactured by JASCO Corporation). The absorption spectrum of mmtBuPh-mPyPTzn in the dichloromethane solution was obtained by subtracting the spectrum of dichloromethane alone in a quartz cell from the absorption spectrum of mmtBuPh-mPyPTzn in the dichloromethane solution in the quartz cell. As a result, the absorption peak was observed at 268 nm, revealing that no absorption was shown in the visible region range from 440 nm to 700 nm.

Next, mmtBuPh-mPyPTzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBuPh-mPyPTzn in an organic solvent at an arbitrary concentration, and the injection amount was 5.0 μL.

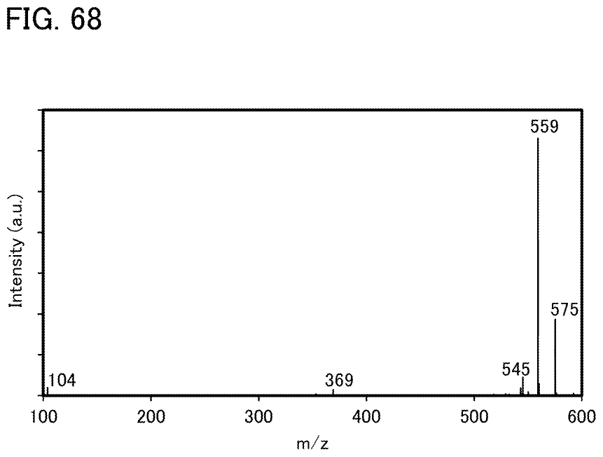

The MS/MS measurement of m/z=602.34 corresponding to the exact mass of mmtBuPh-mPyPTzn was performed by a PRM method. For setting of the PRM, the mass range of a target ion was set to m/z=602.34±2.0 (isolation window=4) and detection was performed in a positive mode. The measurement was performed with energy (normalized collision energy: NCE) for accelerating a target ion in a collision cell set to 60. The MS spectrum obtained by the MS/MS measurement is shown in FIG. 68.

Fragment ions at m/z of 104.05 and m/z of 369.23 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. The fragment ion at m/z of 369.23 is probably a fragment in which one carbon atom and one nitrogen atom each derived from triazine are bonded to a substituent other than a phenyl group. These fragments can each be regarded as a feature of a compound having a triazine skeleton.

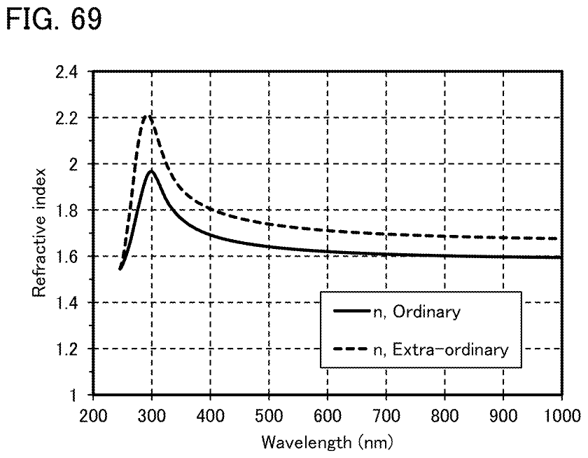

FIG. 69 shows the results of measuring the refractive index of mmtBuPh-mPyPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 69.

FIG. 69 shows that mmtBuPh-mPyPTzn is a material with a low refractive index: the ordinary refractive index of mmtBuPh-mPyPTzn in the entire blue emission region (from 455 nm to 465 nm) is 1.66, which is in the range of 1.50 to 1.75, and the ordinary refractive index of mmtBuPh-mPyPTzn at a wavelength of 633 nm is 1.62, which is in the range of 1.45 to 1.70.

Note that favorable light emission can be obtained from a light-emitting device fabricated using the organic compound as an electron-transport material.

### Example 18

### Synthesis Example 14

In this example, a method for synthesizing 2-[3-(2,6-dimethylpyridin-3-yl)-5-{3′,5,5′-tri-tert-butylbiphenyl}phenyl]-4,6-diphenyl-1,3,5-triazine (abbreviation: mmtBuBP-mDMePyPTzn), which is the organic compound of one embodiment of the present invention, is described. The structural formula of mmtBuBP-mDMePyPTzn is shown below.

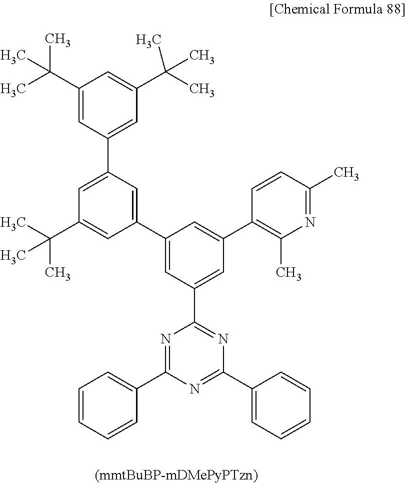 [SMILES: Cc1ccc(-c2cc(-c3cc(-c4cc(C(C)(C)C)cc(C(C)(C)C)c4)cc(C(C)(C)C)c3)cc(-c3nc(-c4ccccc4)nc(-c4ccccc4)n3)c2)c(C)n1]

### Step 1: Synthesis of 2-{3-chloro-5-(3′,5,5′-tri-tert-butylbiphenyl)phenyl}-4,6-diphenyl-1,3,5-triazine

Into a three-neck flask were put 4.0 g (9.5 mmol) of 2-(3-bromo-5-chlorophenyl)-4,6-diphenyl-1,3,5-triazine, 4.7 g (10 mmol) of 2-(3′,5,5′-tri-tert-butyl[1,1′-biphenyl]-3-yl)-4,4,5,5-tetramethyl-1,3,2-dioxaborolane, 10 mL of an aqueous solution of potassium carbonate (2 mol/L), 36 mL of toluene, and 18 mL of ethanol (EtOH), and the mixture was degassed. To this mixture were added 0.12 g (0.38 mmol) of tris(2-methylphenyl)phosphine and 21 mg (0.095 mmol) of palladium(II) acetate, and the mixture was heated and stirred at 80° C. for 10 hours. After the reaction, extraction was performed with toluene and the organic layer was dried with magnesium sulfate. This mixture was gravity-filtered, and the obtained filtrate was concentrated to give a gray solid. This solid was purified by silica gel column chromatography with a developing solvent of toluene and hexane in a ratio of 1:7 to give 5.9 g of a target white solid in a yield of 93%. The synthesis scheme of Step 1 is shown below.

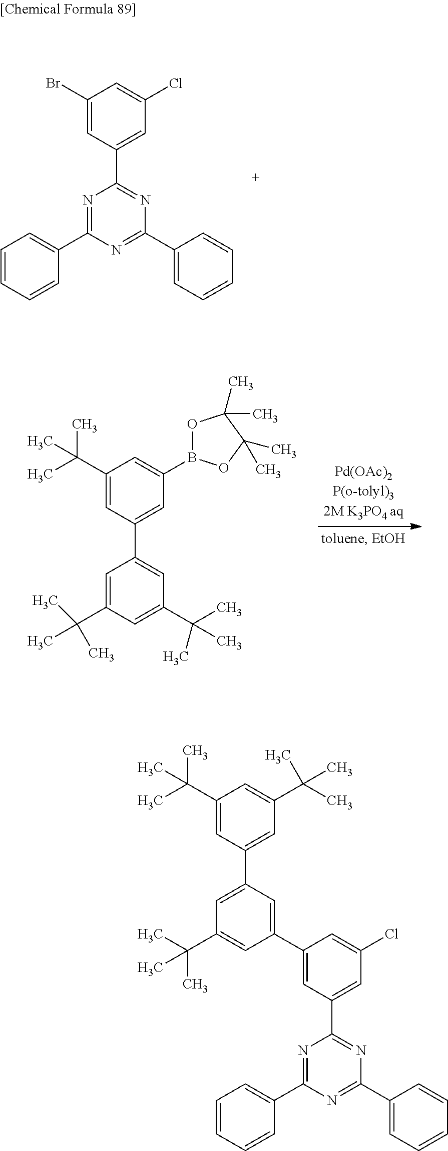 [SMILES: Clc1cc(Br)cc(-c2nc(-c3ccccc3)nc(-c3ccccc3)n2)c1 ; CC(C)(C)c1cc(B2OC(C)(C)C(C)(C)O2)cc(-c2cc(C(C)(C)C)cc(C(C)(C)C)c2)c1 ; CC(C)(C)c1cc(-c2cc(Cl)cc(-c3nc(-c4ccccc4)nc(-c4ccccc4)n3)c2)cc(-c2cc(C(C)(C)C)cc(C(C)(C)C)c2)c1]

### Step 2: Synthesis of 2-{5-(3′,5,5′-tri-tert-butylbiphenyl)-3-(4,4,5,5-tetramethyl-1,3,2-dioxaborolan-2-yl)phenyl}-4,6-diphenyl-1,3,5-triazine

Into a three-neck flask were put 5.9 g (8.8 mmol) of 2-{3-chloro-5-(3′,5,5′-tri-tert-butylbiphenyl)phenyl}-4,6-diphenyl-1,3,5-triazine, 3.4 g (13 mmol) of bis(pinacolato)diboron, 2.6 g (26 mmol) of potassium acetate, and 120 mL of 1,4-dioxane, and the mixture was degassed. To this mixture were added 84 mg (0.18 mmol) of 2-dicyclohexylphosphino-2′,4′,6′-triisopropylbiphenyl and 20 mg (0.088 mmol) of palladium(II) acetate, and reaction was caused at 100° C. for 7.5 hours. After the reaction, extraction was performed with toluene and the organic layer was dried with magnesium sulfate. This mixture was gravity-filtered, and the obtained filtrate was concentrated to give a gray oil. This oil was purified by silica gel column chromatography with a developing solvent of toluene and hexane in a ratio of 1:2, which was then changed to only toluene, to give 5.0 g of a target substance of a transparent oil and a white solid in a yield of 75%. The synthesis scheme of Step 2 is shown below.

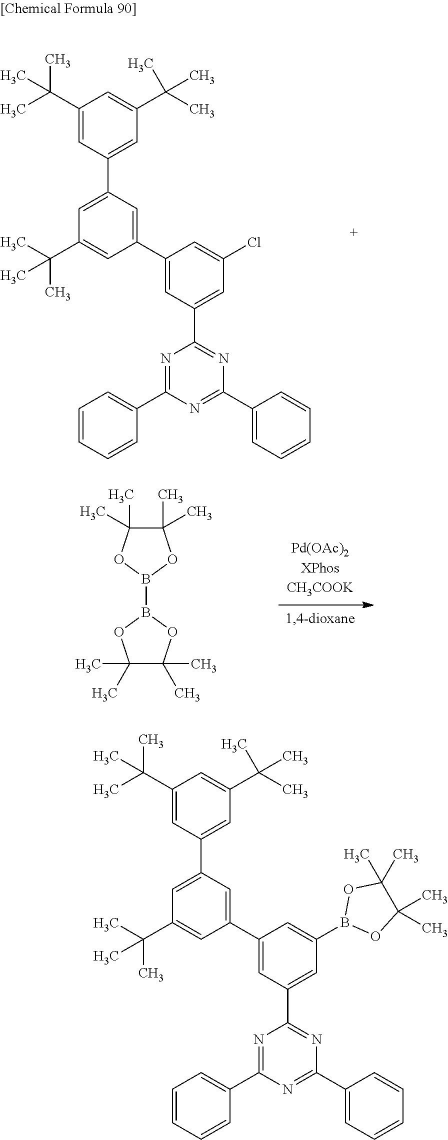

### Step 3: Synthesis of mmtBuBP-mDMePyPTzn

Into a three-neck flask were put 5.0 g (6.6 mmol) of 2-{5-(3′,5,5′-tri-tert-butylbiphenyl)-3-(4,4,5,5-tetramethyl-1,3,2-dioxaborolan-2-yl)phenyl}-4,6-diphenyl-1,3,5-triazine, 1.1 g (6.0 mmol) of 3-bromo-2,6-dimethylpyridine, 35 mL of tetrahydrofuran, and 9 mL (3.8 g, 18 mmol) of tripotassium phosphate (2 mol/L), and the mixture was degassed. To this mixture were added 14 mg (0.060 mmol) of palladium(II) acetate and 57 mg (0.12 mmol) of 2-dicyclohexylphosphino-2′,4′,6′-triisopropylbiphenyl, and the mixture was heated at 65° C. for 14 hours. After the reaction, extraction was performed with toluene and the organic layer was dried with magnesium sulfate. This mixture was gravity-filtered, and the obtained filtrate was concentrated to give a pale yellow solid. This solid was purified by silica gel column chromatography with a developing solvent of ethyl acetate and hexane in a ratio of 1:5 to give a pale yellow solid. After that, purification was performed by high performance liquid chromatography using chloroform as a mobile phase. The obtained solid was recrystallized with toluene and ethanol to give 3.2 g of a target white solid in a yield of 72%. The synthesis scheme of Step 3 is shown below.

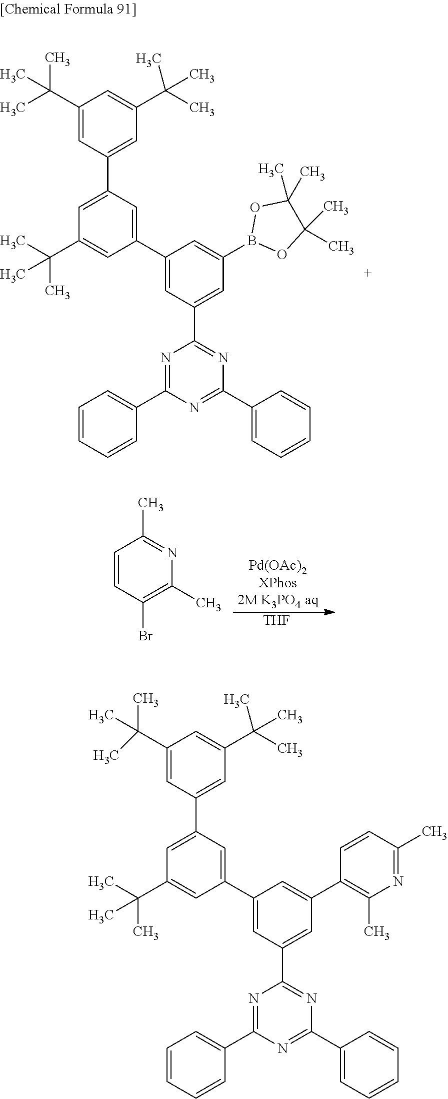

Then, 3.2 g of the obtained white solid was purified by a train sublimation method. In the purification by sublimation, the solid was heated at 260° C. for 17 hours and then at 265° C. for 23 hours under a pressure of 5.5 Pa while an argon gas was made to flow. After the purification by sublimation, 2.6 g of a target white solid was obtained at a collection rate of 81%.

Analysis results by nuclear magnetic resonance (¹H-NMR) spectroscopy of the obtained white solid are shown below. These results reveal that the organic compound of one embodiment of the present invention, mmtBuBP-mDMePyPTzn, was obtained in this example.

¹H NMR (CDCl₃, 300 MHz): δ=1.42 (s, 18H), 1.48 (s, 9H), 2.65 (s, 6H), 7.16 (d, 1H), 7.50 (s, 3H), 7.54-7.65 (m, 8H), 7.73 (d, 2H), 7.81 (s, 1H), 8.72 (s, 1H), 8.78 (d, 4H), 9.04 (s, 1H).

Next, the absorption spectrum of mmtBuBP-mDMePyPTzn was measured with an ultraviolet-visible light spectrophotometer (V550, manufactured by JASCO Corporation). The absorption spectrum of mmtBuBP-mDMePyPTzn in the dichloromethane solution was obtained by subtracting the spectrum of dichloromethane alone in a quartz cell from the absorption spectrum of mmtBuBP-mDMePyPTzn in the dichloromethane solution in the quartz cell. As a result, the absorption peak was observed at 265 nm, revealing that no absorption was shown in the visible region range from 440 nm to 700 nm.

Next, mmtBuBP-mDMePyPTzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBuBP-mDMePyPTzn in an organic solvent at an arbitrary concentration, and the injection amount was 5.0 μL.

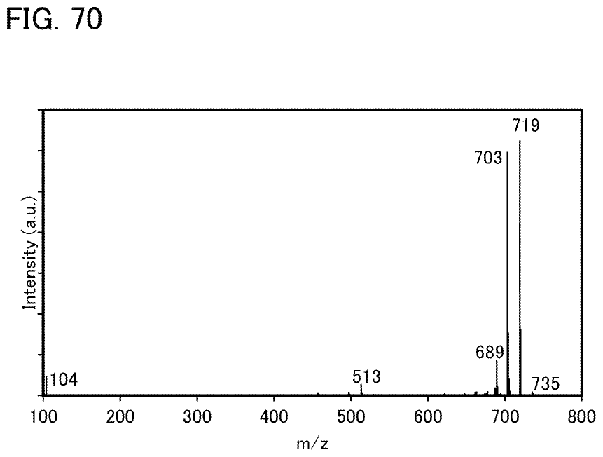

The MS/MS measurement of m/z=734.43 corresponding to the exact mass of mmtBuBP-mDMePyPTzn was performed by a PRM method. For setting of the PRM, the mass range of a target ion was set to m/z=734.43±2.0 (isolation window=4) and detection was performed in a positive mode. The measurement was performed with energy (NCE) for accelerating a target ion in a collision cell set to 70. The MS spectrum obtained by the MS/MS measurement is shown in FIG. 70.

A fragment ion at m/z of 104.05 was observed. This fragment ion is probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. This fragment can be regarded as a feature of a compound having a triazine skeleton.

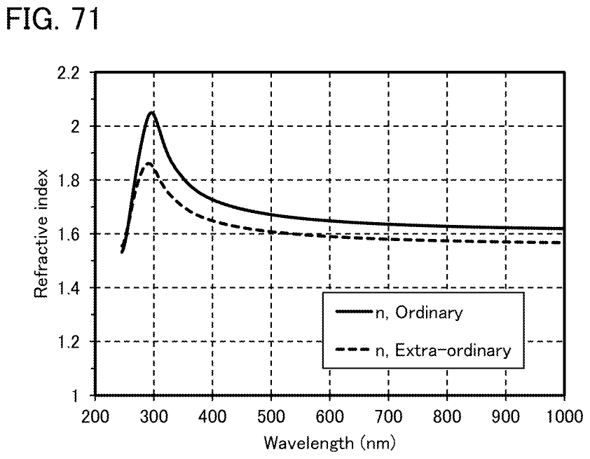

FIG. 71 shows the results of measuring the refractive index of mmtBuBP-mDMePyPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 71.

FIG. 71 shows that mmtBuBP-mDMePyPTzn is a material with a low refractive index: the ordinary refractive index of mmtBuBP-mDMePyPTzn in the entire blue emission region (from 455 nm to 465 nm) is 1.69, which is in the range of 1.50 to 1.75, and the ordinary refractive index of mmtBuBP-mDMePyPTzn at a wavelength of 633 nm is 1.64, which is in the range of 1.45 to 1.70.

Note that favorable light emission can be obtained from a light-emitting device fabricated using the organic compound as an electron-transport material.

### Example 19

In this example, light-emitting devices 8 and 9 of one embodiment of the present invention and a comparative light-emitting device 4 are described. Structural formulae of organic compounds used in this example are shown below.

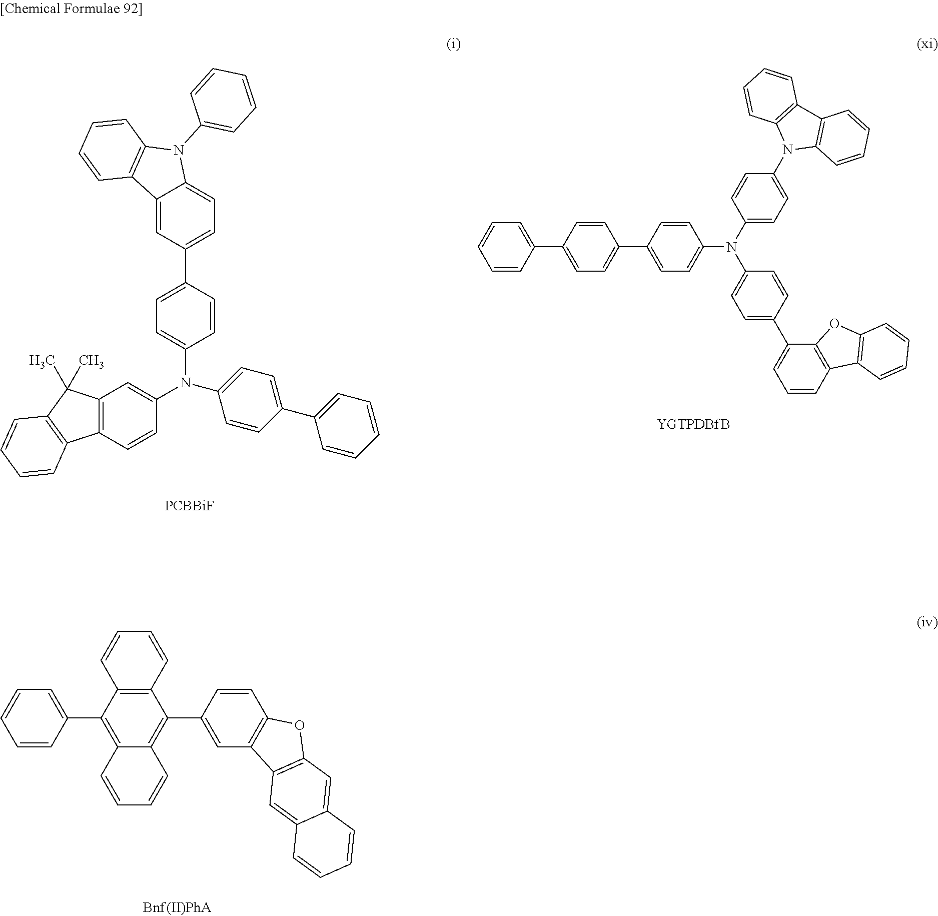

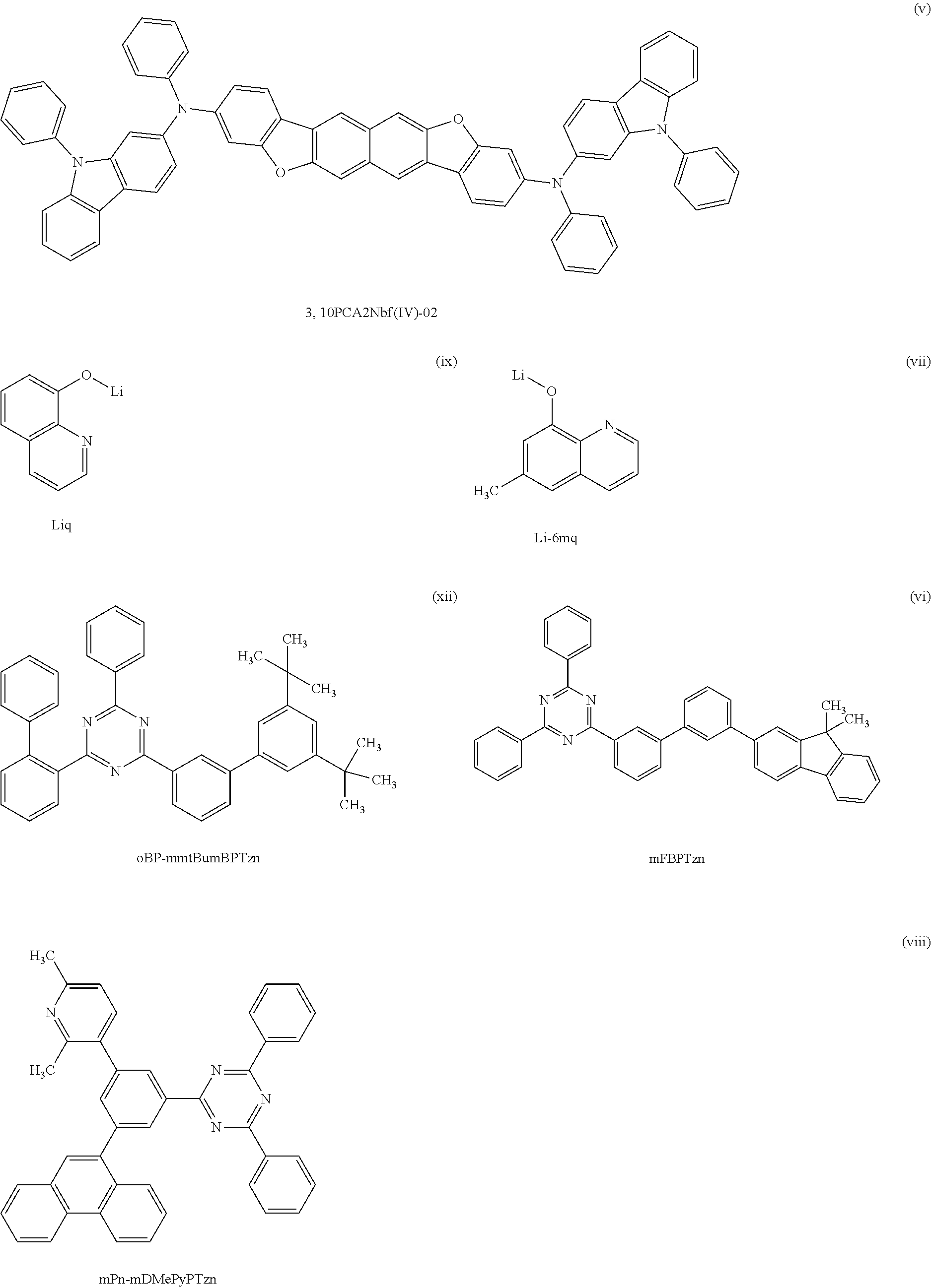

### (Fabrication Method of Light-Emitting Device 8)

First, as a transparent electrode, indium tin oxide containing silicon oxide (ITSO) was formed over a glass substrate to a thickness of 110 nm by a sputtering method, whereby the first electrode 101 was formed. The electrode area was 4 mm² (2 mm×2 mm).

Next, in pretreatment for forming the light-emitting device over a substrate, a surface of the substrate was washed with water and baked at 200° C. for one hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structure Formula (i) and an electron acceptor material (OCHD-001) were deposited by co-evaporation to a thickness of 10 nm such that the weight ratio of PCBBiF to OCHD-001 was 1:0.05, whereby the hole-injection layer 111 was formed.

Over the hole-injection layer 111, PCBBiF was deposited by evaporation to a thickness of 20 nm, whereby the hole-transport layer 112 was formed.

Next, over the hole-transport layer 112, N-[4-(9H-carbazol-9-yl)phenyl]-N-[4-(4-dibenzofuranyl)phenyl]-[1,1′:4′,1″-terphenyl]-4-amine (abbreviation: YGTPDBfB) represented by Structural Formula (xi) was deposited by evaporation to a thickness of 10 nm to form the electron-blocking layer.

Then, 2-(10-phenyl-9-anthracenyl)-benzo[b]naphtho[2,3-d]furan (abbreviation: Bnf(II)PhA) represented by Structural Formula (iv) and 3,10-bis[N-(9-phenyl-9H-carbazol-2-yl)-N-phenylamino]naphtho[2,3-b;6,7-b]bisbenzofuran (abbreviation: 3,10PCA2Nbf(IV)-02) represented by Structural Formula (v) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, 2-[(1,1′-biphenyl)-2-yl]-4-{(3′,5′-di-tert-butyl)-1,1′-biphenyl-3-yl}-6-phenyl-1,3,5-triazine (abbreviation: oBP-mmtBumBPTzn), which is the organic compound of one embodiment of the present invention represented by Structural Formula (xii), was deposited by evaporation to a thickness of 10 nm to form the hole-blocking layer. Then, oBP-mmtBumBPTzn and 8-quinolinolato-lithium (abbreviation: Liq) represented by Structural Formula (ix) were deposited by co-evaporation to a thickness of 20 nm such that the weight ratio of oBP-mmtBumBPTzn to Liq was 1:1, whereby the electron-transport layer 114 was formed.

After the electron-transport layer 114 was formed, Liq was deposited by evaporation to a thickness of 1 nm to form the electron-injection layer 115. Lastly, aluminum was deposited by evaporation to a thickness of 200 nm to form the second electrode 102. In the above manner, the light-emitting device was fabricated. The second electrode 102 is a reflective electrode having a function of reflecting light; thus, the light-emitting device of this example is a bottom-emission device in which light is extracted through the first electrode 101.

### (Fabrication Method of Light-Emitting Device 9)

The light-emitting device 9 was fabricated in the same manner as the light-emitting device 8 except that 6-methyl-8-quinolinolato-lithium (abbreviation: Li-6mq) represented by Structural Formula (vii) was used instead of Liq.

### (Fabrication Method of Comparative Light-Emitting Device 4)

The comparative light-emitting device 4 was fabricated in the same manner as the light-emitting device 8 except that the hole-blocking layer was formed using 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (vi), and 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (viii) was used instead of oBP-mmtBumBPTzn for the electron-transport layer.

The structures of the light-emitting devices 8 and 9 and the comparative light-emitting device 4 are listed in Table 13 below.

TABLE 13

| | film | Light- | Light- | Comparative light- |
|---|---|---|---|---|
| | thickness | emitting device 8 | emitting device 9 | emitting device 4 |
| electron-injection layer | 1 nm | LiF | | |
| electron-transport layer | 20 nm | oBP- | oBP- | mPn- |
| | | mmtBumBPTzn:Liq | mmtBumBPTzn:Li-6mq | mDMePyPTzn:Liq |
| | | (1:1) | (1:1) | (1:1) |

| | | | |
|---|---|---|---|
| hole-blocking layer | 10 nm | oBP-mmtBumBPTzn | mFBPTzn |

| | | |
|---|---|---|
| light-emitting layer | 25 nm | Bnf(II)PhA:3,10PCA2Nbf(IV)-02 |
| | | (1:0.015) |
| electron-blocking layer | 10 nm | YGTPDBfB |
| hole-transport layer | 20 nm | PCBBiF |
| hole-injection layer | 10 nm | PCBBiF:OCHD-001 |
| | | (1:0.05) |

Table 14 shows the refractive indices of oBP-mmtBumBPTzn, mPn-mDMePyPTzn, Li-6mq, and Liq at a wavelength of 456 nm. The refractive indices were measured with a spectroscopic ellipsometer (M-2000U, produced by J.A. Woollam Japan Corp.). A sample used for the measurement was formed to a thickness of approximately 50 nm with the material of each layer over a quartz substrate by a vacuum evaporation method.

TABLE 14

| | ordinary refractive index (n, Ordinary) |
|---|---|
| | at 456 nm |
| oBP-mmtBumBPTzn | 1.65 |
| mPn-mDMePyPTzn | 1.81 |
| mFBPTzn | 1.80 |
| Li-6mq | 1.67 |
| Liq | 1.72 |

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

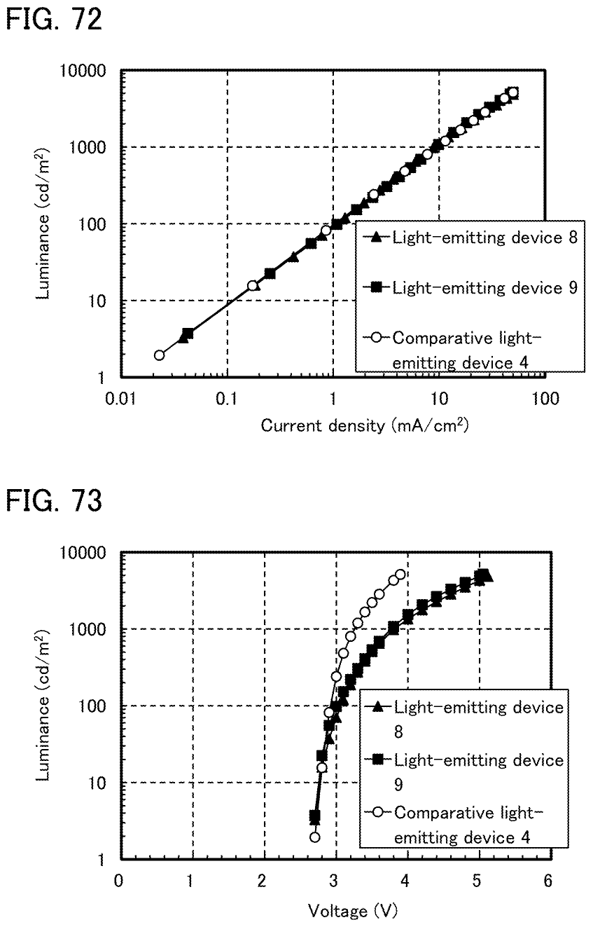

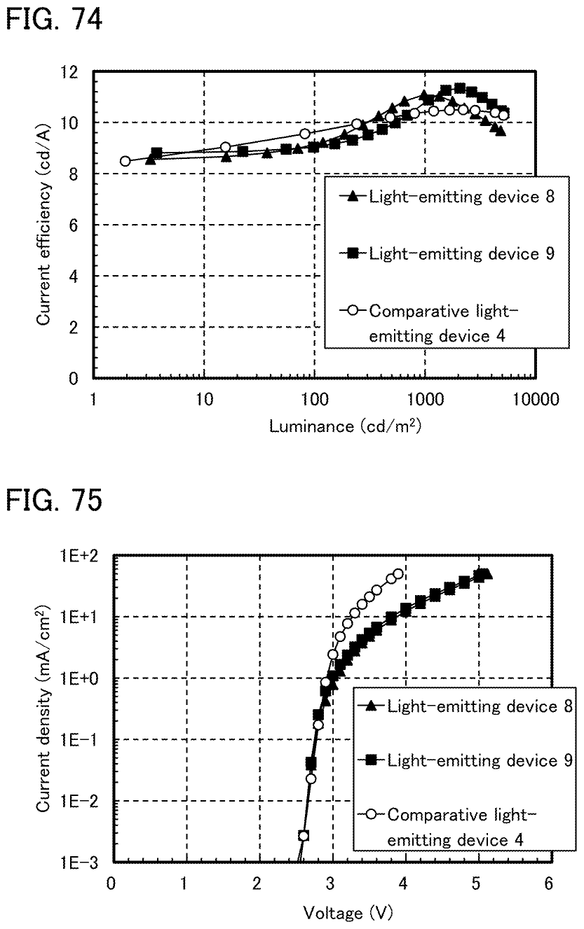

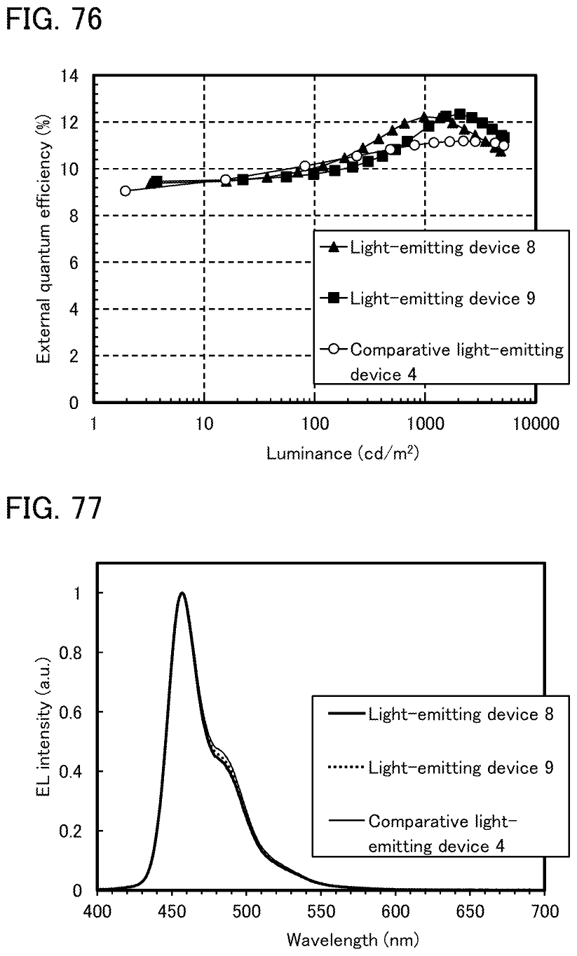

FIG. 72 shows the luminance-current density characteristics of the light-emitting devices 8 and 9 and the comparative light-emitting device 4. FIG. 73 shows the luminance-voltage characteristics thereof. FIG. 74 shows the current efficiency-luminance characteristics thereof. FIG. 75 shows the current density-voltage characteristics thereof. FIG. 76 shows the external quantum efficiency-luminance characteristics thereof. FIG. 77 shows the emission spectra thereof. Table 15 shows the main characteristics of the light-emitting devices 8 and 9 and the comparative light-emitting device 4 at a luminance of about 1000 cd/m². Luminance, CIE chromaticity, and emission spectra were measured at normal temperature with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 15

| | | | | | | current | external |
|---|---|---|---|---|---|---|---|
| | voltage | current | current density | | | efficiency | quantum |
| | (V) | (mA) | (mA/cm²) | chromaticity x | chromaticity y | (cd/A) | efficiency (%) |
| Light-emitting device 8 | 3.8 | 0.35 | 8.8 | 0.14 | 0.10 | 11.1 | 12 |
| Light-emitting device 9 | 3.8 | 0.40 | 10 | 0.14 | 0.11 | 10.9 | 12 |
| Comparative light-emitting device 4 | 3.3 | 0.46 | 11 | 0.14 | 0.11 | 10.4 | 11 |

The results in FIG. 72 to FIG. 77 and Table 15 reveal that a device having high current efficiency and external quantum efficiency can be obtained with the use of oBP-mmtBumBPTzn, which is the organic compound of one embodiment of the present invention, for an electron-transport layer and a hole-blocking layer. With the use of a mixed film of oBP-mmtBumBPTzn and Li-6mq for the electron-transport layer, a device having higher current efficiency and external quantum efficiency can be obtained.

### Example 20

In this example, a light-emitting device 10 of one embodiment of the present invention and a comparative light-emitting device 5 are described. Structural formulae of organic compounds used in this example are shown below.

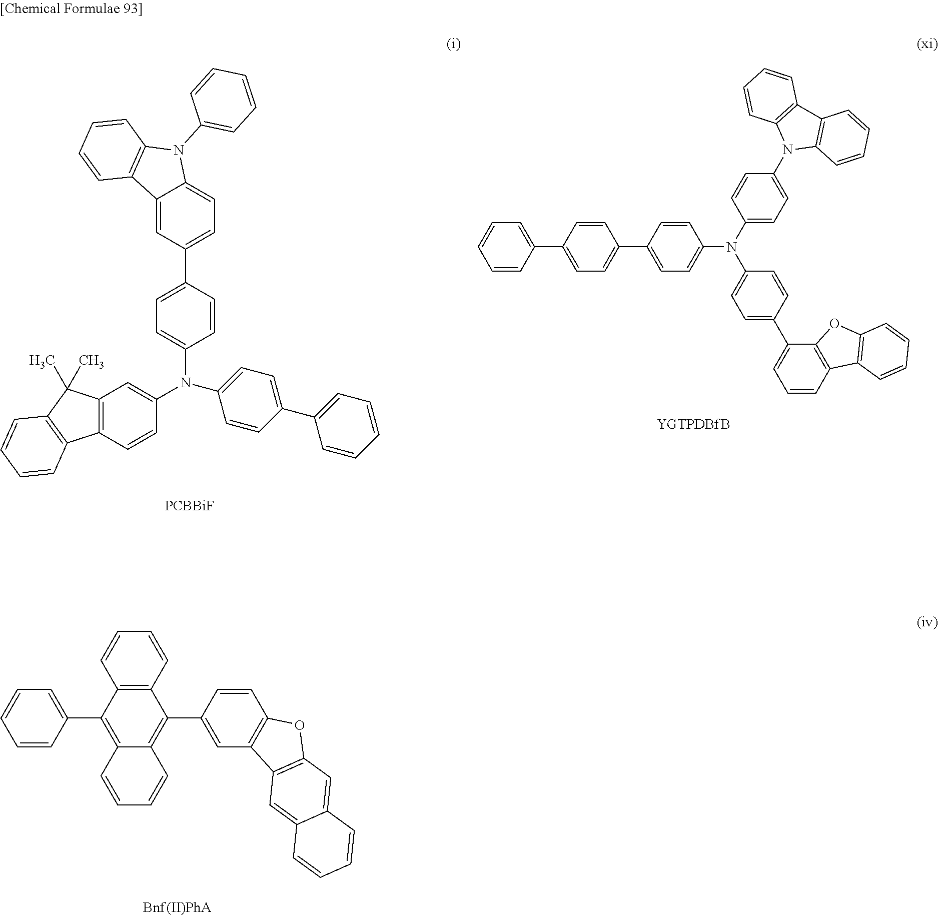

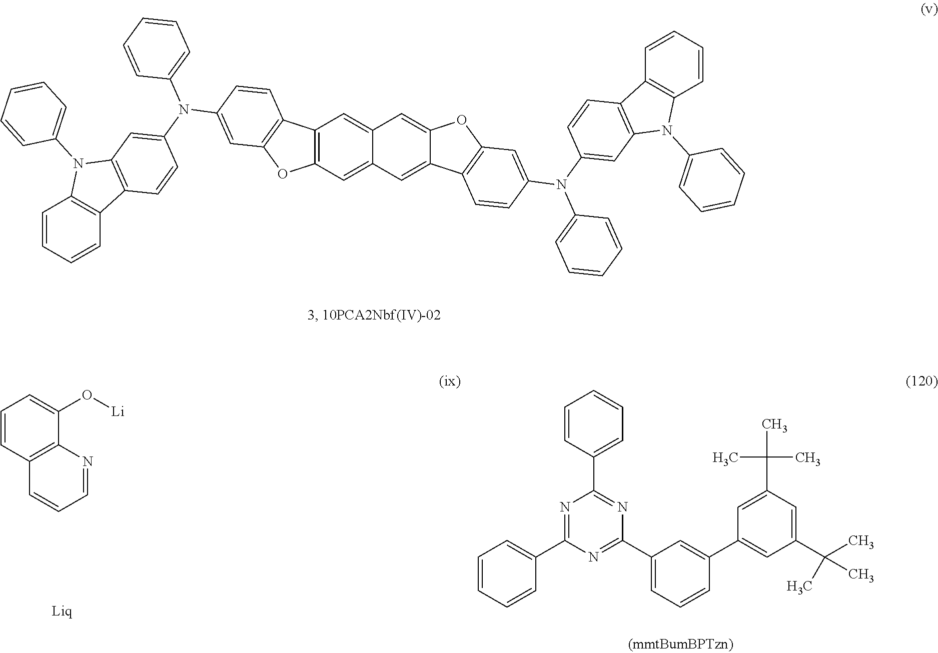

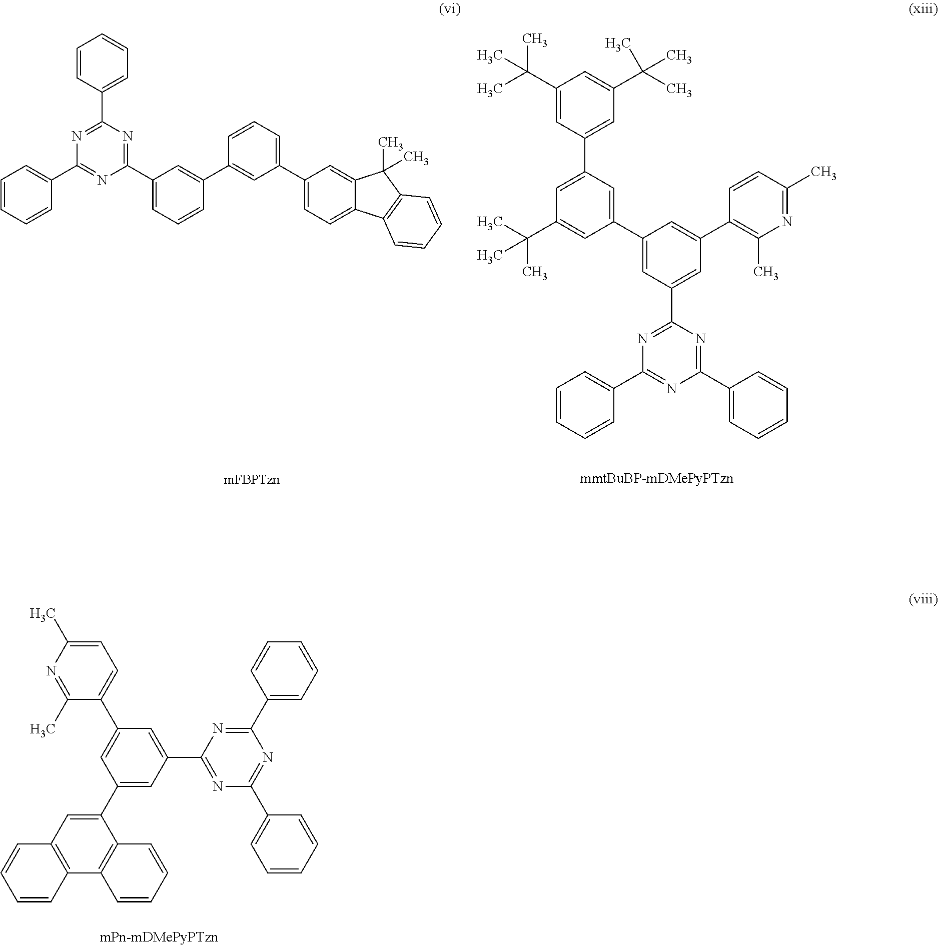 [SMILES: CC1(C)c2ccccc2-c2ccc(-c3cccc(-c4cccc(-c5nc(-c6ccccc6)nc(-c6ccccc6)n5)c4)c3)cc21 ; Cc1ccc(-c2cc(-c3cc(-c4cc(C(C)(C)C)cc(C(C)(C)C)c4)cc(C(C)(C)C)c3)cc(-c3nc(-c4ccccc4)nc(-c4ccccc4)n3)c2)c(C)n1 ; Cc1ccc(-c2cc(-c3nc(-c4ccccc4)nc(-c4ccccc4)n3)cc(-c3cc4ccccc4c4ccccc34)c2)c(C)n1]

### (Fabrication Method of Light-Emitting Device 10)

First, as a transparent electrode, indium tin oxide containing silicon oxide (ITSO) was formed over a glass substrate to a thickness of 110 nm by a sputtering method, whereby the first electrode 101 was formed. The electrode area was 4 mm² (2 mm×2 mm).

Next, in pretreatment for forming the light-emitting device over a substrate, a surface of the substrate was washed with water and baked at 200° C. for one hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Then, the substrate provided with the first electrode 101 was fixed to a substrate holder provided in the vacuum evaporation apparatus such that the surface on which the first electrode 101 was formed faced downward. Over the first electrode 101, N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structure Formula (i) and an electron acceptor material (OCHD-001) were deposited by co-evaporation to a thickness of 10 nm such that the weight ratio of PCBBiF to OCHD-001 was 1:0.05, whereby the hole-injection layer 111 was formed.

Over the hole-injection layer 111, PCBBiF was deposited by evaporation to a thickness of 20 nm, whereby the hole-transport layer 112 was formed.

Next, over the hole-transport layer 112, N-[4-(9H-carbazol-9-yl)phenyl]-N-[4-(4-dibenzofuranyl)phenyl]-[1,1′:4′,1″-terphenyl]-4-amine (abbreviation: YGTPDBfB) represented by Structural Formula (xi) was deposited by evaporation to a thickness of 10 nm to form the electron-blocking layer.

Then, 2-(10-phenyl-9-anthracenyl)-benzo[b]naphtho[2,3-d]furan (abbreviation: Bnf(II)PhA) represented by Structural Formula (iv) and 3,10-bis[N-(9-phenyl-9H-carbazol-2-yl)-N-phenylamino]naphtho[2,3-b;6,7-b]bisbenzofuran (abbreviation: 3,10PCA2Nbf(IV)-02) represented by Structural Formula (v) were deposited by co-evaporation to a thickness of 25 nm such that the weight ratio of Bnf(II)PhA to 3,10PCA2Nbf(IV)-02 was 1:0.015, whereby the light-emitting layer 113 was formed.

Next, 2-{(3′,5′-di-tert-butyl)-1,1′-biphenyl-3-yl}-4,6-diphenyl-1,3,5-triazine (abbreviation: mmtBumBPTzn), which is the organic compound of one embodiment of the present invention represented by Structural Formula (120), was deposited by evaporation to a thickness of 10 nm to form the hole-blocking layer. Then, 2-[3-(2,6-dimethylpyridin-3-yl)-5-{3′,5,5′-tri-tert-butylbiphenyl}phenyl]-4,6-diphenyl-1,3,5-triazine (abbreviation: mmtBuBP-mDMePyPTzn), which is the organic compound of one embodiment of the present invention represented by Structural Formula (xiii), and 8-quinolinolato-lithium (abbreviation: Liq) represented by Structural Formula (ix) were deposited by co-evaporation to a thickness of 20 nm such that the weight ratio of mmtBuBP-mDMePyPTzn to Liq was 1:1, whereby the electron-transport layer 114 was formed.

After the electron-transport layer 114 was formed, Liq was deposited by evaporation to a thickness of 1 nm to form the electron-injection layer 115. Lastly, aluminum was deposited by evaporation to a thickness of 200 nm to form the second electrode 102. In the above manner, the light-emitting device was fabricated. The second electrode 102 is a reflective electrode having a function of reflecting light; thus, the light-emitting device of this example is a bottom-emission device in which light is extracted through the first electrode 101.

### (Fabrication Method of Comparative Light-Emitting Device 5)

The comparative light-emitting device 5 was fabricated in the same manner as the light-emitting device 10 except that the hole-blocking layer was formed using 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (vi), and 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (viii) was used instead of mmtBuBP-mDMePyPTzn for the electron-transport layer.

The structures of the light-emitting device 10 and the comparative light-emitting device 5 are listed in Table 16 below.

TABLE 16

| | film | Light- | Comparative light- |
|---|---|---|---|
| | thickness | emitting device 10 | emitting device 5 |
| electron-injection layer | 1 nm | LiF | |
| electron-transport layer | 20 nm | mmtBuBP-mDMePyPTzn:Liq | mPn-mDMePyPTzn:Liq |
| | | (1:1) | (1:1) |
| hole-blocking layer | 10 nm | mmtBumBPTzn | mFBPTzn |

| | | |
|---|---|---|
| light-emitting layer | 25 nm | Bnf(II)PhA:3,10PCA2Nbf(IV)-02 |
| | | (1:0.015) |
| electron-blocking layer | 10 nm | YGTPDBfB |
| hole-transport layer | 20 nm | PCBBiF |
| hole-injection layer | 10 nm | PCBBiF:OCHD-001 |
| | | (1:0.05) |

Table 17 shows the refractive indices of mmtBuBP-mDMePyPTzn, mPn-mDMePyPTzn, Li-6mq, and Liq at a wavelength of 456 nm. The refractive indices were measured with a spectroscopic ellipsometer (M-2000U, produced by J.A. Woollam Japan Corp.). A sample used for the measurement was formed to a thickness of approximately 50 nm with the material of each layer over a quartz substrate by a vacuum evaporation method.

TABLE 17

| | ordinary refractive index (n, Ordinary) |
|---|---|
| | at 456 nm |
| mmtBuBP-mDMePyPTzn | 1.69 |
| mPn-mDMePyPTzn | 1.81 |
| mFBPTzn | 1.80 |
| Li-6mq | 1.67 |
| Liq | 1.72 |

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the initial characteristics of the light-emitting devices were measured. Note that the sealed glass substrate was not subjected to particular treatment for improving outcoupling efficiency.

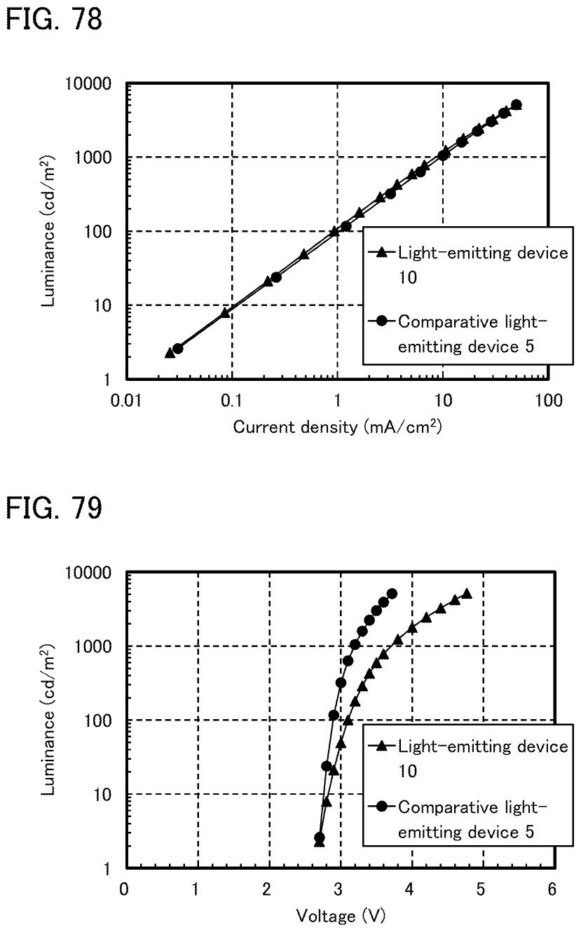

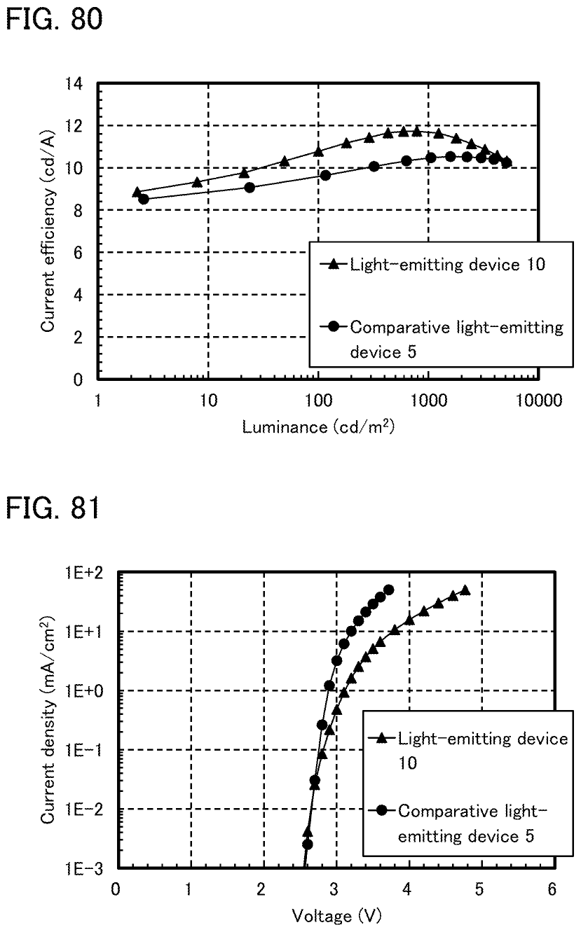

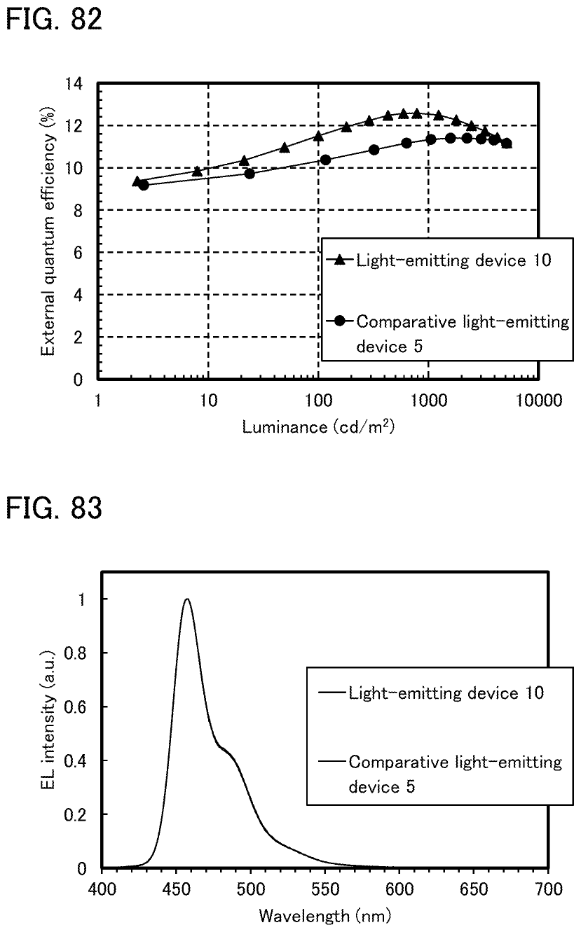

FIG. 78 shows the luminance-current density characteristics of the light-emitting device 10 and the comparative light-emitting device 5. FIG. 79 shows the luminance-voltage characteristics thereof. FIG. 80 shows the current efficiency-luminance characteristics thereof. FIG. 81 shows the current density-voltage characteristics thereof. FIG. 82 shows the external quantum efficiency-luminance characteristics thereof. FIG. 83 shows the emission spectra thereof. Table 18 shows the main characteristics of the light-emitting device 10 and the comparative light-emitting device 5 at a luminance of about 1000 cd/m². Luminance, CIE chromaticity, and emission spectra were measured at normal temperature with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 18

| | | | | | | current | external |
|---|---|---|---|---|---|---|---|
| | voltage | current | current density | | | efficiency | quantum |
| | (V) | (mA) | (mA/cm²) | chromaticity x | chromaticity y | (cd/A) | efficiency (%) |
| Light-emitting device 10 | 3.6 | 0.27 | 6.7 | 0.14 | 0.11 | 11.7 | 13 |
| Comparative light-emitting device 5 | 3.2 | 0.40 | 10 | 0.14 | 0.11 | 10.5 | 11 |

The results in FIG. 78 to FIG. 83 and Table 18 reveal that a device having high current efficiency and external quantum efficiency can be obtained with the use of mmtBuBP-mDMePyPTzn and mmtBumBPTzn, each of which is the organic compound of one embodiment of the present invention, for an electron-transport layer and a hole-blocking layer, respectively.

### Example 21

### Synthesis Example 15

In this example, a method for synthesizing 2-[3-(2,6-dimethylpyridin-3-yl)-5-{(3′,5′-di-tert-butyl)-1,1′-biphenyl-3-yl}phenyl]-4,6-diphenyl-1,3,5-triazine (abbreviation: mmtBuBP-mDMePyPTzn-02), which is the organic compound of one embodiment of the present invention, is described. The structure of mmtBuBP-mDMePyPTzn-02 is shown below.

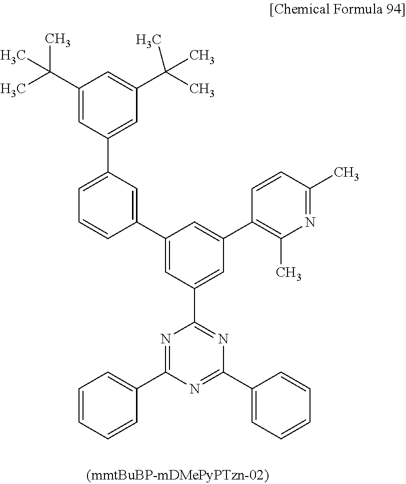 [SMILES: Cc1ccc(-c2cc(-c3cccc(-c4cc(C(C)(C)C)cc(C(C)(C)C)c4)c3)cc(-c3nc(-c4ccccc4)nc(-c4ccccc4)n3)c2)c(C)n1]

### Step 1: Synthesis of 2-(3′,5′-di-tert-butylbiphenyl-3-yl)-4,4,5,5-tetramethyl-1,3,2-dioxaborolane

This synthesis step is similar to Step 2 in Synthesis example 1.

### Step 2: Synthesis of 2-{3-chloro-5-(3′,5′-tri-tert-butylbiphenyl)phenyl}-4,6-diphenyl-1,3,5-triazine

Into a three-neck flask were put 4.12 g (9.75 mmol) of 2-(3-bromo-5-chlorophenyl)-4,6-diphenyl-1,3,5-triazine, 4.21 g (10.7 mmol) of 2-(3′,5′-di-tert-butylbiphenyl-3-yl)-4,4,5,5-tetramethyl-1,3,2-dioxaborolane, 36 mL of toluene, 18 mL of ethanol, and 10 mL (2.70 g, 19.5 mmol) of an aqueous solution of potassium carbonate (2 mol/L), and the mixture was degassed. To this mixture were added 0.12 g (0.39 mmol) of tris(2-methylphenyl)phosphine and 22 mg (0.098 mmol) of palladium(II) acetate, and the mixture was stirred at 80° C. for 14 hours. After the reaction, extraction was performed with toluene and the organic layer was dried with magnesium sulfate. This mixture was gravity-filtered, and the obtained filtrate was concentrated to give a brown solid. This solid was recrystallized with toluene and ethanol to give 5.16 g of a target white solid in a yield of 87%. The synthesis scheme of Step 2 is shown below.

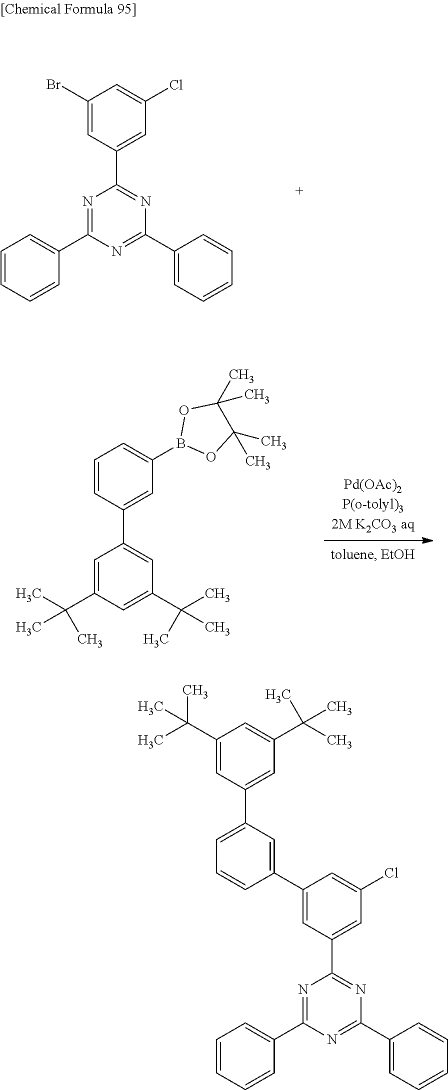 [SMILES: Clc1cc(Br)cc(-c2nc(-c3ccccc3)nc(-c3ccccc3)n2)c1 ; CC(C)(C)c1cc(-c2cccc(B3OC(C)(C)C(C)(C)O3)c2)cc(C(C)(C)C)c1 ; CC(C)(C)c1cc(-c2cccc(-c3cc(Cl)cc(-c4nc(-c5ccccc5)nc(-c5ccccc5)n4)c3)c2)cc(C(C)(C)C)c1]

### Step 3: Synthesis of 2-{5-(3′,5′-tri-tert-butylbiphenyl)-3-(4,4,5,5-tetramethyl-1,3,2-dioxaborolan-2-yl)phenyl}-4,6-diphenyl-1,3,5-triazine

Into a three-neck flask were put 5.1 g (8.40 mmol) of 2-{3-chloro-5-(3′,5′-tri-tert-butylbiphenyl)phenyl}-4,6-diphenyl-1,3,5-triazine, 3.20 g (12.6 mmol) of bis(pinacolato)diboron, 2.47 g (25.2 mmol) of potassium acetate, and 110 mL of 1,4-dioxane, and the mixture was degassed. To this mixture were added 80.1 mg (0.168 mmol) of 2-dicyclohexylphosphino-2′,4′,6′-triisopropylbiphenyl and 18.9 mg (0.084 mmol) of palladium(II) acetate, and the mixture was stirred at 100° C. for 13.5 hours. After the reaction, extraction was performed with toluene and the organic layer was dried with magnesium sulfate. This mixture was gravity-filtered, and the obtained filtrate was concentrated to give a whitish pale yellow solid. This solid was purified by silica gel column chromatography with a developing solvent of toluene and hexane in a ratio of 2:1, which was then changed to only toluene, to give 5.52 g of a target white solid in a yield of 93.9%. The synthesis scheme of Step 3 is shown below.

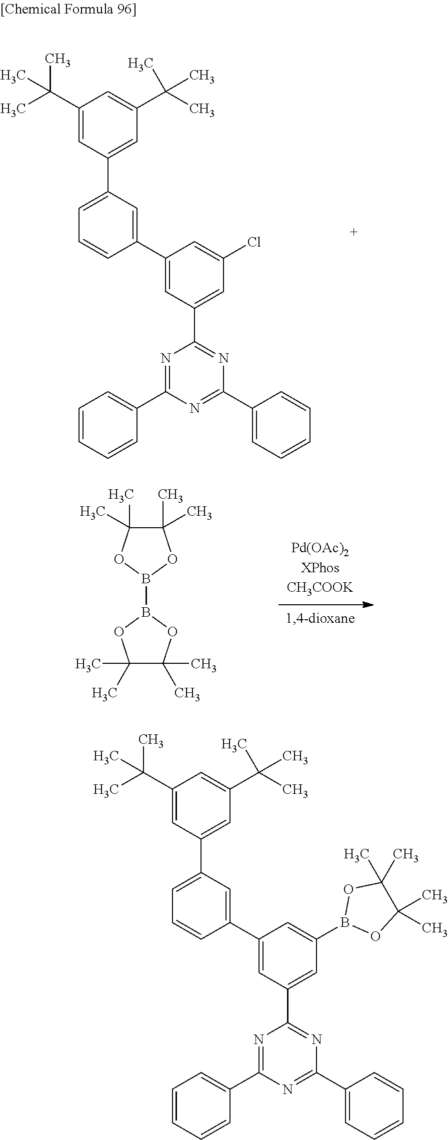

### Step 4: Synthesis method of mmtBuBP-mDMePyPTzn-02

Into a three-neck flask were put 5.5 g (7.89 mmol) of 2-{5-(3′,5′-tri-tert-butylbiphenyl)-3-(4,4,5,5-tetramethyl-1,3,2-dioxaborolan-2-yl)phenyl}-4,6-diphenyl-1,3,5-triazine, 1.33 g (7.17 mmol) of 3-bromo-2,6-dimethylpyridine, 40 mL of tetrahydrofuran, and 11 mL (4.57 g, 21.5 mmol) of tripotassium phosphate (2 mol/L), and the mixture was degassed. To this mixture were added 16.1 mg (0.072 mmol) of palladium(II) acetate and 68.4 mg (0.14 mmol) of 2-dicyclohexylphosphino-2′,4′,6′-triisopropylbiphenyl, and the mixture was stirred at 65° C. for 15 hours. After the reaction, extraction was performed with toluene and the organic layer was dried with magnesium sulfate. This mixture was gravity-filtered, and the obtained filtrate was concentrated to give a pale yellow solid. This solid was purified by silica gel column chromatography with a developing solvent of toluene, which was then changed to toluene and ethyl acetate in a ratio of 10:1, to give a pale yellow solid. This solid was recrystallized with toluene and ethanol to give 4.7 g of a target white solid in a yield of 96.6%. The synthesis scheme of Step 4 is shown below.

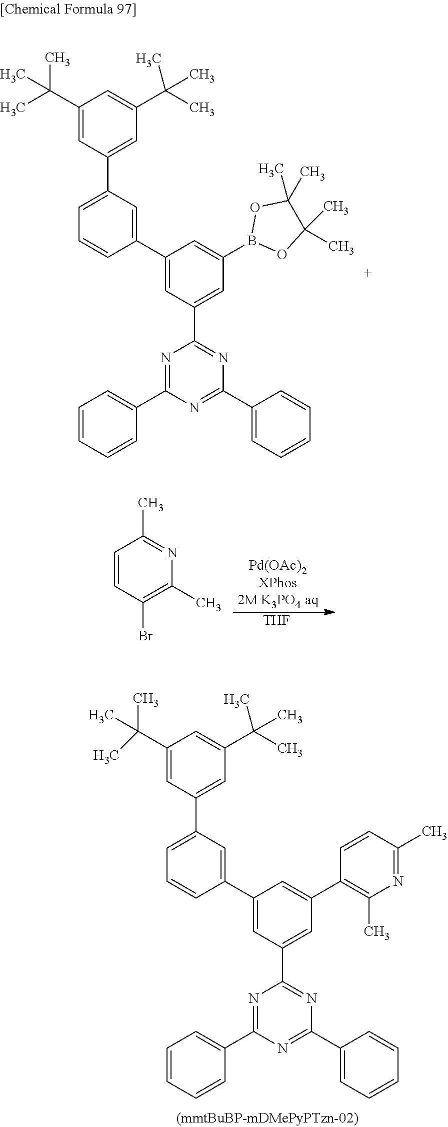

Then, 4.3 g of the obtained white solid was purified by a train sublimation method (heating was performed at 270° C. for 21 hours and then at 275° C. for 19.5 hours under a pressure of 6.6 Pa while an argon gas was made to flow). After the purification by sublimation, 3.8 g of a target white solid was obtained at a collection rate of 88%.

Analysis results by nuclear magnetic resonance (¹H-NMR) spectroscopy of the white solid obtained above are shown below. These results reveal that the organic compound of one embodiment of the present invention, mmtBuBP-mDMePyPTzn-02, was obtained in this example.

¹H NMR (CDCl₃, 300 MHz): δ=1.41 (s, 18H), 2.64 (s, 6H), 7.15 (s, 1H), 7.49-7.68 (m, 12H), 7.73 (d, 1H), 7.83 (s, 1H), 7.95 (s, 1H), 8.74 (s, 1H), 8.76-8.80 (m, 4H), 9.04 (s, 1H).

Next, the absorption spectrum of mmtBuBP-mDMePyPTzn-02 was measured with an ultraviolet-visible light spectrophotometer (V550, manufactured by JASCO Corporation). The absorption spectrum of mmtBuBP-mDMePyPTzn-02 in the dichloromethane solution was obtained by subtracting the spectrum of dichloromethane alone in a quartz cell from the absorption spectrum of mmtBuBP-mDMePyPTzn-02 in the dichloromethane solution in the quartz cell. As a result, the absorption peaks were observed at 278 nm and 311 nm, revealing that no absorption was shown in the visible region range from 440 nm to 700 nm.

Next, mmtBuBP-mDMePyPTzn-02 obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mmtBuBP-mDMePyPTzn-02 in an organic solvent at an arbitrary concentration, and the injection amount was 5.0 μL.

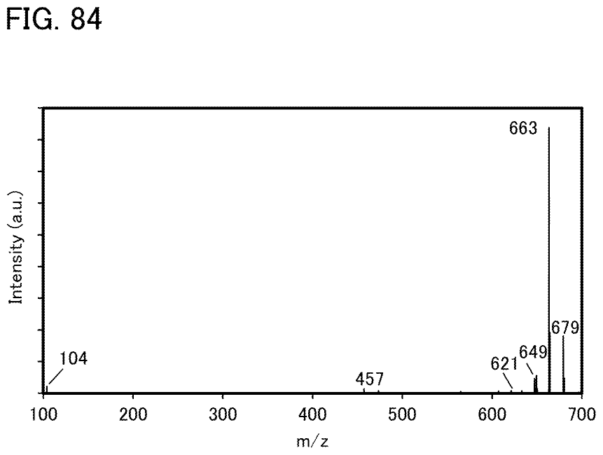

The MS/MS measurement of m/z=679.38 corresponding to an ion derived from mmtBuBP-mDMePyPTzn-02 was performed by a PRM method. For setting of the PRM, the mass range of a target ion was set to m/z=679.38±2.0 (isolation window=4) and detection was performed in a positive mode. The measurement was performed with energy (normalized collision energy: NCE) for accelerating a target ion in a collision cell set to 60. The MS spectrum obtained by the MS/MS measurement is shown in FIG. 84.

As a result of the MS/MS measurement, a fragment ion at m/z of 104.05 was observed. This fragment ion is probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. This fragment can be regarded as a feature of a compound having a triazine skeleton.

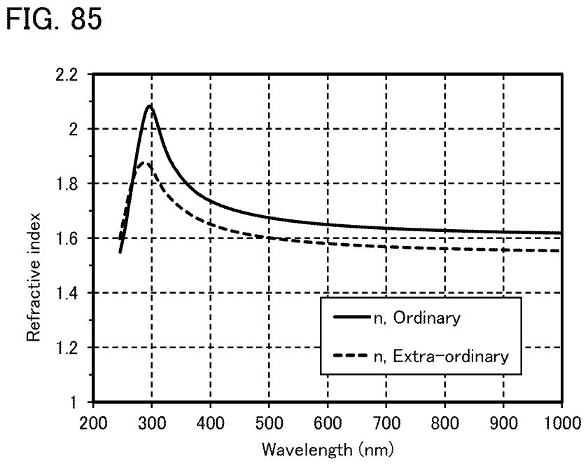

FIG. 85 shows the results of measuring the refractive index of mmtBuBP-mDMePyPTzn-02 with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 85.

FIG. 85 shows that mmtBuBP-mDMePyPTzn-02 is a material with a low refractive index: the ordinary refractive index of mmtBuBP-mDMePyPTzn-02 in the entire blue emission region (from 455 nm to 465 nm) is 1.69, which is in the range of 1.50 to 1.75, and the ordinary refractive index of mmtBuBP-mDMePyPTzn-02 at a wavelength of 633 nm is 1.64, which is in the range of 1.45 to 1.70.

Note that favorable light emission can be obtained from a light-emitting device fabricated using the organic compound as an electron-transport material.

### Example 22

### Synthesis Example 16

In this example, a method for synthesizing 2,4-[(1,1′-biphenyl)-2-yl]-6-[3-(2,6-dimethylpyridin-3-yl)-5-{(3,5-di-tert-butyl)phenyl}]phenyl-1,3,5-triazine (abbreviation: oBP2-mmtBuPh-mDMePyPTzn), which is the organic compound of one embodiment of the present invention, is described. The structure of oBP2-mmtBuPh-mDMePyPTzn is shown below.

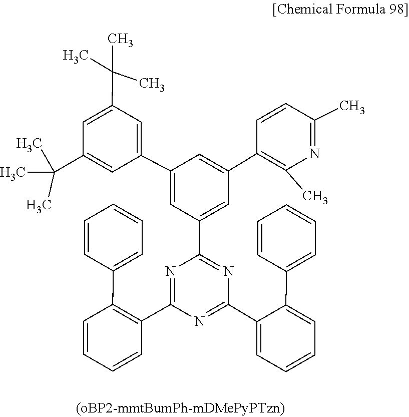 [SMILES: Cc1ccc(-c2cc(-c3cc(C(C)(C)C)cc(C(C)(C)C)c3)cc(-c3nc(-c4ccccc4-c4ccccc4)nc(-c4ccccc4-c4ccccc4)n3)c2)c(C)n1]

### Step 1: Synthesis of 2,4-bis{(1,1′-biphenyl)-2-yl}-6-(3-bromo-5-chlorophenyl)-1,3,5-triazine

Into a three-neck flask were put 6.0 g (14 mmol) of 2,4-bis{(1,1-biphenyl)-2-yl}-6-chloro-1,3,5-triazine, 3.7 g (16 mmol) of (3-bromo-5-chlorophenyl)boronic acid, 3.0 g (29 mmol) of sodium carbonate, 55 mL of toluene, 11 mL of ethanol, and 14 mL of water, and the mixture was degassed. To this mixture was added 0.33 g (0.29 mmol) of tetrakis(triphenylphosphine)palladium(0), and the mixture was heated and stirred at 80° C. for 9 hours. After the reaction, extraction was performed with toluene and the organic layer was dried with magnesium sulfate. This mixture was gravity-filtered, and the obtained filtrate was concentrated to give a yellow solid. This solid was purified by silica gel column chromatography with a developing solvent of toluene and hexane in a ratio of 1:1, which was then changed to 1:2. After that, purification was performed by high performance liquid chromatography using chloroform as a mobile phase to give 5.0 g of a target white solid in a yield of 61%. The synthesis scheme of Step 1 is shown below.

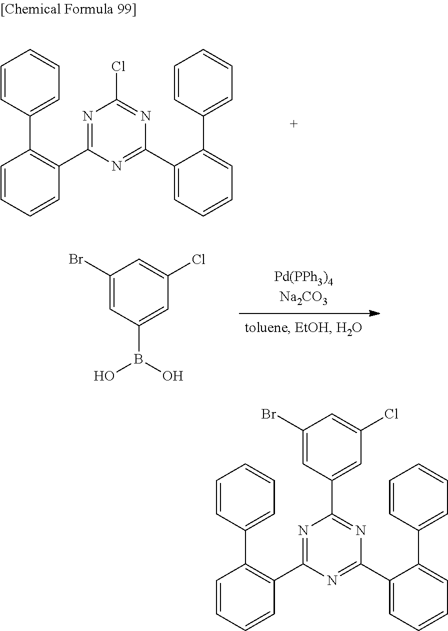

### Step 2: Synthesis of 2,4-bis{(1,1′-biphenyl)-2-yl}-6-{3-chloro-5-(3,5-di-tert-butylphenyl)phenyl}-1,3,5-triazine

Into a three-neck flask were put 5.0 g (8.7 mmol) of 2,4-bis{(1,1′-biphenyl)-2-yl}-6-(3-bromo-5-chlorophenyl)-1,3,5-triazine, 2.2 g (9.6 mmol) of 3,5-di-tert-butylphenylboronic acid, 8.7 mL of an aqueous solution of potassium carbonate (2 mol/L), 34 mL of toluene, and 17 mL of ethanol, and the mixture was degassed. To this mixture were added 0.11 g (0.35 mmol) of tris(2-methylphenyl)phosphine and 20 mg (0.087 mmol) of palladium(II) acetate, and reaction was caused at 80° C. for 13 hours. After the reaction, extraction was performed with toluene and the organic layer was dried with magnesium sulfate. This mixture was gravity-filtered, and the obtained filtrate was concentrated to give a pink solid. This solid was purified by silica gel column chromatography with a developing solvent of toluene and hexane in a ratio of 1:2, which was then changed to 1:1, to give 6.6 g of a target white solid containing toluene. The synthesis scheme of Step 2 is shown below.

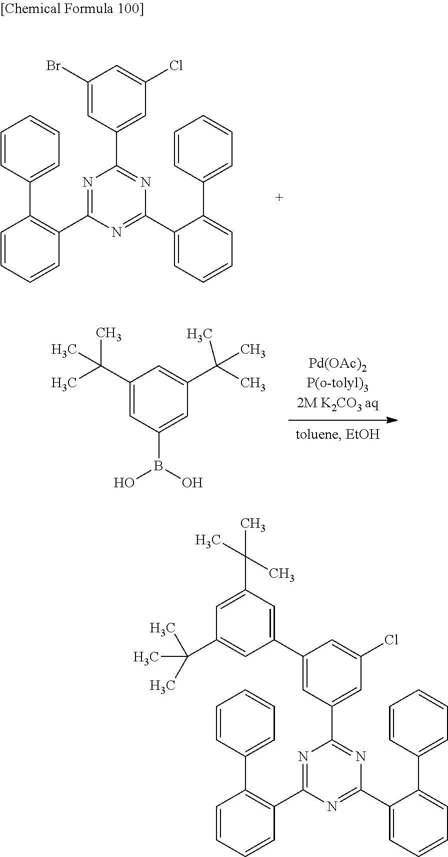 [SMILES: Clc1cc(Br)cc(-c2nc(-c3ccccc3-c3ccccc3)nc(-c3ccccc3-c3ccccc3)n2)c1 ; CC(C)(C)c1cc(B(O)O)cc(C(C)(C)C)c1 ; CC(C)(C)c1cc(-c2cc(Cl)cc(-c3nc(-c4ccccc4-c4ccccc4)nc(-c4ccccc4-c4ccccc4)n3)c2)cc(C(C)(C)C)c1]

### Step 3: Synthesis of 2,4-bis{(1,1′-biphenyl)-2-yl}-6-[3-{(3,5-di-tert-butyl)phenyl}-5-(4,4,5,5-tetramethyl-1,3,2-dioxaborolan-2-yl)phenyl]-1,3,5-triazine

Into a three-neck flask were put 6.5 g (9.5 mmol) of 2,4-bis{(1,1′-biphenyl)-2-yl}-6-{3-chloro-5-(3,5-di-tert-butylphenyl)phenyl}-1,3,5-triazine, 3.6 g (14 mmol) of bis(pinacolato)diboron, 2.8 g (29 mmol) of potassium acetate, and 150 mL of 1,4-dioxane, and the mixture was degassed. To this mixture were added 91 mg (0.19 mmol) of 2-dicyclohexylphosphino-2′,4′,6′-triisopropylbiphenyl and 21 mg (0.095 mmol) of palladium(II) acetate, and the mixture was stirred at 100° C. for 14 hours. After the reaction, extraction was performed with toluene and the organic layer was dried with magnesium sulfate. This mixture was gravity-filtered, and the obtained filtrate was concentrated to give a gray solid. This solid was purified by silica gel column chromatography with a developing solvent of toluene and hexane in a ratio of 1:2, which was then changed to only toluene, to give 5.98 g of a target white solid in a yield of 81%. The synthesis scheme of Step 3 is shown below.

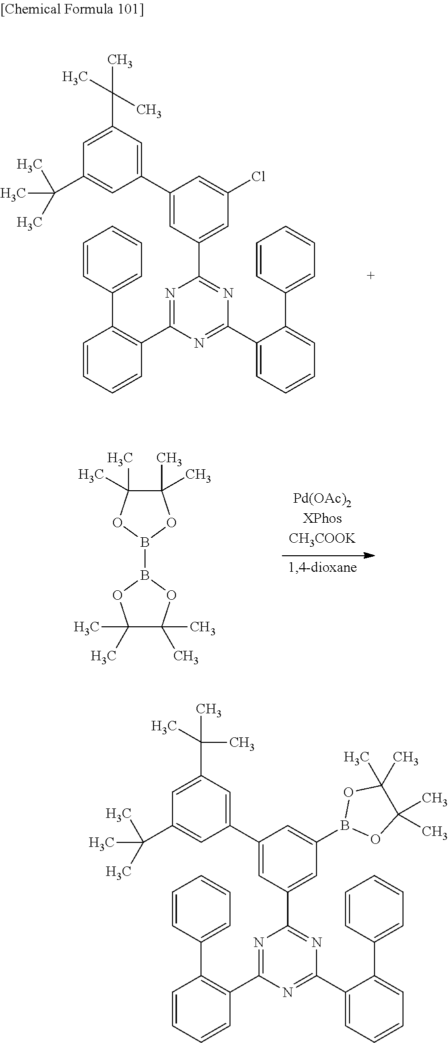

### Step 4: Synthesis of oBP2-mmtBuPh-mDMePyPTzn

Into a three-neck flask were put 6.0 g (7.7 mmol) of 2,4-bis{(1,1′-biphenyl)-2-yl}-6-[3-{(3,5-di-tert-butyl)phenyl}-5-(4,4,5,5-tetramethyl-1,3,2-dioxaborolan-2-yl)phenyl]-1,3,5-triazine, 1.25 g (6.7 mmol) of 3-bromo-2,6-dimethylpyridine, 40 mL of tetrahydrofuran, and 10 mL of tripotassium phosphate (2 mol/L), and the mixture was degassed. To this mixture were added 64 mg (0.13 mmol) of 2-dicyclohexylphosphino-2′,4′,6′-triisopropylbiphenyl and 15 mg (0.067 mmol) of palladium(II) acetate, and the mixture was stirred at 65° C. for 13.5 hours. After the reaction, extraction was performed with toluene and the organic layer was dried with magnesium sulfate. This mixture was gravity-filtered, and the obtained filtrate was concentrated to give a pale yellow solid. This solid was purified by silica gel column chromatography with a developing solvent of ethyl acetate and toluene in a ratio of 1:8 to give a pale yellow solid. Subsequently, this solid was purified by silica gel column chromatography with a developing solvent of ethyl acetate and toluene in a ratio of 1:20, which was then changed to 1:15, to give a pale yellow solid. After that, purification was performed by high performance liquid chromatography using chloroform as a mobile phase to give a white solid. This solid was recrystallized with toluene and ethanol to give 4.2 g of a target white solid in a yield of 83%. The synthesis scheme of Step 4 is shown below.

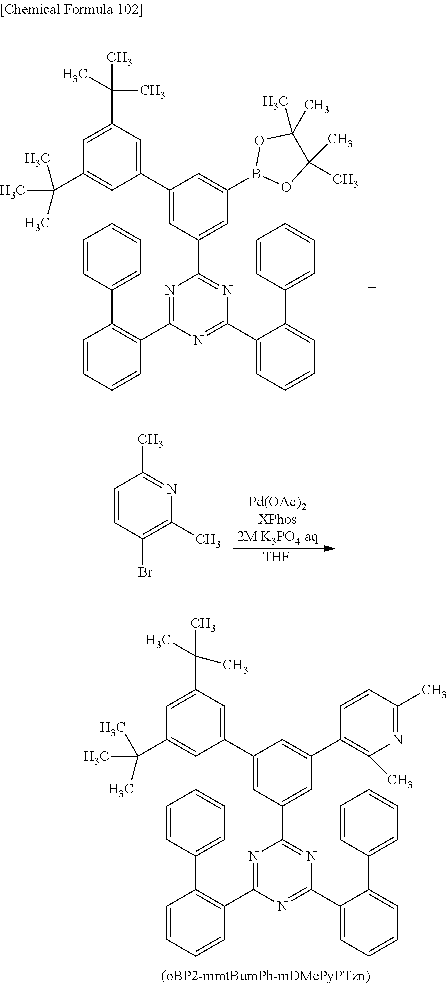

Then, 4.2 g of the obtained white solid was purified by a train sublimation method. In the purification by sublimation, the solid was heated at 260° C. for 23 hours and then at 265° C. for 18 hours under a pressure of 6.0 Pa while an argon gas was made to flow. After the purification by sublimation, 3.7 g of a target white solid was obtained at a collection rate of 88%.

Analysis results by nuclear magnetic resonance (¹H-NMR) spectroscopy of the white solid obtained above are shown below. These results reveal that the organic compound of one embodiment of the present invention, oBP2-mmtBuPh-mDMePyPTzn, was obtained in this example.

¹H NMR (CDCl₃, 300 MHz): δ=1.41 (s, 18H), 2.46 (s, 3H), 2.66 (s, 3H), 6.98 (t, 1H), 7.13 (t, 5H), 7.22 (d, 4H), 7.37-7.62 (m, 14H), 8.22 (s, 1H).

Next, the absorption spectrum of oBP2-mmtBuPh-mDMePyPTzn was measured with an ultraviolet-visible light spectrophotometer (V550, manufactured by JASCO Corporation). The absorption spectrum of oBP2-mmtBuPh-mDMePyPTzn in the dichloromethane solution was obtained by subtracting the spectrum of dichloromethane alone in a quartz cell from the absorption spectrum of oBP2-mmtBuPh-mDMePyPTzn in the dichloromethane solution in the quartz cell. As a result, the absorption peak was observed at 270 nm, revealing that no absorption was shown in the visible region range from 440 nm to 700 nm.

Next, oBP2-mmtBuPh-mDMePyPTzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving oBP2-mmtBuPh-mDMePyPTzn in an organic solvent at an arbitrary concentration, and the injection amount was 5.0 μL.

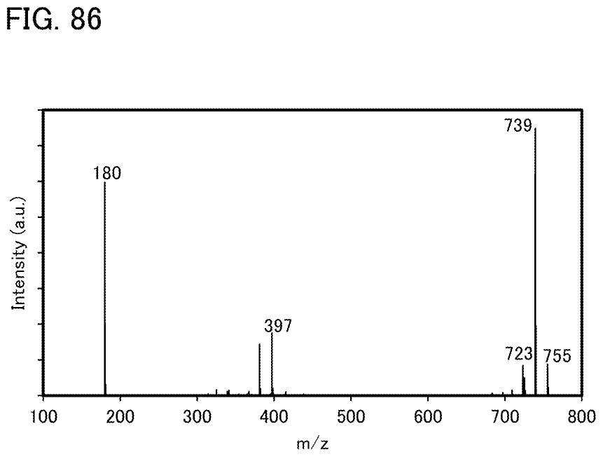

The MS/MS measurement of m/z=679.38 corresponding to an ion derived from oBP2-mmtBuPh-mDMePyPTzn was performed by a PRM method. For setting of the PRM, the mass range of a target ion was set to m/z=679.38±2.0 (isolation window=4) and detection was performed in a positive mode. The measurement was performed with energy (normalized collision energy: NCE) for accelerating a target ion in a collision cell set to 60. The MS spectrum obtained by the MS/MS measurement is shown in FIG. 86.

As a result of the MS/MS measurement, fragment ions at m/z of 180.08 and m/z of 397.26 were observed. These fragment ions are each probably a fragment formed of one substituent bonded to triazine and carbon and nitrogen derived from triazine. For example, the fragment ion at m/z of 180.08 is probably a fragment in which one carbon atom and one nitrogen atom each derived from triazine are bonded to a biphenyl group. The fragment ion at m/z of 397.26 is probably a fragment in which one carbon atom and one nitrogen atom each derived from triazine are bonded to a substituent other than a biphenyl group. These fragments can each be regarded as a feature of a compound having a triazine skeleton.

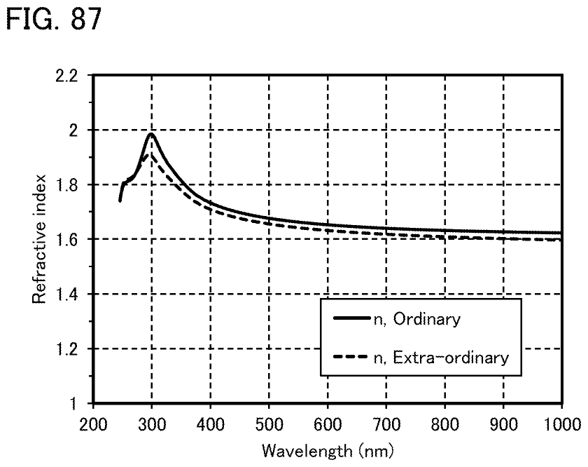

FIG. 87 shows the results of measuring the refractive index of oBP2-mmtBuPh-mDMePyPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 87.

FIG. 87 shows that oBP2-mmtBuPh-mDMePyPTzn is a material with a low refractive index: the ordinary refractive index of oBP2-mmtBuPh-mDMePyPTzn in the entire blue emission region (from 455 nm to 465 nm) is 1.69, which is in the range of 1.50 to 1.75, and the ordinary refractive index of oBP2-mmtBuPh-mDMePyPTzn at a wavelength of 633 nm is 1.65, which is in the range of 1.45 to 1.70.

Note that favorable light emission can be obtained from a light-emitting device fabricated using the organic compound as an electron-transport material.

### Example 23

### Synthesis Example 17

In this example, a method for synthesizing 2-[(1,1′-biphenyl)-2-yl]-4-[3-(2,6-dimethylpyridin-3-yl)-5-{(3,5-di-tert-butyl)phenyl}]phenyl-6-phenyl-1,3,5-triazine (abbreviation: oBP-mmtBuPh-mDMePyPTzn), which is the organic compound of one embodiment of the present invention, is described. The structure of oBP-mmtBuPh-mDMePyPTzn is shown below.

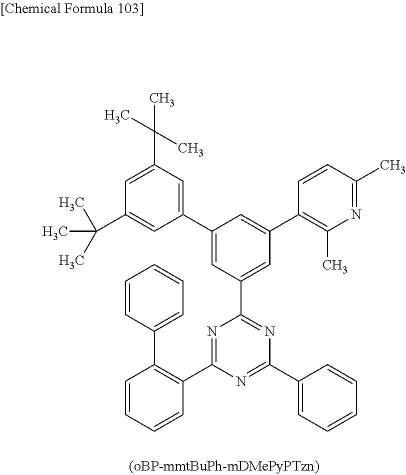 [SMILES: Cc1ccc(-c2cc(-c3cc(C(C)(C)C)cc(C(C)(C)C)c3)cc(-c3nc(-c4ccccc4)nc(-c4ccccc4-c4ccccc4)n3)c2)c(C)n1]

### Step 1: Synthesis of 2-{(1,1′-biphenyl)-2-yl}-4-(3-bromo-5-chlorophenyl)-6-phenyl-1,3,5-triazine

Into a three-neck flask were put 15 g (44 mmol) of 2-{(1,1′-biphenyl)-2-yl}-4-chloro-6-phenyl-1,3,5-triazine, 9.2 g (39 mmol) of (3-bromo-5-chlorophenyl)boronic acid, 9.25 g (87 mmol) of sodium carbonate, 170 mL of toluene, 35 mL of ethanol, and 143 mL of water, and the mixture was degassed. To this mixture was added 1.0 g (0.87 mmol) of tetrakis(triphenylphosphine)palladium(0), and the mixture was heated and stirred at 80° C. for 16 hours. After the reaction, extraction was performed with toluene and the organic layer was dried with magnesium sulfate. This mixture was gravity-filtered, and the obtained filtrate was concentrated to give a yellow solid. This solid was purified by silica gel column chromatography with a developing solvent of toluene and hexane in a ratio of 1:2. After that, purification was performed by high performance liquid chromatography using chloroform as a mobile phase to give 18 g of a target white solid in a yield of 92%. The synthesis scheme of Step 1 is shown below.

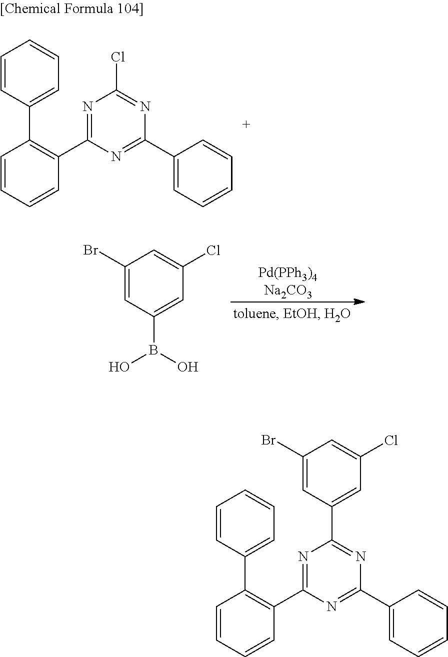

### Step 2: Synthesis of 2-{(1,1′-biphenyl)-2-yl}-4-{3-chloro-5-(3,5-di-tert-butylphenyl)phenyl}-6-phenyl-1,3,5-triazine

Into a three-neck flask were put 9.0 g (18 mmol) of 2-{(1,1′-biphenyl)-2-yl}-4-(3-bromo-5-chlorophenyl)-6-phenyl-1,3,5-triazine, 4.7 g (20 mmol) of 3,5-di-tert-butylphenylboronic acid, 18 mL of an aqueous solution of potassium carbonate (2 mol/L), 68 mL of toluene, and 34 mL of ethanol, and the mixture was degassed. To this mixture were added 0.22 g (0.72 mmol) of tris(2-methylphenyl)phosphine and 41 mg (0.18 mmol) of palladium(II) acetate, and reaction was caused at 80° C. for 14.5 hours. After the reaction, extraction was performed with toluene and the organic layer was dried with magnesium sulfate. This mixture was gravity-filtered, and the obtained filtrate was concentrated to give a pink solid. This solid was purified by silica gel column chromatography with a developing solvent of toluene and hexane in a ratio of 1:5, which was then changed to 1:3, to give 12 g of a target white solid containing toluene in a yield of 108% including toluene. The synthesis scheme of Step 2 is shown below.

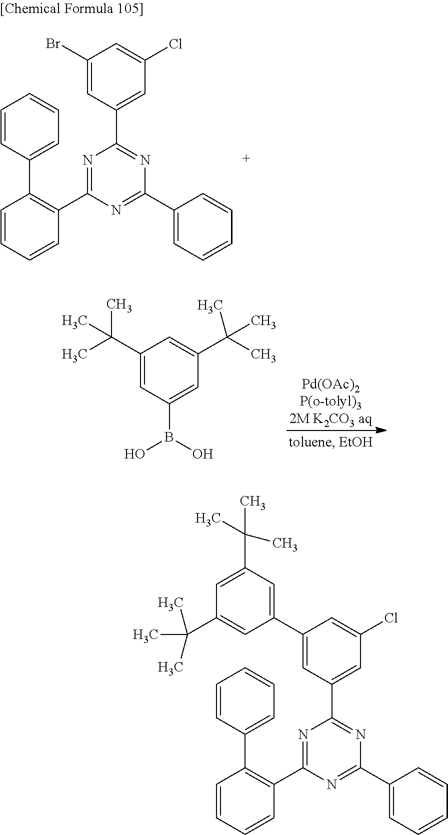 [SMILES: Clc1cc(Br)cc(-c2nc(-c3ccccc3)nc(-c3ccccc3-c3ccccc3)n2)c1 ; CC(C)(C)c1cc(B(O)O)cc(C(C)(C)C)c1 ; CC(C)(C)c1cc(-c2cc(Cl)cc(-c3nc(-c4ccccc4)nc(-c4ccccc4-c4ccccc4)n3)c2)cc(C(C)(C)C)c1]

### Step 3: Synthesis of 2-{(1,1′-biphenyl)-2-yl}-4-[3-{(3,5-di-tert-butyl)phenyl}-5-(4,4,5,5-tetramethyl-1,3,2-dioxaborolan-2-yl)phenyl]-6-phenyl-1,3,5-triazine

Into a three-neck flask were put 12 g (19 mmol) of 2-{(1,1′-biphenyl)-2-yl}-4-{3-chloro-5-(3,5-di-tert-butylphenyl)phenyl}-6-phenyl-1,3,5-triazine, 7.4 g (29 mmol) of bis(pinacolato)diboron, 5.7 g (58 mmol) of potassium acetate, and 250 mL of 1,4-dioxane, and the mixture was degassed. To this mixture were added 0.19 g (0.39 mmol) of 2-dicyclohexylphosphino-2′,4′,6′-triisopropylbiphenyl and 44 mg (0.19 mmol) of palladium(II) acetate, and the mixture was stirred at 100° C. for 14.5 hours. After the reaction, extraction was performed with toluene and the organic layer was dried with magnesium sulfate. This mixture was gravity-filtered, and the obtained filtrate was concentrated to give a black oil. This oil was purified by silica gel column chromatography with a developing solvent of toluene and hexane in a ratio of 1:2, which was then changed to only toluene, to give 12 g of a target white solid in a yield of 89%. The synthesis scheme of Step 3 is shown below.

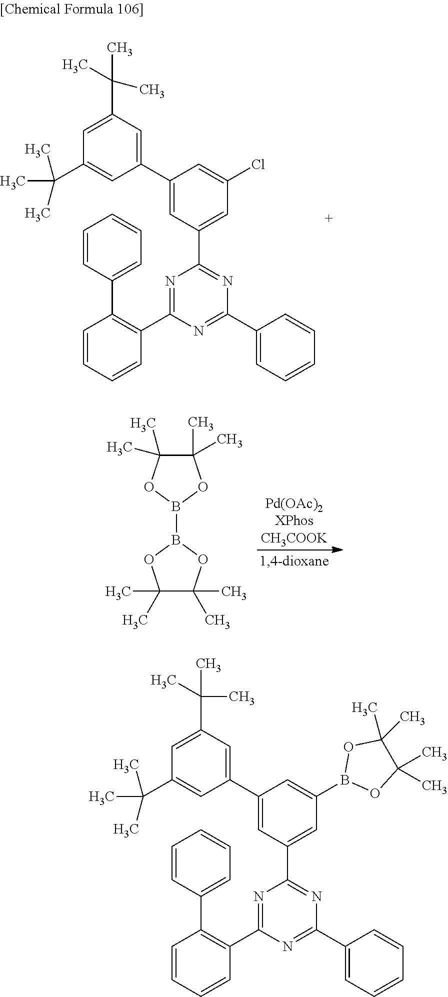

### Step 4: Synthesis of oBP-mmtBuPh-mDMePyPTzn

Into a three-neck flask were put 5.5 g (7.9 mmol) of 2-{(1,1′-biphenyl)-2-yl}-4-[3-{(3,5-di-tert-butyl)phenyl}-5-(4,4,5,5-tetramethyl-1,3,2-dioxaborolan-2-yl)phenyl]-6-phenyl-1,3,5-triazine, 1.3 g (6.8 mmol) of 3-bromo-2,6-dimethylpyridine, 40 mL of tetrahydrofuran, and 10 mL of tripotassium phosphate (2 mol/L), and the mixture was degassed. To this mixture were added 65 mg (0.14 mmol) of 2-dicyclohexylphosphino-2′,4′,6′-triisopropylbiphenyl and 15 mg (0.068 mmol) of palladium(II) acetate, and the mixture was stirred at 65° C. for 26 hours. After the reaction, extraction was performed with toluene and the organic layer was dried with magnesium sulfate. This mixture was gravity-filtered, and the obtained filtrate was concentrated to give an orange solid. This solid was purified by silica gel column chromatography with a developing solvent of toluene, which was then changed to ethyl acetate and toluene in a ratio of 1:20, to give a pale yellow solid. Subsequently, this solid was purified by silica gel column chromatography with a developing solvent of toluene, which was then changed to ethyl acetate and toluene in a ratio of 1:20, to give a white solid. The obtained solid was recrystallized with a mixed solvent of toluene and ethanol to give 4.0 g of a target white solid in a yield of 87%. The synthesis scheme of Step 4 is shown below.

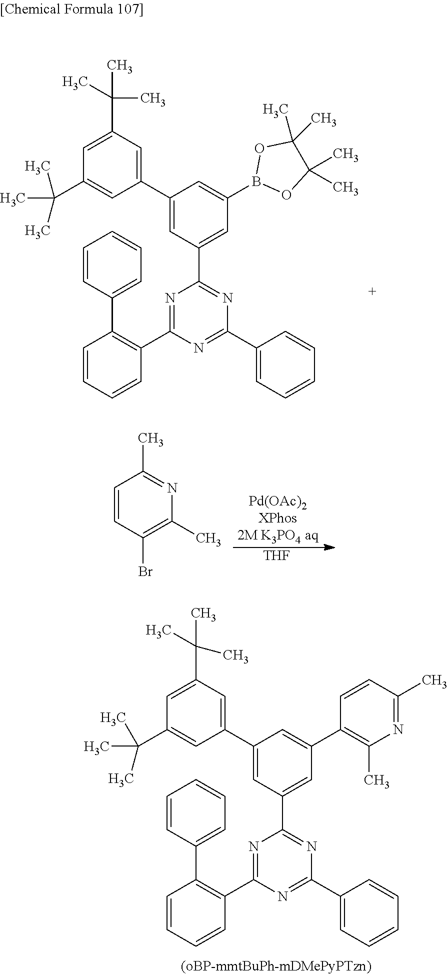

Then, 4.0 g of the obtained white solid was purified by a train sublimation method. In the purification by sublimation, the solid was heated at 250° C. for 21 hours and then at 255° C. for 18 hours under a pressure of 6.0 Pa while an argon gas was made to flow. After the purification by sublimation, 3.4 g of a target white solid was obtained at a collection rate of 85%.

Analysis results by nuclear magnetic resonance (¹H-NMR) spectroscopy of the white solid obtained above are shown below. These results reveal that the organic compound of one embodiment of the present invention, oBP-mmtBuPh-mDMePyPTzn, was obtained in this example.

¹H NMR (CD₂Cl₂, 300 MHz): δ=1.42 (s, 18H), 5.31 (s, 3H), 5.32 (s, 3H), 7.01 (t, 1H), 7.15 (t, 2H), 7.28 (d, 2H), 7.45-7.68 (m, 12H), 8.09 (s, 1H), 8.34-8.40 (m, 3H), 8.71 (s, 1H).

Next, the absorption spectrum of oBP-mmtBuPh-mDMePyPTzn was measured with an ultraviolet-visible light spectrophotometer (V550, manufactured by JASCO Corporation). The absorption spectrum of oBP-mmtBuPh-mDMePyPTzn in the dichloromethane solution was obtained by subtracting the spectrum of dichloromethane alone in a quartz cell from the absorption spectrum of oBP-mmtBuPh-mDMePyPTzn in the dichloromethane solution in the quartz cell. As a result, the absorption peak was observed at 268 nm, revealing that no absorption was shown in the visible region range from 440 nm to 700 nm.

Next, oBP-mmtBuPh-mDMePyPTzn obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving oBP-mmtBuPh-mDMePyPTzn in an organic solvent at an arbitrary concentration, and the injection amount was 5.0 μL.

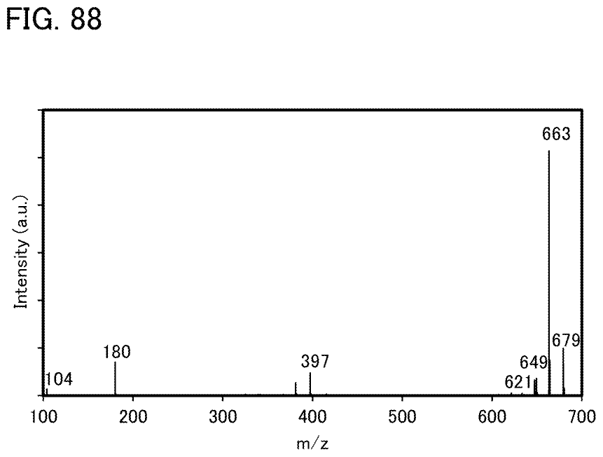

The MS/MS measurement of m/z=678.37 corresponding to the exact mass of oBP-mmtBuPh-mDMePyPTzn was performed by a PRM method. For setting of the PRM, the mass range of a target ion was set to m/z=678.37±2.0 (isolation window=4) and detection was performed in a positive mode. The measurement was performed with energy (normalized collision energy: NCE) for accelerating a target ion in a collision cell set to 60. The MS spectrum obtained by the MS/MS measurement is shown in FIG. 88.

As a result of the MS/MS measurement, fragment ions at m/z of 104.05, m/z of 180.08, and m/z of 397.26 were observed. These fragment ions are each probably a fragment formed of one substituent bonded to triazine and carbon and nitrogen derived from triazine. For example, the fragment ion at m/z of 104.05 is probably a fragment in which one carbon atom and one nitrogen atom each derived from triazine are bonded to a phenyl group. In addition, the fragment ion at m/z of 180.08 is probably a fragment in which one carbon atom and one nitrogen atom each derived from triazine are bonded to a biphenyl group. Furthermore, the fragment ion at m/z of 397.26 is probably a fragment in which one carbon atom and one nitrogen atom each derived from triazine are bonded to a substituent other than a phenyl group and a biphenyl group. These fragments can each be regarded as a feature of a compound having a triazine skeleton.

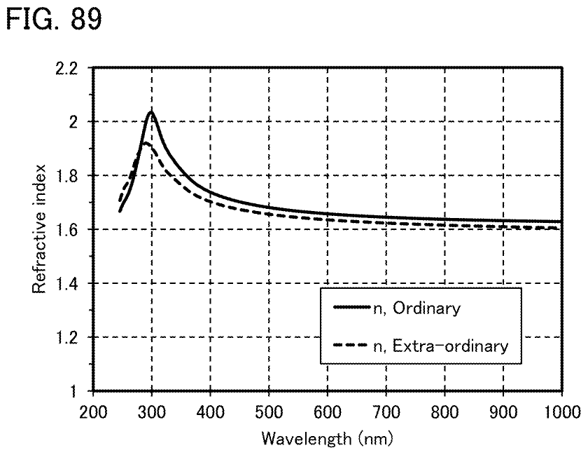

FIG. 89 shows the results of measuring the refractive index of oBP-mmtBuPh-mDMePyPTzn with a spectroscopic ellipsometer (M-2000U, produced by J.A. Woollam Japan Corp.). A film used for the measurement was formed to a thickness of approximately 50 nm with the material of each layer over a quartz substrate by a vacuum evaporation method. Note that a refractive index of an ordinary ray, n, Ordinary, and a refractive index of an extraordinary ray, n, Extra-ordinary, are shown in FIG. 89.

FIG. 89 shows that oBP-mmtBuPh-mDMePyPTzn is a material with a low refractive index: the ordinary refractive index of oBP-mmtBuPh-mDMePyPTzn in the entire blue emission region (from 455 nm to 465 nm) is 1.70, which is in the range of 1.50 to 1.75, and the ordinary refractive index of oBP-mmtBuPh-mDMePyPTzn at a wavelength of 633 nm is 1.65, which is in the range of 1.45 to 1.70.

Note that favorable light emission can be obtained from a light-emitting device fabricated using the organic compound as an electron-transport material.

### Example 24

### Synthesis Example 18

In this example, a method for synthesizing 2,4,6-tris{3′-(pyridin-3-yl)-5′-tert-butyl-1,1′-biphenyl-3-yl}-1,3,5-triazine (abbreviation: tBu-TmPPPyTz), which is the organic compound of one embodiment of the present invention, is described. The structural formula of tBu-TmPPPyTz is shown below.

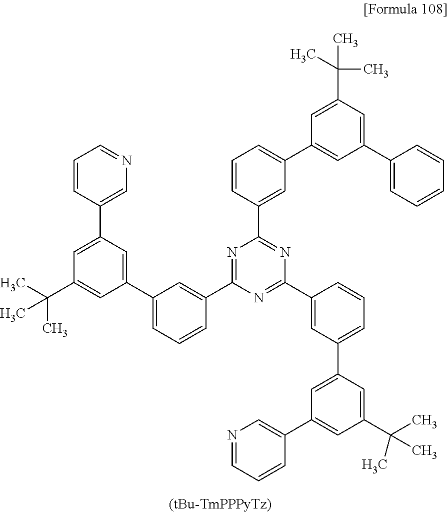 [SMILES: CC(C)(C)c1cc(-c2ccccc2)cc(-c2cccc(-c3nc(-c4cccc(-c5cc(-c6cccnc6)cc(C(C)(C)C)c5)c4)nc(-c4cccc(-c5cc(-c6cccnc6)cc(C(C)(C)C)c5)c4)n3)c2)c1]

### Step 1: Synthesis of 3-(3-bromo-5-tert-butylphenyl)pyridine

Into a three-neck flask were put 6.3 g (51 mmol) of 3-pyridylboronic acid, 30 g (103 mmol) of 1,3-dibromo-5-tert-butylbenzene, 87 mL (24 g, 174 mmol) of an aqueous solution of potassium carbonate (2 mol/L), 380 mL of toluene, and 120 mL of ethanol, and the mixture was degassed. After the reaction solution was heated to 50° C., 3.4 g (2.9 mmol) of tetrakis(triphenylphosphine)palladium(0) was added, and reaction was caused at 100° C. for 11.5 hours. After the reaction, extraction was performed with toluene and the organic layer was dried with magnesium sulfate. This mixture was gravity-filtered, and the obtained filtrate was concentrated to give a brown oil. This oil was purified by silica gel column chromatography with a developing solvent of hexane, which was then changed to hexane and ethyl acetate in a ratio of 1:2, to give 14 g of a target white solid in a yield of 94%. The synthesis scheme of Step 1 is shown below.

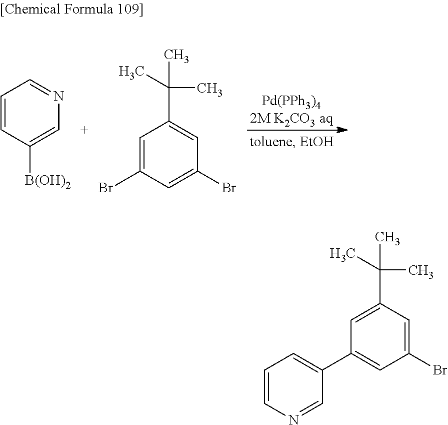 [SMILES: OB(O)c1cccnc1 ; CC(C)(C)c1cc(Br)cc(Br)c1 ; CC(C)(C)c1cc(Br)cc(-c2cccnc2)c1]

### Step 2: Synthesis of 3-[3-tert-butyl-5-(4,4,5,5-tetramethyl-1,3,2-dioxaborolan-2-yl)phenyl]pyridine

Into a flask were put 16 g (55 mmol) of 3-(3-bromo-5-tert-butylphenyl)pyridine, 15 g (61 mmol) of bis(pinacolato)diboron, 16 g (165 mmol) of potassium acetate, and 190 mL of N,N-dimethylformamide, and the mixture was degassed. To this mixture was added 2.3 g (2.8 mmol) of [1,1′-bis(diphenylphosphino)ferrocene]dichloropalladium(II) dichloromethane adduct, and the mixture was heated and stirred at 100° C. for 21 hours. After the reaction, extraction was performed with toluene and the organic layer was dried with magnesium sulfate. This mixture was gravity-filtered, and the obtained filtrate was concentrated to give a brown solid. This solid was purified by silica gel column chromatography with a developing solvent of toluene, which was then changed to toluene and ethyl acetate in a ratio of 1:3, to give 14 g of a white solid in a yield of 77%. The synthesis scheme of Step 2 is shown below.

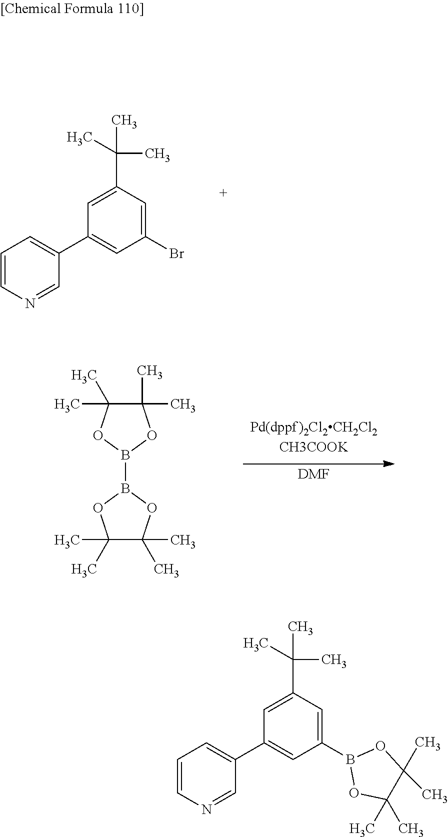

### Step 3: Synthesis of tBu-TmPPPyTz

Into a three-neck flask were put 3.2 g (5.9 mmol) of 1,3,5-tris(3-bromophenyl)benzene, 7.0 g (21 mmol) of 3-[3-tert-butyl-5-(4,4,5,5-tetramethyl-1,3,2-dioxaborolan-2-yl)phenyl]pyridine, 60 mL (17 g, 119 mmol) of an aqueous solution of potassium carbonate (2 mol/L), 300 mL of toluene, and 60 mL of ethanol, and the mixture was degassed. To this mixture was added 0.41 g (0.36 mmol) of tetrakis(triphenylphosphine)palladium(0), and the mixture was stirred at 90° C. for 13.5 hours. After the reaction, extraction was performed with chloroform and the organic layer was dried with magnesium sulfate. This mixture was gravity-filtered, and the obtained filtrate was concentrated to give an orange oil. This oil was purified by silica gel column chromatography with a developing solvent of chloroform and methanol in a ratio of 50:1, which was then changed to 20:1. After that, purification was performed by high performance liquid chromatography using chloroform as a mobile phase. The obtained solid was purified with ethyl acetate to give 2.6 g of a target white solid in a yield of 47%. The synthesis scheme of Step 3 is shown below.

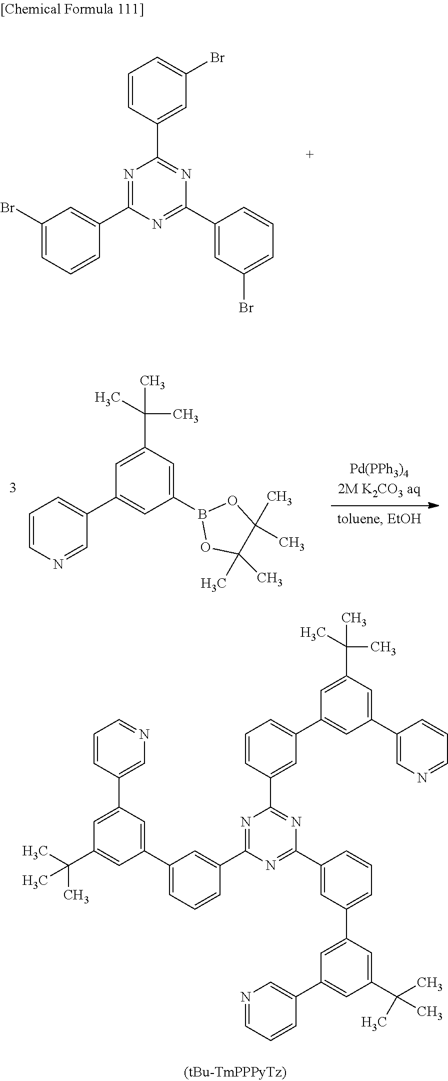 [SMILES: Brc1cccc(-c2nc(-c3cccc(Br)c3)nc(-c3cccc(Br)c3)n2)c1 ; CC(C)(C)c1cc(B2OC(C)(C)C(C)(C)O2)cc(-c2cccnc2)c1 ; CC(C)(C)c1cc(-c2cccnc2)cc(-c2cccc(-c3nc(-c4cccc(-c5cc(-c6cccnc6)cc(C(C)(C)C)c5)c4)nc(-c4cccc(-c5cc(-c6cccnc6)cc(C(C)(C)C)c5)c4)n3)c2)c1]

Then, 2.6 g of the obtained white solid was purified by a train sublimation method (heating was performed at 365° C. for 5.5 hours and then at 370° C. for 33 hours under a pressure of 2.8 Pa and under an argon gas stream). After the purification by sublimation, 2.1 g of a target white solid was obtained at a collection rate of 81%.

Analysis results by nuclear magnetic resonance (¹H-NMR) spectroscopy of the obtained white solid are shown below. These results reveal that the organic compound of one embodiment of the present invention, tBu-TmPPPyTz, was obtained in this example.

¹H NMR (CDCl₃, 300 MHz): δ=1.43 (s, 27H), 7.35 (dd, 3H), 7.62-7.75 (m, 12H), 7.87 (d, 3H), 7.93 (d, 3H), 8.60 (d, 3H), 8.78 (d, 3H), 8.91 (s, 3H), 9.01 (s, 3H).

Next, an absorption spectrum of tBu-TmPPPyTz in a dichloromethane solution was measured. The absorption spectrum of tBu-TmPPPyTz in the dichloromethane solution was measured with an ultraviolet-visible light spectrophotometer (V550, manufactured by JASCO Corporation), and the spectrum of dichloromethane alone in a quartz cell was subtracted. As a result, the absorption peak was observed at 275 nm, revealing that no absorption was shown in the visible region range from 440 nm to 700 nm.

Next, tBu-TmPPPyTz obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving tBu-TmPPPyTz in an organic solvent at an arbitrary concentration, and the injection amount was 5.0 pt.

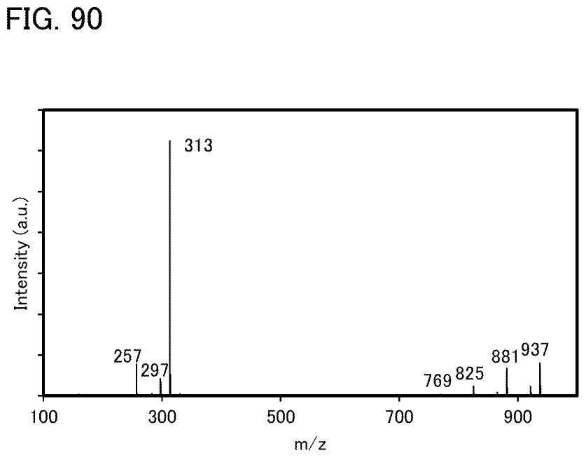

The MS/MS measurement of m/z=936.49 corresponding to the exact mass of tBu-TmPPPyTz was performed by a PRM method. For setting of the PRM, the mass range of a target ion was set to m/z=936.49±2.0 (isolation window=4) and detection was performed in a positive mode. The measurement was performed with energy (NCE) for accelerating a target ion in a collision cell set to 50. The MS spectrum obtained by the MS/MS measurement is shown in FIG. 90.

As a result of the measurement, a fragment ion at m/z of 313.17 was observed. This is probably a fragment formed of one substituent bonded to triazine and carbon and nitrogen derived from triazine. For example, the fragment ion at m/z of 313.17 is probably a fragment in which one carbon atom and one nitrogen atom each derived from triazine are bonded to 3′-(pyridin-3-yl)-5′-tert-butyl-1,1′-biphenyl. This fragment can be regarded as a feature of a compound having a triazine skeleton.

Note that favorable light emission can be obtained from a light-emitting device fabricated using the organic compound as an electron-transport material.

### Example 25

### Synthesis Example 19

In this example, a method for synthesizing 2,4,6-tris{3′-(pyridin-3-yl)-5′-tert-butyl-1,1′-biphenyl-4-yl}-1,3,5-triazine (abbreviation: tBu-TmPPPyTz-02), which is the organic compound of one embodiment of the present invention, is described. The structural formula of tBu-TmPPPyTz-02 is shown below.

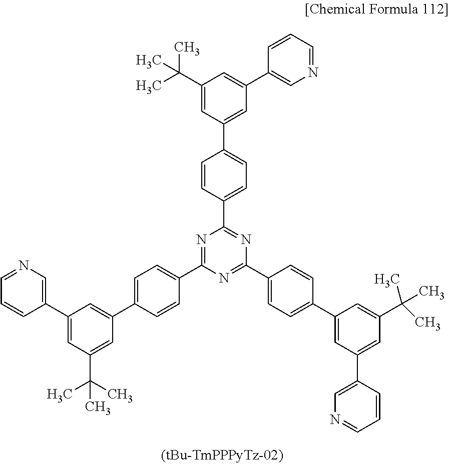 [SMILES: CC(C)(C)c1cc(-c2ccc(-c3nc(-c4ccc(-c5cc(-c6cccnc6)cc(C(C)(C)C)c5)cc4)nc(-c4ccc(-c5cc(-c6cccnc6)cc(C(C)(C)C)c5)cc4)n3)cc2)cc(-c2cccnc2)c1]

### Step 1: Synthesis of 3-(3-bromo-5-tert-butylphenyl)pyridine

This synthesis step is similar to Step 1 in Synthesis example 18 in Example 24.

### Step 2: Synthesis of 3-[3-tert-butyl-5-(4,4,5,5-tetramethyl-1,3,2-dioxaborolan-2-yl)phenyl]pyridine

This synthesis step is similar to Step 2 in Synthesis example 18 in Example 24.

### Step 3: Synthesis of tBu-TmPPPyTz-02

Into a three-neck flask were put 3.2 g (5.76 mmol) of 2,4,6-tris(4-bromophenyl)-1,3,5-triazine, 6.6 g (19.6 mmol) of 3-[3-tert-butyl-5-(4,4,5,5-tetramethyl-1,3,2-dioxaborolan-2-yl)phenyl]pyridine, 58 mL (17 g, 120 mmol) of an aqueous solution of potassium carbonate (2 mol/L), 290 mL of toluene, and 58 mL of ethanol, and the air in the flask was replaced with nitrogen. To this mixture was added 0.60 g (0.518 mmol) of tetrakis(triphenylphosphine)palladium(0), and the mixture was stirred at 90° C. for 20.5 hours. After the reaction, the reaction solution was filtered and extraction was performed with toluene. The solution of the extract was dehydrated with magnesium sulfate, which was then concentrated to give a brown solid containing a target substance. The obtained solid was purified by silica gel column chromatography with a developing solvent of dichloromethane and methanol in a ratio of 50:1, which was then changed to 40:1, to give a target white solid. The resulting solid was further purified by high performance liquid column chromatography. The high performance liquid column chromatography was performed using chloroform as a developing solvent. The obtained fraction was concentrated to give a target white solid. The obtained solid was recrystallized with toluene to give 3.6 g of a target white solid in a yield of 66%. The synthesis scheme of Step 3 is shown below.

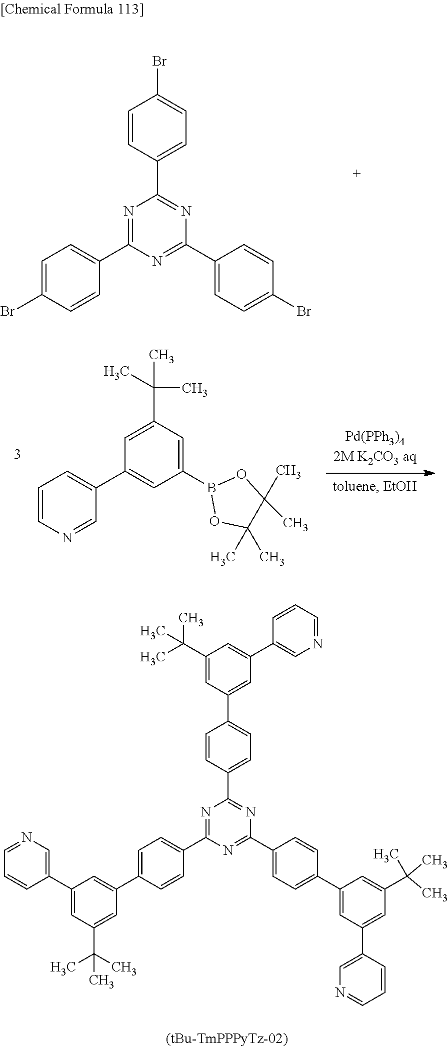 [SMILES: Brc1ccc(-c2nc(-c3ccc(Br)cc3)nc(-c3ccc(Br)cc3)n2)cc1 ; CC(C)(C)c1cc(B2OC(C)(C)C(C)(C)O2)cc(-c2cccnc2)c1 ; CC(C)(C)c1cc(-c2ccc(-c3nc(-c4ccc(-c5cc(-c6cccnc6)cc(C(C)(C)C)c5)cc4)nc(-c4ccc(-c5cc(-c6cccnc6)cc(C(C)(C)C)c5)cc4)n3)cc2)cc(-c2cccnc2)c1]

Then, 3.6 g of the obtained white solid was purified by a train sublimation method. The solid was heated under a pressure of 0.6 Pa at 365° C. for 16 hours. After the purification by sublimation, 2.6 g of a target white solid was obtained at a collection rate of 72%.

Analysis results by nuclear magnetic resonance (¹H-NMR) spectroscopy of the obtained white solid are shown below. These results reveal that the organic compound of one embodiment of the present invention, tBu-TmPPPyTz-02, was obtained in this example.

¹H NMR (CDCl₃, 300 MHz): δ=1.43 (s, 27H), 7.35 (dd, 3H), 7.62-7.75 (m, 12H), 7.87 (d, 3H), 7.93 (d, 3H), 8.60 (d, 3H), 8.78 (d, 3H), 8.91 (s, 3H), 9.01 (s, 3H).

Next, an absorption spectrum of tBu-TmPPPyTz-02 in a dichloromethane solution was measured. The absorption spectrum of tBu-TmPPPyTz-02 in the dichloromethane solution was measured with an ultraviolet-visible light spectrophotometer (V550, manufactured by JASCO Corporation), and the spectrum of dichloromethane alone in a quartz cell was subtracted. As a result, the absorption peak was observed at 318 nm, revealing that no absorption was shown in the visible region range from 440 nm to 700 nm.

Next, tBu-TmPPPyTz-02 obtained in this example was subjected to LC/MS analysis.

In the LC/MS analysis, LC separation was performed with UltiMate 3000 produced by Thermo Fisher Scientific K.K., and MS analysis was performed with Q Exactive produced by Thermo Fisher Scientific K.K.

In the LC separation, a given column was used at a column temperature of 40° C., and solution sending was performed in such a manner that an appropriate solvent was selected, the sample was prepared by dissolving tBu-TmPPPyTz-02 in an organic solvent at an arbitrary concentration, and the injection amount was 5.0 μL.

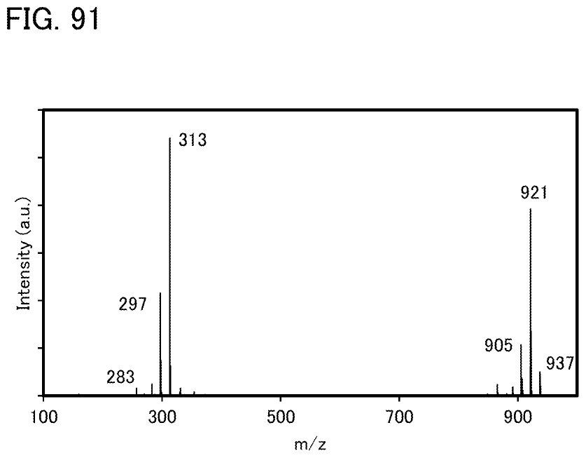

The MS/MS measurement of m/z=936.49 corresponding to the exact mass of tBu-TmPPPyTz-02 was performed by a PRM method. For setting of the PRM, the mass range of a target ion was set to m/z=936.49±2.0 (isolation window=4) and detection was performed in a positive mode. The measurement was performed with energy (normalized collision energy: NCE) for accelerating a target ion in a collision cell set to 60. The MS spectrum obtained by the MS/MS measurement is shown in FIG. 91.

As a result of the measurement, a fragment ion at m/z of 313.17 was observed. This is probably a fragment formed of one substituent bonded to triazine and carbon and nitrogen derived from triazine. For example, the fragment ion at m/z of 313.17 is probably a fragment in which one carbon atom and one nitrogen atom each derived from triazine are bonded to 3′-(pyridin-3-yl)-5′-tert-butyl-1,1′-biphenyl. This fragment can be regarded as a feature of a compound having a triazine skeleton.

Note that favorable light emission can be obtained from a light-emitting device fabricated using the organic compound as an electron-transport material.

### Reference Synthesis Example

In this synthesis example, a method for synthesizing 6-methyl-8-quinolinolato-lithium (abbreviation: Li-6mq), which is a metal complex represented by Structural Formula (vii) and is used for some of the light-emitting devices in Examples in this specification, is described. The structural formula of Li-6mq is shown below.

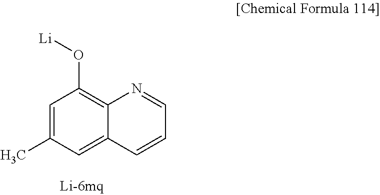

Into a three-neck flask were put 2.0 g (12.6 mmol) of 8-hydroxy-6-methylquinoline and 130 mL of dehydrated tetrahydrofuran (abbreviation: THF), and the mixture was stirred. To this solution was added 10.1 mL (10.1 mmol) of a 1M THF solution of lithium-tert-butoxide (abbreviation: tBuOLi), and the mixture was stirred at room temperature for 47 hours. The reaction solution was concentrated to give a yellow solid. Acetonitrile was added to this solid, and the mixture was irradiated with ultrasonic waves and then subjected to filtration to give a pale yellow solid. This washing operation was performed twice. As a residue, 1.6 g of a pale yellow solid of Li-6mq was obtained in a yield of 95%. The synthesis scheme is shown below.

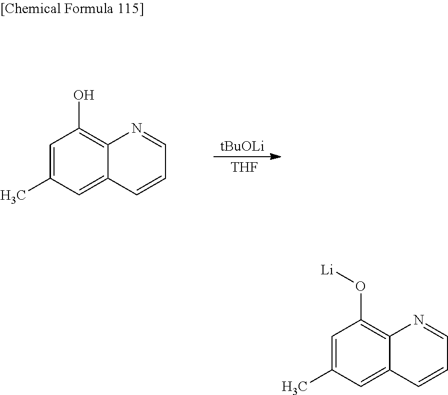

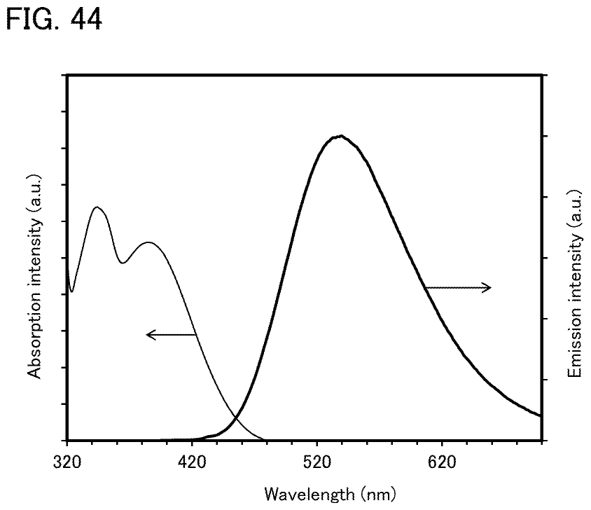

FIG. 44 shows measurement results of an absorption spectrum and an emission spectrum of Li-6mq in a dehydrated acetone solution. The absorption spectrum of Li-6mq in the dehydrated acetone solution was measured with an ultraviolet-visible light spectrophotometer (V550, manufactured by JASCO Corporation), and the spectrum of dehydrated acetone alone in a quartz cell was subtracted. The emission spectrum was measured with a fluorescence spectrophotometer (FP-8600, manufactured by JASCO Corporation).

FIG. 44 reveals that Li-6mq in the dehydrated acetone solution has an absorption peak at 390 nm and an emission wavelength peak at 540 nm (excitation wavelength: 385 nm).

This application is based on Japanese Patent Application Serial No. 2020-078909 filed with Japan Patent Office on Apr. 28, 2020, the entire contents of which are hereby incorporated by reference.

What is claimed is:

1. A light-emitting device material comprising an organic compound, the organic compound comprising:
at least one six-membered heteroaromatic ring having 1 to 3 nitrogen atoms;
a plurality of aromatic hydrocarbon rings each having 6 to 14 carbon atoms in a ring; and
a plurality of hydrocarbon groups forming a bond by sp³ hybrid orbitals,
wherein at least two of the plurality of aromatic hydrocarbon rings are benzene rings, and
wherein an ordinary refractive index of a layer comprising the organic compound with respect to light with a wavelength in a range from 455 nm to 465 nm is higher than or equal to 1.5 and lower than or equal to 1.75.

2. A light-emitting device material comprising an organic compound, the organic compound comprising:
at least one six-membered heteroaromatic ring having 1 to 3 nitrogen atoms;
a plurality of aromatic hydrocarbon rings each having 6 to 14 carbon atoms in a ring; and
a plurality of hydrocarbon groups forming a bond by sp³ hybrid orbitals,
wherein at least two of the plurality of aromatic hydrocarbon rings are benzene rings, and
wherein a proportion of carbon atoms forming a bond by the sp³ hybrid orbitals in a total number of carbon atoms in a molecule of the organic compound is higher than or equal to 10% and lower than or equal to 60%.

3. The light-emitting device material according to claim 2,
wherein the proportion of carbon atoms forming the bond by the sp³ hybrid orbitals in the total number of carbon atoms in the molecule of the organic compound is higher than or equal to 10% and lower than or equal to 50%.

4. A light-emitting device material comprising an organic compound, the organic compound comprising:
at least one six-membered heteroaromatic ring having 1 to 3 nitrogen atoms;
a plurality of aromatic hydrocarbon rings each having 6 to 14 carbon atoms in a ring; and
a plurality of hydrocarbon groups forming a bond by sp³ hybrid orbitals,
wherein at least two of the plurality of aromatic hydrocarbon rings are benzene rings, and
wherein an integral value of signals at lower than 4 ppm is more than or equal to half an integral value of signals at 4 ppm or higher in results of ¹H-NMR measurement conducted on the organic compound.

5. The light-emitting device material according to claim 1,
wherein a molecular weight of the organic compound is greater than or equal to 500 and less than or equal to 2000.

6. The light-emitting device material according to claim 1,
wherein the plurality of hydrocarbon groups are each independently bonded to any of the plurality of aromatic hydrocarbon rings, and
wherein an isovalue of LUMO distribution density in each of the plurality of aromatic hydrocarbon rings to which the plurality of hydrocarbon groups are bonded is less than 0.06 electrons/au³.

7. The light-emitting device material according to claim 6,
wherein at least one of the plurality of aromatic hydrocarbon rings to which the plurality of hydrocarbon groups are bonded is a benzene ring.

8. The light-emitting device material according to claim 1,
wherein the organic compound comprises at least three benzene rings, and
wherein the three benzene rings are each bonded to the six-membered heteroaromatic ring.

9. The light-emitting device material according to claim 1,
wherein the organic compound comprises at least three benzene rings,
wherein the three benzene rings are each bonded to the six-membered heteroaromatic ring, and
wherein two of the three benzene rings are substituted or unsubstituted phenyl groups and have no hydrocarbon group.

10. The light-emitting device material according to claim 1,
wherein the organic compound comprises a substituted or unsubstituted pyridyl group.

11. The light-emitting device material according to claim 1,
wherein the six-membered heteroaromatic ring is any one of a triazine ring and a pyrimidine ring.

12. The light-emitting device material according to claim 1,
wherein the plurality of hydrocarbon groups are each independently an alkyl group or a cycloalkyl group.

13. The light-emitting device material according to claim 12,
wherein the alkyl group is a branched alkyl group having 3 to 5 carbon atoms.

14. An organic compound represented by General Formula (G1): 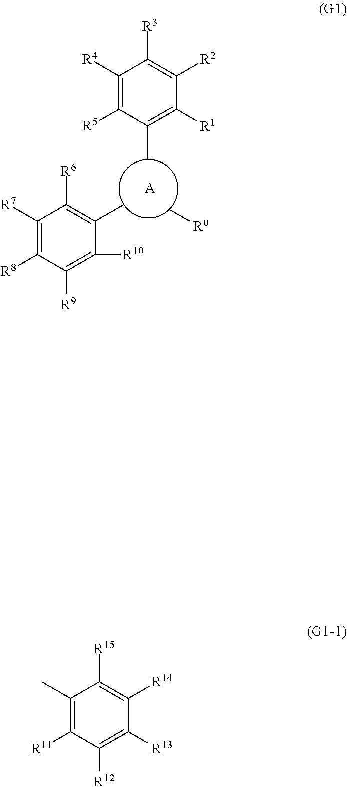
wherein A represents a six-membered heteroaromatic ring having 1 to 3 nitrogen atoms,
wherein R⁰ represents any of hydrogen, an alkyl group having 1 to 6 carbon atoms, an alicyclic group having 3 to 10 carbon atoms, and a substituent represented by Formula (G1-1),
wherein at least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group,
wherein the phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring, and
wherein the organic compound represented by General Formula (G1) comprises a plurality of hydrocarbon groups selected from an alkyl group having 1 to 6 carbon atoms and an alicyclic group having 3 to 10 carbon atoms, and a proportion of carbon atoms forming a bond by sp³ hybrid orbitals in a total number of carbon atoms in a molecule of the organic compound is higher than or equal to 10% and lower than or equal to 60%.

15. An organic compound represented by General Formula (G2): 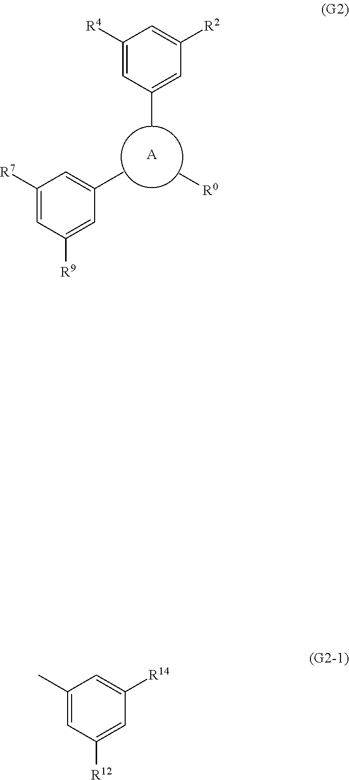
wherein A represents a six-membered heteroaromatic ring having 1 to 3 nitrogen atoms,
wherein R⁰ represents any of hydrogen, an alkyl group having 1 to 6 carbon atoms, an alicyclic group having 3 to 10 carbon atoms, and a substituent represented by Formula (G2-1),
wherein at least one of R², R⁴, R⁷, R⁹, R¹², and R¹⁴ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group,
wherein the phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring, and
wherein the organic compound represented by General Formula (G2) comprises a plurality of hydrocarbon groups selected from an alkyl group having 1 to 6 carbon atoms and an alicyclic group having 3 to 10 carbon atoms, and a proportion of carbon atoms forming a bond by sp³ hybrid orbitals in a total number of carbon atoms in a molecule of the organic compound is higher than or equal to 10% and lower than or equal to 60%.

16. An organic compound represented by General Formula (G3): 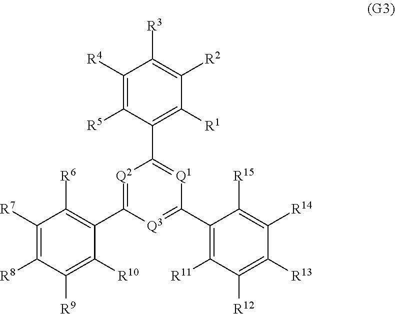
wherein two or three of Q¹ to Q³ represent N, and when two of Q¹ to Q³ represent N, the remaining one of Q¹ to Q³ represents CH,
wherein at least one of R¹ to R¹⁵ is a phenyl group having a substituent, and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group,
wherein the phenyl group having a substituent has one or two substituents, and the substituents are each independently any of an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted heteroaromatic ring group having 3 to 9 carbon atoms in a ring, and
wherein the organic compound represented by General Formula (G3) comprises a plurality of hydrocarbon groups selected from an alkyl group having 1 to 6 carbon atoms and an alicyclic group having 3 to 10 carbon atoms, and a proportion of carbon atoms forming a bond by sp³ hybrid orbitals in a total number of carbon atoms in a molecule of the organic compound is higher than or equal to 10% and lower than or equal to 60%.

17. The organic compound according to claim 14,
wherein the phenyl group having a substituent is represented by Formula (G1-2): 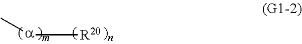
wherein α represents a substituted or unsubstituted phenylene group,
wherein R²⁰ represents an alkyl group having 1 to 6 carbon atoms, an alicyclic group having 3 to 10 carbon atoms, or a substituted or unsubstituted aromatic hydrocarbon group having 6 to 14 carbon atoms in a ring,
wherein m and n each represent 1 or 2,
wherein, when m is 2, a plurality of a represent the same phenylene group or different phenylene groups, and
wherein, when n is 2, a plurality of R²⁰ represent the same substituent or different substituents.

18. The organic compound according to claim 17,
wherein the phenyl group having a substituent that is represented by Formula (G1-2) is used as one or both of R² and R⁴, and
wherein, when the phenyl group having a substituent that is represented by Formula (G1-2) is used as both of R² and R⁴, the two phenyl groups represented by Formula (G1-2) are the same or different from each other.

19. The organic compound according to claim 14,
wherein the phenyl group having a substituent is represented by Formula (G1-3): 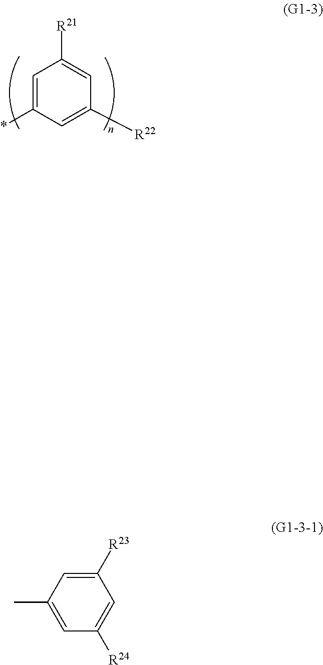
wherein R²¹ represents any one of hydrogen, an alkyl group having 1 to 6 carbon atoms, an alicyclic group having 3 to 10 carbon atoms, and a substituent represented by Formula (G1-3-1),
wherein R²² represents the substituent represented by Formula (G1-3-1),
wherein R²³ and R²⁴ each represent any one of hydrogen, an alkyl group having 1 to 6 carbon atoms, and an alicyclic group having 3 to 10 carbon atoms, and at least one of R²³ and R²⁴ represents an alkyl group having 1 to 6 carbon atoms or an alicyclic group having 3 to 10 carbon atoms,
wherein n represents 0 to 2,
wherein, when n is 2, a plurality of R²¹ are the same or different from each other, and
wherein, when n is 0, at least one of R²³ and R²⁴ is any one of an alkyl group having 1 to 6 carbon atoms and an alicyclic group having 3 to 10 carbon atoms.

20. The organic compound according to claim 19,
wherein the phenyl group having a substituent that is represented by Formula (G1-3) is used as one or both of R² and R⁴, and
wherein, when the phenyl group having a substituent that is represented by Formula (G1-3) is used as both of R² and R⁴, the two phenyl groups represented by Formula (G1-3) are the same or different from each other.

21. The organic compound according to claim 14,
wherein, when the aromatic hydrocarbon group having 6 to 14 carbon atoms in a ring has a substituent, the substituent is any one of an alkyl group having 1 to 6 carbon atoms, an alicyclic group having 3 to 10 carbon atoms, an unsubstituted aromatic hydrocarbon group having 6 to 14 carbon atoms, and an aromatic hydrocarbon group having 6 to 14 carbon atoms in a ring, to which an alkyl group having 1 to 6 carbon atoms or an alicyclic group having 3 to 10 carbon atoms is bonded.

22. The organic compound according to claim 14,
wherein the aromatic hydrocarbon group having 6 to 14 carbon atoms in a ring is any one of a phenyl group, a naphthyl group, a phenanthrenyl group, and a fluorenyl group.

23. The organic compound according to claim 14,
wherein the aromatic hydrocarbon group having 6 to 14 carbon atoms in a ring is represented by any one of Formulae (ra-1) to (ra-15):

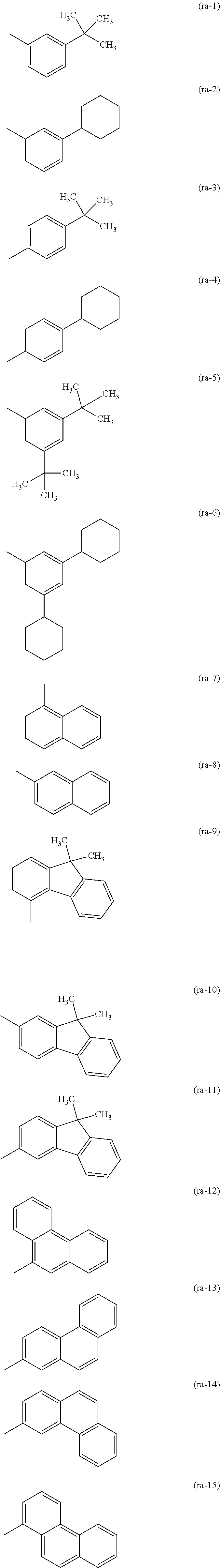

24. The organic compound according to claim 14,
wherein the substituted or unsubstituted pyridyl group is an unsubstituted pyridyl group or a pyridyl group to which one or more methyl groups are bonded.

25. The organic compound according to claim 14,
wherein the alicyclic group is a cycloalkyl group having 3 to 6 carbon atoms.

26. The organic compound according to claim 14,
wherein the alkyl group having 1 to 6 carbon atoms is a branched alkyl group having 3 to 5 carbon atoms.

27. An organic compound represented by any one of Structural Formulae (100), (120), (121), (123), (200), and (412):

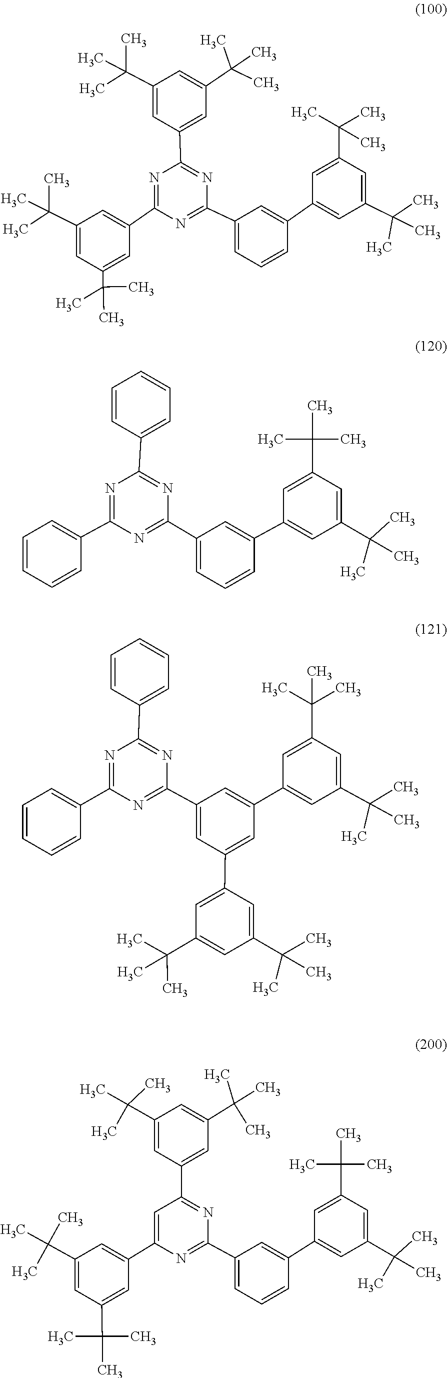

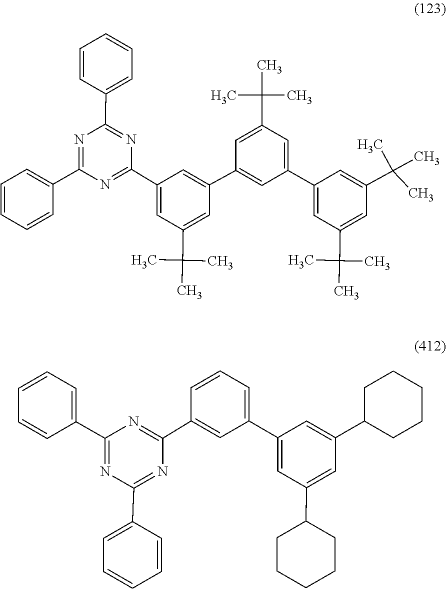 [SMILES: CC(C)(C)c1cc(-c2cc(-c3nc(-c4ccccc4)nc(-c4ccccc4)n3)cc(C(C)(C)C)c2)cc(-c2cc(C(C)(C)C)cc(C(C)(C)C)c2)c1 ; c1ccc(-c2nc(-c3ccccc3)nc(-c3cccc(-c4cc(C5CCCCC5)cc(C5CCCCC5)c4)c3)n2)cc1]

28. Alight-emitting device comprising the organic compound according to claim 14.

29. An electronic device comprising:
the light-emitting device according to claim 28; and
at least one of a sensor, an operation button, a speaker, and a microphone.

30. A light-emitting apparatus comprising:
the light-emitting device according to claim 28; and
at least one of a transistor and a substrate.

31. A lighting device comprising:
the light-emitting device according to claim 28; and
a housing.
